(19) United States
(12) Patent Application Publication
(10) Pub. No.: US 2012/0101032 A1
(43) Pub. Date: Apr. 26, 2012

(54) NOVEL MACROCYCLIC INHIBITORS OF HEPATITIS C VIRUS REPLICATION

(75) Inventors: Brad BUCKMAN, Oakland, CA (US); Vladimir SEREBRYANY, Burlingame, CA (US); John B. NICHOLAS, Redwood City, CA (US); Scott SEIWERT, Pacifica, CA (US)

(73) Assignee: INTERMUNE, INC., Brisbane, CA (US)

(21) Appl. No.: 13/278,984

(22) Filed: Oct. 21, 2011

Related U.S. Application Data
(60) Provisional application No. 61/406,058, filed on Oct. 22, 2010.
(60) Provisional application No. 61/473,568, filed on Apr. 8, 2011.

(51) Int. Cl. A61K 38/06; A61P 1/16; A61P 31/14; C07K 5/083; C12N 9/99

(52) U.S. Cl. 514/4.3; 530/331; 514/20.1; 435/184

(57) ABSTRACT

The embodiments provide compounds of the general Formulae I, Ia, II, IIa, III, IIIa, IV, IVa, V, Va, VI and VIa, as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATIONS

This application claims the benefit of U.S. Provisional Application Nos. 61/406,058, filed Oct. 22, 2010, and 61/473,568, filed Apr. 8, 2011, all of which are incorporated herein by reference in their entirety.

### BACKGROUND OF THE INVENTION

1. Field of the Invention

The present invention relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders (NR) or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for N53-mediated processing events and enhances proteolytic efficiency at all sites recognized by NS3. The NS3 protease also exhibits nucleoside triphosphatase and RNA helicase activities. NS5B is an RNA-dependent RNA polymerase involved in the replication of HCV RNA. In addition, compounds that inhibit the action of NS5A in viral replication are potentially useful for the treatment of HCV.

### SUMMARY OF THE INVENTION

The present embodiments provide compounds of the general Formula I:

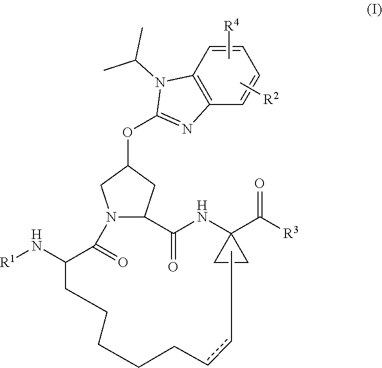

or a pharmaceutically acceptable salt or prodrug thereof, wherein R¹ is selected from hydrogen, —C(O)OR^{1e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{1a}R^{1b}, —NHC(O) NR^{1a}R^{1b}, —C(O)OR^{1c}, and heteroaryl.

R^{1e} is selected from the group consisting of C₁₋₆ alkyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl; and R^{1c} and R^{1d} are each separately selected from the group consisting of —H (hydrogen), C₁₋₄ alkoxy, C₁₋₆ alkyl, C₃₋₇ cycloalkyl, aryl, arylalkyl and heteroaryl.

R² is selected from the group consisting of

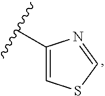

pyrazinyl, and pyrimidinyl, each optionally substituted with R^{2a}; or R² is

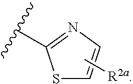

R^{2a} is phenyl substituted with one or more R^{2b} or benzyl optionally substituted with one or more R^{2b}; wherein R^{2b} is halo, —CF₃, —OCF₃, C₁₋₆ alkyl, C₁₋₆ alkoxy, or phenyl; or R^{2a} is optionally substituted C₁₋₆ alkyl, optionally substituted C₃₋₇ cycloalkyl, optionally substituted dihydrobenzodioxinyl, optionally substituted piperidinyl, optionally substituted piperazinyl, optionally substituted tetrahydropyranyl, or optionally substituted pyrrolidinyl.

R³ is —OH, —NHS(O)₂R^{3a}, —NHS(O)₂OR^{3a} or —NHS(O)₂NR^{3a}R^{3c}, wherein R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

R⁴ is selected from the group consisting of hydrogen, halo, optionally substituted C₁₋₆ alkyl, optionally substituted C₁₋₆ alkoxy, and optionally substituted C₂₋₆ alkenyl.

Each t is independently 0, 1 or 2; each q is independently 0, 1 or 2; and any bond represented by a dashed and solid line represents a bond selected from the group consisting of a single bond and a double bond.

Some embodiments include the proviso that when R¹ is —C(O)O-t-butyl, R³ is —NHS(O)₂-methylcyclopropyl or —NHS(O)₂N(CH₃)₂, R⁴ is H, and R² is

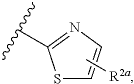

then R^{2a} is not cyclopropyl, cyclobutyl, cyclohexyl, unsubstituted benzyl,

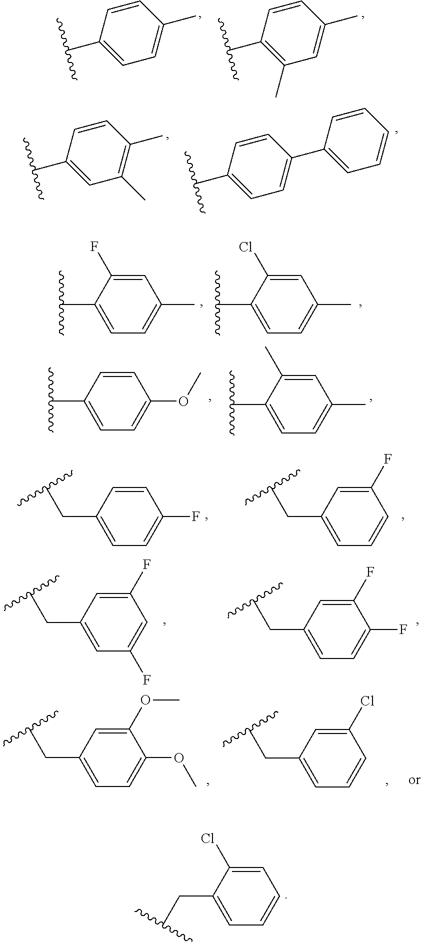

Some embodiments include the proviso that the compound is not selected from the group consisting of

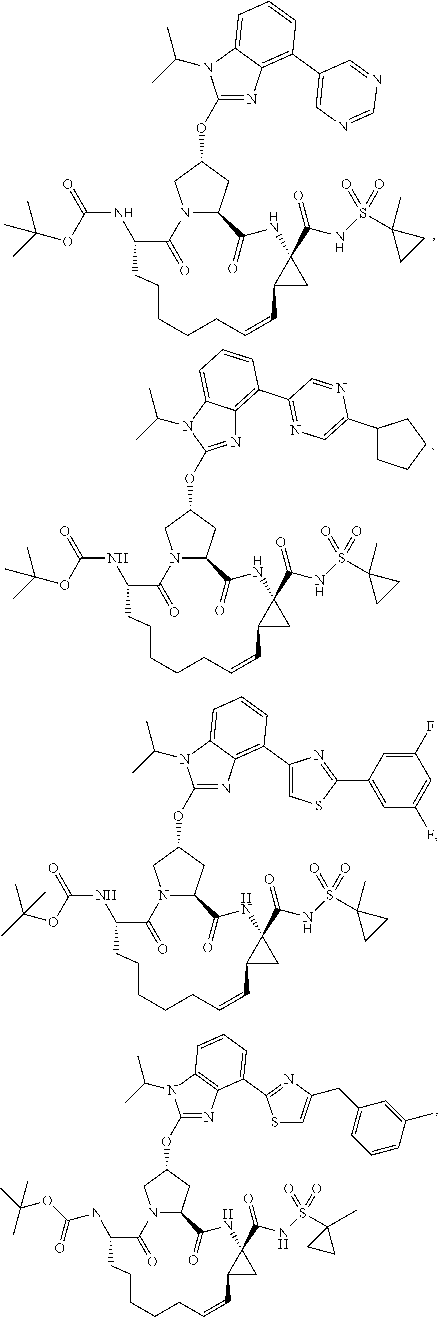

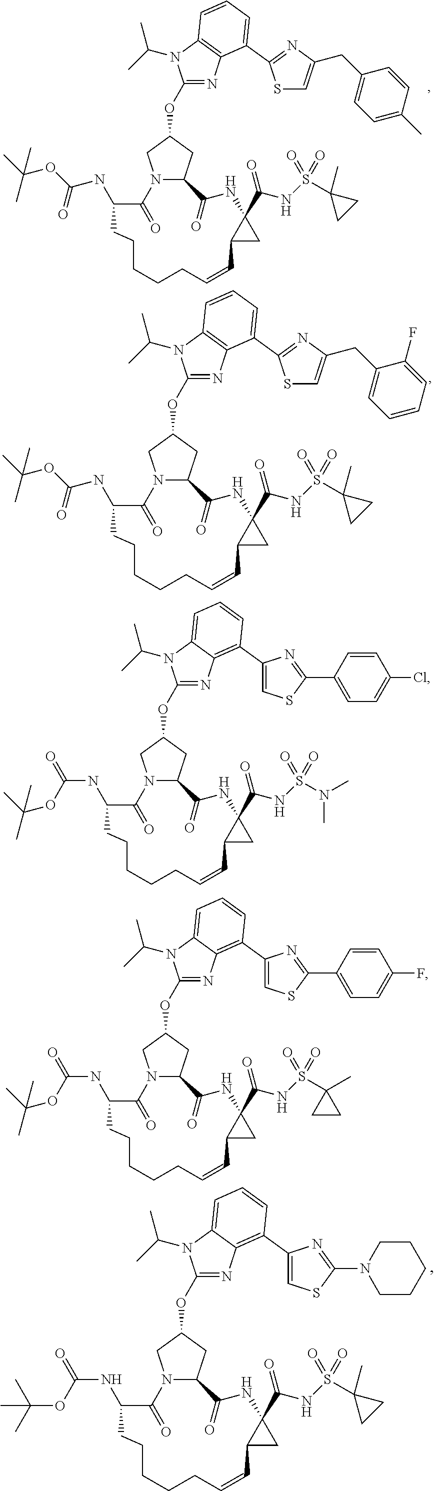

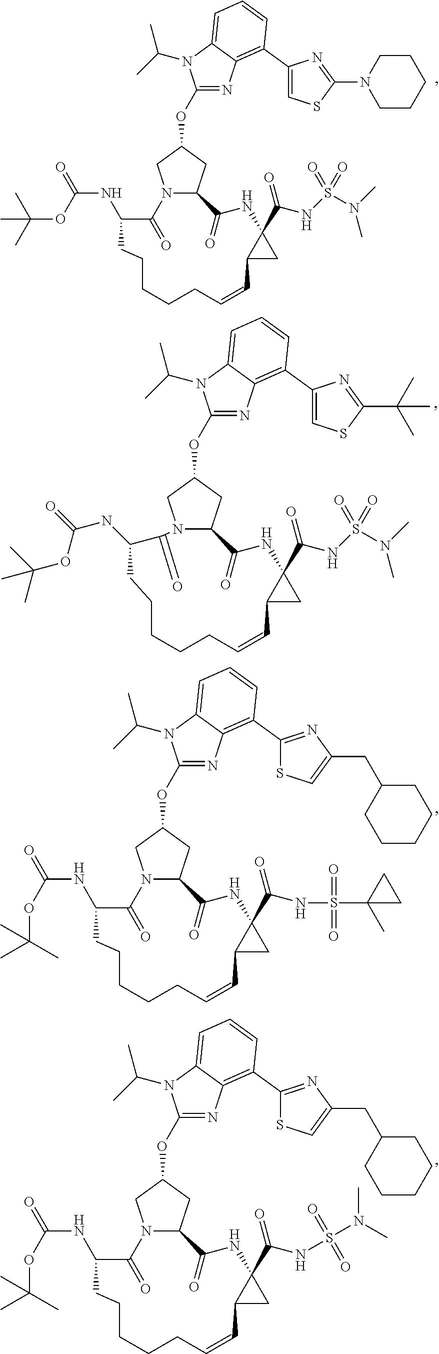

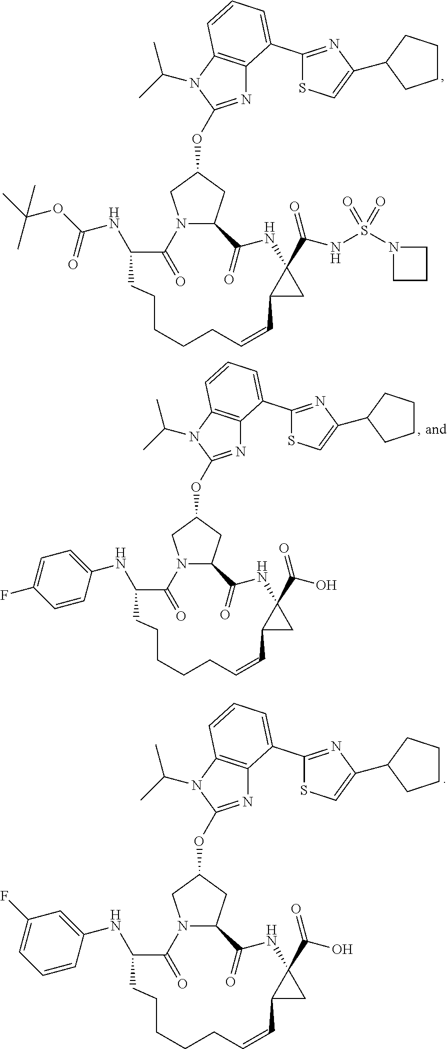

Some embodiments provide a compound having the structure of Formula II:

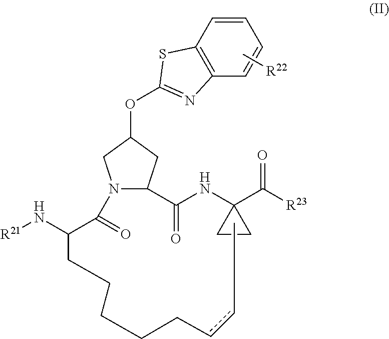

or a pharmaceutically acceptable salt or prodrug thereof wherein R²¹ is selected from —C(O)OR^{1e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{2la}R^{21b}, —NHC(O)NR^{2la}R^{21b}, —C(O)OR^{21c}, and heteroaryl.

R^{21e} is selected from the group consisting of C₁₋₆ alkyl, cycloalkyl, and heterocyclyl; R^{21a} and R^{21b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{21c}, —C(O)R^{21d}, optionally substituted aryl, and optionally substituted heteroaryl; and R^{21c} and R^{21d} are each separately selected from the group consisting of —H (hydrogen), C₁₋₄ alkoxy, C₁₋₆ alkyl, C₃₋₇ cycloalkyl, aryl, arylalkyl and heteroaryl.

R²² is heteroaryl optionally substituted with one or more R^{22a}; each R^{22a} is independently selected from the group consisting of C₁₋₆ alkyl, C₃₋₇ cycloalkyl, C₁₋₆ alkoxy, heteroaryl, heterocyclyl, arylalkyl, aryl, halo, —CN, —CF₃, —OCF₃, —C(O)NR′R″ and —NR′R″, wherein said C₁₋₆ alkyl, C₃₋₇ cycloalkyl, C₁₋₆ alkoxy, heteroaryl, heterocyclyl, arylalkyl, and aryl are each optionally substituted with one or more R^{22b}; each R^{22b} is independently selected from the group consisting of halo, —CF₃, —OCF₃, C₁₋₆ alkyl, C₁₋₆ alkoxy, and aryl.

Each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl.

R²³ is —OH, —NHS(O)₂R^{23a}, —NHS(O)₂OR^{23a} or —NHS(O)₂NR^{23b}R^{23c}; where R^{23a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro.

R^{23b} and R^{23c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{23b} and R^{23c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula III:

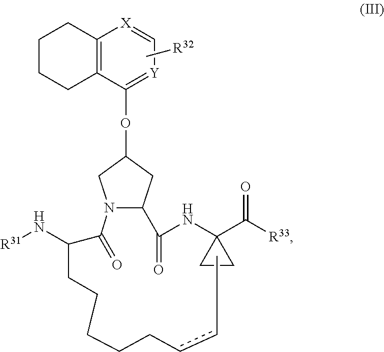

or a pharmaceutically acceptable salt or prodrug thereof wherein X and Y are each N or CH. R³¹ is selected from —C(O)OR^{31e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{31a}R^{31b}, —NHC(O)NR^{31a}R^{31b}, —C(O)OR^{31c}, and heteroaryl; R^{31e} is selected from the group consisting of C₁₋₆ alkyl, cycloalkyl, and heterocyclyl; R^{31a} and R^{31b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{31c}, —C(O)R^{31d}, optionally substituted aryl, and optionally substituted heteroaryl; and R^{31c} and R^{31d} are each separately selected from the group consisting of —H (hydrogen), C₁₋₄ alkoxy, C₁₋₆ alkyl, C₃₋₇ cycloalkyl, aryl, arylalkyl and heteroaryl.

R³² is hydrogen, optionally substituted C₁₋₆ alkyl, optionally substituted aryl, or optionally substituted heteroaryl.

R³³ is —OH, —NHS(O)₂R^{33a}, —NHS(O)₂OR^{33a} or —NHS(O)₂NR^{33b}R^{33c}; where R^{33a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro; R^{33b} and R^{33c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{33b} and R^{33c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula IV or V:

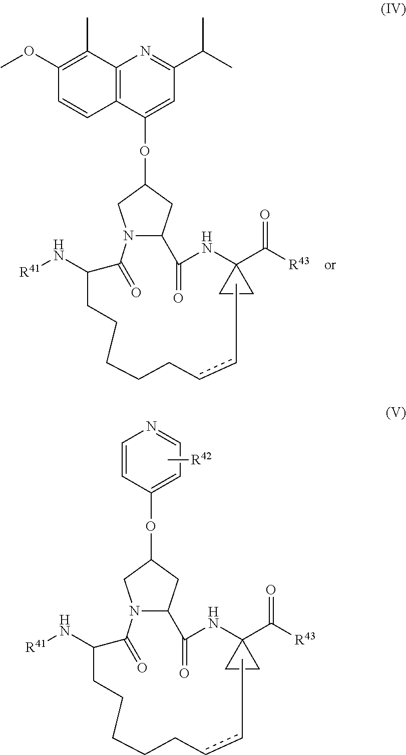

or a pharmaceutically acceptable salt or prodrug thereof wherein R⁴¹ is selected from —C(O)OR^{41e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{41a}R^{41b}, —NHC(O)NR^{41a}R^{41b}, —C(O)OR^{41c}, and heteroaryl; R^{41e} is selected from the group consisting of C₁₋₆ alkyl, cycloalkyl, and heterocyclyl; R^{41a} and R^{41b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{41c}, —C(O)R^{41d}, optionally substituted aryl, and optionally substituted heteroaryl; and R^{41c} and R^{41d} are each separately selected from the group consisting of —H (hydrogen), C₁₋₄ alkoxy, C₁₋₆ alkyl, C₃₋₇ cycloalkyl, aryl, arylalkyl and heteroaryl.

R⁴² is heteroaryl or aryl, optionally substituted by one or more R^{42a}; wherein each R^{42a} is independently selected from the group consisting of —H (hydrogen), optionally substituted C₁₋₆ alkyl, optionally substituted C₁₋₆ alkoxy, optionally substituted aryl, optionally substituted arylalkyl, optionally substituted heteroaryl, optionally substitute C₃₋₇ cycloalkyl, and optionally substituted heterocycloalkyl.

R⁴³ is —OH, —NHS(O)₂R^{43a}, —NHS(O)₂OR^{43a} or —NHS(O)₂NR^{43b}R^{43c}; where R^{43a} is selected from the group consisting of C₁₋₆ alkyl, —(CH₂)^{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro; R^{43b} and R^{43c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{43b} and R^{43c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

Some embodiments provide a compound having the structure of Formula VI:

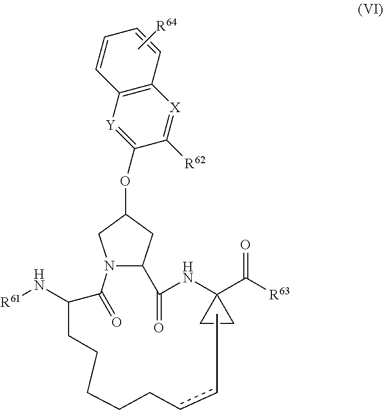

or a pharmaceutically acceptable salt or prodrug thereof wherein X and Y are each N or CH; R⁶¹ is selected from —C(O)OR^{61e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{61a}R^{61b}, —NHC(O)NR^{61a}R^{61b}, C(O)OR^{61c}, and heteroaryl.

Wherein R^{61a} and R^{61b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{61c}, —C(O)R^{61d}, optionally substituted aryl, and optionally substituted heteroaryl; R^{61c} and R^{61d} are each separately selected from the group consisting of —H (hydrogen), C₁₋₄ alkoxy, C₁₋₆ alkyl, C₃₋₇ cycloalkyl, aryl, arylalkyl and heteroaryl; R^{61e} is selected from the group consisting of C₁₋₆ alkyl, cycloalkyl, and heterocyclyl.

R⁶² is selected from the group consisting of —H, —C(O)OR^{62a}, C₁₋₆ alkyl optionally substituted with up to 5 fluoro, C₂₋₆ alkenyl, C₃₋₇ cycloalkyl, optionally substituted aryl and optionally substituted heteroaryl; wherein R^{62a} is selected from the group consisting of —H (hydrogen), C₁₋₄ alkoxy, C₁₋₆ alkyl, C₃₋₇ cycloalkyl, aryl, arylalkyl and heteroaryl.

R⁶³ is —OH, —NHS(O)₂R^{63a}, —NHS(O)₂OR^{63a} or —NHS(O)₂NR^{63b}R^{63e}; where R^{63a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro; R^{63b} and R^{63c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{63b} and R^{63c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl; each t is independently 0, 1 or 2; and each q is independently 0, 1 or 2.

R⁶⁴ is selected from the group consisting of optionally substituted C₁₋₆ alkyl, optionally substituted aryl, or optionally substituted heteroaryl; and any bond represented by a dashed and solid line represents a bond selected from the group consisting of a single bond and a double bond.

Some embodiments provide a pharmaceutical composition comprising a pharmaceutically acceptable excipient and a compound of any one of Formulas I, Ia, II, IIa, III, IIIa, IV, IVa, V, Va, VI and VIa, or any compounds disclosed herein.

Some embodiments provide a method of inhibiting NS3/NS4 protease activity comprising contacting a NS3/NS4 protease with a compound of any one of Formulas I, Ia, II, IIa, III, IIIa, IV, IVa, V, Va, VI and VIa, any compounds disclosed herein, or a pharmaceutical composition disclosed herein.

Some embodiments provide a method of treating liver fibrosis in an individual, the method comprising administering to the individual an effective amount of a compound of any one of Formulas I, Ia, II, IIa, III, IIIa, IV, IVa, V, Va, VI and VIa, any compounds disclosed herein, or a pharmaceutical composition disclosed herein.

Some embodiments provide a method of increasing liver function in an individual having a hepatitis C virus infection, the method comprising administering to the individual an effective amount of a compound of any one of Formulas I, Ia, II, IIa, III, IIIa, IV, IVa, V, Va, VI and VIa, any compounds disclosed herein, or a pharmaceutical composition disclosed herein.

### DETAILED DESCRIPTION OF THE PREFERRED EMBODIMENT

### Definitions

As used herein, common organic abbreviations are defined as follows:
Ac Acetyl
Ac₂O Acetic anhydride
aq. Aqueous
Bn Benzyl
Bz Benzoyl
BOC or Boc tert-Butoxycarbonyl
Bu n-Butyl
cat. Catalytic
Cbz Carbobenzyloxy
CDI 1,1′-carbonyldiimidazole
Cy(c-C₆H₁₁ Cyclohexyl
° C. Temperature in degrees Centigrade
DBU 1,8-Diazabicyclo[5.4.0]undec-7-ene
DCE 1,2-Dichloroethane
DCM methylene chloride
DIEA Diisopropylethylamine
DMA Dimethylacetamide
DMAP 4-(Dimethylamino)pyridine
DME Dimethoxyethane
DMF N,N′-Dimethylformamide
DMSO Dimethylsulfoxide
Et Ethyl
EtOAc Ethyl acetate
g Gram(s)
h Hour (hours)
HATU 2-(1H-7-azabenzotriazol-1-yl)-1,1,3,3-tetramethyl uronium hexafluorophosphate
HOBT 1-Hydroxybenzotriazole
HPLC High performance liquid chromatography
iPr Isopropyl
IU International Units
LCMS Liquid chromatography-mass spectrometry
LDA Lithium diisopropylamide
mCPBA meta-Chloroperoxybenzoic Acid
MeOH Methanol
MeCN Acetonitrile
mL Milliliter(s)
MTBE Methyl tertiary-butyl ether
NH₄OAc Ammonium acetate
PG Protecting group
Pd/C Palladium on activated carbon
ppt Precipitate
PyBOP (Benzotriazol-1-yloxy)tripyrrolidinophosphonium hexafluorophosphate
RCM Ring closing metathesis
rt Room temperature
sBuLi sec-Butylithium
TEA Triethylamine
TCDI 1,1′-Thiocarbonyl diimidazole
Tert, t tertiary
TFA Trifluoracetic acid
THF Tetrahydrofuran
TLC Thin-layer chromatography
TMEDA Tetramethylethylenediamine
μL Microliter(s)

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

As used herein, the term “alkyl” refers to a branched or unbranched fully saturated acyclic aliphatic hydrocarbon group (i.e. composed of carbon and hydrogen containing no double or triple bonds). In some embodiments, alkyls may be substituted or unsubstituted. Alkyls include, but are not limited to, methyl, ethyl, propyl, isopropyl, butyl, isobutyl, tertiary butyl, pentyl, hexyl, and the like, each of which may be optionally substituted in some embodiments.

As used herein, the term “heteroalkyl” refers to a branched or unbrached fully saturated acyclic aliphatic hydrocarbon group containing one or more heteroatoms in the carbon back bone (i.e., an alkyl group in which one or more carbon atoms is replaced with a heteroatom). In some embodiments, heteroalkyls may be substituted or unsubstituted. Heteroalkyls include, but are not limited to, ethers, thioethers, and alkyl-amino-alkyls.

The term “halo” used herein refers to fluoro, chloro, bromo, or iodo.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “aryl” used herein refers to homocyclic aromatic radical whether one ring or multiple fused rings. Examples of aryl groups include, but are not limited to, phenyl, naphthyl, phenanthrenyl, naphthacenyl, and the like.

The term “cycloalkyl” used herein refers to saturated aliphatic ring system radical having three to twenty carbon atoms including, but not limited to, cyclopropyl, cyclopentyl, cyclohexyl, cycloheptyl, and the like.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, and the like.

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantyl, norbornyl, and the like.

The term “polycycloalkenyl” used herein refers to aliphatic ring system radical having at least two rings that are fused with or without bridgehead carbons in which at least one of the rings has a carbon-carbon double bond. Examples of polycycloalkenyl groups include, but are not limited to, norbornylenyl, 1,1′-bicyclopentenyl, and the like.

The term “polycyclic hydrocarbon” used herein refers to a ring system radical in which all of the ring members are carbon atoms. Polycyclic hydrocarbons can be aromatic or can contain less than the maximum number of non-cumulative double bonds. Examples of polycyclic hydrocarbon include, but are not limited to, naphthyl, dihydronaphthyl, indenyl, fluorenyl, and the like.

The term “heterocyclic” or “heterocyclyl” or “heterocycloalkyl” used herein refers to cyclic non-aromatic ring system radical having at least one ring in which one or more ring atoms are not carbon, namely heteroatom. Monocyclic “heterocyclic” or “heterocyclyl” moieties are non-aromatic. Bicyclic “heterocyclic” or “heterocyclyl” moieties include one non-aromatic ring wherein at least one heteroatom is present in the non-aromatic ring. Examples of heterocyclic groups include, but are not limited to, morpholinyl, tetrahydrofuranyl, dioxolanyl, pyrrolidinyl, oxazolyl, pyranyl, pyrrolyl, isoindoline and the like.

The term “heteroaryl” used herein refers to an aromatic ring system radical in which one or more ring atoms are not carbon, namely heteroatoms, whether one ring or multiple fused rings. When two or more heteroatoms are present, they may be the same or different. In fused ring systems, the one or more heteroatoms may be present in only one of the rings. Examples of heteroaryl groups include, but are not limited to, benzothiazyl, benzoxazyl, quinazolinyl, quinolinyl, isoquinolinyl, quinoxalinyl, pyridinyl, pyrrolyl, oxazolyl, indolyl, and the like.

The term “heteroatom” used herein refers to S (sulfur), N (nitrogen), and O (oxygen).

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like.

The term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage. Examples of alkylthio groups include, but are not limited to, methanesulfide, ethanesulfide, propanesulfide, isopropanesulfide, butanesulfide, n-butanesulfide, sec-butanesulfide, tert-butanesulfide and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “hydroxyalkyl” used herein refers to one or more hydroxy groups appended to an alkyl radical.

The term “aminoalkyl” used herein refers to one or more amino groups appended to an alkyl radical.

The term “arylalkyl” used herein refers to one or more aryl groups appended to an alkyl radical.

The term “carbamyl” used herein refers to RNHC(O)O—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO₂—.

The term “thiocarbonyl” used herein refers to C═S.

The term “thiocarboxy” used herein refers to CSOH.

The term “sulfonamide” used herein refers to —SO₂NR′₂ where each R′ is individually selected from H (hydrogen), C₁-C₆ alkyl, C₃-C₇ cycloalkyl, arylalkyl and aryl optionally substituted with C₁-C₆ alkyl.

As used herein, a radical indicates species with one or more, unpaired electron such that the species containing the radical can be covalently bonded to one or more other species. Hence, in this context, a radical is not necessarily a free radical. Rather, a radical indicates a specific portion of a larger molecule. The term “radical” can be used interchangeably with the terms “group” and “moiety.”

As used herein, a substituted group is derived from the unsubstituted parent structure in which there has been an exchange of one or more hydrogen atoms for another atom or group. When substituted, the substituent group(s) is (are) one or more group(s) individually and independently selected from C₁-C₆ alkyl, C₁-C₆ alkenyl, C₁-C₆ alkynyl, C₃-C₇ cycloalkyl (optionally substituted with halo, alkyl, alkoxy, carboxyl, haloalkyl, CN, —CF₃, and —OCF₃), cycloalkyl geminally attached, C₁-C₆ heteroalkyl, C₃-C₁₀ heterocycloalkyl (e.g., tetrahydrofuryl) (optionally substituted with halo, alkyl, alkoxy, carboxyl, CN, —CF₃, and —OCF₃), aryl (optionally substituted with halo, alkyl, aryl optionally substituted with C₁-C₆ alkyl, arylalkyl, alkoxy, carboxyl, CN, —CF₃, and —OCF₃), arylalkyl (optionally substituted with halo, alkyl, alkoxy, aryl, carboxyl, CN, —CF₃, and —OCF₃), heteroaryl (optionally substituted with halo, alkyl, alkoxy, aryl, aralkyl, carboxyl, CN, —CF₃, and —OCF₃), halo (e.g., chloro, bromo, iodo and fluoro), cyano, hydroxy, —CF₃, C₁-C₆ alkoxy, aryloxy, sulfhydryl(mercapto), halo(C₁-C₆)alkyl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, keto (oxy), carbonyl, carboxy, glycolyl, glycyl, hydrazino, guanyl, sulfamyl, sulfonyl, sulfinyl, thiocarbonyl, thiocarboxy, sulfonamide, ester, C-amide, N-amide, N-carbamate, O-carbamate, urea and combinations thereof. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

The term “pharmaceutically acceptable salt,” as used herein, and particularly when referring to a pharmaceutically acceptable salt of a compound, including a compound of Formulas I, II, III, IV, or V, as produced and synthesized by the methods disclosed herein, refers to any pharmaceutically acceptable salts of a compound, and preferably refers to an acid addition salt of a compound. With respect to compounds synthesized by the method of this embodiment that contain a basic nitrogen, the preferred examples of pharmaceutically acceptable salts are acid addition salts of pharmaceutically acceptable inorganic or organic acids, for example, hydrohalic, sulfuric, phosphoric acid or aliphatic or aromatic carboxylic or sulfonic acid. Examples of pharmaceutically acceptable inorganic or organic acids as a component of an addition salt, include but are not limited to, hydrochloric acid, hydrobromic acid, phosphoric acid, sulfuric acid acetic acid, succinic acid, lactic acid, malic acid, tartaric acid, citric acid, ascorbi acid c, nicotinic acid, methanesulfonic acid, p-toluensulfonic acid or naphthalenesulfonic acid acid. With respect to compounds synthesized by the method of this embodiment that contain an acidic functional group, the preferred examples of pharmaceutically acceptable salts include, but are not limited to, alkali metal salts (sodium or potassium), alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

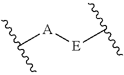

includes the substituent being oriented such that the A is attached at the leftmost attachment point of the molecule as well as attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(O)— that requires two points of attachment, includes di-radicals such as

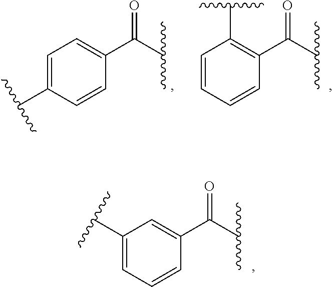

and the like.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

### Compounds

The present embodiments provide compounds of Formulae I, Ia, II, IIa, III, IIIa, IV, IVa, V, Va, VI and VIa, as well as pharmaceutical compositions and formulations comprising any compound of Formulae I, Ia, II, IIa, III, IIIa, IV, IVa, V, Va, VI and VIa. A subject compound is useful for treating HCV infection and other disorders, as discussed below.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Formula I

The embodiments provide a compound having the structure of Formula I:

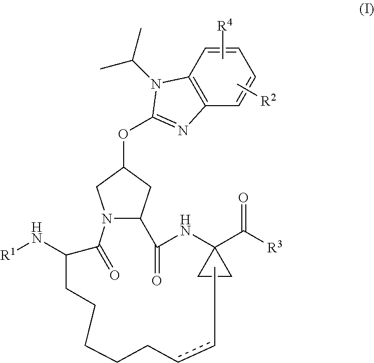

or a pharmaceutically acceptable salt or prodrug thereof, wherein R¹ is selected from hydrogen, —C(O)OR^{1e} or optionally substituted heteroaryl; or R¹ is aryl optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl.

R^{1e} is selected from the group consisting of alkyl, cycloalkyl, and heterocyclyl; R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl; and R^{1c} and R^{1d} are each separately selected from the group consisting of —H (hydrogen), C₁₋₄ alkoxy, C₁₋₆ alkyl, C₃₋₇ cycloalkyl, aryl, arylalkyl and heteroaryl.

R² is selected from the group consisting of

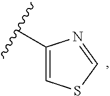

pyrazinyl, and pyrimidinyl, each optionally substituted with R^{2a}; or R² is

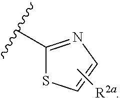

R^{2a} is phenyl substituted with one or more R^{2b} or benzyl optionally substituted with one or more R^{2b}; wherein R^{2b} is halo, —CF₃, —OCF₃, C₁₋₆ alkyl, C₁₋₆ alkoxy, or phenyl; or R^{2a} is optionally substituted C₁₋₆ alkyl, optionally substituted C₃₋₇ cycloalkyl, optionally substituted dihydrobenzodioxinyl, optionally substituted piperidinyl, optionally substituted piperazinyl, optionally substituted tetrahydropyranyl, or optionally substituted pyrrolidinyl.

R³ is —OH, —NHS(O)₂R^{3a}, —NHS(O)₂OR^{3a} or —NHS(O)₂NR^{3b}R^{3c}, wherein R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro.

R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

R⁴ is selected from the group consisting of hydrogen, halo, optionally substituted C₁₋₆ alkyl, optionally substituted C₁₋₆ alkoxy, and optionally substituted C₂₋₆ alkenyl.

Each t is independently 0, 1 or 2; each q is independently 0, 1 or 2; and any bond represented by a dashed and solid line represents a bond selected from the group consisting of a single bond and a double bond.

Some embodiments include the proviso that when R¹ is —C(O)O-t-butyl, R³ is —NHS(O)₂-methylcyclopropyl or —NHS(O)₂N(CH₃)₂, R⁴ is H, and R² is

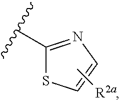

then R^{2a} is not cyclopropyl, cyclobutyl, cyclohexyl, unsubstituted benzyl,

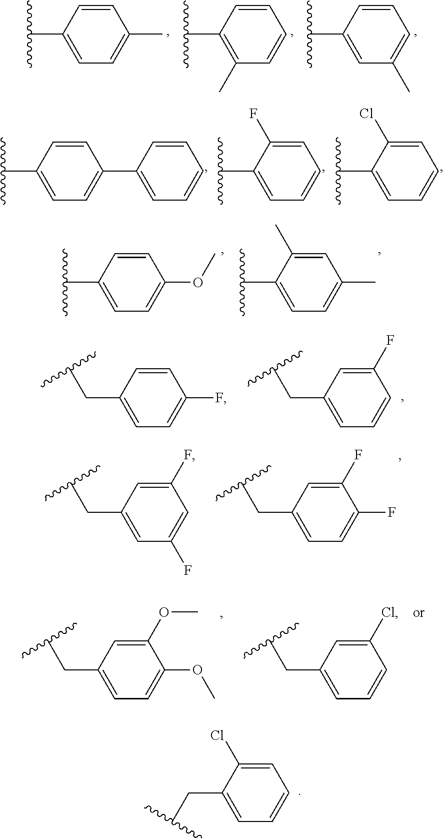

Some embodiments include the proviso that the compound is not selected from the group consisting of

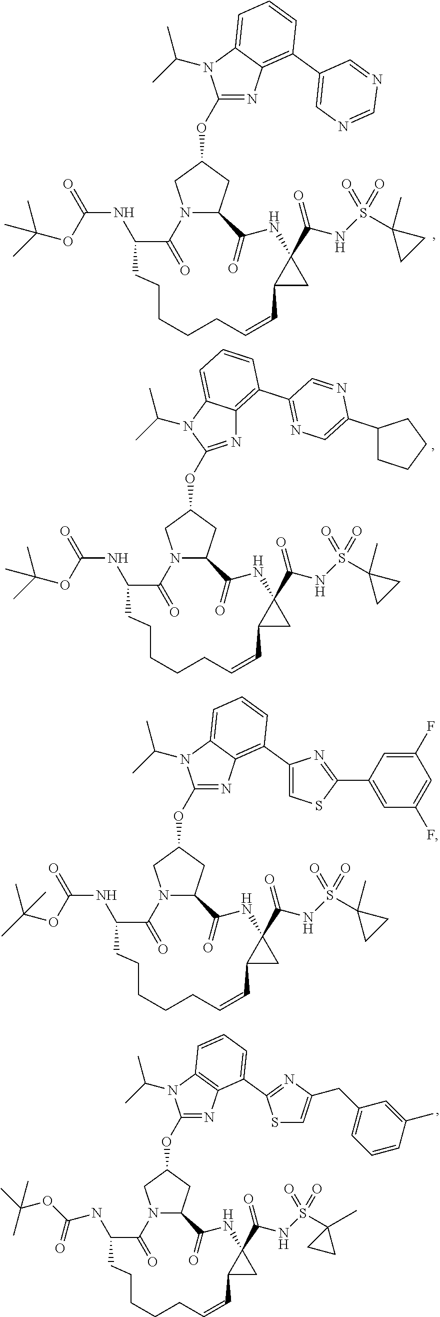

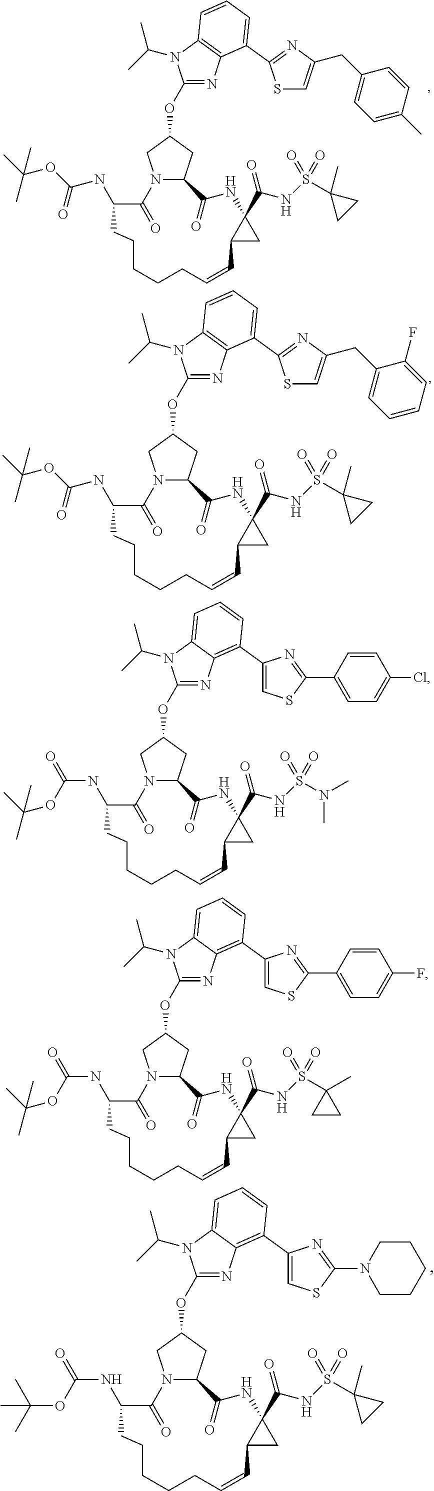

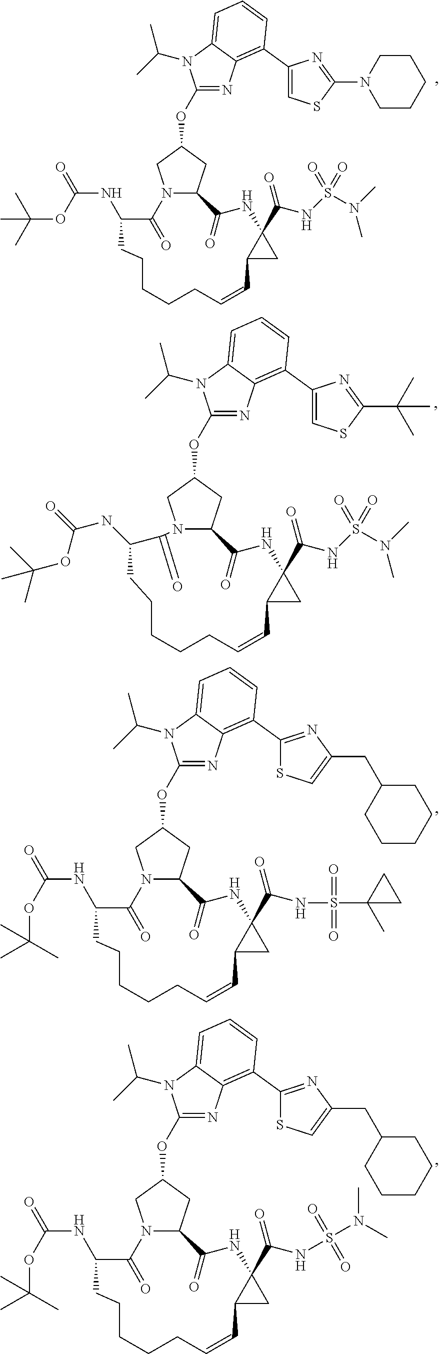

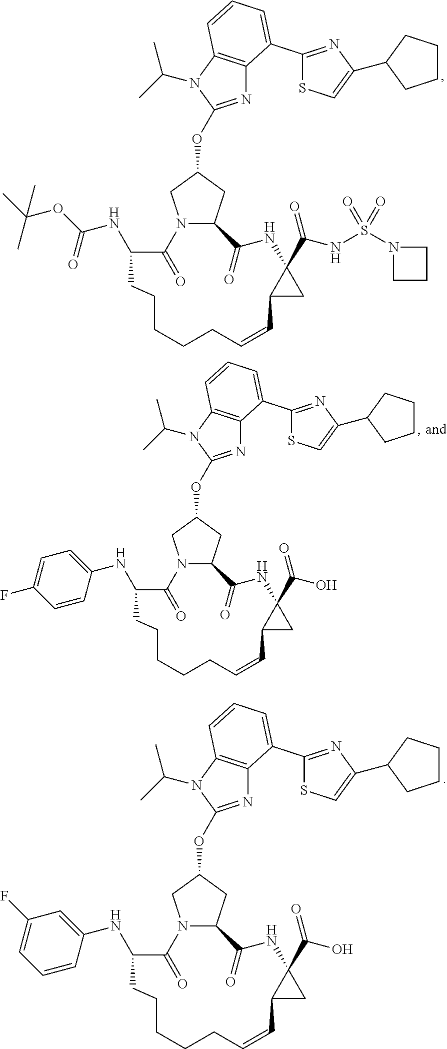

In some embodiments, compounds of Formula I have the structure of Formula Ia:

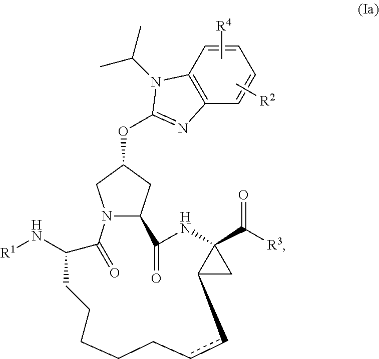

wherein R¹, R², R³, and R⁴ are the same as defined above.

Some embodiments provide compounds of Formula I or Formula Ia, in which R^{2a} is phenyl substituted with one or more R^{2b} or benzyl optionally substituted with one or more R^{2b}; wherein R^{2b} is halo, —CF₃, —OCF₃, C₁₋₆ alkyl, C₁₋₆ alkoxy, or phenyl; or R^{2a} is optionally substituted C₁₋₆ alkyl, optionally substituted C₃₋₇ cycloalkyl, optionally substituted dihydrobenzodioxinyl, optionally substituted piperidinyl, optionally substituted piperazinyl, optionally substituted tetrahydropyranyl, or optionally substituted pyrrolidinyl, and R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

Some embodiments provide compounds of Formula I or Formula Ia, in which wherein R^{2a} is phenyl substituted with one or more R^{2b} or benzyl optionally substituted with one or more R^{2b}; wherein R^{2b} is halo, —CF₃, —OCF₃, methyl, propyl, butyl, methoxy, or phenyl. In some embodiments, R¹ is —C(O)OR^{1e}, wherein R^{1e} is t-butyl, C₃₋₇ cycloalkyl, or pyrrolidinyl; and R³ is selected from the group consisting of —NHS(O)₂-methylcyclopropyl, —NHS(O)₂-cyclopropyl, and —NHS(O)₂—N(CH₃)₂.

Some embodiments provide compounds of Formula I or Formula Ia, in which R^{2a} is C₁₋₆ alkyl, C₃₋₇ cycloalkyl, dihydrobenzodioxinyl, optionally substituted piperazinyl, or optionally substituted pyrrolidinyl. In some embodiments, R¹ is —C(O)OR^{1e}, wherein lee is t-butyl, C₃₋₇ cycloalkyl, or pyrrolidinyl; and R³ is selected from the group consisting of —NHS(O)₂-methylcyclopropyl, —NHS(O)₂-cyclopropyl, and —NHS(O)₂—N(CH₃)₂.

Some embodiments provide compounds of Formula I or Formula Ia, in which R¹ is —C(O)OR^{1e}, wherein lee is t-butyl, C₃₋₇ cycloalkyl, or pyrrolidinyl; and R³ is selected from the group consisting of —NHS(O)₂-methylcyclopropyl, —NHS(O)₂-cyclopropyl, —NHS(O)₂-ethynylcyclopropyl, —NHS(O)₂-propynylcyclopropyl and —NHS(O)₂—N(CH₃)₂. In some embodiments, R¹ is —C(O)O-t-butyl and R³ is —NHS(O)₂-methylcyclopropyl or —NHS(O)₂—N(CH₃)₂.

Some embodiments provide compounds of Formula I or Formula Ia, in which R² is

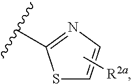

R^{2a} is optionally substituted C₁₋₆ alkyl or optionally substituted C₃₋₇ cycloalkyl, and R⁴ is halo, optionally substituted C₁₋₆ alkyl, or optionally substituted C₁₋₆ alkoxy.

In some embodiments, the compound of Formula I is selected from the group consisting of compounds 101-190 as described in the Example section.

### Formula II

Some embodiments provide compounds of Formula II:

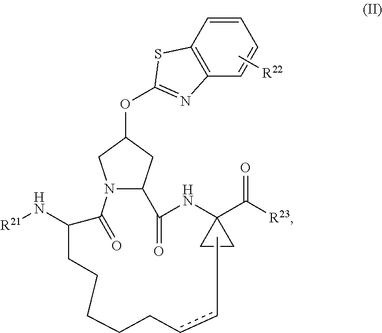

or a pharmaceutically acceptable salt or prodrug thereof wherein R²¹ is selected from —C(O)OR^{2le} or optionally substituted heteroaryl; or R²¹ is aryl optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{2la}R^{21b}, —NHC(O)NR^{2la}R^{21b}, —C(O)OR^{21c}, and heteroaryl.

R^{21e} is selected from the group consisting of C₁₋₆ alkyl, cycloalkyl, and heterocyclyl; R^{21a} and R^{21b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{21c}, —C(O)R^{21d}, optionally substituted aryl, and optionally substituted heteroaryl; and R^{2le} and R^{21d} are each separately selected from the group consisting of —H (hydrogen), C₁₋₄ alkoxy, C₁₋₆ alkyl, C₃₋₇ cycloalkyl, aryl, arylalkyl and heteroaryl.

R²² is heteroaryl optionally substituted with one or more R^{22a}; each R^{22a} is independently selected from the group consisting of C₁₋₆ alkyl, C₃₋₇ cycloalkyl, C₁₋₆ alkoxy, heteroaryl, heterocyclyl, arylalkyl, aryl, halo, —CN, —CF₃, —OCF₃, —C(O)NR′R″ and —NR′R″, wherein said C₁₋₆ alkyl, C₃₋₇ cycloalkyl, C₁₋₆ alkoxy, heteroaryl, heterocyclyl, arylalkyl, and aryl are each optionally substituted with one or more R^{22b}; each R^{22b} is independently selected from the group consisting of halo, —CF₃, —OCF₃, C₁₋₆ alkyl, C₁₋₆ alkoxy, and aryl.

Each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl.

R²³ is —OH, —NHS(O)₂R^{23a}, —NHS(O)₂OR^{23a} or —NHS(O)₂NR^{23b}R^{23c}; where R^{23a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro.

R^{23b} and R^{23c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{23b} and R^{23c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

In some embodiments, compounds of Formula II have the structure of Formula IIa:

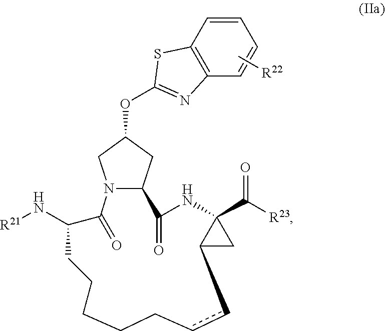

wherein R²¹, R²², and R²³ are the same as defined above.

In some embodiments, R^{23a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

In some embodiments, R²² is thiazyl, pyrazinyl or pyrimidinyl, each optionally substituted with one or more R^{22a}. In some embodiments, each R^{22a} is independently selected from the group consisting of C₃₋₇ cycloalkyl, aryl, heteroaryl, arylalkyl, and heterocyclyl, each optionally substituted with one or more R^{22b}.

In some embodiments, R²¹ is —C(O)OR^{21e}, wherein R^{21e} is t-butyl, C₃₋₇ cycloalkyl, or pyrrolidinyl; and R²³ is selected from the group consisting of —NHS(O)₂-methylcyclopropyl, —NHS(O)₂-cyclopropyl, and —NHS(O)₂—N(CH₃)₂.

In some embodiments, the compound of Formula II is compound 201 as shown in the Example below.

### Formula III

Some embodiments provide a compound having the structure of Formula III:

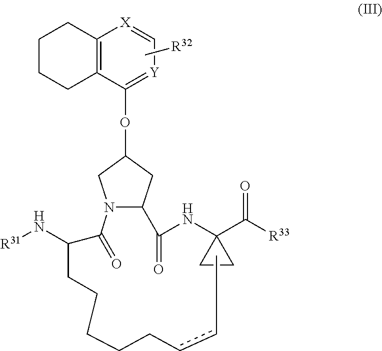

or a pharmaceutically acceptable salt or prodrug thereof wherein X and Y are each N or CH.

R³¹ is selected from —C(O)OR^{31e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{31a}R^{31b}, —NHC(O)NR^{31a}R^{31b}, —C(O)OR^{31c}, and heteroaryl; R^{31e} is selected from the group consisting of C₁₋₆ alkyl, cycloalkyl, and heterocyclyl; R^{31a} and R^{31b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{31c}, —C(O)R^{31d}, optionally substituted aryl, and optionally substituted heteroaryl; and R^{31c} and R^{31d} are each separately selected from the group consisting of —H (hydrogen), C₁₋₄ alkoxy, C₁₋₆ alkyl, C₃₋₇ cycloalkyl, aryl, arylalkyl and heteroaryl.

R³² is hydrogen, optionally substituted C₁₋₆ alkyl, optionally substituted aryl, or optionally substituted heteroaryl.

R³³ is —OH, —NHS(O)₂R^{33a}, —NHS(O)₂OR^{33a} or —NHS(O)₂NR^{33b}R^{33c}; where R^{33a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro; R^{33b} and R^{33c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{33b} and R^{33c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and any bond represented by a dashed and solid line represents a bond selected from the group consisting of a single bond and a double bond.

In some embodiments, compounds of Formula III have the structure of Formula IIIa:

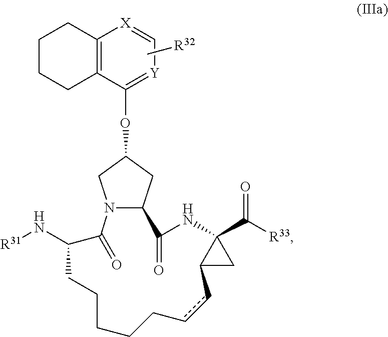

wherein R³¹, R³², and R³³ are the same as defined above.

In some embodiments, wherein R^{33a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

In some embodiments, R³² is hydrogen, aryl, or substituted heteroaryl. In some embodiments, is hydrogen, phenyl, or substituted thiazyl. In some embodiments, at least one of X and Y in Formula (III) or (IIIa) is N.

In some embodiments, R³¹ is —C(O)OR^{31e}, wherein R^{31e} is t-butyl, C₃₋₇ cycloalkyl, or pyrrolidinyl; and R³³ is selected from the group consisting of —NHS(O)₂-methylcyclopropyl, —NHS(O)₂-cyclopropyl, and —NHS(O)₂—N(CH₃)₂.

In some embodiments, the compound of Formula III or IIIa is selected from the group consisting of compounds 301-304 and 401-406 as shown in the Example below.

### Formula IV

Some embodiments provide a compound having the structure of Formula IV:

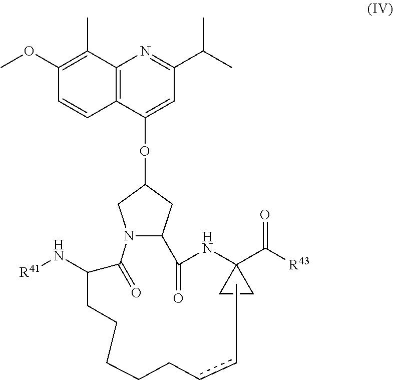

or a pharmaceutically acceptable salt or prodrug thereof wherein R⁴¹ is selected from —C(O)OR^{41e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{41a}R^{41b}, —NHC(O)NR^{41a}R^{41b}, —C(O)OR^{41c}, and heteroaryl; R^{41e} is selected from the group consisting of C₁₋₆ alkyl, cycloalkyl, and heterocyclyl; R^{41a} and R^{41b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{41c}, —C(O)R^{41d}, optionally substituted aryl, and optionally substituted heteroaryl; and R^{41c} and R^{41d} are each separately selected from the group consisting of —H (hydrogen), C₁₋₄ alkoxy, C₁₋₆ alkyl, C₃₋₇ cycloalkyl, aryl, arylalkyl and heteroaryl.

R⁴³ is —OH, —NHS(O)₂R^{43a}, —NHS(O)₂OR^{43a} or —NHS(O)₂NR^{43b}R^{43c}; where R^{43a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro; R^{43b} and R^{43c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{43b} and R^{43c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and (d) any bond represented by a dashed and solid line represents a bond selected from the group consisting of a single bond and a double bond.

In some embodiments, compounds of Formula IV have the structure of Formula IVa:

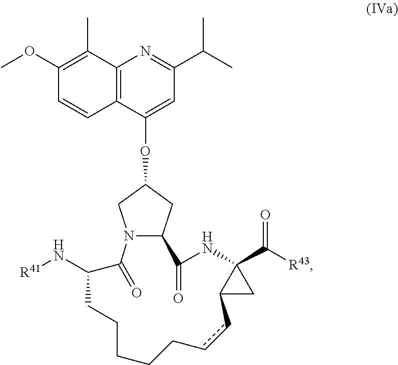

wherein R⁴¹ and R⁴³ are the same as defined above.

In some embodiments, wherein R^{43a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

In some embodiments, R⁴¹ is hydrogen or —C(O)OR^{41e}, wherein R^{41e} is t-butyl, C₃₋₇ cycloalkyl, or pyrrolidinyl. In some embodiments, R⁴³ is selected from the group consisting of —NHS(O)₂-methylcyclopropyl, —NHS(O)₂-cyclopropyl, and —NHS(O)₂—N(CH₃)₂.

In some embodiments, the compound of Formula IV or IVa is selected from compounds 501 and 502 as shown in the Example below.

### Formula V

Some embodiments provide a compound having the structure of Formula V:

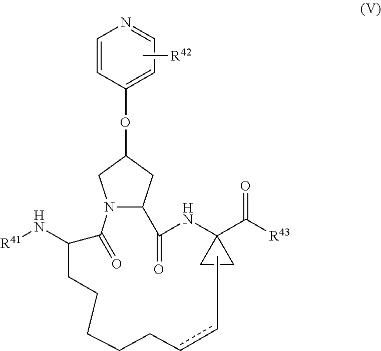

or a pharmaceutically acceptable salt or prodrug thereof wherein R⁴¹ is selected from —C(O)OR^{41e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{41a}R^{41b}, —NHC(O)NR^{41a}R^{41b}, —C(O)OR^{41c}, and heteroaryl; R^{41c} is selected from the group consisting of C₁₋₆ alkyl, cycloalkyl, and heterocyclyl; R^{41a} and R^{41b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{41c}, —C(O)R^{41d}, optionally substituted aryl, and optionally substituted heteroaryl; and R^{41c} and R^{41d} are each separately selected from the group consisting of —H (hydrogen), C₁₋₄ alkoxy, C₁₋₆ alkyl, C₃₋₇ cycloalkyl, aryl, arylalkyl and heteroaryl.

R⁴² is heteroaryl or aryl, optionally substituted by one or more R^{42a}; wherein each R^{42a} is independently selected from the group consisting of —H (hydrogen), optionally substituted C₁₋₆ alkyl, optionally substituted C₁₋₆ alkoxy, optionally substituted aryl, optionally substituted arylalkyl, optionally substituted heteroaryl, optionally substitute C₃₋₇ cycloalkyl, and optionally substituted heterocycloalkyl.

R⁴³ is —OH, —NHS(O)₂R^{43a}, —NHS(O)₂OR^{43a} or —NHS(O)₂NR^{43b}R^{43c}; where R^{43a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)_{q}C₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro; R^{43b} and R^{43c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{43b} and R^{43c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl.

Each t is independently 0, 1 or 2; each q is independently 0, 1 or 2; and (d) any bond represented by a dashed and solid line represents a bond selected from the group consisting of a single bond and a double bond.

In some embodiments, compounds of Formula V have the structure of Formula Va:

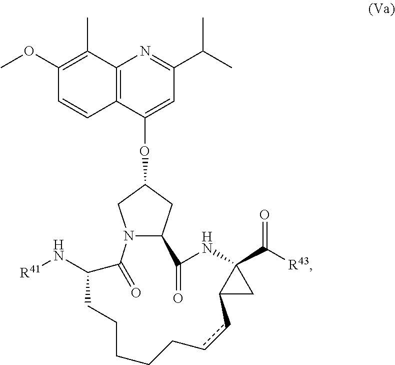

wherein R⁴¹ and R⁴³ are the same as defined above.

In some embodiments, wherein R^{43a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

In some embodiments of the compound of the Formula V, R⁴² is heteroaryl optionally substituted by R^{42a}. In some embodiments, R⁴² is thiazyl optionally substituted by R^{42a}.

In some embodiments of the compound of Formula V or Va, R⁴¹ is hydrogen or —C(O)OR^{41e}, wherein R^{41e} is t-butyl, C₃₋₇ cycloalkyl, or pyrrolidinyl; and R⁴³ is selected from the group consisting of —NHS(O)₂-methylcyclopropyl, —NHS(O)₂-cyclopropyl, and —NHS(O)₂—N(CH₃)₂.

In some embodiments, the compound of Formula V or Va is selected from compounds 601 and 602 as shown in the Example below.

### Formula VI

Some embodiments provide a compound having the structure of Formula VI:

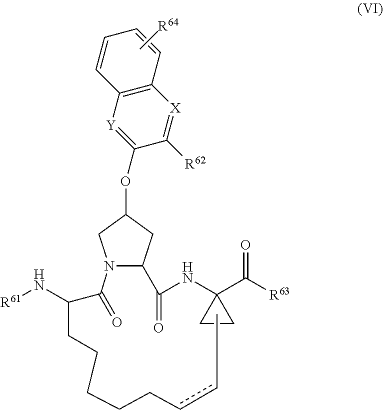

or a pharmaceutically acceptable salt or prodrug thereof wherein X and Y are each N or CH; R⁶¹ is selected from —C(O)OR^{61e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{61a}R^{61b}, —NHC(O)NR^{61a}R^{61b}, C(O)OR^{61c}, and heteroaryl.

Wherein R^{61a} and R^{61b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{61c}, —C(O)R^{61d}, optionally substituted aryl, and optionally substituted heteroaryl; R^{61e} and R^{61d} are each separately selected from the group consisting of —H (hydrogen), C₁₋₄ alkoxy, C₁₋₆ alkyl, C₃₋₇ cycloalkyl, aryl, arylalkyl and heteroaryl; R^{61e} is selected from the group consisting of C₁₋₆ alkyl, cycloalkyl, and heterocyclyl.

R⁶² is selected from the group consisting of —H, —C(O)OR^{62a}, C₁₋₆ alkyl optionally substituted with up to 5 fluoro, C₂₋₆ alkenyl, C₃₋₇ cycloalkyl, optionally substituted aryl and optionally substituted heteroaryl; wherein R^{62a} is selected from the group consisting of —H (hydrogen), C₁₋₄ alkoxy, C₁₋₆ alkyl, C₃₋₇ cycloalkyl, aryl, arylalkyl and heteroaryl.

R⁶³ is —OH, —NHS(O)₂R^{63a}, —NHS(O)₂OR^{63a} or —NHS(O)₂NR^{63b}R^{63c}; where R^{63a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro; R^{63b} and R^{63c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro; or R^{63b} and R^{63c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl; each t is independently 0, 1 or 2; and each q is independently 0, 1 or 2.

R⁶⁴ is selected from the group consisting of optionally substituted C₁₋₆ alkyl, optionally substituted aryl, or optionally substituted heteroaryl; and any bond represented by a dashed and solid line represents a bond selected from the group consisting of a single bond and a double bond.

In some embodiments, compounds of Formula VI have the structure of Formula VIa:

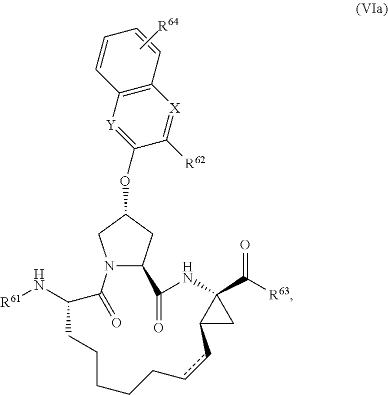

wherein R⁶¹, R⁶², R⁶³ and R⁶⁴ are the same as defined above.

In some embodiments of the compound of the Formula VI or VIa, R⁶² is C₁₋₆ alkyl optionally substituted with up to 5 fluoro. In some embodiments, R⁶² is methyl or —CF₃.

In some embodiments of the compound of the Formula VI, at least one of X and Y is N. In some embodiments, both X and Y are N.

In some embodiments of the compound of the Formula VI, R⁶¹ is —C(O)OR^{61e}, wherein R^{61e} is t-butyl, C₃₋₇ cycloalkyl, or pyrrolidinyl; and R⁶³ is selected from the group consisting of —NHS(O)₂-methylcyclopropyl, —NHS(O)₂-cyclopropyl, and —NHS(O)₂—N(CH₃)₂.

In some embodiments of the compound of the Formula VI, R⁶⁴ is optionally substituted heteroaryl. In some embodiments, R⁶⁴ is thiazyl optionally substituted with: C₃₋₇ cycloalkyl, optionally substituted C₃₋₇ heterocycloalkyl or C₁₋₆ alkyl optionally substituted with up to 5 fluoro.

In some embodiments, the compound of Formula VI or VIa is selected from compounds 701-703 as shown in the Example below.

### Compositions

The present embodiments further provide compositions, including pharmaceutical compositions, comprising compounds of the general Formulae I, Ia, II, IIa, III, IIIa, IV, IVa, V, Va, VI and VIa or any compounds disclosed herein.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} e a Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Example compounds of Formulae I, Ia, II, IIa, III, IIIa, IV, IVa, V, Va, VI and VIa include Compound Numbers 101-190, 201, 301-304, 401-406, 501-502, 601-602, and 701-703 as set forth herein.

### Treating a Hepatitis Virus Infection

The methods and compositions described herein are generally useful in treatment of an of HCV infection.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formulae I, Ia, II, IIa, III, IIIa, IV, IVa, V, Va, VI or VIa, or any compounds disclosed herein, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound of Formulae I, Ia, II, IIa, III, IIIa, IV, IVa, V, Va, VI or VIa, or any compounds disclosed herein, optionally in combination with an effective amount of one or more additional antiviral agents. In some embodiments, an effective amount of a compound of Formulae I, Ia, II, IIa, III, IIIa, IV, IVa, V, Va, VI or VIa, or any compounds disclosed herein, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formulae I, Ia, II, IIa, III, IIIa, IV, IVa, V, Va, VI or VIa, or any compounds disclosed herein, and optionally one or more additional antiviral agents, is an amount that is effective to reduce viral load to lower than 100 genome copies/mL serum.

In some embodiments, an effective amount of a compound of Formulae I, Ia, II, IIa, III, IIIa, IV, IVa, V, Va, VI or VIa, or any compounds disclosed herein, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formulae I, Ia, II, IIa, III, IIIa, IV, IVa, V, Va, VI or VIa, or any compounds disclosed herein, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formulae I, Ia, II, IIa, III, IVa, IV, IVa, V, Va, VI or VIa, or any compounds disclosed herein, and optionally one or more additional antiviral agents, is an amount effective to reduce ALT levels to less than about 45 IU/mL serum.

A therapeutically effective amount of a compound of Formulae I, Ia, II, IIa, III, IVa, IV, IVa, V, Va, VI or VIa, or any compounds disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formulae I, Ia, II, IIa, III, IVa, IV, IVa, V, Va, VI or VIa, or any compounds disclosed herein and an additional antiviral agent is a synergistic amount. The additional antiviral agent may itself be a combination of antiviral agents, e.g., a combination of pegylated interferon-alfa and ribavirin. As used herein, a “synergistic combination” or a “synergistic amount” of a compound of Formulae I, Ia, II, IIa, III, IVa, IV, IVa, V, Va, VI or VIa, or any compounds disclosed herein and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formulae I, Ia, II, IIa, III, IVa, IV, IVa, V, Va, VI or VIa, or any compounds disclosed herein when administered at that same dosage as a monotherapy and (ii) the therapeutic or prophylactic benefit of the additional antiviral agent when administered at the same dosage as a monotherapy.

In some embodiments, a selected amount of a compound of Formulae I, Ia, II, IIa, III, IVa, IV, IVa, V, Va, VI or VIa, or any compounds disclosed herein and a selected amount of an additional antiviral agent are effective when used in combination therapy for a disease, but the selected amount of the compound of Formulae I, Ia, II, IIa, III, IVa, IV, IVa, V, Va, VI or VIa, or any compounds disclosed herein and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of the compound of Formulae I, Ia, II, IIa, III, IVa, IV, IVa, V, Va, VI or VIa, or any compounds disclosed herein when used in combination therapy for a disease, where the selected amount of the additional antiviral agent provides no therapeutic benefit when used in monotherapy for the disease (2) regimens in which a selected amount of the compound of Formulae I, Ia, II, IIa, III, IVa, IV, IVa, V, Va, VI or VIa, or any compounds disclosed herein enhances the therapeutic benefit of a selected amount of the additional antiviral agent when used in combination therapy for a disease, where the selected amount of the compound of Formulae I, Ia, II, IIa, III, IVa, IV, IVa, V, Va, VI or VIa, or any compounds disclosed herein provides no therapeutic benefit when used in monotherapy for the disease and (3) regimens in which a selected amount of the compound of Formulae I, Ia, II, IIa, III, IVa, IV, IVa, V, Va, VI or VIa, or any compounds disclosed herein and a selected amount of the additional antiviral agent provide a therapeutic benefit when used in combination therapy for a disease, where each of the selected amounts of the compound of Formulae I, Ia, II, IIa, III, IVa, IV, IVa, V, Va, VI or VIa, or any compounds disclosed herein and the additional antiviral agent, respectively, provides no therapeutic benefit when used in monotherapy for the disease. As used herein, a “synergistically effective amount” of a compound of Formulae I, Ia, II, IIa, III, IIIa, IV, IVa, V, Va, VI or VIa, or any compounds disclosed herein and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formulae I, Ia, II, IIa, III, IIIa, IV, IVa, V, Va, VI or VIa, or any compounds disclosed herein, and optionally one or more additional antiviral agents. Effective amounts of compounds of Formulae I, Ia, II, IIa, III, IIIa, IV, IVa, V, Va, VI or VIa, or any compounds disclosed herein, with and without one or more additional antiviral agents, as well as dosing regimens, are as discussed below.

Whether treatment with a compound of Formulae I, Ia, II, IIa, III, IIIa, IV, IVa, V, Va, VI or VIa, or any compounds disclosed herein, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formulae I, Ia, II, IIa, III, IVa, IV, IVa, V, Va, VI or VIa, or any compounds disclosed herein, and optionally one or more additional antiviral agents, is an amount that effects a change of one unit or more in the fibrosis stage based on pre- and post-therapy liver biopsies. In particular embodiments, a therapeutically effective amount of a compound of Formulae I, Ia, II, IIa, III, IVa, IV, IVa, V, Va, VI or VIa, or any compounds disclosed herein, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formulae I, Ia, II, IIa, III, IVa, IV, IVa, V, Va, VI or VIa, or any compounds disclosed herein. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formulae I, Ia, II, IIa, III, IVa, IV, IVa, V, Va, VI or VIa, or any compounds disclosed herein, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formulae I, Ia, II, IIa, III, IVa, IV, IVa, V, Va, VI or VIa, or any compounds disclosed herein, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formulae I, Ia, II, IIa, III, IVa, IV, IVa, V, Va, VI or VIa, or any compounds disclosed herein, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formulae I, Ia, II, IIa, III, IVa, IV, IVa, V, Va, VI or VIa, or any compounds disclosed herein, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound of Formulae I, Ia, II, IIa, III, IIIa, IV, IVa, V, Va, VI or VIa, or any compounds disclosed herein, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formulae I, Ia, II, IIa, III, IIIa, IV, IVa, V, Va, VI or VIa, or any compounds disclosed herein, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formulae I, Ia, II, IIa, III, IIIa, IV, IVa, V, Va, VI or VIa, or any compounds disclosed herein, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formulae I, Ia, II, IIa, III, IIIa, IV, IVa, V, Va, VI or VIa, or any compounds disclosed herein, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formulae I, Ia, II, IIa, III, IIIa, IV, IVa, V, Va, VI or VIa, or any compounds disclosed herein, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Other Antiviral or Antifibrotic Agents

As discussed above, a subject method will in some embodiments be carried out by administering an NS3 inhibitor that is a compound of Formulae I, Ia, II, IIa, III, IVa, IV, IVa, V, Va, VI or VIa, or any compounds disclosed herein, and optionally one or more additional antiviral agent(s).

In some embodiments, the method further includes administration of one or more interferon receptor agonist(s). [0222] In other embodiments, the method further includes administration of pirfenidone or a pirfenidone analog.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′3′,-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carb oxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl ester[5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man. In some embodiments, the method further includes administration of a protease inhibitor. In some embodiments, the method further includes administration of another NS5A inhibitor. In some embodiments, the method further includes administration of a helicase inhibitor. In some embodiments, the method further includes administration of a polymerase inhibitor.

In some embodiments, an additional antiviral agent is administered during the entire course of NS3 inhibitor compound treatment. In other embodiments, an additional antiviral agent is administered for a period of time that is overlapping with that of the NS3 inhibitor compound treatment, e.g., the additional antiviral agent treatment can begin before the NS3 inhibitor compound treatment begins and end before the NS3 inhibitor compound treatment ends; the additional antiviral agent treatment can begin after the NS3 inhibitor compound treatment begins and end after the NS3 inhibitor compound treatment ends; the additional antiviral agent treatment can begin after the NS3 inhibitor compound treatment begins and end before the NS3 inhibitor compound treatment ends; or the additional antiviral agent treatment can begin before the NS3 inhibitor compound treatment begins and end after the NS3 inhibitor compound treatment ends.

### Methods of Treatment

### Monotherapies

The NS3 inhibitor compounds described herein may be used in acute or chronic therapy for HCV disease. In many embodiments, the NS3 inhibitor compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS3 inhibitor compound can be administered 5 times per day, 4 times per day, tid, bid, qd, qod, biw, tiw, qw, qow, three times per month, or once monthly. In other embodiments, the NS3 inhibitor compound is administered as a continuous infusion.

In many embodiments, an NS3 inhibitor compound of the embodiments is administered orally.

In connection with the above-described methods for the treatment of HCV disease in a patient, an NS3 inhibitor compound as described herein may be administered to the patient at a dosage from about 0.01 mg to about 100 mg/kg patient bodyweight per day, in 1 to 5 divided doses per day. In some embodiments, the NS3 inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS3 inhibitor compound, the severity of the symptoms and the susceptibility of the subject to side effects. Preferred dosages for a given NS3 inhibitor compound are readily determinable by those of skill in the art by a variety of means. A preferred means is to measure the physiological potency of a given interferon receptor agonist.

In many embodiments, multiple doses of NS3 inhibitor compound are administered. For example, an NS3 inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination therapies with a TNF-α antagonist and an interferon

Some embodiments provide a method of treating an HCV infection in an individual having an HCV infection, the method comprising administering an effective amount of an NS3 inhibitor, and effective amount of a TNF-α antagonist, and an effective amount of one or more interferons.

### Subjects Suitable for Treatment

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Any of the above treatment regimens can be administered to individuals who have been diagnosed with an HCV infection. Any of the above treatment regimens can be administered to individuals having advanced or severe stage liver fibrosis as measured by a Knodell score of 3 or 4 or no or early stage liver fibrosis as measured by a Knodell score of 0, 1, or 2. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (MBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### Preparation of NS3 Inhibitors

### Methodology

The HCV protease inhibitors in the following sections can be prepared according to the procedures and schemes shown in each section. The numberings in each of the following Preparation of NS3 Inhibitor sections including the General Method or General Procedure designations, are meant for that specific section only, and should not be construed or confused with the same numberings, if any, in other sections.

### Preparation of NS3 Inhibitors

### Section I

### Example 1

### P2 Benzimidazole Analogs

### 1.1 Preparation of P2 Precursors—Substituted Phenyl-Thiazole Benzimidazole

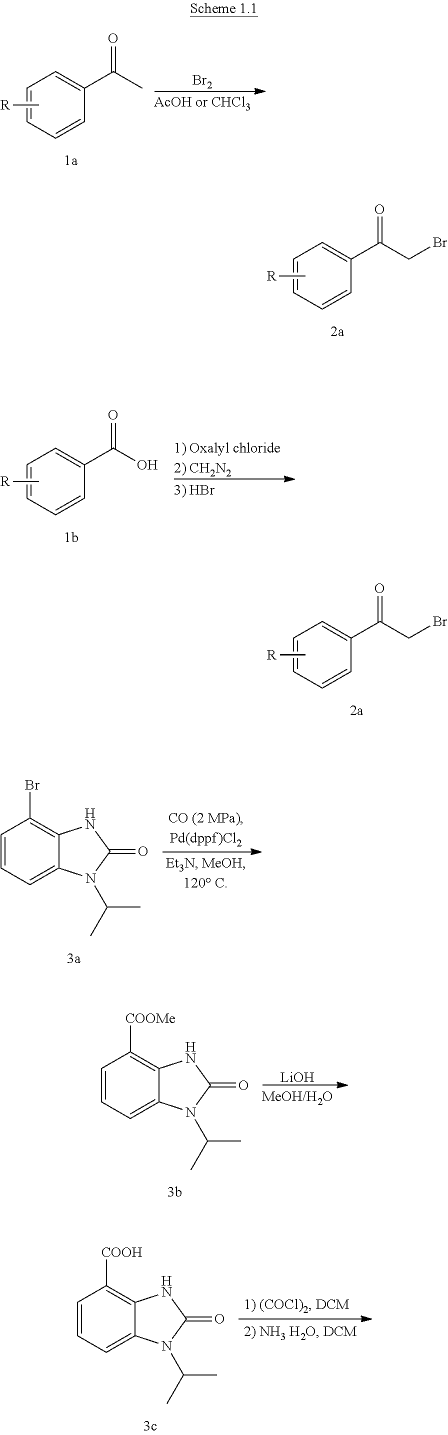

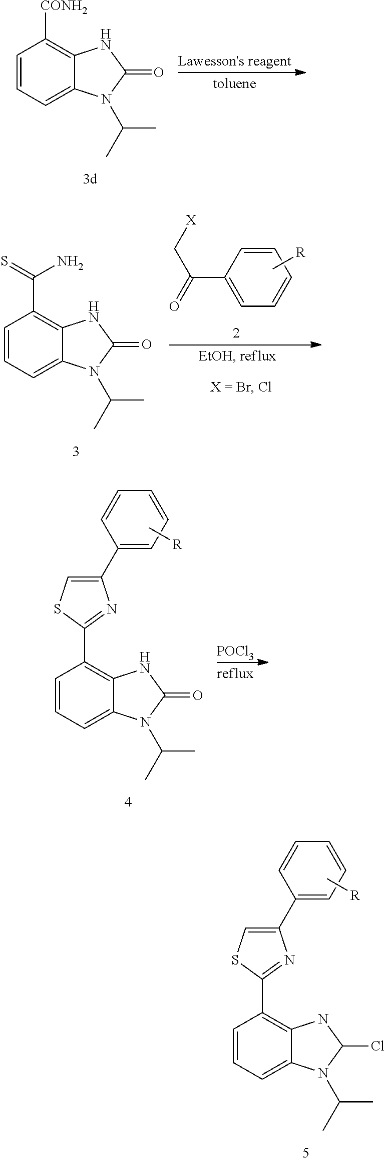

Compound 2a can be prepared from compound 1a or 1b as shown above in Scheme 1.1.

The autoclave was charged with compound 3a (10.85 g, 42.55 mmol), Pd(dppf)Cl₂ (3.1 g, 4.26 mmol), Et₃N (8.88 mL, 63.83 mmol) and MeOH (500 mL), then degassed with CO. The mixture was stirred at 120° C. under CO (2 MPa) for 2 days. The reaction mixture was filtered and the filtrate was concentrated under reduced pressure. The residue (16 g) was used directly in next step without further purification. MS (ESI) m/z (M+H)⁺ 235.0.

To a solution of compound 3b (4.0 g, 17.1 mmol) in MeOH (40 mL) and water (30 mL) was added LiOH (4.0 g, 171 mmol). The mixture was stirred for 12 hrs at rt After that, the solvent was removed under reduced pressure, the aqueous layer was acidified to pH=3 with aq. HCl (2 M) and then extracted with EtOAc. The combined organic layer was washed with brine, dried over anhydrous Na₂SO₄ and concentrated to give a crude product compound 3c, which was used directly in next step. (3.6 g, yield 96%).

To a solution of compound 3c (3.6 g, 16 mmol) in anhydrous DCM (80 mL) was added oxalyl chloride (2.7 g, 21 mmol) at 0° C., and followed by DMF (two drops) at 0° C. The mixture was stirred for 15 min at 0° C. and then stirred for 30 min at rt After completion of the reaction, the solvent was evaporated under reduced pressure to give a crude product. To a solution of the resulting product in anhydrous DCM (80 mL) was added ammonia (14 mL) and then the mixture was stirred for 12 hrs at rt After that, solids were filtered off, washed with DCM, and dried over vacuum freeze-drier to give a white solid, compound 3d which was used directly in next step (3.2 g, yield 91%). MS (ESI) m/z (M+H)⁺ 220.8.

A flask was charged with compound 3d (3.2 g, 14.6 mmol), Lawesson's reagent (3.0 g, 7.3 mmol) and anhydrous toluene (60 mL). The mixture was refluxed under nitrogen. After completion of the reaction, solids were filtered off and washed with EtOAc to give a yellow crude product compound 3, which was used directly in next step (2.14 g, yield 62%).

To a solution of compound 3 in EtOH was added compound 2. The mixture was refluxed under nitrogen. After removal of the solvent, the reaction mixture was diluted with EtOAc, washed with water and brine, dried over anhydrous Na₂SO₄, and then concentrated in vacuo. The residue was purified by column chromatography to give compound 4.

Compound 4 was dissolved in POCl₃ and then the mixture was refluxed under nitrogen. After the reaction completion, the reaction mixture was taken up with ice-water, neutralized with ammonia under cooling, extracted with EtOAc. The combined organic layer was washed with brine, dried over anhydrous Na₂SO₄ and concentrated to give a crude compound 5, which was used directly in next step

### 1.2 Preparation of Macrocycle (Compounds 101-113)

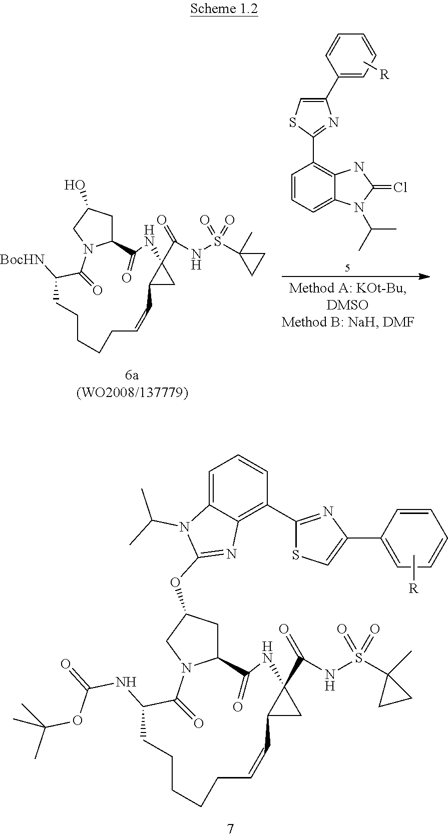

General formula 7 can be prepared from compound 6 as shown in Scheme 1.2 using either method A or method B. Compound 6a was synthesized according to the method described in WO2008/137779.

Method A: To a solution of compound 6 (1 eq.) in 2 mL of DMSO was added KOt-Bu (4.5 eq.) in portions at ambient temperature, then the mixture was stirred for 2 hrs at ambient temperature. After that, compound 5 (1.1 eq.) was added, the resulting mixture was stirred at rt for 20 hrs, the reaction was monitored by LCMS. The de-Boc product was detected, the mixture was cooled by ice water, acidified by aq. HCl (0.1 M) to pH=7-8. Then Boc₂O (1 eq.) and NaHCO₃ (1.5 eq.) were added. The mixture was stirred for another 2 hrs and then was acidified to pH=5˜6 with aq. HCl (0.1M), extracted with ethyl acetate, the organic layers were combined, washed by brine, dried over anhydrous sodium sulfate, concentrated under reduced pressure, the residue was purified by prep-TLC or prep-HPLC to provide general formula 7. Compounds 101-104 were synthesized using this method.

Method B: To a suspension of NaH (60% dispersion in mineral oil, 8 eq.) in DMF (2 mL) was added compound 6 (1 eq.) at 0° C. After stirring for 2 hrs at 0-5° C., compound 5 (1.2 eq) was added, the resulting mixture was warmed to room temperature and stirred for 12 hrs. After completion of the reaction, the mixture was cooled by ice water, acidified with aq HCl (0.1 M) to pH=5-6, then the mixture was extracted with ethyl acetate, the organic layers were combined, washed with brine, dried over anhydrous sodium sulfate, the solvent was removed under reduced pressure, the residue was purified by prep-TLC or prep-HPLC to give compound 7. Compounds 105-113 were synthesized using this method.

TABLE 1
Compounds 101-113 prepared according to Scheme 1.2.

| Compound | Structure | Method | Yield |
|---|---|---|---|
| 101 | 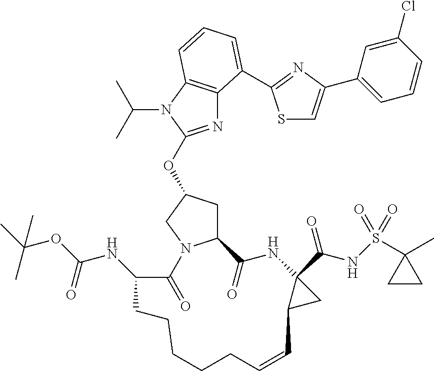 | A | 14 mg, yield 22%. MS (ESI) m/z (M + H)⁺ 934.4 |
| 102 | 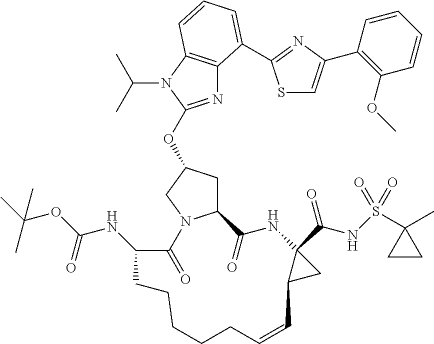 | A | 13 mg, yield 21%. MS (ESI) m/z (M + H)⁺ 930.5 |
| 103 | 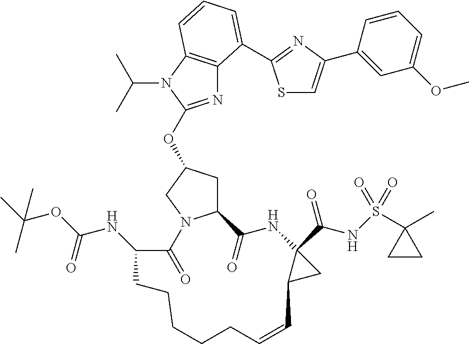 | A | 12 mg, yield 19%. MS (ESI) m/z (M + H)⁺ 930.4 |
| 104 | 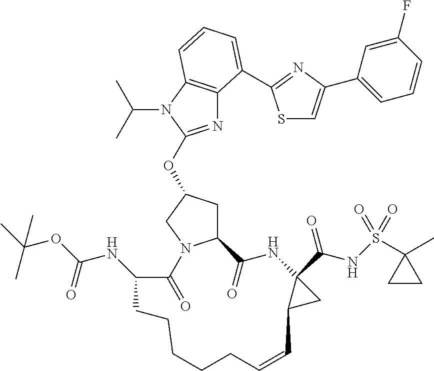 | A | 8 mg, yield 13%. MS (ESI) m/z (M + H)⁺ 918.5 |
| 105 | 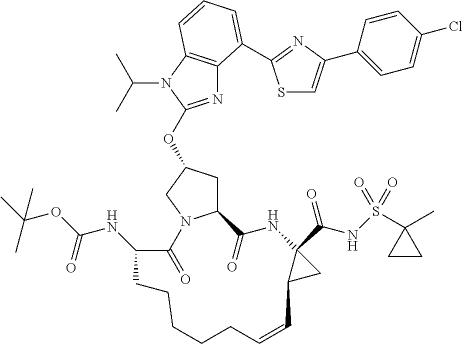 | B | 12 mg, yield 19%. MS (ESI) m/z (M + H)⁺ 918.5 |
| 106 | 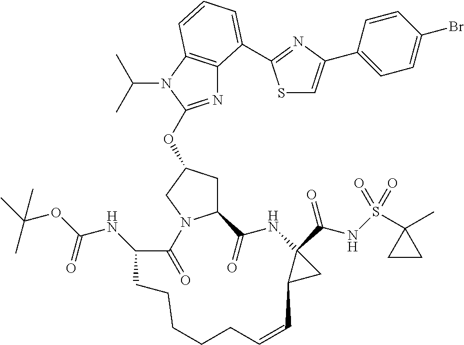 | B | 15 mg, yield 19%. MS (ESI) m/z (M + H)⁺ 980.3 |
| 107 | 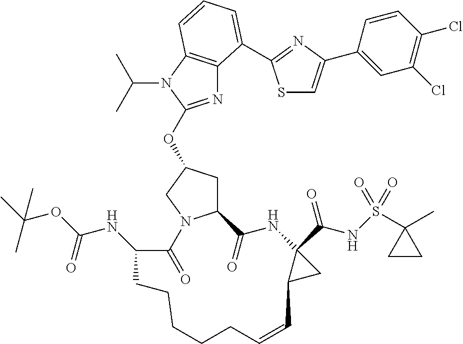 | B | 16 mg, yield 24%. MS (ESI) m/z (M + H)⁺ 968.4 |
| 108 | 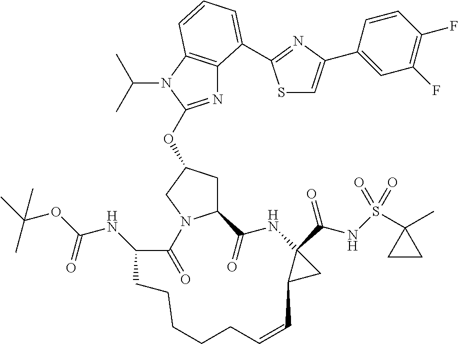 | B | 12 mg, yield 19%. MS (ESI) m/z (M + H)⁺ 936.3 |
| 109 | 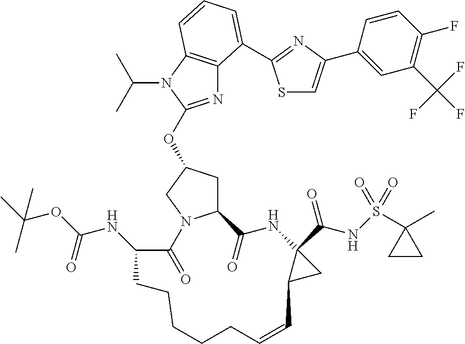 | B | 13 mg, yield 19%. MS (ESI) m/z (M + H)⁺ 986.2 |
| 110 | 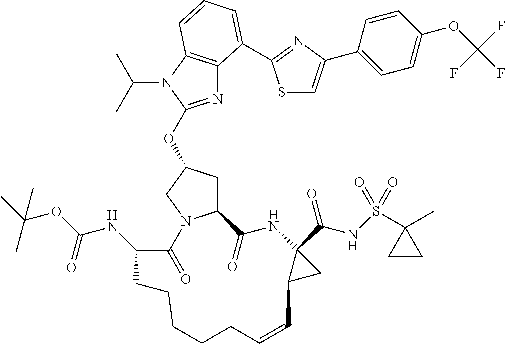 | B | 15 mg, yield 22%. MS (ESI) m/z (M + H)⁺ 984.4 |
| 111 | 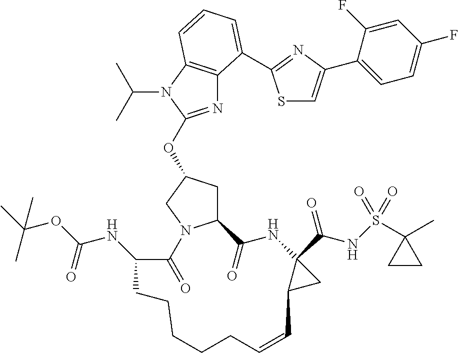 | B | 16 mg, yield 25%. MS (ESI) m/z (M + H)⁺ 936.4 |
| 112 | 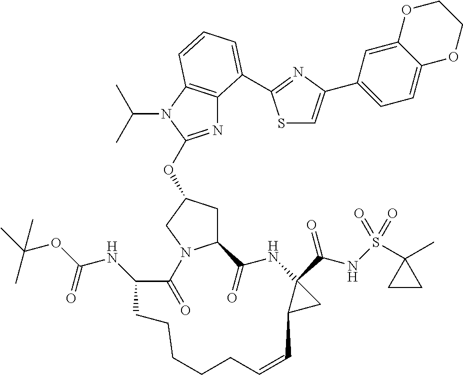 | B | 14 mg, yield 22%. MS (ESI) m/z (M + H)⁺ 958.4 |
| 113 | 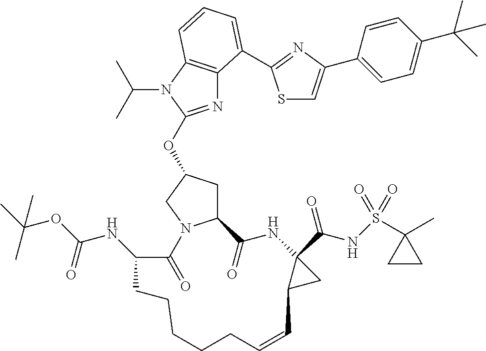 | B | 14 mg, yield 22%. MS (ESI) m/z (M + H)⁺ 956.5 |

### 1.3 Preparation of P2 Precursors—Substituted Benzyl-Thiazolyl Benzimidazole

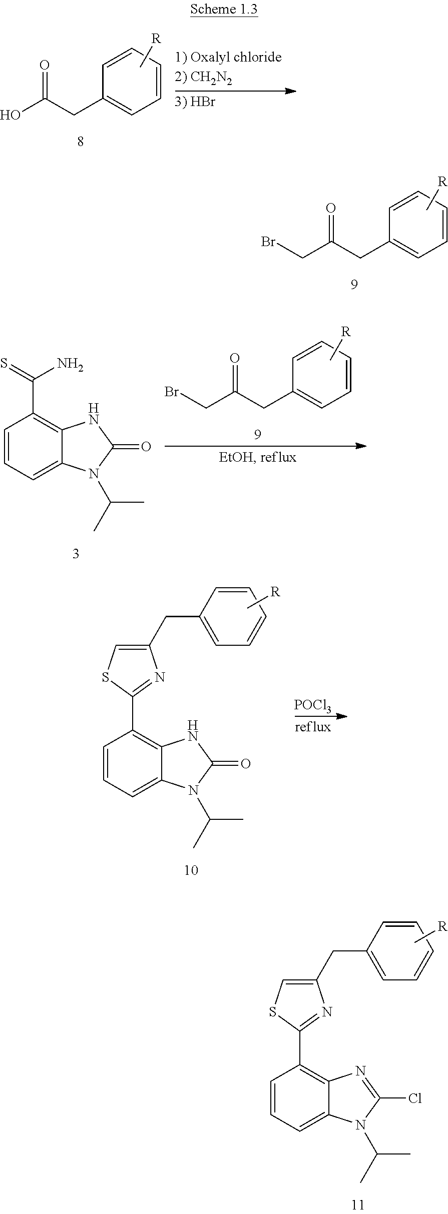

Substituted benzyl-thiazolyl benzimidazole precursors 11 were synthesized according to Scheme 1.3.

### 1.4 Preparation of Macrocycle (Compounds 114-124)

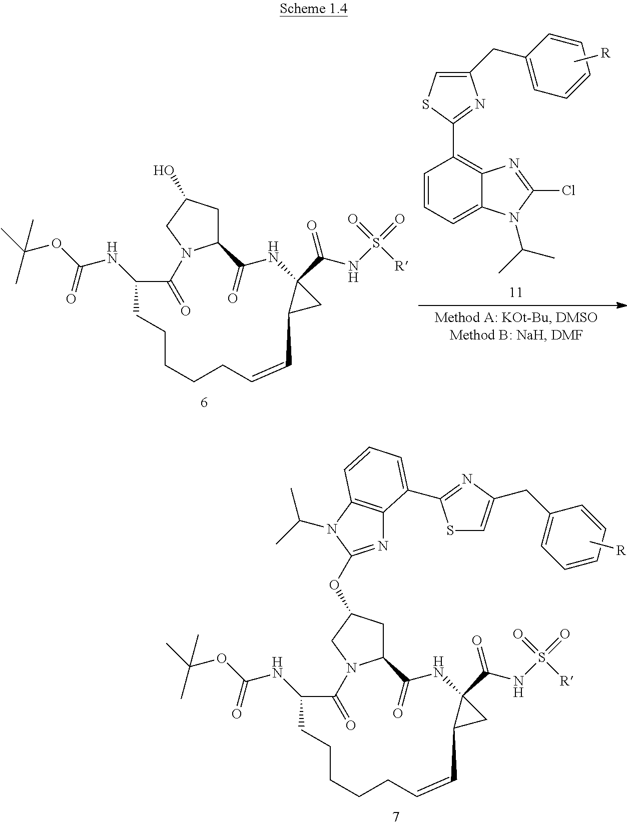

Formula 7 can be prepared using the same methods as described in Scheme 1.2 (methods A and B) using precursors 11. Compounds 114-121 were synthesized according to method A. Compounds 122-124 were made using method B.

TABLE 2
Compounds 114-124 prepared according to Scheme 1.4.

| Compound | Structure | Method | Yield |
|---|---|---|---|
| 114 | 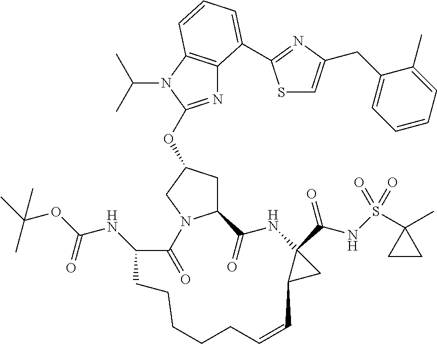 | A | 34 mg, yield 42%. MS (ESI) m/z (M + H)⁺ 928.4 |
| 115 | 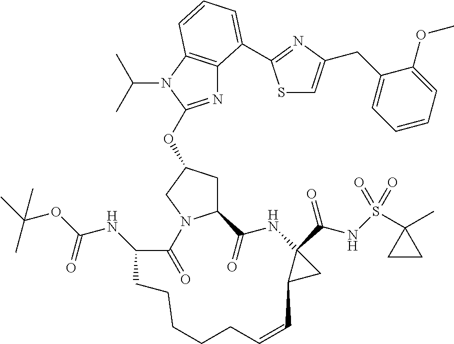 | A | 21 mg, yield 26%. MS (ESI) m/z (M + H)⁺ 944.5 |
| 116 | 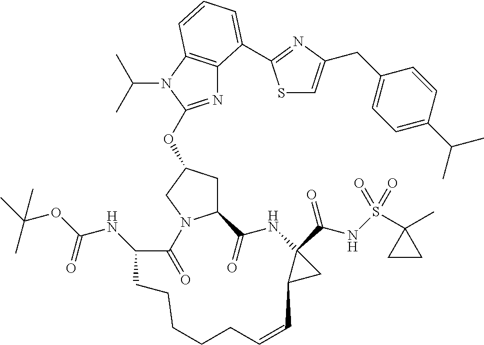 | A | 21 mg, yield 26%. MS (ESI) m/z (M + H)⁺ 956.4 |
| 117 | 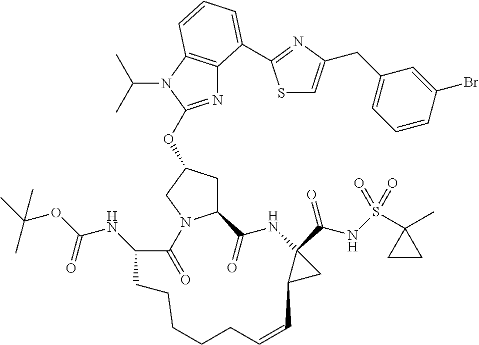 | A | 10 mg, yield 12%. MS (ESI) m/z (M + H)⁺ 991.9 |
| 118 | 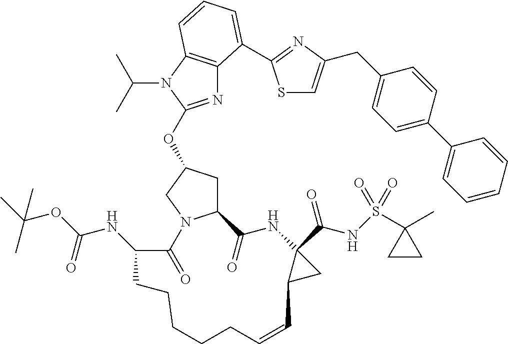 | A | 16.5 mg, yield 20%. MS (ESI) m/z (M + H)⁺ 990.6 |
| 119 | 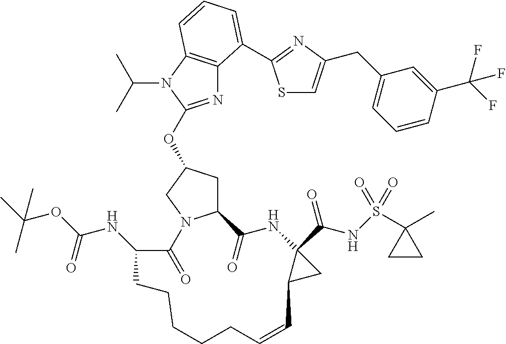 | A | 13 mg, yield 16%. MS (ESI) m/z (M + H)⁺ 982.5 |
| 120 | 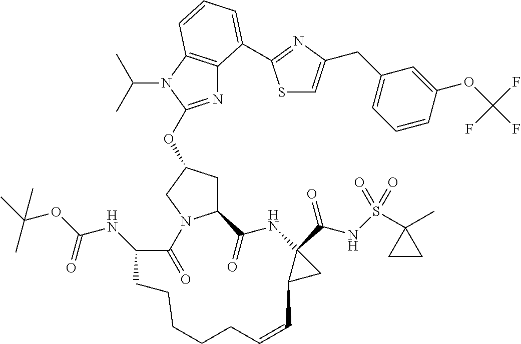 | A | 17 mg, yield 20%. MS (ESI) m/z (M + H)⁺ 998.5 |
| 121 | 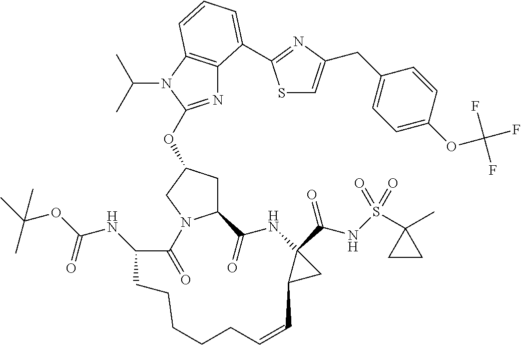 | A | 15.5 mg, yield 18%. MS (ESI) m/z (M + H)⁺ 998.7 |
| 122 | 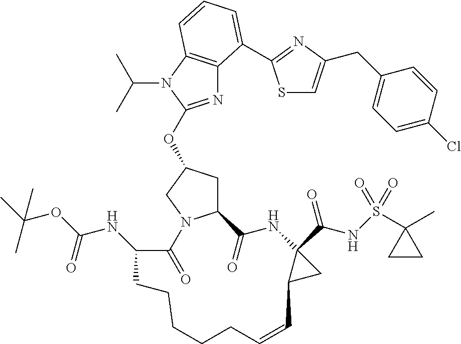 | B | 15.5 mg, yield 19%. MS (ESI) m/z (M + H)⁺ 948.3 |
| 123 | 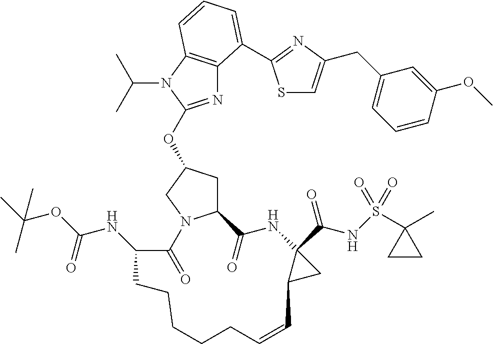 | B | 14 mg, yield 18%. MS (ESI) m/z (M + H)⁺ 944.3 |
| 124 | 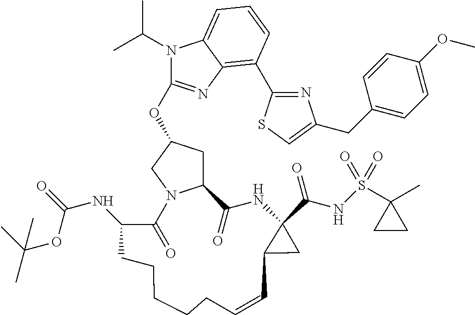 | B | 15.5 mg, yield 19%. MS (ESI) m/z (M + H)⁺ 944.5 |

### 1.5 Synthesis of Compound 125

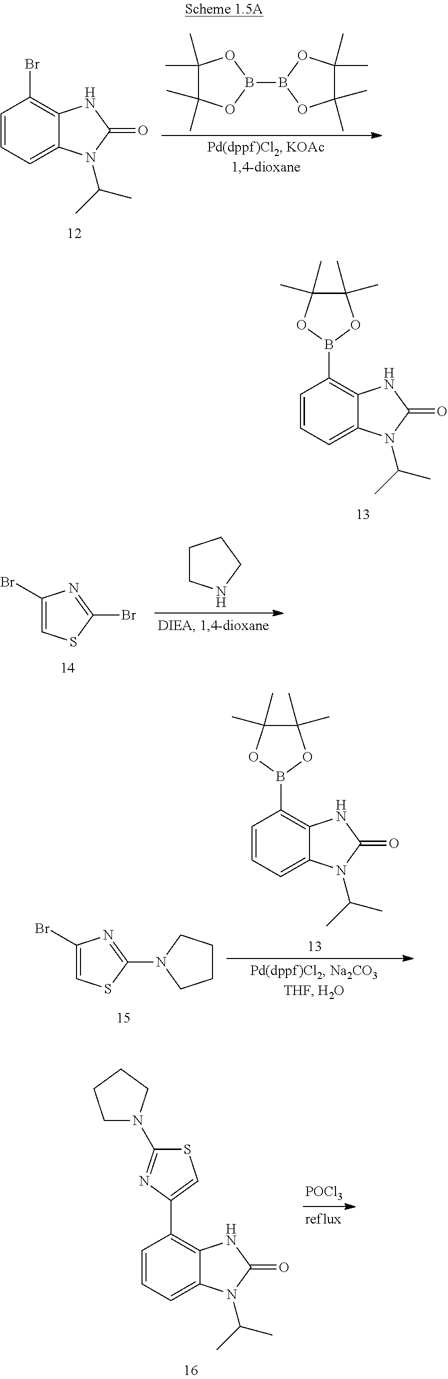

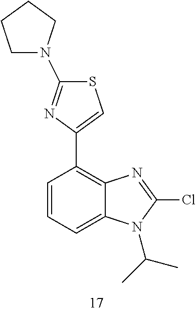

A flask was charged with compound 12 (1 g, 3.9 mmol), bis(pinacolato)diboron (1.99 g, 7.8 mmol), KOAc (1.2 g, 11.7 mmol) and 10 mL of 1,4-dioxane, the mixture was purged with nitrogen. After that, Pd(dppf)Cl₂ (100 mg, 0.12 mmol) was added. Then the mixture was heated to 90° C.-100° C., and stirred for 17 hrs. LCMS showed the reaction completed. All the volatiles were removed under reduced pressure. The residue was diluted with water, and extracted with ethyl acetate (80 mL×3), the organic layers were combined, washed by brine, dried over anhydrous sodium sulfate, concentrated under reduced pressure, the residue was purified by column chromatography on silica gel to give compound 13 (1.1 g, yield 91%). MS (ESI) m/z (M+H)⁺ 303.1. ¹H NMR (300 MHz, CDCl₃): δ 8.43 (s, 1H), 7.40 (d, J=7.2 Hz, 1H), 7.21 (d, J=7.2 Hz, 1H), 7.04 (t, 1H), 4.73 (m, 1H), 1.52 (d, J=6.9 Hz, 6H), 1.35 (s, 12H).

To a solution of 14 (250 mg, 1 mmol) in 8 mL of 1,4-dioxane was added pyrrolidine (210 mg, 3 mmol) and DIEA (260 mg, 2 mmol), the resulting solution was stirred at 105° C. for 1.5 hours. The reaction was monitored by LCMS. After completion of the reaction, the solvent was removed under reduced pressure, diluted with ethyl acetate (50 mL), washed with water and brine, filtered and concentrated. The crude product 15 was used directly in the next step without further purification (brown solid, 230 mg, yield 98%). MS (ESI) m/z (M+H)⁺ 234.9. ¹H NMR (300 MHz, CDCl₃): δ 6.31 (s, 1H), 3.44 (m, 4H), 2.04 (m, 4H).

To a solution of compound 15 (233 mg, 1 mmol) in THF (10 mL) and water (2 mL) was added Na₂CO₃ (212 mg, 2 mmoL), compound 13 (392 mg, 1.3 mmol), Pd(dppf)Cl₂ (73.5 mg, 0.1 mmol). The flask was purged with nitrogen for three times and the mixture was heated at reflux for 16 hrs. The reaction was monitored by LCMS. Then the mixture was cooled to room temperature, the solvent was removed under reduced pressure, and extracted with EtOAc (100 mL×2). The combined extracts was washed with brine, dried over anhydrous Na₂SO₄, filtered and concentrated under reduced pressure. The residue was purified by prep-TLC to give compound 16 as a light yellow solid (85 mg, yield 26%). MS (ESI) m/z (M+H)⁺ 329.1.

A flask was charged with compound 16 (85 mg, 0.26 mmol) and POCl₃ (2 mL). The mixture was heated under reflux for 16 hours. TLC showed the reaction was completed. After cooling to rt, the mixture was poured into ice-water. Neutralized by NaHCO₃ and extracted with EtOAc (30 mL×3). The combined organic layers were dried over sodium sulfate, filtered and concentrated in vacuo. The resulting residue was purified by prep-TLC to give compound 17 as a white solid (86 mg, yield 96%). (ESI) m/z (M+H)⁺ 346.8. ¹H NMR (400 MHz, CDCl₃): δ 8.07 (m, 1H), 7.94 (s, 1H), 7.39 (m, 1H), 7.29 (t, 1H), 4.93 (m, 1H), 3.55 (m, 4H), 2.05 (m, 4H), 1.67 (d, 6H).

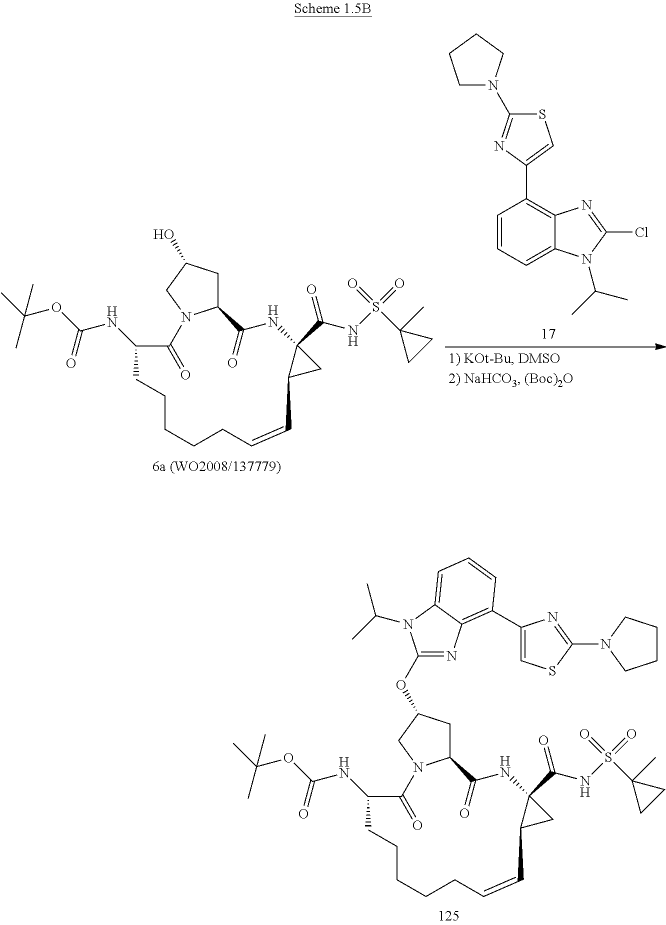

Compound 125 was synthesized using compound 17 according to method A as described in Scheme 1.2. (14 mg, yield 22%). MS (ESI) m/z [M+H]⁺ 893.5.

### 1.6 Synthesis of Compound 126

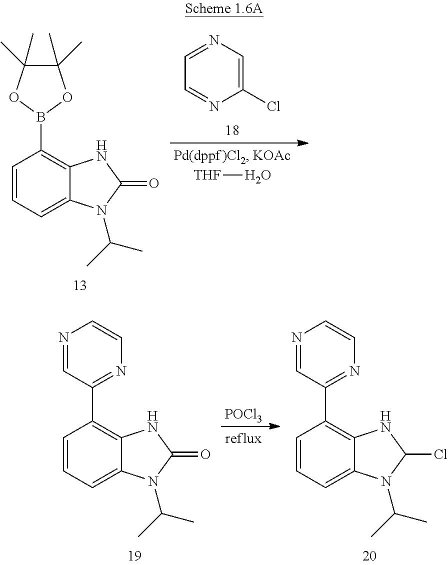

A flask was charged with compound 13 (500 mg, 1.65 mmol), compound 18 (210 mg, 1.82 mmol), Pd(dppf)Cl₂ (138 mg, 0.166 mmol), KOAc (195 mg, 1.984 mmol), THF (21 mL) and water (7 mL). The mixture was degassed with nitrogen and then stirred overnight at 100° C. under nitrogen. After that, the mixture was cooled down to r.t and filtered. The filtrate was taken into EtOAc (50 mL). Organic phase was washed with brine, dried over anhydrous Na₂SO₄ and concentrated in vacuo to give a crude product, which was purified by prep-TLC (PE:EtOAc=1:1) to yield compound 19 (162 mg, yield 39%). MS (ESI) m/z [M+H]⁺ 254.9. Compound 20 was prepared according to the same method for preparing compound 17 in Scheme 1.5A. (159 mg, 92%). MS (ESI) m/z (M+H)⁺ 272.9.

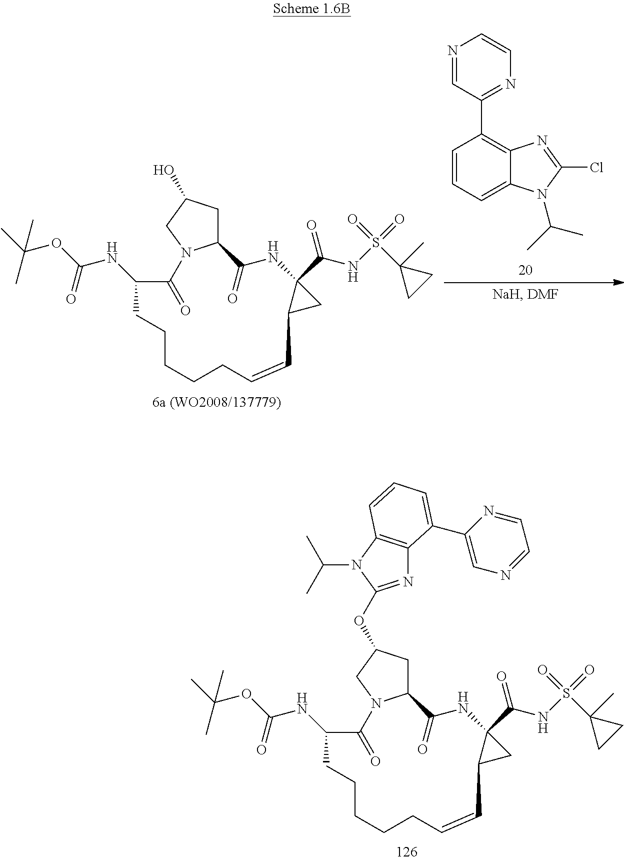

Compound 6a (50 mg, 0.086 mmol) was dissolved in DMF (3 mL) and the solution was degassed with nitrogen. Then NaH (60% dispersion in mineral oil, 8 eq) was added at 0° C. and the mixture was stirred for 1 h at 0° C. under nitrogen. Then compound 20 (30.5 mg, 0.086 mmol) was added and then the mixture was stirred at r.t under nitrogen. The reaction was monitored by LCMS. After completion of the reaction, the reaction was quenched with ice-water. Then the mixture was neutralized with 1M HCl (aq.) and extracted with EtOAc (30 mL×3). The combined organic layer was washed with brine and concentrated to give a crude product, which was purified by prep-HPLC to provide compound 126 (15.1 mg, yield 22%). MS (ESI) m/z (M+H)⁺ 819.5.

### 1.7 Synthesis of Compound 127

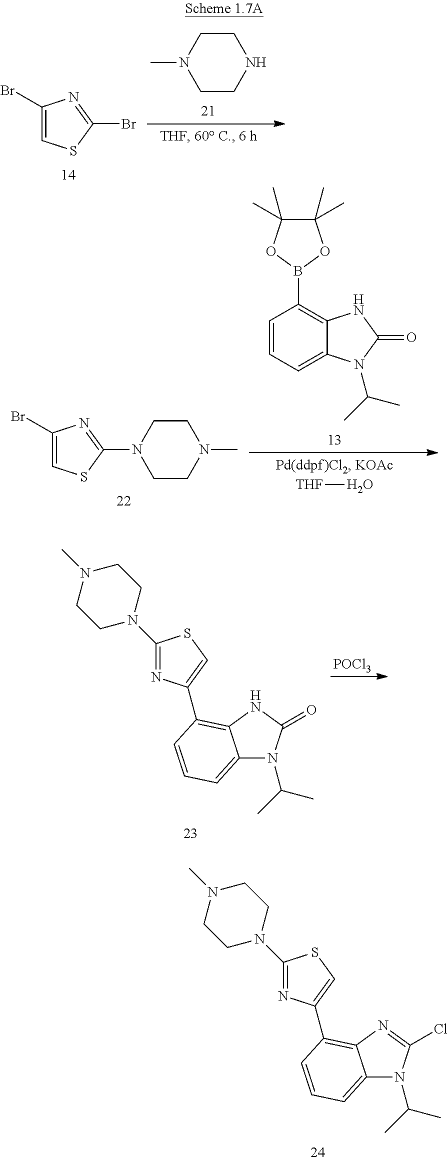

A flask was charged with compound 21 (205 mg, 2.1 mmol), compound 14 (200 mg, 0.82 mmol) and 5 mL of THF. The resulting mixture was stirred at 60° C. for 6 hrs. The reaction was monitored by LCMS. After completion of the reaction, the solvent was removed under reduced pressure, the residue was diluted with ethyl acetate, washed with water and brine, dried over anhydrous sodium sulfate, filtered and concentrated. The crude product was purified with prep-TLC to afford 22 as a light yellow solid (180 mg, yield 84%). MS (ESI) m/z [M+H]⁺ 263.9.

To a solution of compound 22 (180 mg, 0.68 mmol, 1 eq) in 6 mL of THF and 3 mL of water was added KOAc (202 mg, 2.1 mL, 3 eq), compound 13 (249 mg, 0.82 mmol, 1.2 eq), Pd(dppf)Cl₂ (57 mg, 0.07 mmol, 0.1 eq). The flask was purged with nitrogen and the mixture was heated at reflux for 16 hrs. The reaction was monitored by LCMS. Then the mixture was cooled to rt and extracted with EtOAc (50 mL×3), and washed with brine, the organic layer was dried over anhydrous Na₂SO₄, filtered and concentrated in vacuo. The residue was purified by prep-TLC to give compound 23 (60 mg, yield 24%). MS (ESI) m/z (M+H)⁺ 258.1. ¹H NMR (400 MHz, CDCl₃): δ 9.46 (s, 1H), 7.31 (m, 1H), 7.05 (m, 1H), 6.89 (s, 1H), 4.75 (m, 1H), 3.63 (m, 4H), 2.69 (m, 4H), 2.45 (s, 3H), 1.55 (d, 6H).

Compound 24 was prepared according to the same method for preparing compound 17 in Scheme 1.5A. (60 mg, yield 95%).

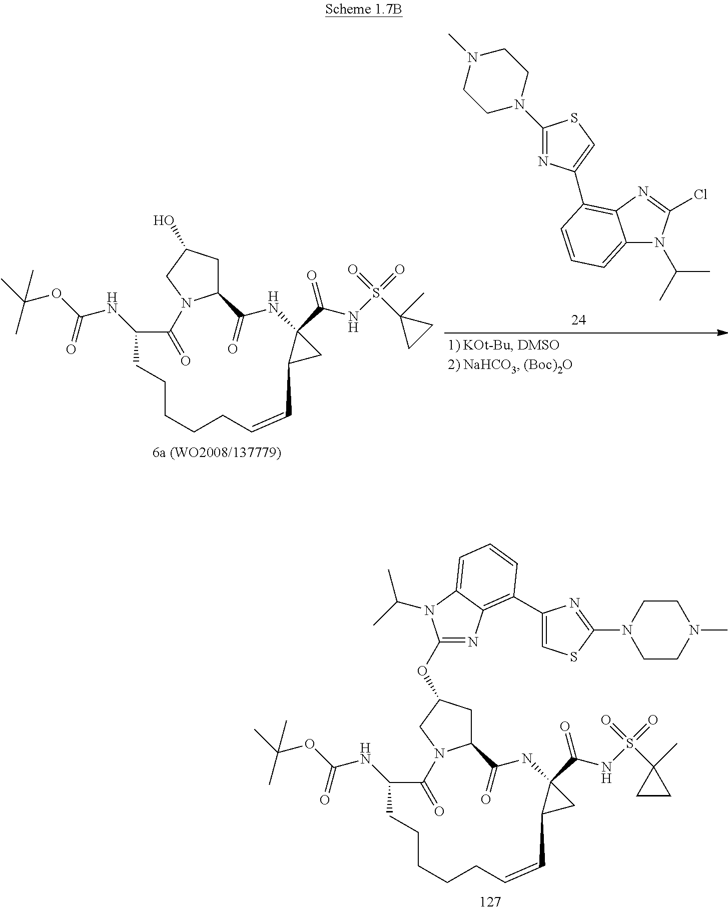

Compound 127 was synthesized using compound 24 according to method A as described in Scheme 1.2. (11.6 mg, yield 15%). MS (ESI) m/z (M+H)⁺ 922.5.

### 1.8 Synthesis of Compound 128

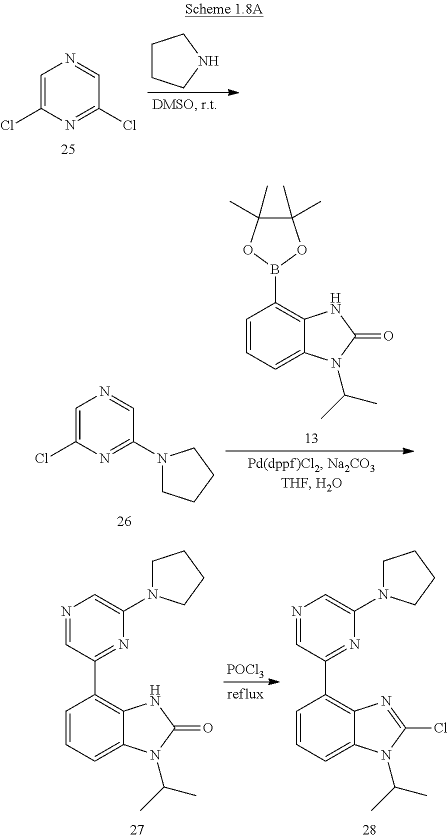

Compound 25 (300 mg, 2 mmol) and pyrrolidine were dissolved in DMSO (10 mL). The mixture was stirred at room temperature for 24 hours. The reaction was monitored by LCMS. After completion of the reaction, the mixture diluted with ethyl acetate (50 mL), washed with water and brine, dried over anhydrous sodium sulfate, filtered and concentrated. The crude product 26 was used directly in the next step without purification. (360 mg, yield 96%). MS (ESI) m/z (M+H)⁺ 183.7. ¹H NMR (300 MHz, CDCl₃): δ 7.72 (d, 2H), 3.48 (t, 4H), 2.02 (m, 4H).

To a solution of compound 26 (340 mg, 1.86 mmol) in THF (15 mL) and water (3 mL) was added Na₂CO₃ (394 mg, 3.72 mmol), compound 13 (673 mg, 2.2 mmol), Pd(dppf)Cl₂ (183 mg, 0.186 mmol). The flask was purged with nitrogen and the mixture was heated at reflux for 16 hrs. The reaction was monitored by LCMS. Then the mixture was cooled to rt and extracted with EtOAc (100 mL×2), and washed with brine, the organic layer was dried over anhydrous Na₂SO₄, filtered and concentrated in vacuo. The residue was purified by prep-TLC to give compound 27 (210 mg, yield 35%). MS (ESI) m/z (M+H)⁺ 324.1. ¹H NMR (300 MHz, CDCl₃): δ 9.94 (s, 1H), 8.39 (s, 1H), 7.81 (s, 1H), 7.57 (d, J=7.5 Hz, 1H), 7.12 (m, 2H), 4.75 (m, 1H), 3.60 (m, 4H), 2.11 (m, 4H), 1.55 (d, J=7.2 Hz, 6H).

Compound 28 was prepared according to the same method for preparing compound 17 in Scheme 1.5A. (60 mg, yield 95%).

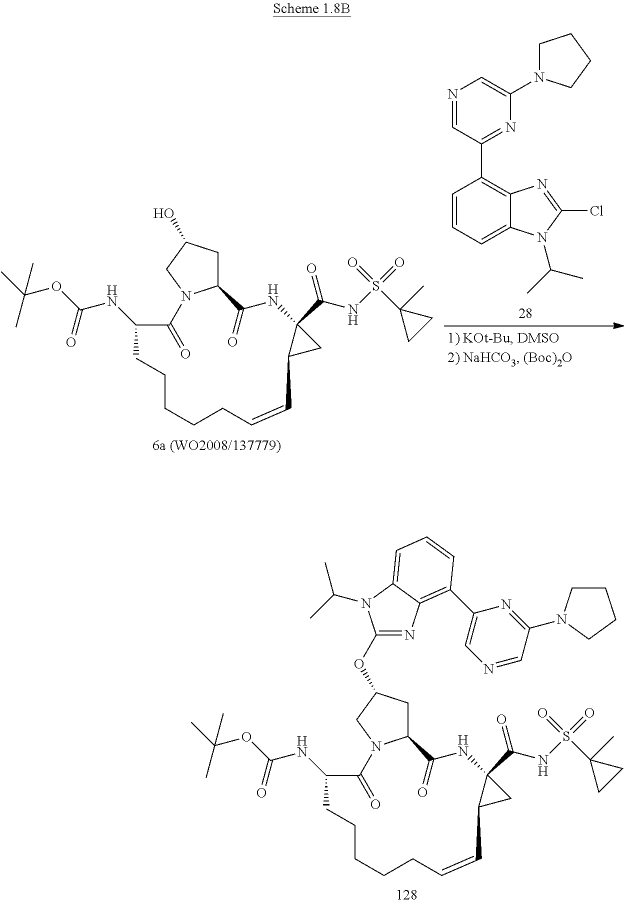

Compound 128 was synthesized using compound 28 according to method A as described in Scheme 1.2. (15.5 mg, yield 25%). MS (ESI) m/z [M+H]⁺ 888.6.

### 1.9 Synthesis of Compound 129

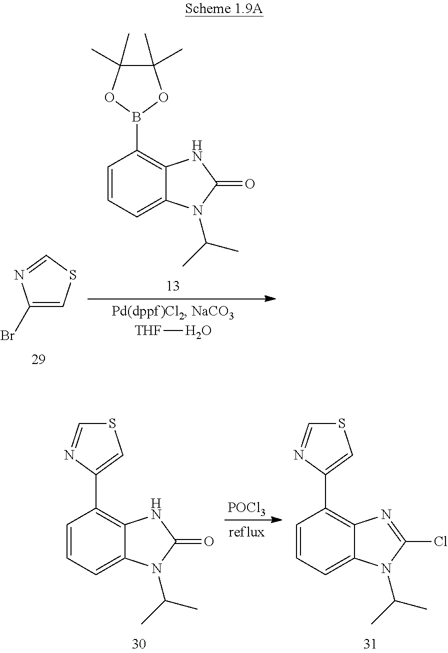

To a solution of compound 29 (400 mg, 2.47 mmol) in THF (15 mL) and water (3 mL) was added Na₂CO₃ (523 mg, 4.94 mmol), compound 13 (600 mg, 2 mmol), Pd(dppf)Cl₂ (146 mg, 0.2 mmol). The flask was purged with nitrogen and the mixture was heated to reflux for 16 hrs. The reaction was monitored by LCMS. Then the mixture was cooled to rt and extracted with EtOAc (60 mL×3), and washed with brine, the organic layer was dried over anhydrous Na₂SO₄, filtered and concentrated in vacuo. The residue was purified by prep-TLC to give compound 30 (160 mg, yield 31%). ¹H NMR (300 MHz, CDCl₃): δ 10.36 (s, 1H), 9.24 (s, 1H), 8.19 (s, 1H), 7.55 (d, J=7.2 Hz, 1H), 7.26 (d, J=7.2 Hz, 1H), 7.07 (t, 1H), 4.60 (m, 1H), 1.44 (d, 6H).

Compound 31 was prepared according to the same method for preparing compound 17 in Scheme 1.5A. (70 mg, yield 67%).

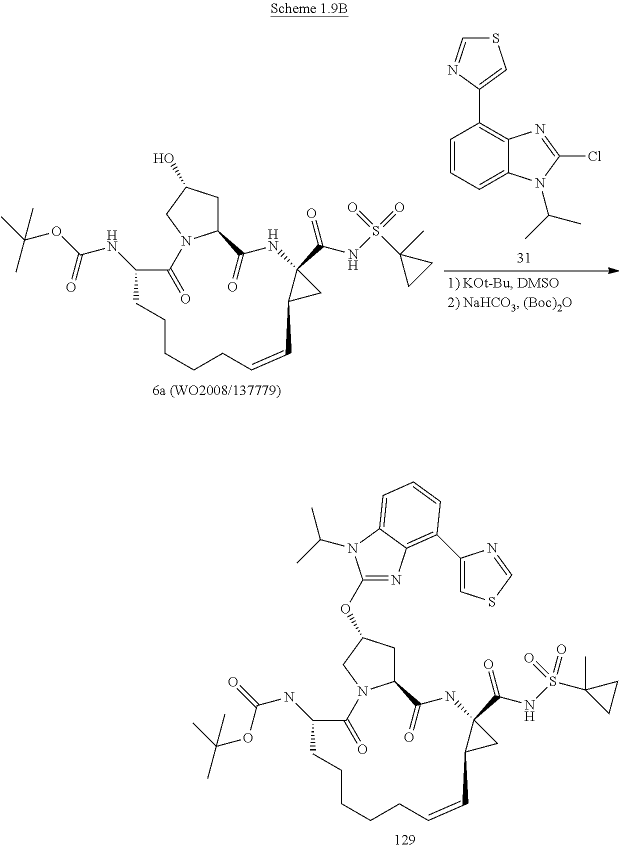

Compound 129 was synthesized from compound 31 according to method A as described in Scheme 1.2. (42 mg, yield 39%). MS (ESI) m/z (M+H)⁺ 824.4.

### 1.10 Preparation of Macrocycle (Compounds 130-143)

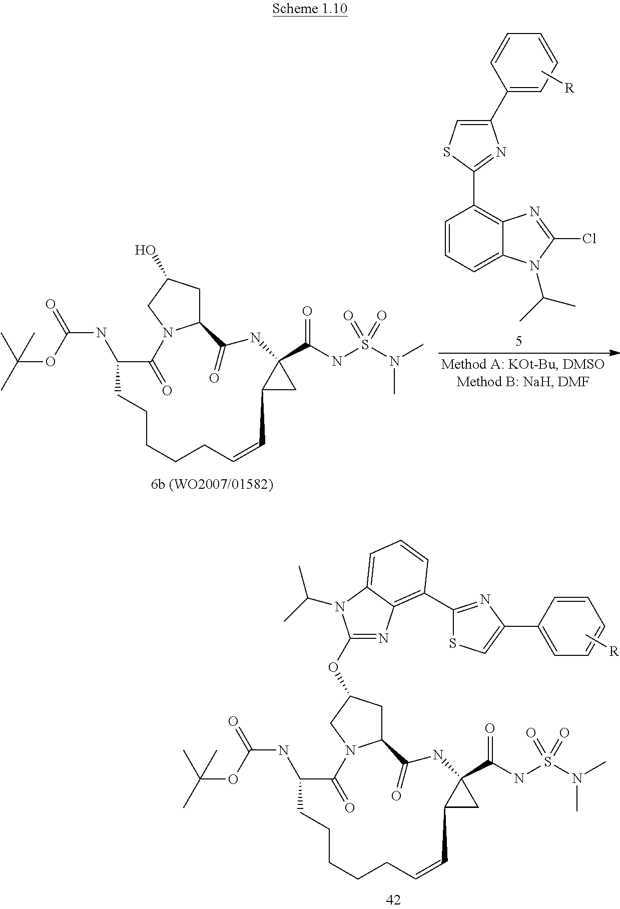

Compound 6a was synthesized according to the method described in WO2007/01582. Formula 42 can be prepared using the same methods as described in Scheme 1.2 (methods A and B) using precursors 5. Compounds 130-134 were synthesized according to method A. Compounds 135-143 were made using method B.

TABLE 3
Compound 130-143 prepared according to Scheme 1.10.

| Compound | Structure | Method | Yield |
|---|---|---|---|
| 130 | 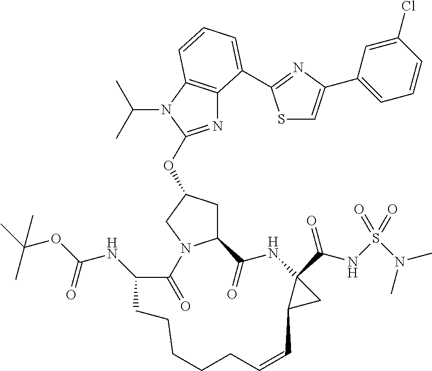 | A | 11 mg, yield 17%. MS (ESI) m/z (M + H)⁺ 923.3. |
| 131 | 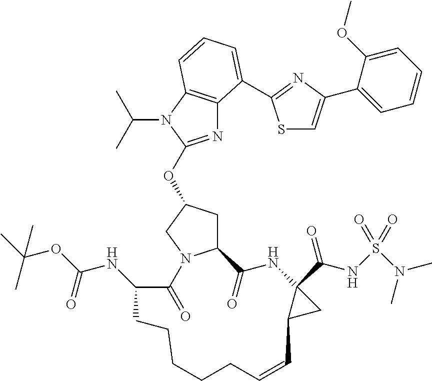 | A | 10 mg, yield 16%. MS (ESI) m/z (M + H)⁺ 919.4. |
| 132 | 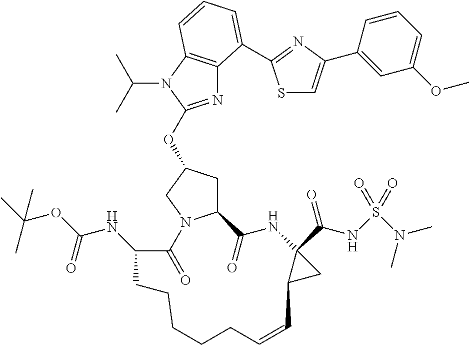 | A | 11.5 mg, yield 18%. MS (ESI) m/z (M + H)⁺ 919.5. |
| 133 | 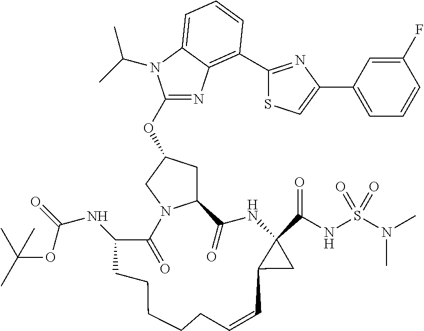 | A | 17 mg, yield 22%. MS (ESI) m/z (M + H)⁺ 907.5. |
| 134 | 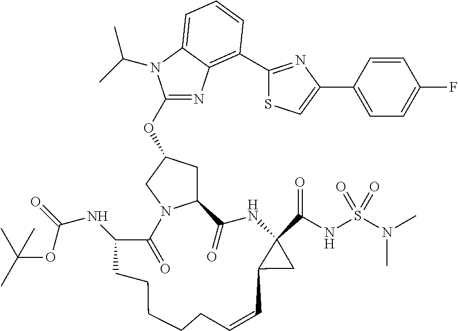 | A | 31 mg, yield 38%. MS (ESI) m/z (M + H)⁺ 907.4. |
| 135 | 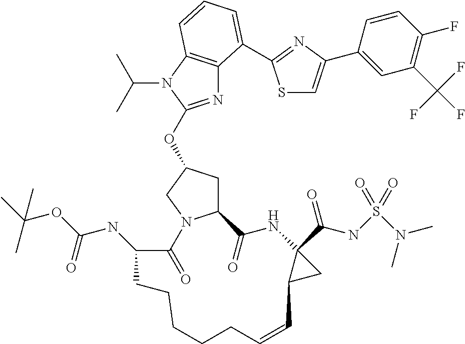 | B | 15.5 mg, yield 23%. MS (ESI) m/z (M + Na)⁺ 975.4. |
| 136 | 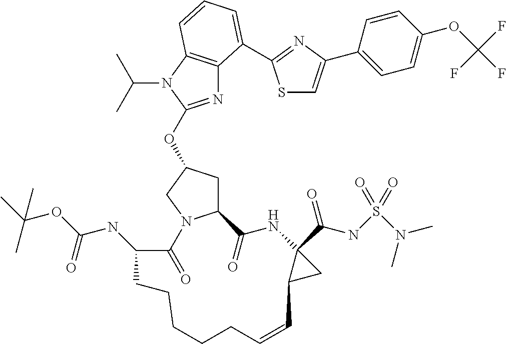 | B | 17 mg, yield 25%. MS (ESI) m/z (M + Na)⁺ 973.3. |
| 137 | 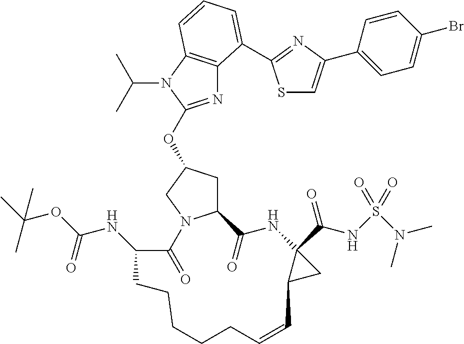 | B | 13 mg, yield 19%. MS (ESI) m/z (M + H)⁺ 969.3. |
| 138 | 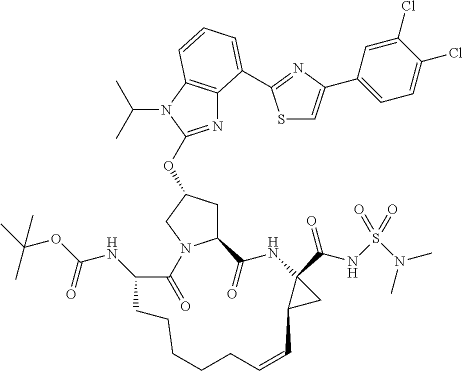 | B | 15 mg, yield 22%. MS (ESI) m/z (M + H)⁺ 957.4. |
| 139 | 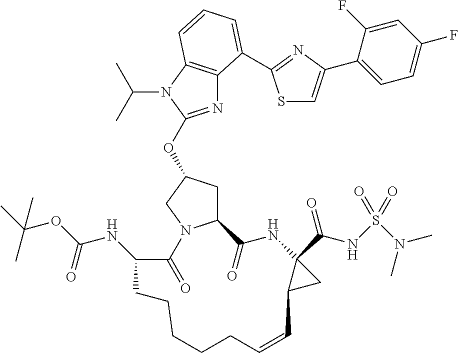 | B | 14 mg, yield 22%. MS (ESI) m/z (M + H)⁺ 925.5. |
| 140 | 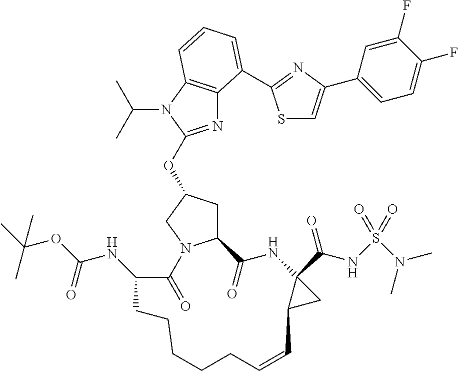 | B | 12 mg, yield 22%. MS (ESI) m/z (M + Na)⁺ 947.4. |
| 141 | 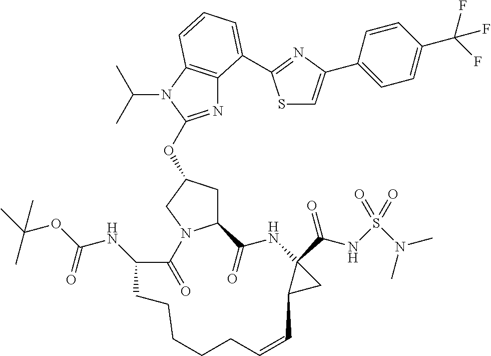 | B | 13 mg, yield 20%. MS (ESI) m/z (M + Na)⁺ 979.4. |
| 142 | 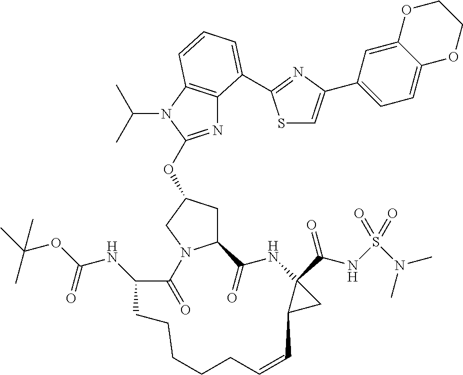 | B | 14 mg, yield 21%. MS (ESI) m/z (M + Na)⁺ 947.2. |
| 143 | 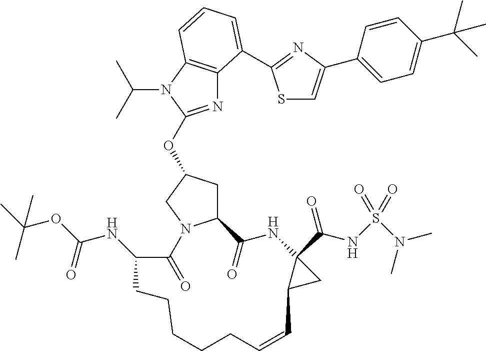 | B | 12 mg, yield 18%. MS (ESI) m/z (M + Na)⁺ 945.3. |

### 1.11 Preparation of Macrocycle (Compounds 144-164)

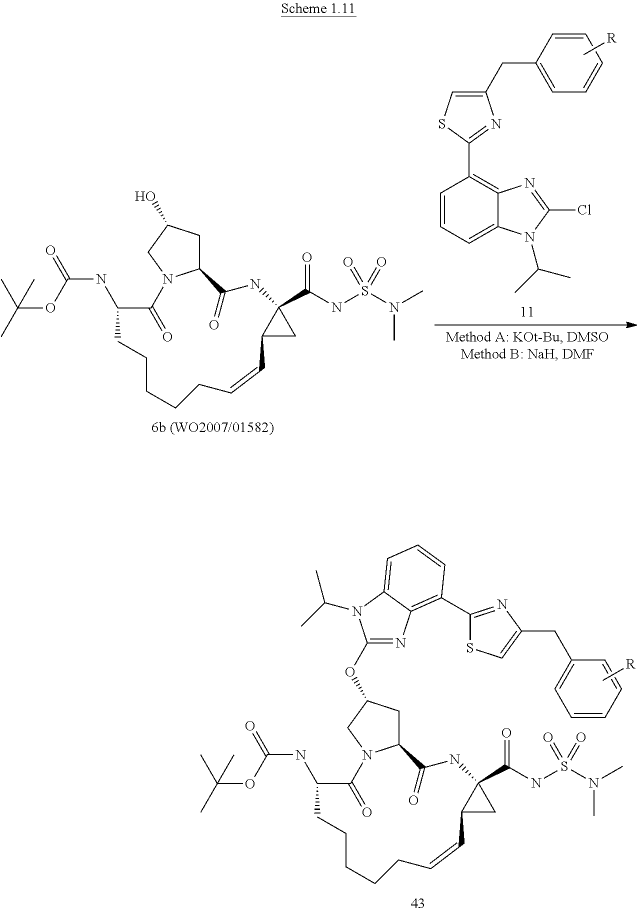

Formula 43 can be prepared using the same methods as described in Scheme 1.2 (methods A and B) using precursors 11. Compounds 144-155 were synthesized according to method A. Compounds 156-157 were made using method B.

TABLE 4
Compounds 144-157 prepared according to Scheme 1.11.

| Compound | Structure | Method | Yield |
|---|---|---|---|
| 144 | 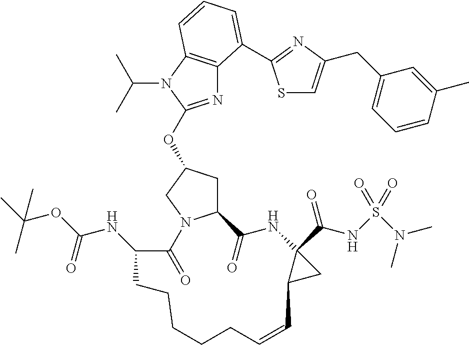 | A | 41 mg, yield 50%. MS (ESI) m/z (M + Na)⁺ 917.4. |
| 145 | 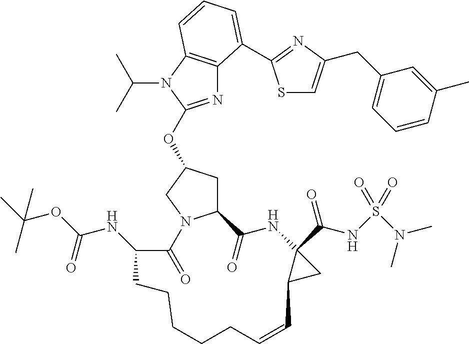 | A | 28.5 mg, yield 35%. MS (ESI) m/z (M + Na)⁺ 917.4. |
| 146 | 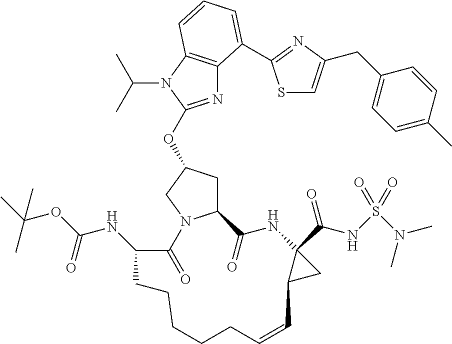 | A | 16.5 mg, yield 20%. MS (ESI) m/z (M + Na)⁺ 917.4. |
| 147 | 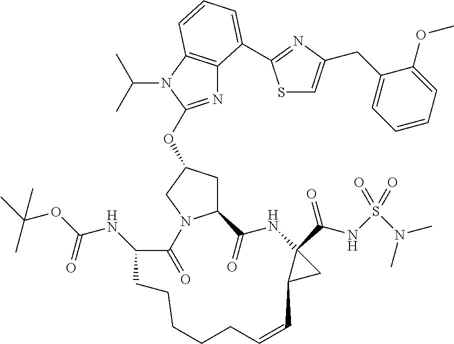 | A | 16.6 mg, yield 25%. MS (ESI) m/z (M + Na)⁺ 933.5. |
| 148 | 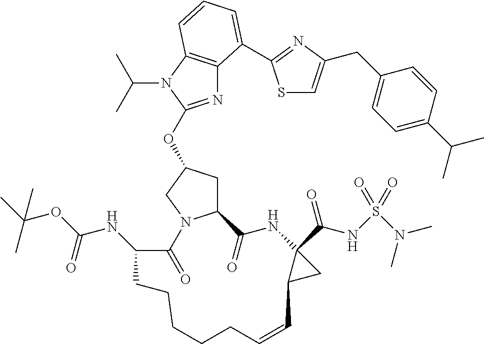 | A | 13.5 mg, yield 20%. MS (ESI) m/z (M + Na)⁺ 945.5. |
| 149 | 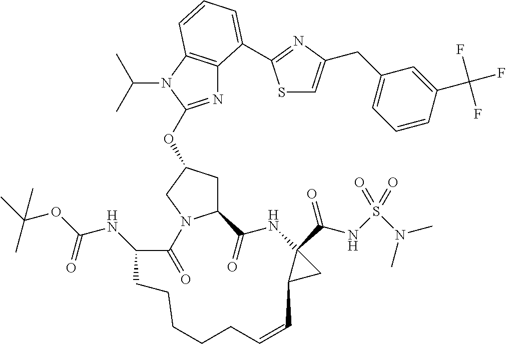 | A | 13 mg, yield 16%. MS (ESI) m/z (M + Na)⁺ 971.4. |
| 150 | 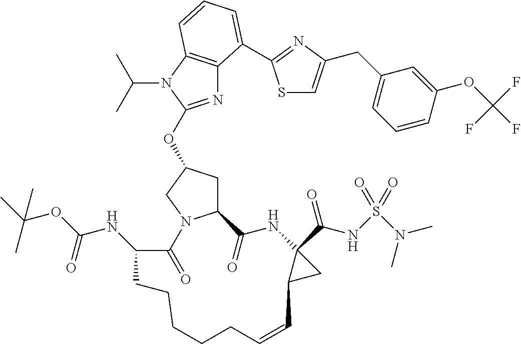 | A | 15.3 mg, yield 18%. MS (ESI) m/z (M + Na)⁺ 987.7. |
| 151 | 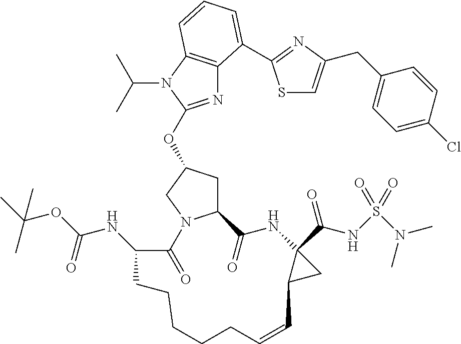 | A | 13.5 mg, yield 20%. MS (ESI) m/z (M + Na)⁺ 937.3. |
| 152 | 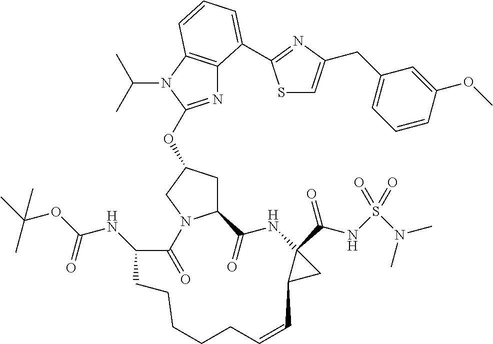 | A | 15 mg, yield 23%. MS (ESI) m/z (M + Na)⁺ 933.3. |
| 153 | 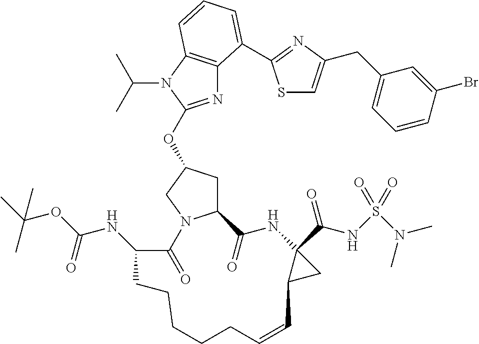 | A | 13 mg, yield 19%. MS (ESI) m/z (M + Na)⁺ 983.3. |
| 154 | 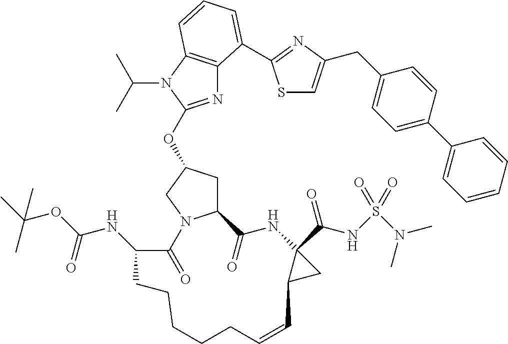 | A | 12 mg, yield 18%. MS (ESI) m/z (M + Na)⁺ 979.4. |
| 155 | 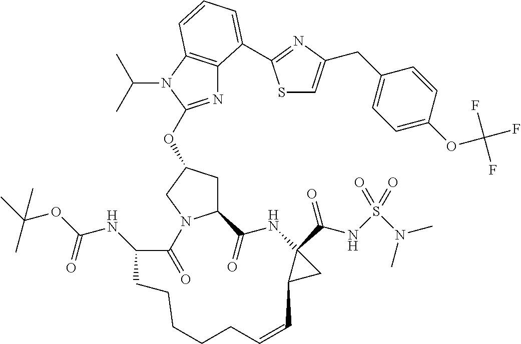 | A | 16.5 mg, yield 19%. MS (ESI) m/z (M + Na)⁺ 987.4. |
| 156 | 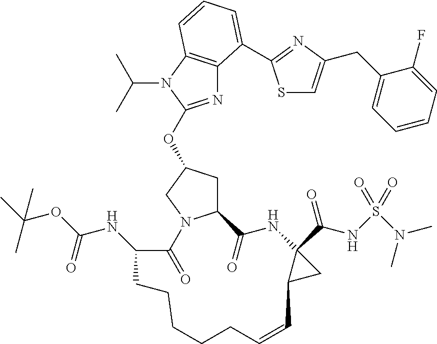 | B | 15 mg, yield 23%. MS (ESI) m/z (M + Na)⁺ 921.3. |
| 157 | 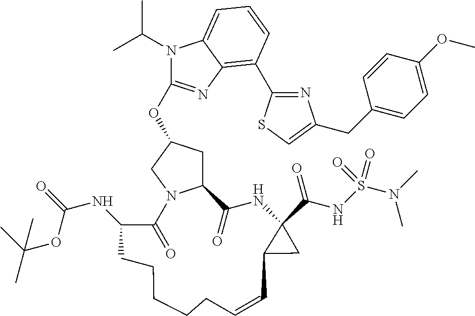 | B | 16 mg, yield 25%. MS (ESI) m/z (M + Na)⁺ 933.5. |

### 1.12 Synthesis of Compound 158

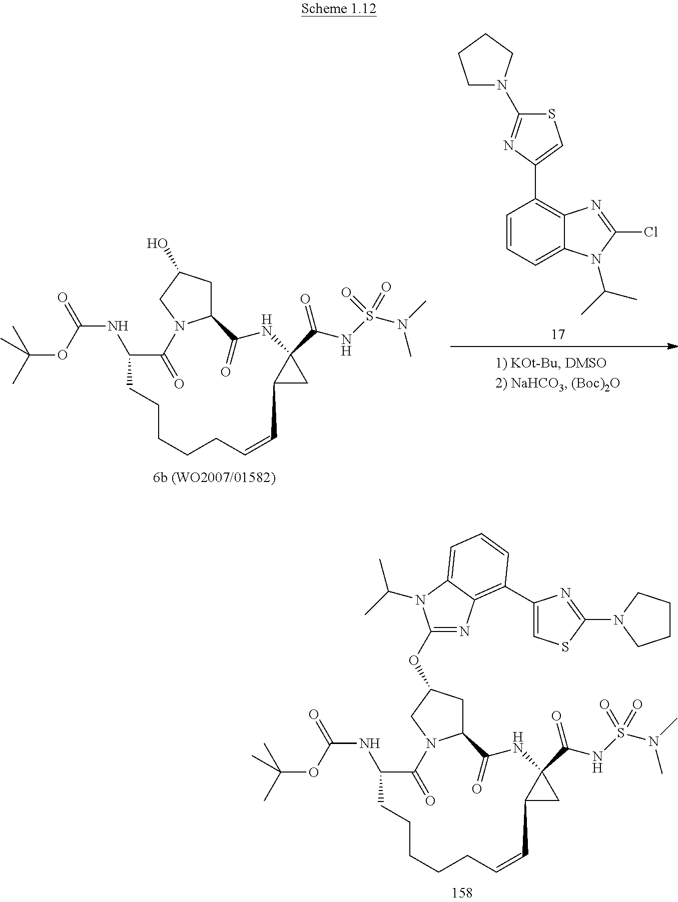

Compound 158 was synthesized from compound 6b according to method A as described in Scheme 1.2. (13 mg, 21%). MS (ESI) m/z (M+H)⁺ 882.5.

### 1.13 Synthesis of Compound 159

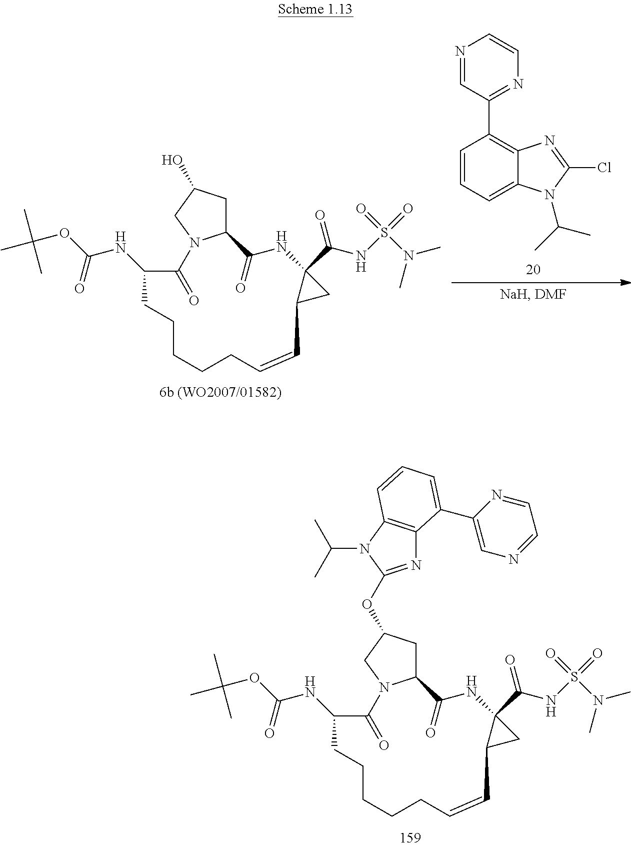

Compound 159 was synthesized from compound 6b according to Scheme 1.6B. (14.3 mg, 21%). MS (ESI) m/z (M+H)⁺ 808.5.

### 1.14 Synthesis of Compound 160

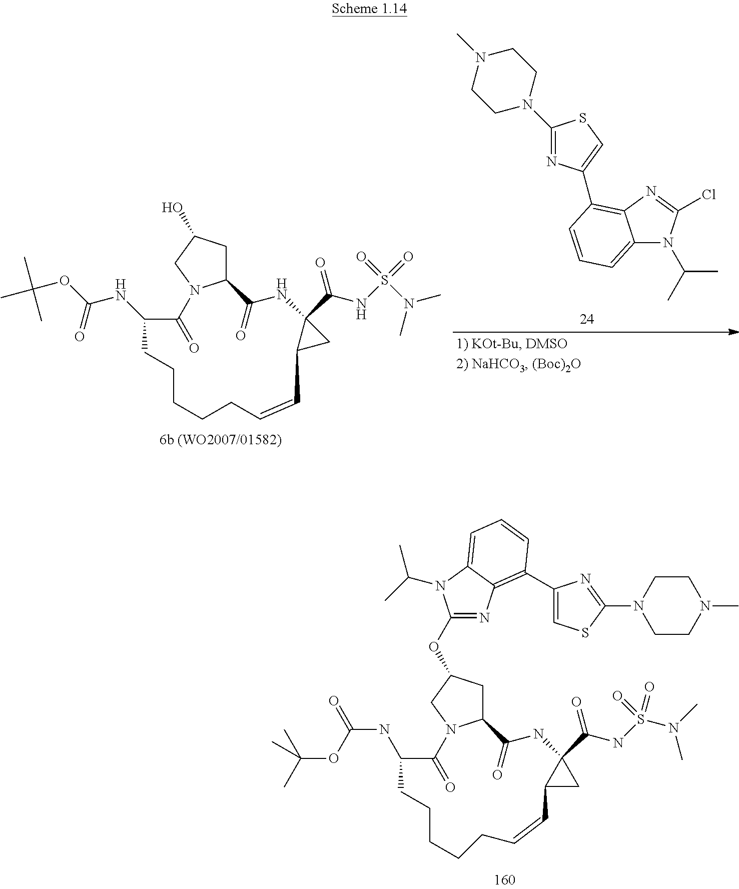

Compound 160 was synthesized using compound 24 according to method A as described in Scheme 1.2. (9.6 mg, yield 12%). MS (ESI) m/z (M+H)⁺ 911.1.

### 1.15 Synthesis of Compound 161

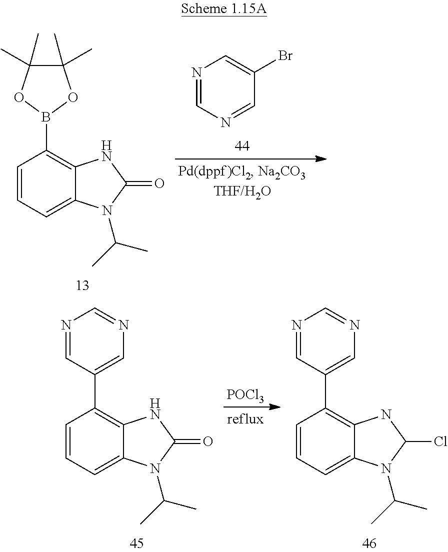

To a solution of compound 13 (287 mg, 0.95 mmol) in 3 mL of THF and 1 mL of water was added Na₂CO₃ (134 mg, 1.26 mmol), compound 44 (100 mg, 0.65 mmol), Pd(dppf)Cl₂ (78 mg, 0.095 mmol). The flask was purged with nitrogen and the mixture was heated at reflux for 16 hrs. The reaction was monitored by LCMS. Then the mixture was cooled down to rt and extracted with EtOAc (50 mL×3), and washed with brine, the organic layer was dried over anhydrous Na₂SO₄, filtered and concentrated in vacuo. The residue was purified by prep-TLC to give compound 45 (100 mg, yield 41%). MS (ESI) m/z (M+H)⁺ 255.2.

Compound 46 was prepared from compound 45 according to the same method for preparing compound 17 in Scheme 1.5A. (100 mg, yield 93%).

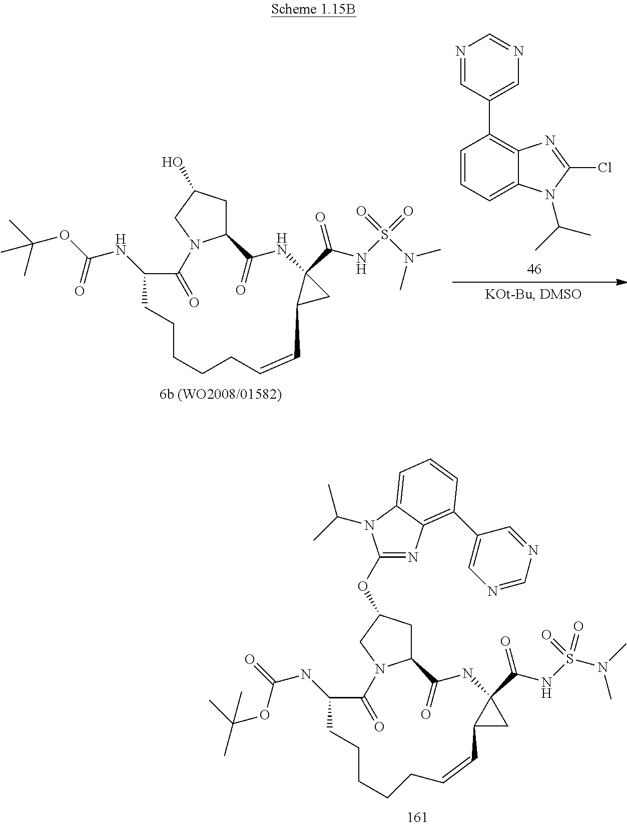

Compound 161 was synthesized using compound 46 according to method A as described in Scheme 1.2. (50 mg, yield 31%). MS (ESI) m/z (M+H)⁺ 808.5.

### 1.16 Synthesis of Compound 162

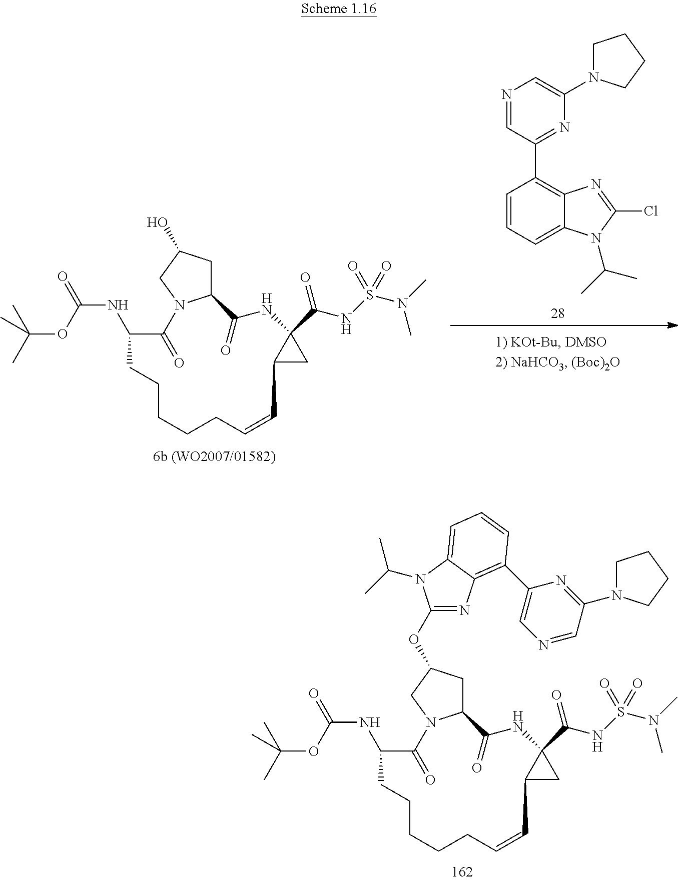

Compound 162 was synthesized using compound 28 according to method A as described in Scheme 1.2. (14 mg, yield 23%). MS (ESI) m/z [M+H]⁺ 877.6.

### 1.17 Synthesis of Compound 163

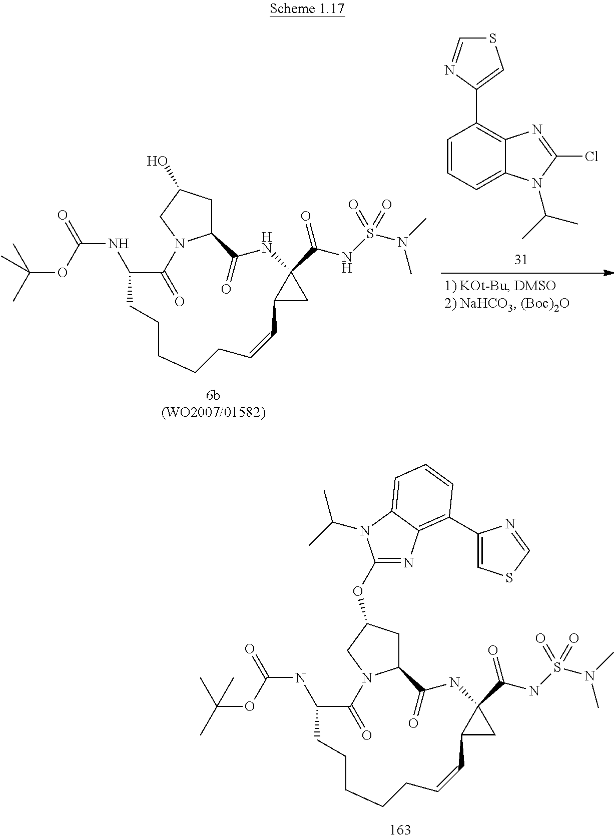

Compound 163 was synthesized using compound 31 according to method A as described in Scheme 1.2. (14.7 mg, yield 17%). MS (ESI) m/z [M+H]⁺ 813.4.

### 1.18 Synthesis of Compound 164

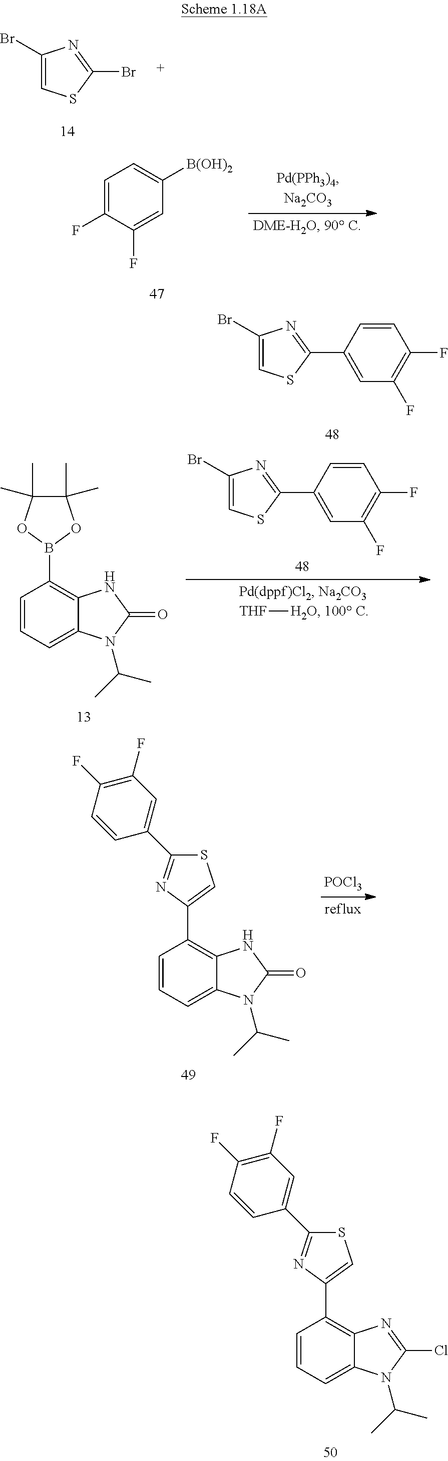

A flask was charged with compound 14 (1.1 g, 4.5 mmol), compound 47 (1.3 g, 8.23 mmol), Na₂CO₃ (1.1 g, 9.45 mmol), 10 mL of DME and 2 mL of water. The flask was purged with nitrogen. After that, Pd(PPh₃)₄ (280 mg, 0.246 mmol) was added. And then the mixture was heated to 90° C. and stirred for 17 hrs. LCMS showed the reaction completed. The solvent was evaporated. The residue was diluted with water, and extracted with ethyl acetate (100 mL×3), the organic layers were combined, washed by brine, dried over anhydrous sodium sulfate, concentrated under reduced pressure, the residue was purified by column chromatography on silica gel to give compound 48 (250 g, yield 21%).

A flask was charged with compound 13 (329 mg, 1.09 mmol), compound 48 (200 mg, 0.73 mol), Na₂CO₃ (153 mg, 1.44 mol) and Pd(dppf)Cl₂ (90 mg, 0.12 mmol) in 6 mL of THF and 1.5 mL of water. The flask was flushed with nitrogen. And then the mixture was heated to 100° C., and stirred for 17 hrs. LCMS showed the reaction completed. All the volatiles were removed under reduced pressure. The residue was diluted with water, and extracted with ethyl acetate (50 mL×3), the organic layers were combined, washed with brine, dried over anhydrous sodium sulfate, concentrated under reduced pressure, the residue was purified by column chromatography on silica gel to provide compound 49 (148 mg, yield 55%).

Compound 50 was prepared from compound 49 according to the same method for preparing compound 17 in Scheme 1.5A. (120 mg, yield 100%).

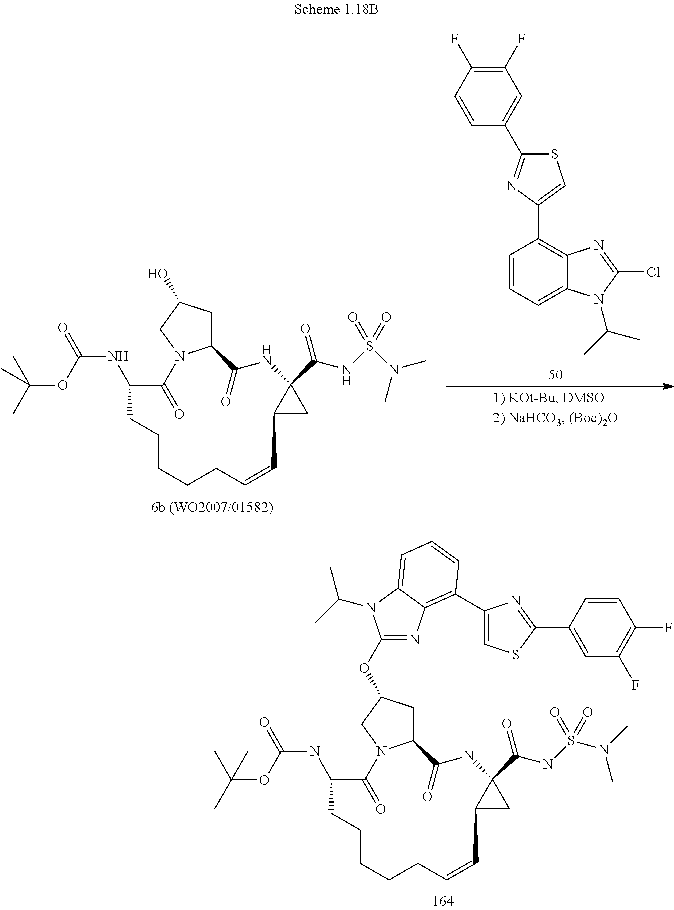

Compound 164 was synthesized using compound 50 according to method A as described in Scheme 1.2. (10.2 mg, yield 9%). MS (ESI) m/z [M+H]⁺ 925.4.

### 1.19 Synthesis of Compounds 165 and 166

### Scheme 1.19A

To a solution of compound 51 (1.0 g, 6.43 mmol) in 5 mL of DCM was added a solution of diazomethane in ether (0.7 M, 27.8 mL, 19.5 mmol) at −5° C., the solution was stirred at rt for 1 h, 8 mL of aq. HBr (40%) was added into the solution at −5° C., and then the mixture was stirred at r.t for 2 h. The reaction mixture was adjusted to pH=7 by addition of saturated aq. NaHCO₃, the organic layer was separated, dried over anhydrous Na₂SO₄ and concentrated under reduced pressure to afford compound 9a (1.3 g, yield 95%) as yellow solid. ¹H NMR (300 MHz, CDCl₃): δ 7.34 (m, 3H), 7.25 (m, 2H), 3.95 (s, 2H), 3.92 (s, 2H).

To a solution of compound 3 (650 mg, 2.74 mmol) in EtOH (15 mL) was added compound 9a (875 mg, 4.11 mmol). The mixture was refluxed under nitrogen. After completion of the reaction, the solvent was evaporated under reduced pressure to provide a crude product, which was purified by prep-TLC (Eluent: PE:EtOAc=3:1) to give compound 10a (412 mg, yield 80%). MS (ESI) m/z (M+H)⁺ 349.7.

Compound 10a (412 mg, 1.18 mmol) was dissolved in POCl₃ (10 mL) and then the mixture was refluxed under nitrogen. After completion of the reaction, the reaction mixture was taken into ice-water, neutralized with ammonia under cooling, extracted with EtOAc (30 mL×3), the organic phase was washed with brine, dried over anhydrous Na₂SO₄A. The combined organic layer was washed with brine, dried over anhydrous Na₂SO₄ and concentrated to give a crude product 11a, which was used directly in next step (425 mg, yield 98%). MS (ESI) m/z (M+H)⁺ 368.0.

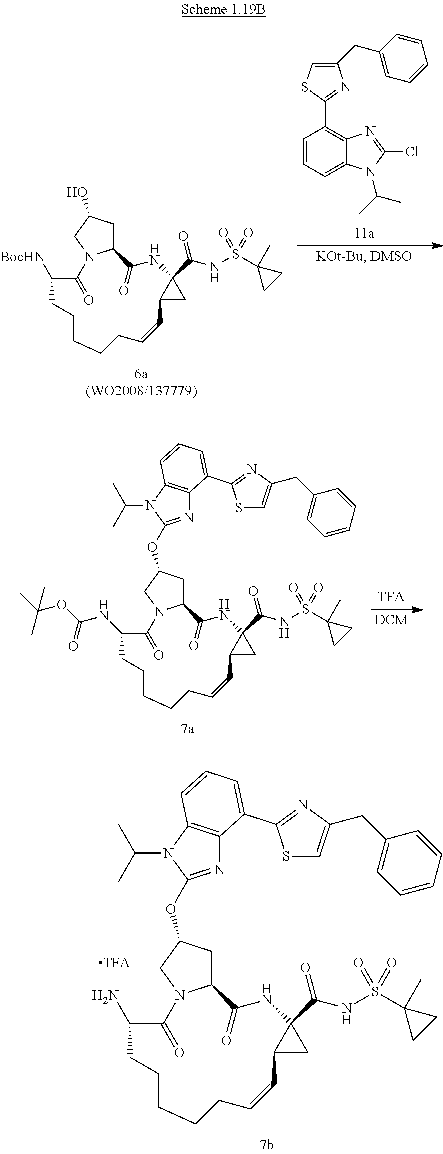

Compound 6a (560 mg, 0.97 mmol) was dissolved in DMSO (10 mL) and the solution was degassed with nitrogen. Then KOt-Bu (455 mg, 4.06 mmol) was added and the mixture was stirred for 1 h at rt under nitrogen. Then compound 11a (356 mg, 0.97 mmol) was added and the reaction mixture was stirred at rt under nitrogen. The reaction was monitored by LCMS. After completion of the reaction, the reaction was quenched with ice-water. Then the mixture was acidified to pH=6-7 with aq. HCl (0.1 M) and then MeOH (5 mL), NaHCO₃ (97.8 mg, 1.16 mmol), (Boc)₂O (212 mg, 0.97 mmol) were added. The mixture was stirred for 2 hrs at rt After that, MeOH was evaporated under reduced pressure and the resulting mixture was acidified o pH=5 with aq. HCl (0.1 M), and then it was extracted with EtOAc (40 mL×3). The combined organic layer was washed with brine, dried over anhydrous Na₂SO₄ and concentrated to give a crude product, which was purified by prep-TLC (Eluent: PE:EtOAc=1:1) to yield compound 7a (367 mg, yield 39%). MS (ESI) m/z (M+H)⁺ 914.2.

Compound 7a (367 mg, 0.41 mmol) was dissolved in DCM (10 mL) and TFA (1.5 mL). The mixture was stirred overnight at rt. After completion of the reaction, the solvents were removed under reduced pressure to afford compound 7b, which was used directly in the next step without purification.

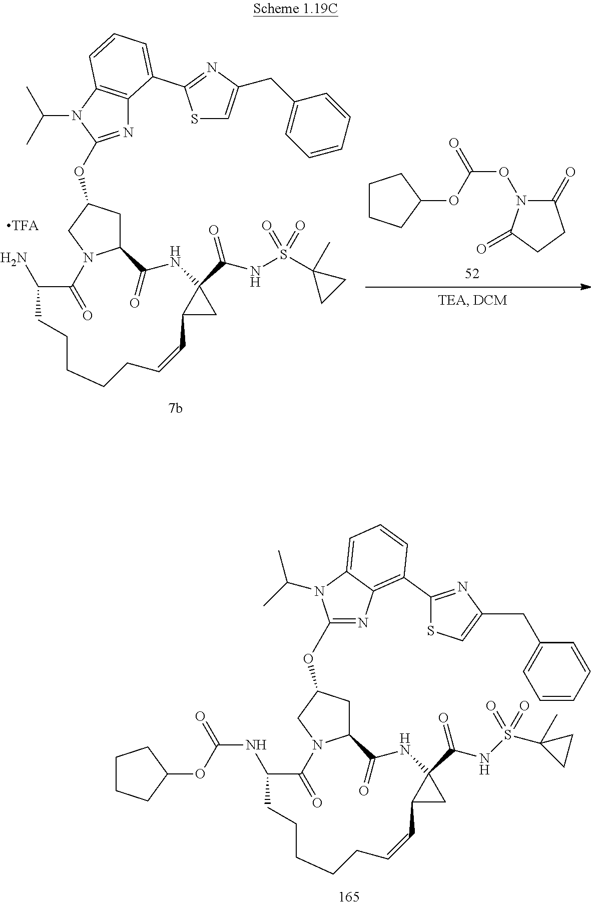

A flask was charged with compound 7b (160 mg, 0.17 mmol), compound 52 (66 mg, 0.29 mmol), TEA (0.08 mL, 0.57 mmol) and anhydrous DCM (4 mL). The resulting mixture was stirred at r.t under Nitrogen. The reaction was monitored by LCMS. After completion of the reaction, the solvent was removed under reduced pressure. The resulting residue was purified with prep-HPLC to provide compound 165 (88.1 mg, yield 56%). MS (ESI) m/z [M+H]⁺ 926.4.

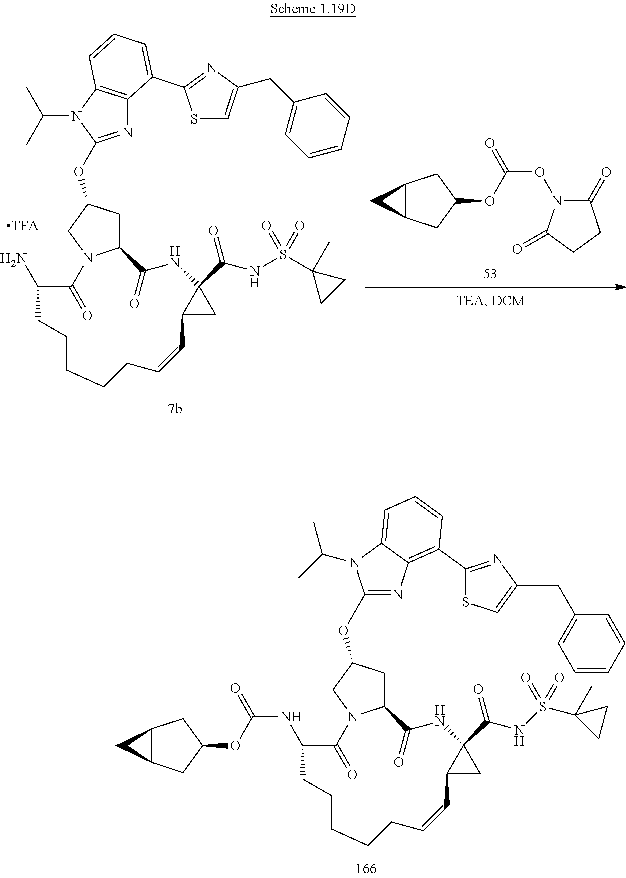

Compound 166 was prepared with a similar method used to prepare compound 165 above. (96.2 mg, yield 35%). MS (ESI) m/z [M+H]⁺ 938.4.

### 1.20 Synthesis of Compounds 167-168

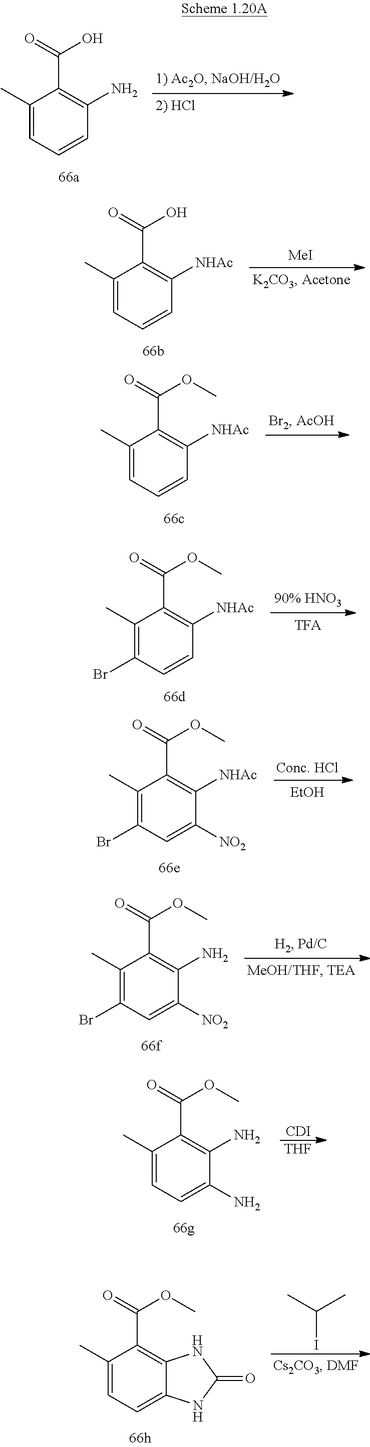

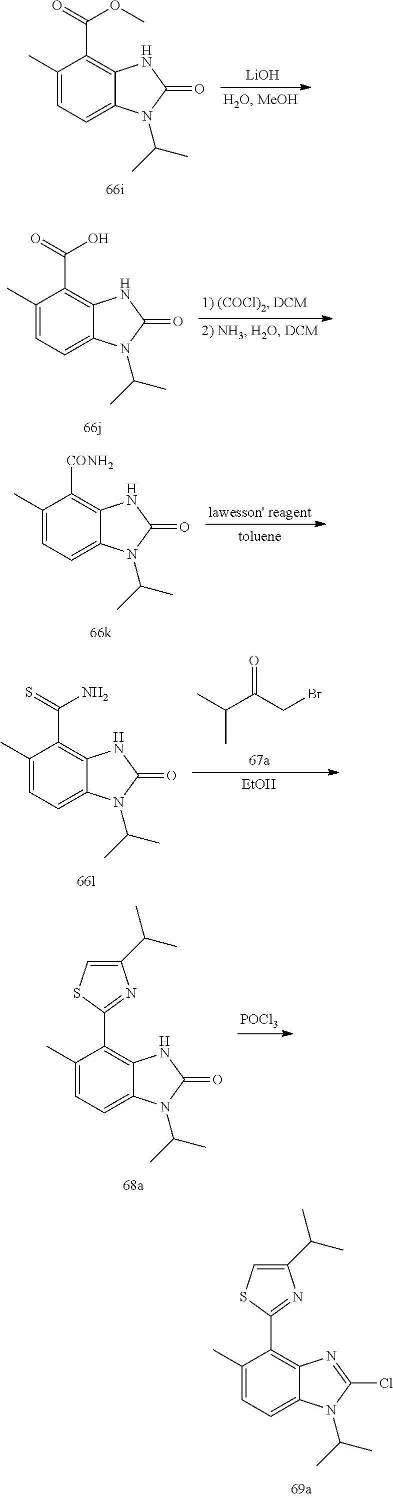

Compound 66a (1.0 g, 6 mmol) was added to a solution of NaOH (0.3 g, 7.5 mmol) in 50 mL of water and acetic anhydride were then added dropwise. After stirring for one hour, the mixture was acidified to pH=3 with conc. HCl with cooling, and the precipitate was filtered off with suction, washed with water and dried to obtain compound 66b (1.24 g, yield 94%).

A suspension of compound 66b (0.5 g, 2.59 mmol), anhydrous K₂CO₃ (0.43 g, 3.03 mmol) and iodomethane (1.46 g, 10.56 mmol) in acetone (15 mL) was refluxed for 15 hrs. The solvent was removed in vacuo and the residual slurry was diluted with water (20 mL) and extracted with EtOAc (30 mL×3). The combined extract was washed with water and brine, dried over anhydrous Na₂SO₄ and concentrated in vacuo to give compound 66c (0.47 g, yield 89%).

To a mixture of compound 66c (0.47 g, 2.26 mmol) in acetic acid (7 mL) was added a solution of Br₂ (0.3 mL, 4.5 mmol) in acetic acid (3 mL) dropwise. The resulting orange solution was allowed to stand overnight at room temperature. The orange suspension was diluted with water followed by aq. NaHSO₃ (10%) to remove the yellow color. The white solid was collected by filtration, washed with water (5 mL) and dried in vacuo to provide compound 66d (0.55 g, yield 85%).

To a stirred solution of compound 66d (0.52 g, 1.82 mmol) in TFA (10 mL) at 0° C., 90% HNO₃ (0.3 mL) was added dropwise. The solution was allowed to warm to room temperature and stirred overnight at room temperature. It was then poured into ice-water (20 mL) and the solid was collected by filtration, washed with water (10 mL) and dried in vacuo to afford compound 66e (0.5 g, yield 83%).

A mixture of compound 66e (0.2 g, 0.63 mmol) and concentrated HCl (2 mL) in EtOH (10 mL) was refluxed for 18 hrs. The solvent was removed in vacuo and the residue was dilute with water (10 mL). The mixture was adjusted with aq. NaOH (2 M) to pH=5 and the shiny yellow solid was collected by filtration, washed with water and dried under vacuum to afford compound 66f (0.16 g, yield 89%).

A mixture of compound 66f (0.45 g, 1.56 mmol), TEA (0.23 mL, 1.66 mmol), and Pa/C (0.09 g) in MeOH (20 mL) and THF (10 mL) was hydrogenated for 4 hrs under a pressure of 50 psi. The catalyst was filtered off (celite) and the filtrate was concentrated. The residue was partitioned between EtOAc and water and organic layer was washed with water, dried over anhydrous Na₂SO₄, and concentrated to provide compound 66g (0.25 g, yield 89%).

To a solution of compound 66g (0.2 g, 1.1 mmol) in anhydrous THF (10 mL) was added CDI (0.87 g, 4.4 mmol). The mixture was heated in microwave at 120° C. for 30 min. Aqueous HCl (2N, 5 mL) was added and the mixture was extracted with EtOAc (30 mL×3). Organic layer was washed with brine, dried over anhydrous Na₂SO₄ and concentrated to provide compound 66h (0.19 g, yield 84%).

A flask was charged with compound 66h (1.5 g, 7.2 mmol), Cs₂CO₃ (216 mg, 10.8 mmol) and DMF (150 mL). To the mixture was added 2-iodopropane (1.5 g, 8.7 mol) dropwise. The resulting mixture was stirred for 16 hrs at room temperature under nitrogen. The reaction was monitored by LCMS. After completion of the reaction, most of the solvent was removed under reduced pressure, the residue was purified by prep-TLC (PE/EA=4/1) to afford compound 66i (490 mg, yield 27%).

A flask was charged with compound 66i (490 mg, 2.0 mmol), NaOH (400 mg, 10 mmol), MeOH (30 mL) and H₂O (30 mL). The reaction mixture was stirred at 40° C. for 16 hrs. The reaction was monitored by LCMS. After completion of the reaction, most of the solvent was removed under reduced pressure, the residue was acidified with aq. HCl (1 M). The resulting solid was filtered and washed with water, dried under vacuum to afford compound 66j (430 mg, yield 91%).

To s solution of compound 66j (430 mg, 1.8 mmol) in and anhydrous DCM (10 mL) was added oxalyl chloride (0.19 mL, 2.2 mmol) dropwise at 0° C. The resulting solution was stirred at room temperature for 1 hour. Then the solvent was removed under reduced pressure, the residue was dissolved in anhydrous DCM (10 mL) and to the solution was added ammonia. Solid was formed. After stirred for 30 min, the solid was filtered and washed by water and dried to provide compound 66k (340 mg, yield 79%).

A flask was charged with compound 66k (340 mg, 1.45 mmol), Lawesson's reagent (360 mg, 0.87 mmol) and anhydrous THF (30 mL). The resulting mixture was heated to reflux and maintained at this temperature for 5 hrs. The reaction was monitored by LCMS. After completion of the reaction, the solvent was removed under reduced pressure, the crude product was purified by prep-TLC (PE/EA=2/1) to give compound 66l (190 mg, yield 52%).

A flask was charged with compound 66l (65 mg, 0.26 mmol), compound 67a (87 mg, 0.52 mmol) and EtOH (15 mL). The resulting mixture was heated to reflux and maintained at this temperature for 16 hrs. The reaction was monitored by LCMS. After completion of the reaction, the solvent was removed under reduced pressure, the crude product was purified by prep-TLC (PE/EA=3/1) to give compound 68a (55 mg, yield 67%).

A flask was charged with compound 68a (85 mg, 0.27 mmol) and POCl₃ (3 g). The resulting mixture was heated to reflux and maintained at this temperature for 4 hrs. The reaction was monitored by LCMS. After completion of the reaction, excess POCl₃ was removed under reduced pressure, the residue was diluted with ice-water, neutralized by saturated aq. NaHCO₃, extracted with EtOAc (20 mL×3). The combined organic layer was washed with brine, dried over anhydrous sodium sulfate, the solvent was removed under reduced pressure to afford compound 69a (80 mg, yield 88%), which was used directly in the next step without further purification.

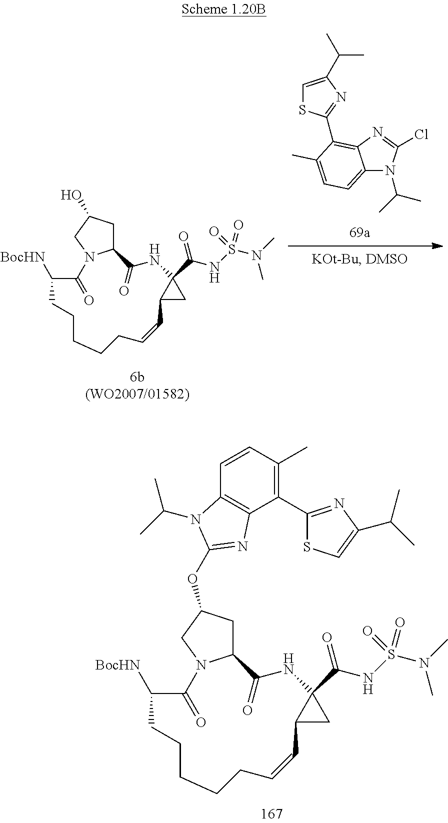

To a solution of compound 6b (137 mg 0.24 mmol) in DMSO (5 ml) was added KOt-Bu (134 mg 1.2 mmol) at room temperature. The mixture was stirred for 1 h at room temperature. Then, compound 69a (80 mg 0.24 mmol) was added. The resulting mixture was stirred for 12 hrs at room temperature. The reaction was monitored by LCMS. After completion of the reaction, the reaction was quenched by ice-water, acidified with aq. HCl (1 M) to pH=5-6. The resulting mixture was extracted with EtOAc (20 mL×3). The combined organic layer was washed with brine, dried over anhydrous sodium sulfate. The solvent was removed under reduced pressure. The crude product was purified by prep-HPLC to provide compound 167 (48.1 mg, yield 23%). MS (ESI) m/z (M+H)⁺ 869.5.

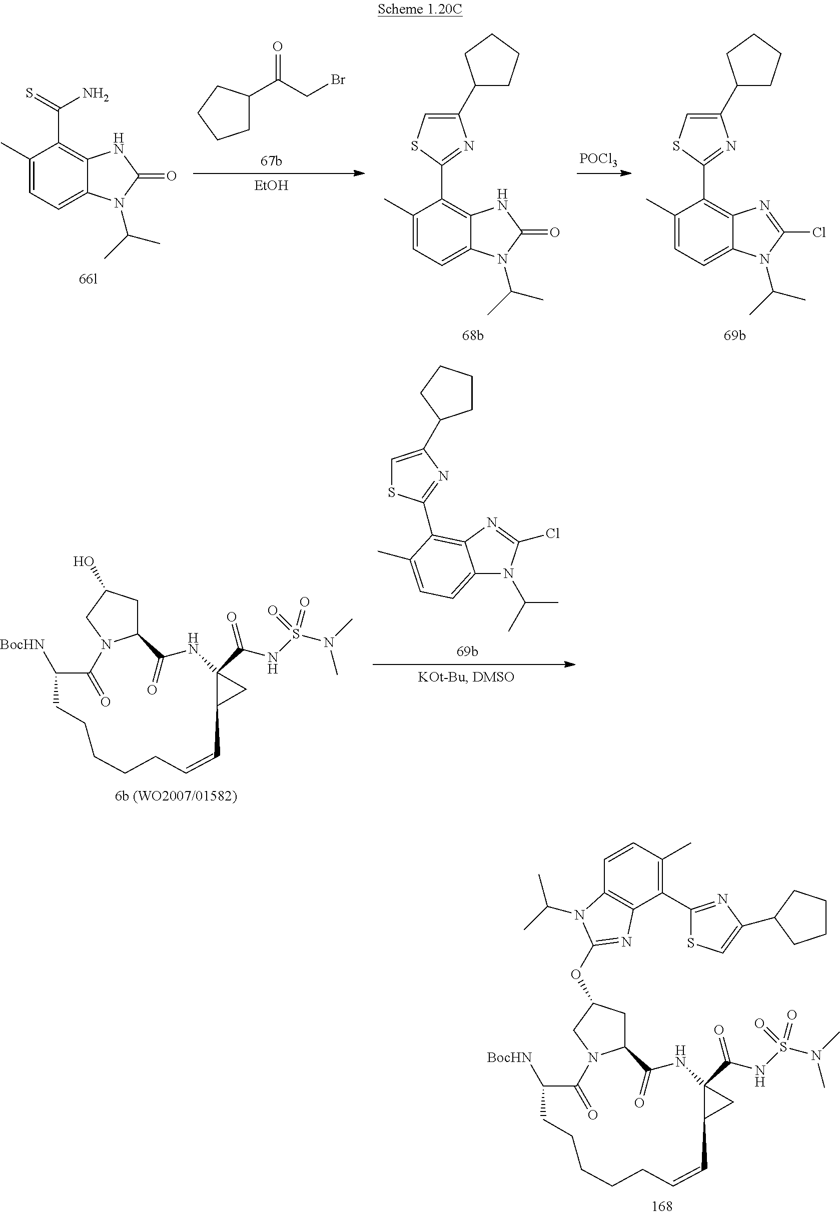

A flask was charged with compound 66l (190 mg, 0.76 mmol), compound 67b (297 mg, 1.52 mmol) and EtOH (30 mL). The resulting mixture was heated to reflux and maintained at this temperature for 4 hrs. The reaction was monitored by LCMS. After completion of the reaction, the solvent was removed under reduced pressure to give crude compound 68b (280 mg, 108%). The crude product was used directly in the next step without further purification.

A flask was charged with compound 68b (280 mg, 0.82 mmol) and POCl₃ (7.5 g). The resulting mixture was heated to reflux and maintained at this temperature for 4 hrs. The reaction was monitored by LCMS. After completion of the reaction, excess POCl₃ was removed under reduced pressure, the residue was diluted with ice-water, neutralized by saturated aq. NaHCO₃, extracted with EtOAc (30 mL×3). The combined organic layer was washed with brine, dried over anhydrous sodium sulfate, the solvent was removed under reduced pressure to afford compound 69b (200 mg, yield 68%), which was used directly in the next step without further purification.

To a solution of compound 6b (318 mg 0.56 mmol) in DMSO (10 ml) was added KOt-Bu (313 mg 2.8 mmol) at room temperature. The mixture was stirred for 1 h at room temperature. Then, compound 69b (200 mg 0.56 mmol) was added. The resulting mixture was stirred for 12 hrs at room temperature. The reaction was monitored by LCMS. After completion of the reaction, the reaction was quenched by ice-water, acidified with aq. HCl (1 M) to pH=5-6. The resulting mixture was extracted with EtOAc (30 mL×3). The combined organic layer was washed with brine, dried over anhydrous sodium sulfate. The solvent was removed under reduced pressure. The crude product was purified by prep-HPLC to give 168 (166.2 mg, yield 33%). MS (ESI) m/z (M+H)⁺ 895.5.

### 1.21 Synthesis of Compound 169

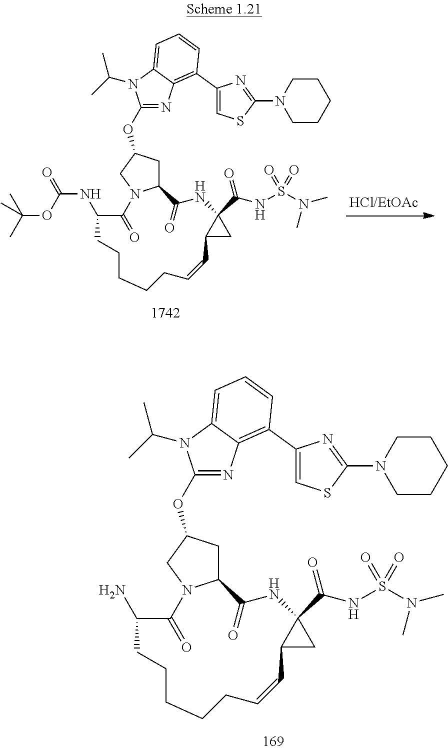

Compound 1742 (20 mg, 0.0223 mmol) was dissolved in a solution of HCl (g) in EtOAC (4 M, 1 mL). The mixture was stirred at rt for 2 hrs. LCMS analysis showed the reaction was completed. The solvent was removed under reduced pressure to afford crude product, which was purified by Prep-HPLC (TFA 0.3% condition) to afford compound 169 (TFA salt, 11.3 mg, yield 50%). MS (ESI) m/z (M+H)⁺ 796.3. Compound 1742 can be synthesized according to Scheme 17J in U.S. patent application Ser. No. 12/890,475 filed on Sep. 24, 2010, which is incorporated by reference herein.

### 1.22 Synthesis of Compound 170

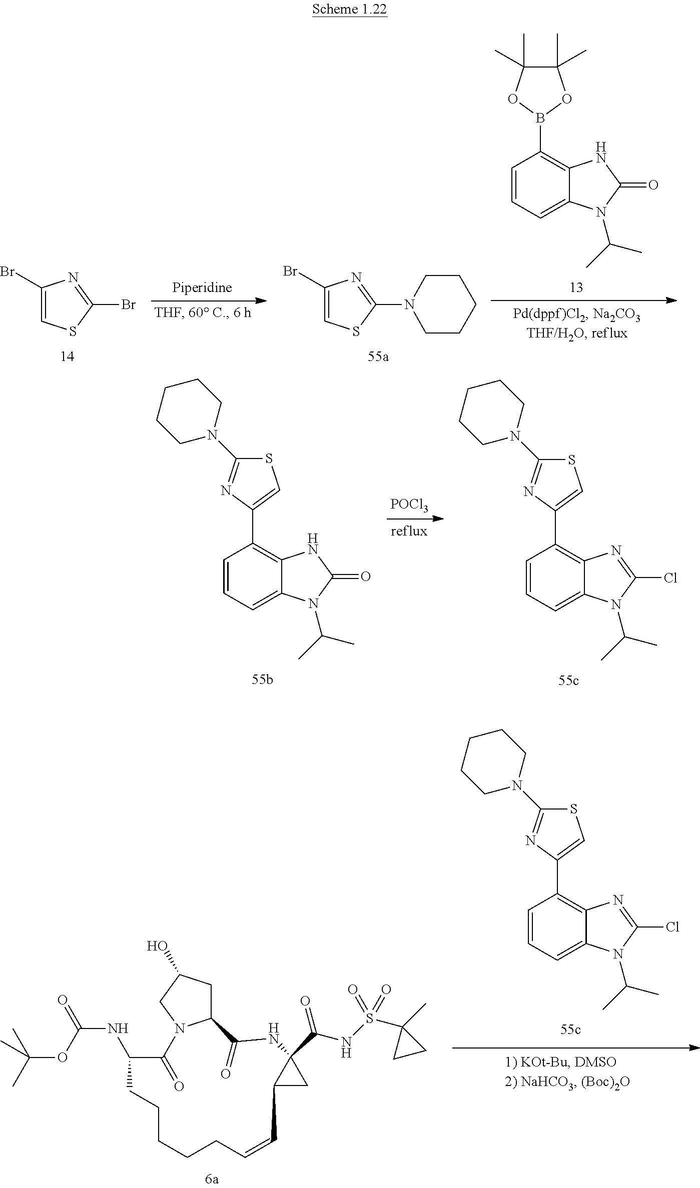

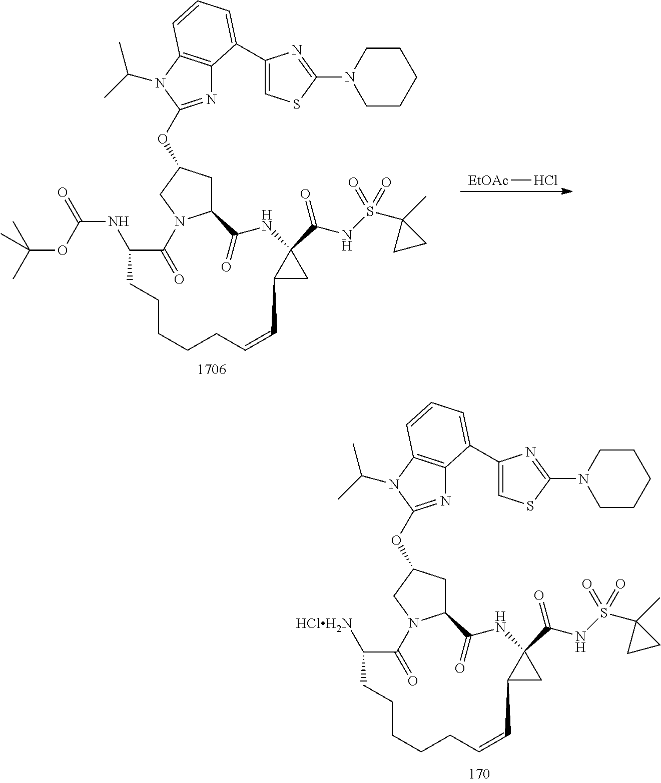

A flask was charged with compound 14 (800 mg, 3.2 mmol), piperidine (699 mg, 8.2 mmol) and THF (10 mL). The resulting mixture was stirred at 60° C. for 6 hrs. The reaction was monitored by LCMS. After completion of the reaction, the solvent was removed under reduced pressure, the residue was diluted with ethyl acetate (50 mL), washed with water and brine. The organic layer was dried over anhydrous sodium sulfate, filtered and concentrated in vauo. The crude product was purified with prep-TLC to afford 55a as a white solid (800 mg, yield 98%). ¹H NMR (400 MHz, CDCl₃): δ 6.37 (s, 1H), 3.44 (m, 4H), 1.65 (m, 6H). MS (ESI) m/z (M+H)⁺ 248.8.

A flask was charged with compound 55a (400 mg, 1.62 mmol), compound 13 (589 mg, 1.94 mmol), Na₂CO₃ (343 mg, 3.24 mmol), THF (10 mL) and water (1 mL), and then it was purged with nitrogen for three times. Followed by addition of Pd(dppf)Cl₂ (237 mg, 0.32 mmol). The mixture was heated to reflux for 16 hrs. The reaction was monitored by LCMS. Then the mixture was cooled to rt and extracted with EtOAc (100 mL×2). The combined organic layer was washed with brine, dried over anhydrous Na₂SO₄, and concentrated in vacuo. The residue was purified by prep-TLC to afford compound 55b (200 mg, yield 36%). MS (ESI) m/z (M+H)⁺ 342.9.

Compound 55b was dissolved in POCl₃ and then the mixture was refluxed under nitrogen. After the completion and cooling to rt, the reaction mixture was taken up with ice-water, neutralized with ammonia under cooling, extracted with EtOAc (30 mL×3). The combined organic layers were washed with brine, dried over anhydrous Na₂SO₄ and concentrated to give a crude compound 55c (80 mg, yield 38%). MS (ESI) m/z (M+H)⁺ 361.0.

To a solution of compound 6a (1 eq.) in 2 mL of DMSO was added KOt-Bu (5 eq.) portionwise at ambient temperature, the mixture was then stirred for 2 hrs at ambient temperature. Subsequently, general compound 55c (1.1 eq.) was added, the resulting mixture was stirred at rt for 20 hrs. The reaction was monitored by LCMS indicating the coupling product with loss of the Boc group. The stirring mixture was cooled in an ice-water bath and acidified by addition of aq. HCl (2 M) to pH=7-8. Subsequently, Boc₂O (1.5 eq.) and NaHCO₃ (1.5 eq.) were added. The mixture was stirred for another 2 hrs and then was acidified to pH=5-6 with aq. HCl (0.1 M) and extracted with ethyl acetate. The organic layers were combined, washed by brine, dried over anhydrous sodium sulfate, concentrated under reduced pressure, and the resulting residue was purified by prep-TLC or prep-HPLC to provide compound 1706 (255.2 mg, yield 32%). MS (ESI) m/z (M+H)⁺ 907.2.

Compound 1706 (12 mg, 0.013 mmol) was dissolved in 1 mL of HCl-EtOAc. The solution was stirred for 2 hrs at 0° C. LCMS analysis showed the reaction was completed. The solvent was removed under reduced pressure. The residual was dried by freezing to afforded light yellow solid compound 170 (9.1 mg, yield 87%). MS (ESI) m/z (M+H)⁺ 807.5.

### 1.23 Synthesis of Compound 171

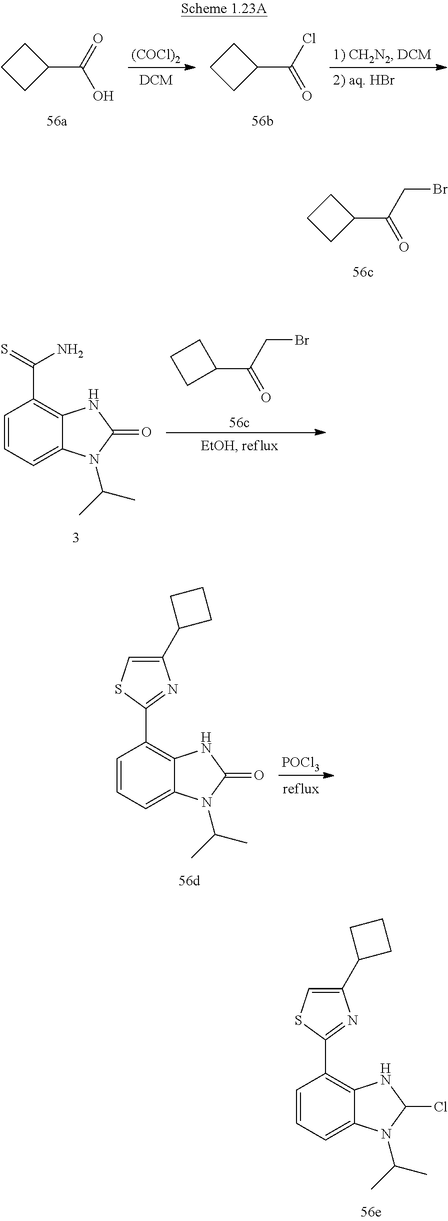

To a solution of compound 56a (3 g, 30 mmol) in DCM (30 mL) was added dropwise oxalyl chloride (3.8 g, 30 mmoL), followed by addition of DMF (2 drops). The solution was stirred at ambient temperature for 3 hrs, the solution of compound 56b was used for next step directly.

To above solution of compound 56b in DCM was added dropwise a solution of diazomethane in ether (59.2 mmoL) at 0° C. The mixture was stirred at ambient temperature for 1 hour. Then a solution of aq. HBr (40%, 4 mL) was added at 0° C., and then the mixture was stirred at rt for 2 hrs. The reaction mixture was adjusted to pH=7 by addition of saturated aq. NaHCO₃, the organic layer was separated, washed with brine, dried over anhydrous Na₂SO₄ and concentrated under reduced pressure to give compound 56c (3.2 g, yield 60% over two steps) as brown oil.

To a solution of compound 3 (500 mg, 2.128 mmol) in EtOH (6 mL) was added compound 56c (414 mg, 2.34 mmol). The mixture was stirred at 85° C. overnight. After completion of the reaction, the solvent was evaporated under reduced pressure. The residue was diluted with EtOAc (100 mL), the resulting solution was neutralized by addition of saturated aq. NaHCO₃, and washed with water and brine, the organic layer was separated, dried over anhydrous Na₂SO₄ and concentrated to give compound 56d (510 mg, yield 77%) as light brown solid. MS (ESI) m/z (M+H)⁺ 313.9.

Compound 56d (500 mg, 1.59 mmol) was dissolved in POCl₃ (3 mL) and then the mixture was refluxed under nitrogen. After completion of reaction, most of POCl₃ was evaporated, and then the mixture was taken up with ice-water, neutralized with saturated aq. NaHCO₃ under cooling, then extracted with EtOAc (20 mL×3). The combined organic layer was washed with brine, dried over anhydrous Na₂SO₄ and concentrated to give a crude product compound 56e, which was used directly in next step (480 mg, yield 91%). MS (ESI) m/z (M+H)⁺ 331.9.

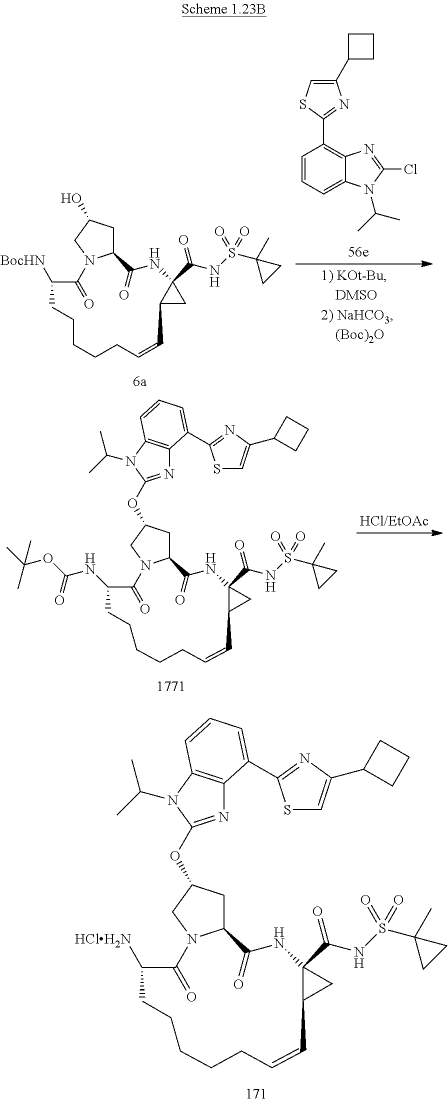

To a solution of compound 6a (350 mg, 0.601 mmol) in DMSO (20 mL) was added KOt-Bu (282 mg, 2.5 mmol), the mixture was stirred at 0° C. for 1 h under nitrogen. After that, compound 56e (200 mg, 0.601 mmol) was added thereto, the reaction mixture was stirred at rt for 12 hrs. After completion of the reaction, the solution was quenched by ice-water. The mixture was neutralized by aq. HCl (1 M), then adjusted to pH=8 by addition of saturated aq. NaHCO₃, and (Boc)₂O (157 mg, 0.721 mmol) was added, the mixture was stirred at rt for additional 1 hour. The mixture was extracted with EtOAc (50 mL×3). The combined organic layer was washed with brine, dried over anhydrous Na₂SO₄ and concentrated in vacuo. The residue was purified by prep-TLC (PE/EA=2/3) to provide compound 1771 as white solid (230 mg, yield 44%). MS (ESI) m/z (M+H)⁺ 878.1.

Compound 1771 (12 mg, 0.0136 mmol) was dissolved in a solution of HCl (g) in EtOAC (1 mL). The mixture was stirred at r.t for 2 hrs. After completion of the reaction, the solvents were removed under reduced pressure, the residue was dried in vacuo to afford compound 171 (HCl salt, 10.4 mg, yield 94%) as a yellow solid.

### 1.24 Synthesis of Compounds 172 and 173

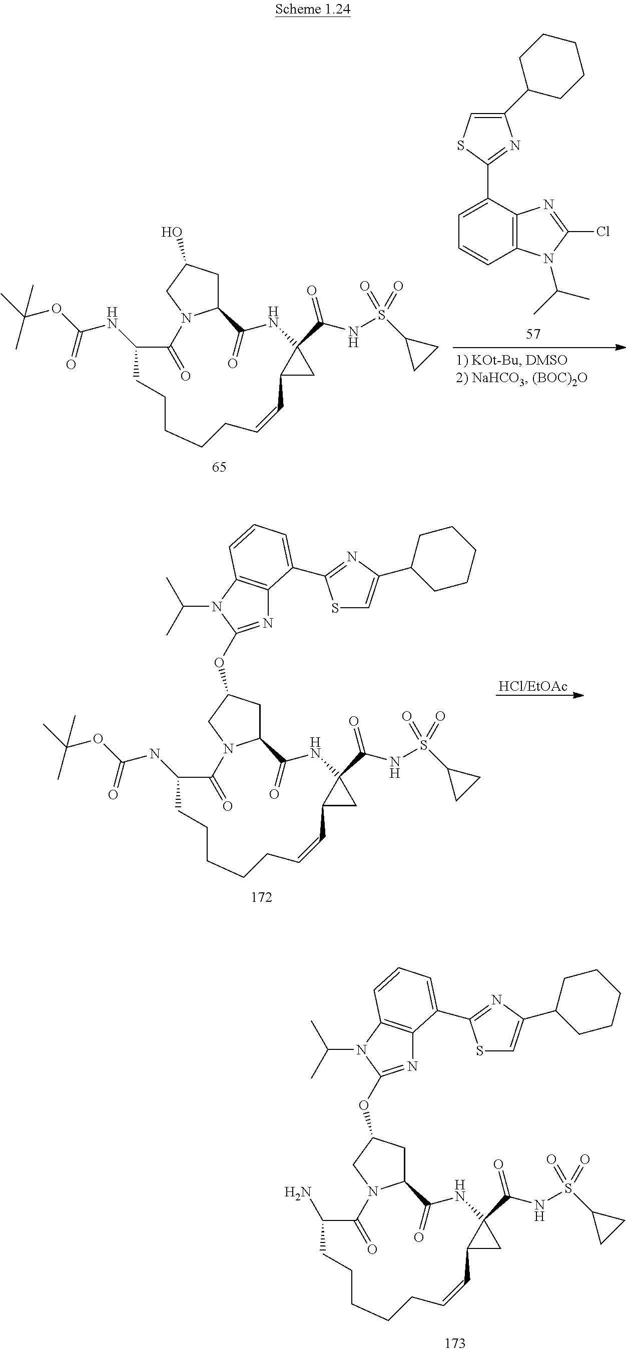

To a solution of compound 65 (1.0 g, 1.176 mmol) in DMSO (30 mL) was added KOt-Bu (985 mg, 8.8 mmol), the mixture was stirred at 0° C. for 1 h under nitrogen. After that, compound 57 (758 mg, 2.11 mmol) was added into the solution, the reaction mixture was stirred at r.t for 1.5 h. After completion of the reaction, the reaction was quenched by ice-water. The mixture was neutralized by aq. HCl (1 M), then adjusted to pH=8 by addition of saturated aq. NaHCO₃, (Boc)₂O (460 mg, 2.11 mmol) was added thereto. The mixture was stirred at rt for 1 hour. The solution was extracted with EtOAc (50 mL×3). The combined organic layer was washed with brine, dried over anhydrous Na₂SO₄ and concentrated in vacuo. The crude product was purified by prep-TLC (DCM/MeOH=20/1) to provide compound 172 (425 mg, yield 40%) as white solid. MS (ESI) m/z (M+H)⁺ 892.3. The synthesis for compound 57 was described in Scheme 17O of U.S. patent application Ser. No. 12/890,475 filed Sep. 24, 2010.

Compound 173 was prepared using the same procedure as that for making compound 171 (TFA salt, 10.3 mg, yield 63%). MS (ESI) m/z (M+H)⁺ 792.2.

### 1.25 Synthesis of Compounds 174 and 175

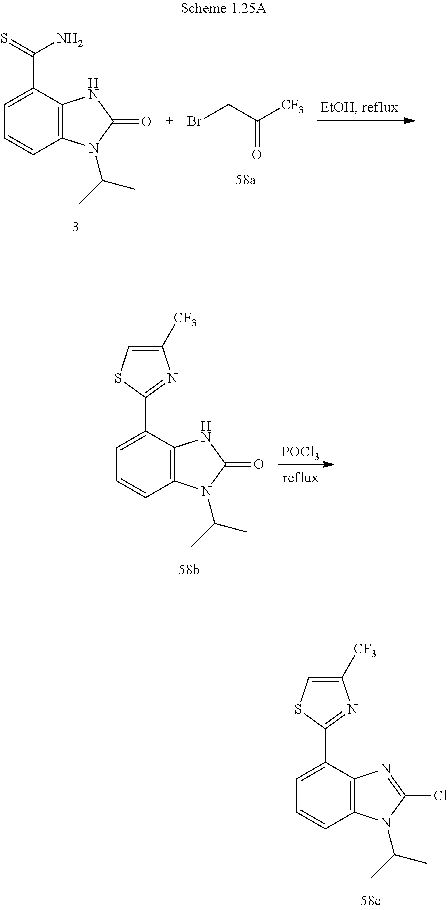

To a solution of compound 3 (200 mg, 0.9 mmol) in EtOH (10 mL) was added compound 58a (294 mg, 1.5 mmol). The mixture was refluxed under nitrogen. After completion of the reaction, the solvent was evaporated under reduced pressure to give a crude product, which was purified by column chromatography on silica gel (PE/EA=2:1) to afford compound 58b (97 mg, yield 35%). MS (ESI) m/z (M+H)⁺ 327.9.

Compound 58b (100 mg, 0.3 mmol) was dissolved in POCl₃ (5 mL) and then the mixture was refluxed under nitrogen. After completion of reaction, excess of POCl₃ was evaporated, and then the mixture was taken up with ice-water, neutralized with ammonia under cooling, then extracted with EtOAc (20 mL×3). The combined organic layer was washed with brine, dried over anhydrous Na₂SO₄ and concentrated to give a crude product 58c, which was used directly in next step (101 mg, yield 96%).

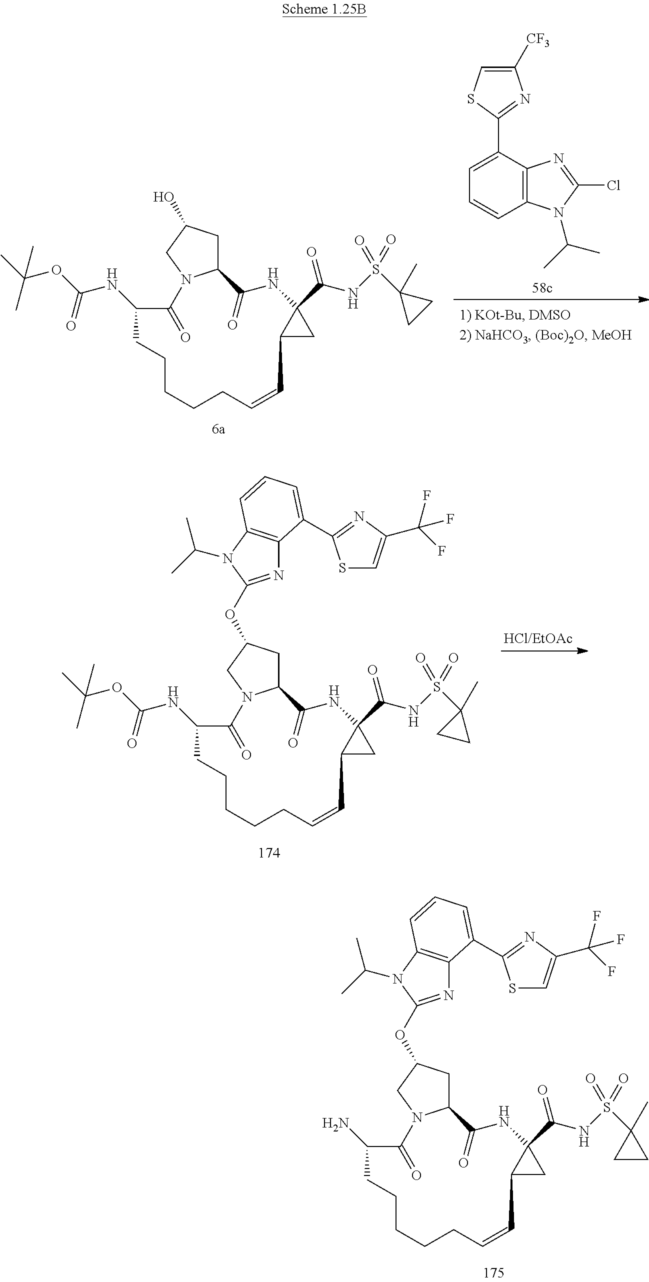

General procedure as described in 1.24 was followed for preparing compound 174 (115 mg, yield 68%, MS (ESI) m/z (M+H)⁺ 892.3) and compound 175 (TFA salt, 10 mg, yield 90%, MS (ESI) m/z (M+H)⁺ 792.3).

### 1.26 Synthesis of Compound 176

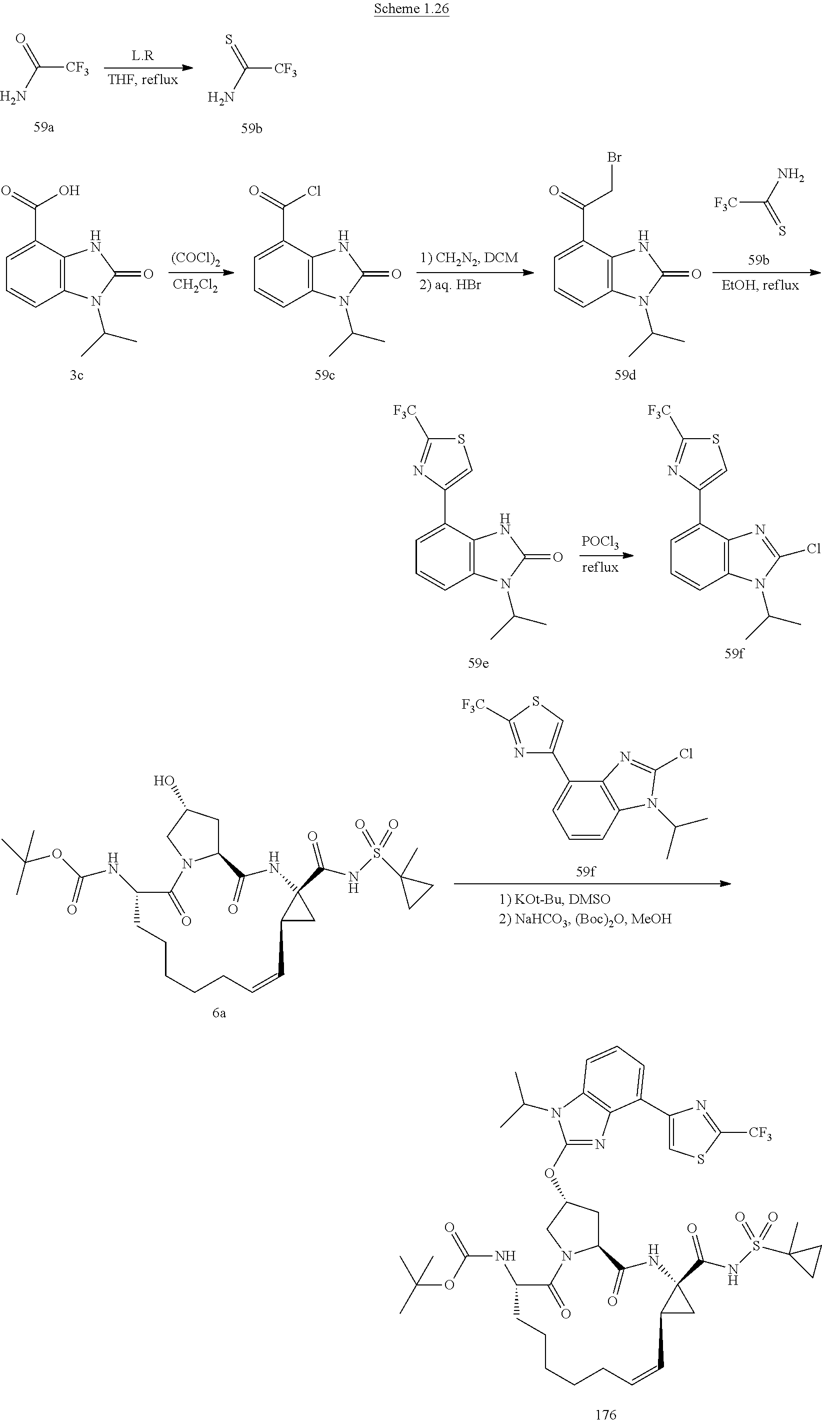

To a solution of compound 59a (0.5 g, 4.4 mmol) in dry THF (60 mL) was added Lawesson reagent (0.99 g, 2.43 mmol). The mixture was refluxed under nitrogen for 4 hrs. After removal of the solvent, the reaction mixture was distilled to give compound 59b (0.52 g, yield 92%) as liquid.

Compound 3c (1 g, 4.5 mmol) was dissolved in dry DCM (20 mL), and oxalyl chloride (0.6 mL, 6.8 mmol) was added dropwise, followed by addition one drop of DMF. The mixture was stirred at rt under nitrogen for 4 hrs. After removal of the solvent, the reaction mixture was evaporated to provide compound 59c (1.07 g, yield 100%), which was used directly for next step.

Compound 59c (1.07 g, 4.5 mmol) was dissolved in CH₂Cl₂ (10 mL). The resulting solution was added dropwise into a solution of CH₂N₂ in ether (0.7 M, 60 mL) at −10° C. The reaction mixture was stirred at 0° C. for 1 hour. And then the reaction mixture was cooled to −10° C. again, to this solution was added dropwise aqueous HBr (48%, 5 mL, 39.8 mmol). The reaction mixture was stirred at the same temperature for 1 hour. After that, the mixture was washed with saturated aqueous NaHCO₃ and brine. The organic layer was separated, dried over anhydrous Na₂SO₄, concentrated in vacuo. The residue was purified by prep-HPLC to afford compound 59d (220 mg, yield 17%). MS (ESI) m/z (M+H)⁺ 296.9.

To a solution of compound 59b (220 mg, 0.74 mmol) in dry EtOH (4 mL) was added compound 59d (115 mg, 0.89 mmol). The mixture was refluxed under nitrogen overnight. After removal of the solvent, the reaction mixture was diluted with EtOAc (100 mL), washed with aq. NaHCO₃ and brine, dried over anhydrous Na₂SO₄ and concentrated in vacuo. The residue was purified by prep-TLC (PE/EA=3/1) to afford compound 59e (60 mg, yield 25%). MS (ESI) m/z (M+H)⁺ 327.8.

General procedure was followed for the preparation of compound 59f (60 mg, yield 90%). MS (ESI) m/z (M+H)⁺ 345.9. Preparation of compound 176 was prepare according to the procedure described for making compound 172. (26 mg, yield: 22%). MS (ESI) m/z (M+H)⁺ 892.4.

### 1.27 Synthesis of Compound 177

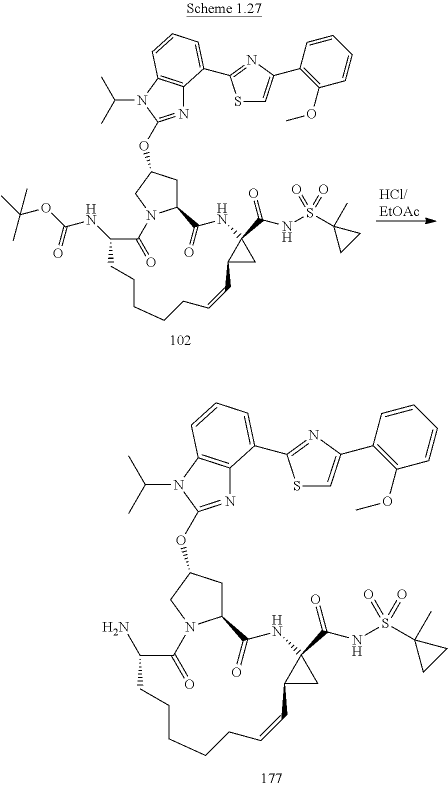

Compound 102 (10 mg, 0.01 mmol) was dissolved in HCl/EtOAC (1 mL). The mixture was stirred at r.t for 2 hrs. Then the solvents were removed under reduced pressure to afford crude product, which was purified by prep-TLC (DCM/MeOH=10/1) to afford compound 177 (3.0 mg, yield 36%) as a white solid. MS (ESI) m/z (M+H)⁺ 830.3.

### 1.28 Synthesis of Compound 178

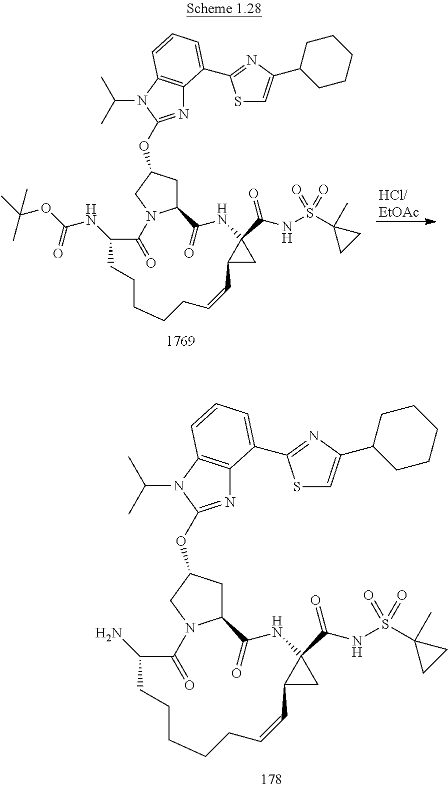

Compound 178 was prepared from compound 1769 (described in U.S. patent application Ser. No. 12/890,475 filed on Sep. 24, 2010) according to the procedure described in 1.27. (TFA salt, 14 mg, yield 77%). MS (ESI) m/z (M+H)⁺ 806.3.

### 1.29 Synthesis of Compound 179

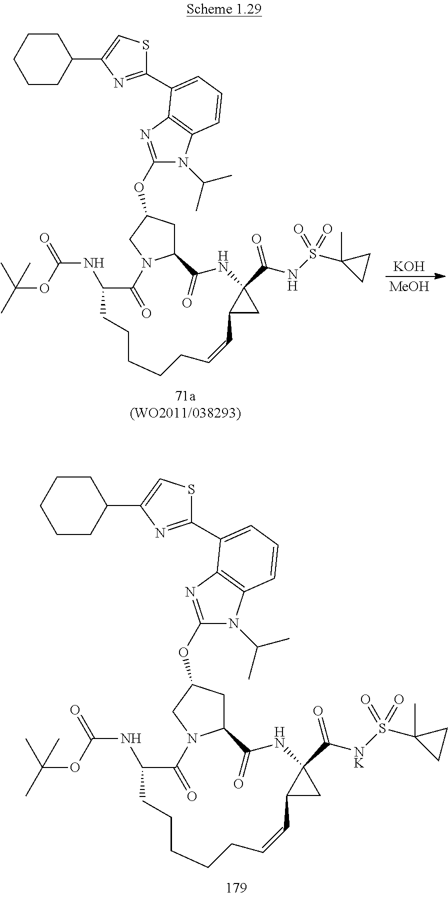

Compound 71a (400 mg, 0.44 mmol) was dissolved in MeOH (10 mL), to the solution was added aq. KOH (0.1M, 4.4 mL, 0.44 mmol). The mixture was stirred at room temperature for 2 hrs, concentrated and freezing-dry to give the corresponding K salt compound 179 (400 mg, 96% yield) as white solid. MS (ESI) m/z (M+H)⁺ 906.3.

### 1.30 Synthesis of Compound 180

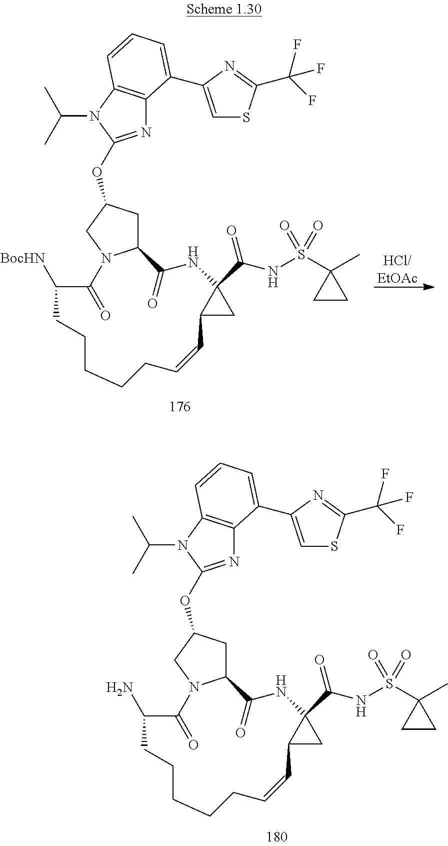

A mixture of compound 176 (12 mg, 0.013 mmol) and HCl/EtOAc (4N, 2 mL) was stirred at room temperature for 2 hrs, and then concentrated. The residue was purified by prep-HPLC (0.3% TFA) to give the desired product as CF₃COOH salt, compound 180 (5 mg, 50% yield). MS (ESI) m/z (M+H)⁺ 792.0.

### 1.31 Synthesis of Compound 181

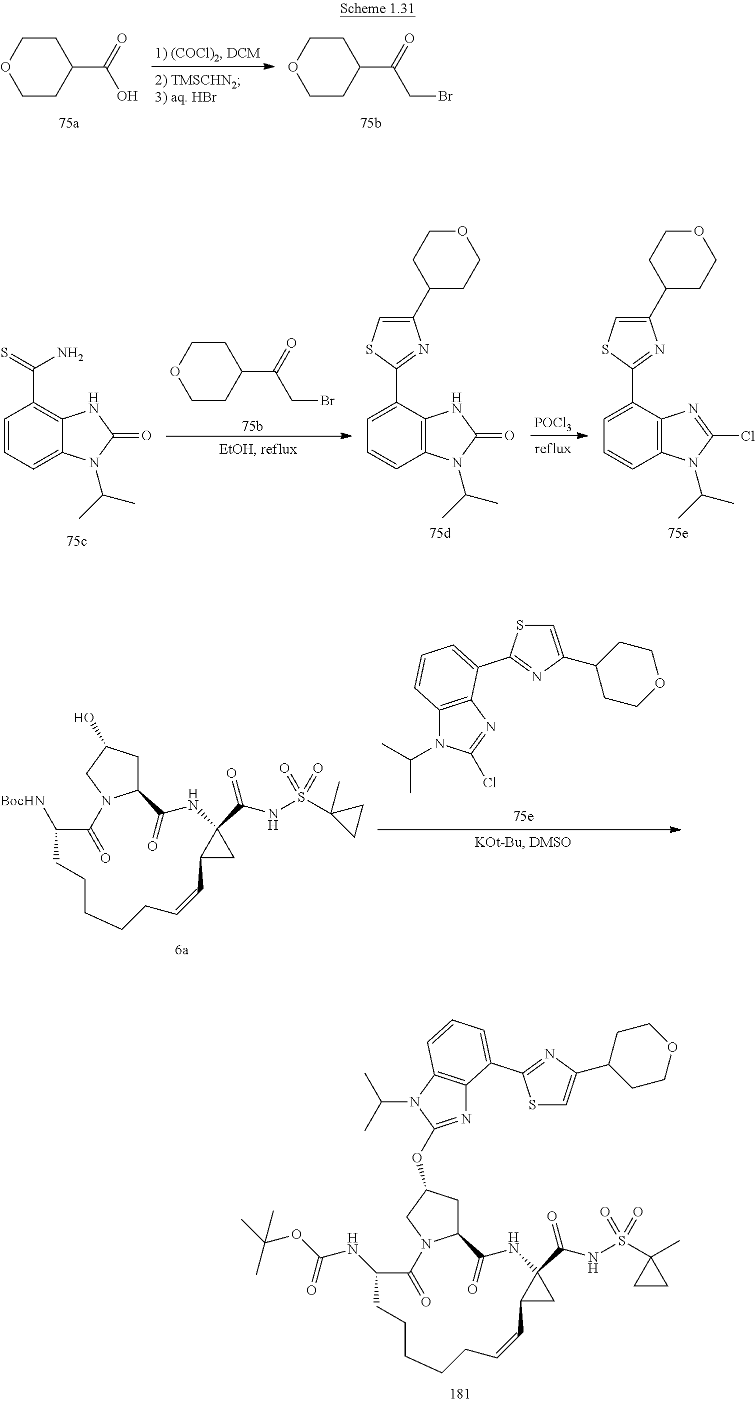

To a solution of compound 75a (3 g, 23 mmol) in dry DCM (20 mL) was added one drop DMF. And then oxalyl chloride (3.23 g, 25.4 mmol) was added at 0° C. The mixture was stirred at r.t under nitrogen for 2 hrs. After removal of the solvent to dryness, the residue was dissolved in CH₂Cl₂ (20 mL) and the resulting solution was added dropwise to a solution of TMSCHN₂ in ether (2 M, 34.5 mL, 69 mmol) at −10° C. The reaction mixture was stirred at r.t overnight. The reaction mixture was cooled to −10° C. again, to this solution was added dropwise aqueous HBr (48%, 4 mL). The reaction mixture was stirred at r.t for 1 h, and then it was washed with saturated aqueous NaHCO₃ and brine. The organic phase was dried over anhydrous Na₂SO₄, concentrated to give crude compound 75b (3.5 g, yield 75%), which was used directly for next step. ¹H NMR (CDCl₃, 400 MHz): δ 4.16 (s, 2H), 4.04-3.95 (m, 2H), 3.43 (dt, J=2.8 Hz, 11.2 Hz, 2H), 3.00-2.88 (m, 1H), 1.81-1.68 (m, 4H).

To a solution of compound 75c (3 g, 12.75 mmol) in EtOH (10 mL) was added compound 75b (2.65 g, 12.8 mmol). The mixture was refluxed for 2 hrs under nitrogen. After removal of the solvent, the reaction mixture was diluted with DCM (100 mL), washed with water and brine, dried over anhydrous Na₂SO₄, and then concentrated. The residue was purified by column chromatography (PE:EtOAc=3:1) to give compound 75d (2.9 g, yield 66%). MS (ESI) m/z (M+H)⁺ 344.1.

Compound 75d (2.9 g, 8.45 mmol) was dissolved in POCl₃ (30 mL), and then the mixture was refluxed under nitrogen. After completion of reaction, the reaction mixture was taken into ice-water, neutralized with NH₃.H₂O under cooling, extracted with EtOAc (40 mL×3). The combined organic layer was washed with brine, dried over anhydrous Na₂SO₄ and concentrated to give a crude product compound 75e, which was used directly in next step (2.6 g, yield 87%).

To a solution of compound 6a (1.343 g, 2.31 mmol) in 10 mL of DMSO was added KOt-Bu (1.03 g, 9.23 mmol) in portions at ambient temperature, then the mixture was stirred for 30 min at ambient temperature. After that, compound 75e (1 g, 2.77 mmol) was added, the resulting mixture was stirred at rt for 20 hrs, the reaction was monitored by LCMS. The mixture was acidified to pH=5-6 with aq. HCl (0.1M), extracted with EtOAc (50 mL×3). The organic layers were combined, washed with brine, dried over anhydrous sodium sulfate, concentrated under reduced pressure. The residue was purified by prep-HPLC to provide compound 181 (753 mg, yield 38%).

### 1.32 Synthesis of Compound 182

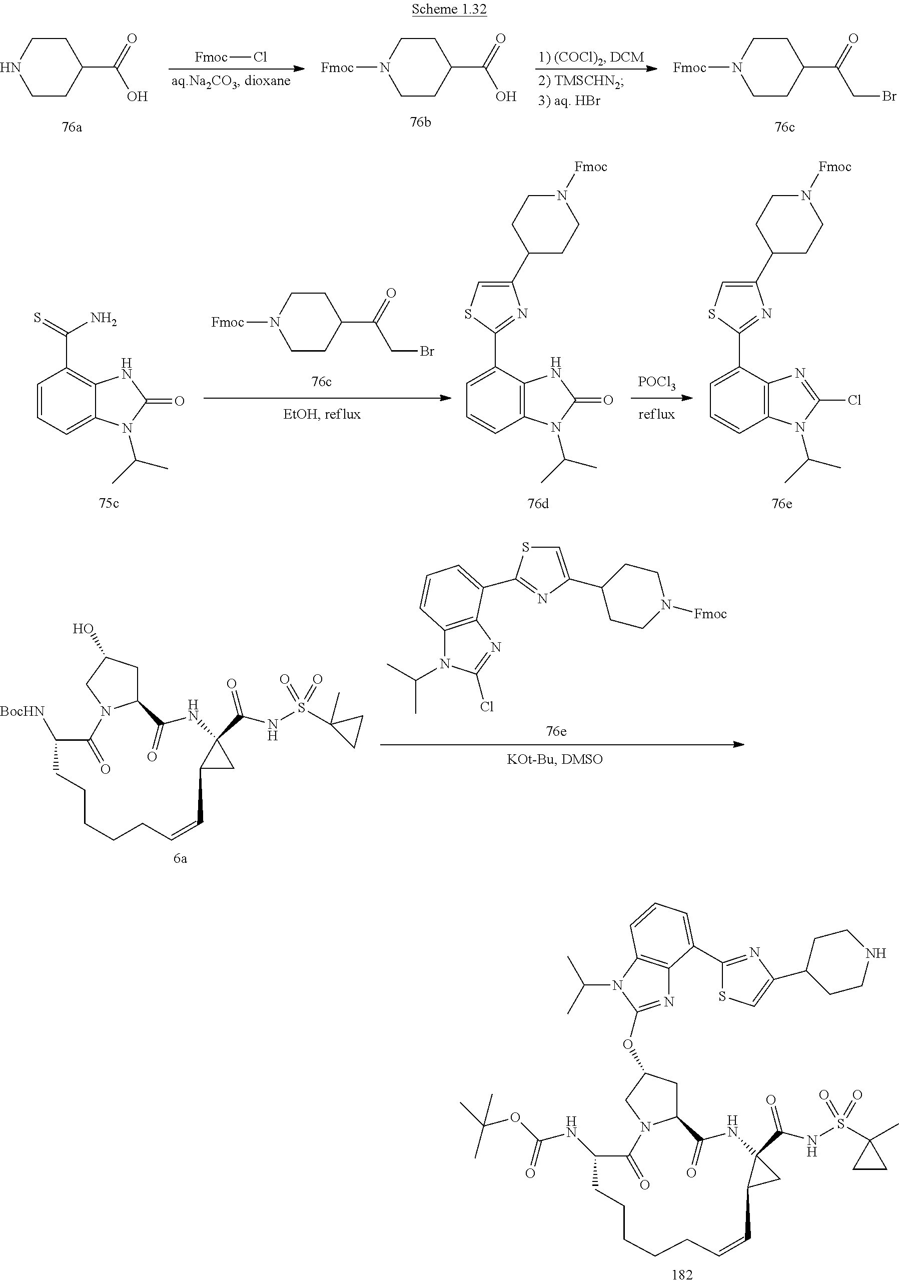

FmocCl (25 g, 96.6 mmol) in dioxane (120 mL) was added to a solution of isonipecotic acid (10 g, 77.4 mmol) 76a and Na₂CO₃ (17.2 g, 162.6 mmol) in water (120 mL). The mixture was stirred for 24 hrs at rt Then it was poured into water (100 mL), and the aqueous layer was extracted with EtOAc (50 mL×3). And then the aqueous layer was acidified with aq. HCl (2 N) to pH=2, and extracted with EtOAc (100 mL×3), the combined extracts was washed with brine, dried over Na₂SO₄, concentrated in vacuo. The product 76b was sufficiently pure for further use (20 g, yield 74%). ¹H NMR (DMSO-d₆, 300 MHz): δ 12.27 (brs, 1H), 7.87 (d, J=7.2 Hz, 2H), 7.60 (d, J=7.2 Hz, 2H), 7.42-7.28 (m, 4H), 4.34 (m, 2H), 4.24 (m, 1H), 3.77 (m, 2H), 2.83 (m, 2H), 2.38 (m, 1H), 1.70 (m, 2H), 1.30 (m, 2H).

To a solution of compound 76b (5 g, 14.2 mmol) and one drop DMF in dry DCM (100 mL) was added oxalyl chloride (1.8 mL, 21.3 mmol) slowly. The mixture was stirred at r.t under nitrogen for 4 hrs. After removal of the solvent to dryness, the residue was dissolved in CH₂Cl₂ (100 mL) and the resulting solution was added dropwise to a solution of TMSCHN₂ in ether (2 M, 22 mL, 44 mmol) at −10° C. The reaction mixture was stirred at r.t overnight. The reaction mixture was cooled to −10° C. again, to this solution was added dropwise aqueous HBr (48%, 15 mL,). The reaction mixture was stirred at r.t for 1 h, washed with saturated aqueous NaHCO₃ and brine. The organic phase was dried over anhydrous Na₂SO₄, concentrated to give crude compound 76c (5.6 g, yield 92%), which was used directly for next step. MS (ESI) m/z (M+H)⁺ 428.0.

To a solution of compound 75c (560 mg, 2.38 mmol) in EtOH (30 mL) was added compound 76c (1.02 g, 2.38 mmol). The mixture was refluxed for 2 hrs under nitrogen. After removal of the solvent, the reaction mixture was diluted with DCM (100 mL), washed with water and brine, dried over anhydrous Na₂SO₄, and then concentrated. The residue was purified by column chromatography (PE:EtOAc=3:1) to give compound 76d (0.95 g, yield 72%). MS (ESI) m/z (M+H)⁺ 565.0.

Compound 76d (0.95 g, 1.68 mmol) was dissolved in POCl₃ (5 mL) and then the mixture was refluxed under nitrogen. After completion of the reaction, the reaction mixture was taken into ice-water, neutralized with NH₃.H₂O under cooling, extracted with EtOAc (40 mL×3). The combined organic layer was washed with brine, dried over anhydrous Na₂SO₄ and concentrated to give a crude product 76e, which was used directly in next step (0.95 g, yield 97%). MS (ESI) m/z (M+H)⁺, 582.9.

Compound 182 was prepared following the general procedure (450 mg, yield 40%). MS (ESI) m/z (M+H)⁺ 907.3.

### 1.33 Synthesis of Compounds 183 and 184

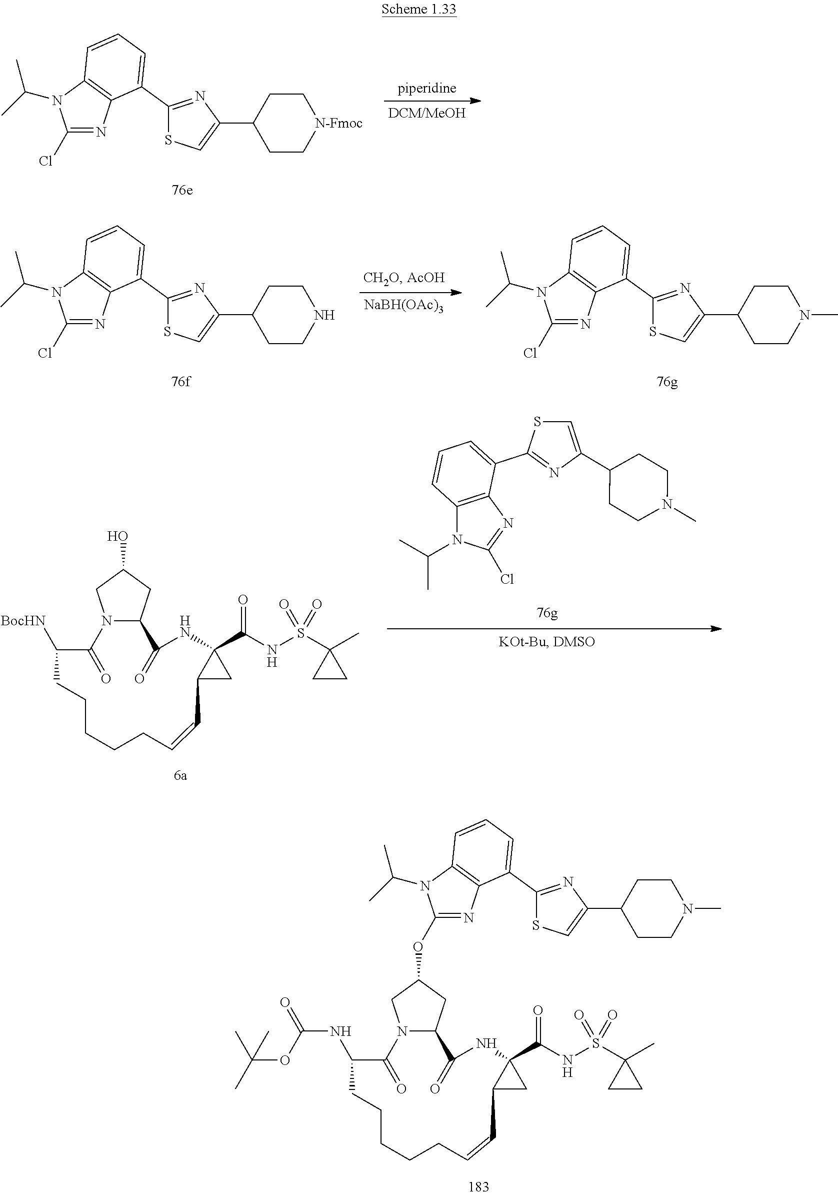

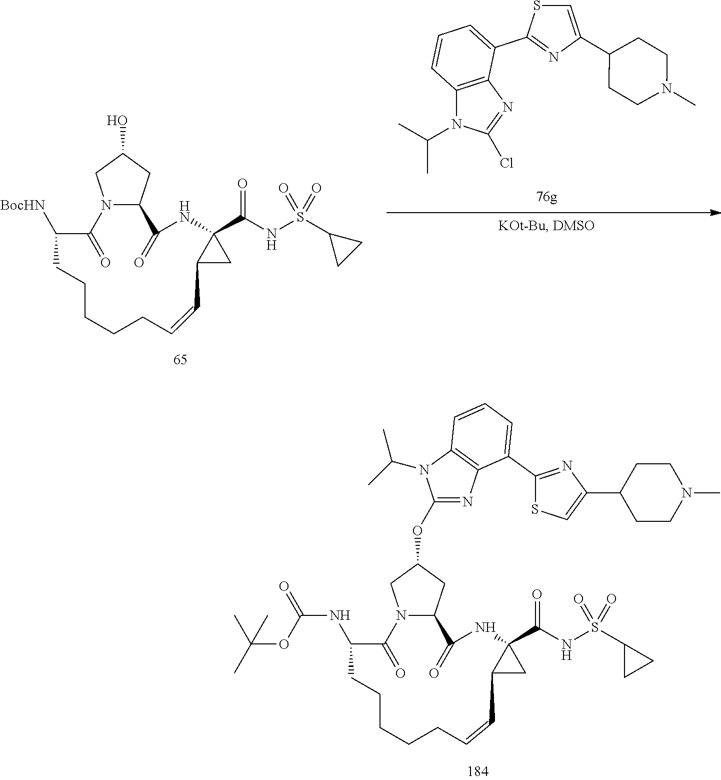

To a solution of compound 76e (4.2 g, 7.2 mmol) in MeOH (10 mL) and DCM (30 mL) was added piperidine (10 mL). The mixture was stirred at r.t for 2 h. After removal of the solvent, the reaction mixture was washed with PE to yield compound 76f (1.5 g, yield 58%). MS (ESI) m/z (M+H)⁺ 360.9.

To a solution of compound 76f (1.5 g, 4.16 mmol) in DCM (60 mL) was added AcOH (0.5 mL) and formaldehyde water solution (0.5 mL) and NaBH(OAc)₃ (1.5 g, 4.16 mmol). The mixture was stirred at rt overnight. The reaction mixture was diluted with DCM (100 mL), washed with saturated aqueous NaHCO₃ and brine. The organic phase was dried over anhydrous Na₂SO₄, and concentrated to give crude compound 76g (1.3 g, crude yield 92%), which was used directly for next step. MS (ESI) m/z (M+H)⁺ 375.0

To a solution of compound 6a (1.25 g, 2.14 mmol) in DMSO (20 mL) was added KOt-Bu (1.32 g, 11.77 mmol, 5.5 e.q.), the mixture was stirred at 0° C. for 0.5 h under nitrogen. After that, compound 76g (0.8 g, 2.74 mmol) was added into the solution, the reaction mixture was stirred at r.t for 1.5 h. After completion of the reaction, the reaction was quenched by ice-water. The mixture was neutralized by aq. HCl (1 M), and then extracted with EtOAc (50 mL×3). The combined organic layers were washed with brine, dried over anhydrous Na₂SO₄ and concentrated in vacuo. The residue was purified by prep-HPLC to afford compound 183 as white solid (950 mg, yield 48%). MS (ESI) m/z [M+H]⁺ 921.3.

To a solution of compound 65 (1.25 g, 2.14 mmol) in DMSO (20 mL) was added KOt-Bu (1.32 g, 11.77 mmol, 5.5 ext.), the mixture was stirred at 0° C. for 0.5 h under nitrogen. After that, compound 76g (0.8 g, 2.74 mmol) was added into the solution, the reaction mixture was stirred at r.t for 1.5 h. After completion of the reaction, the reaction was quenched by ice-water. The mixture was neutralized by aq. HCl (1 M), then extracted with EtOAc (50 mL×3). The combined organic layers were washed with brine, dried over anhydrous Na₂SO₄ and concentrated in vacuo. The residue was purified by prep-HPLC to afford compound 184 as white solid (560 mg, yield 29%). MS (ESI) m/z [M+H]⁺ 907.3.

### 1.34 Synthesis of Compounds 185 and 186

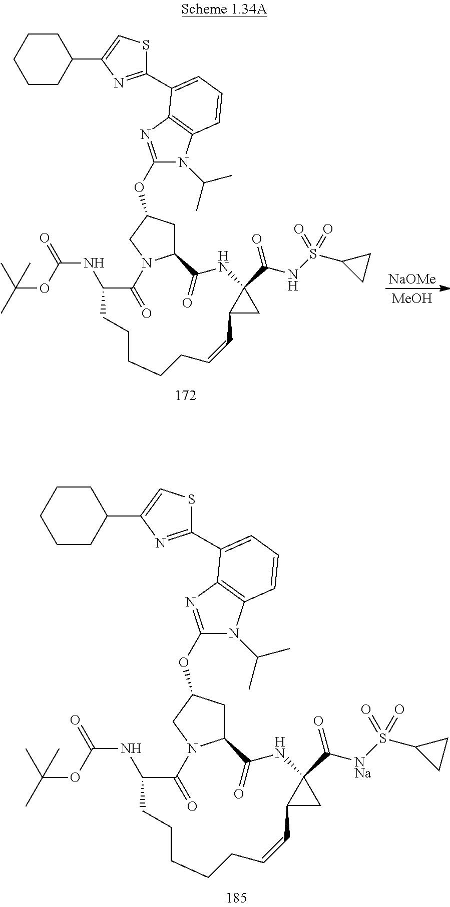

Compound 172 (1.141 g, 1.279 mmol) was dissolved in MeOH (20 mL), to the solution was added a solution of NaOMe in MeOH (0.1 M, 12.79 mL, 1.279 mmol). The mixture was stirred at room temperature for 1 hrs, concentrated and freezing-dry to give the corresponding sodium salt (1.17 g, yield 100%) 185 as white solid. MS (ESI) m/z (M+H)⁺ 892.3.

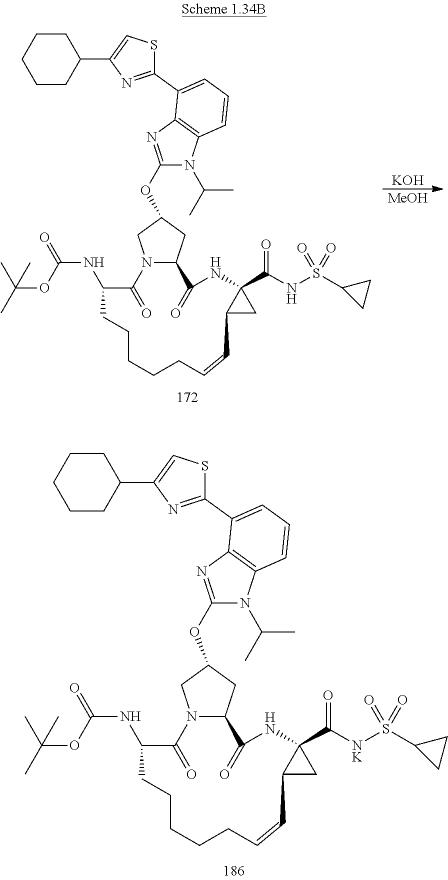

Compound 172 (1.155 g, 1.294 mmol) was dissolved in MeOH (10 mL), to the solution was added aq. KOH (0.1 M, 12.94 mL, 1.294 mmol). The mixture was stirred at room temperature for 2 hrs. The mixture was concentrated and freezing-dry to give the corresponding K salt 186 (1.2 g, yield 100%) as white solid. MS (ESI) m/z (M+H)⁺ 892.2.

### 1.35 Synthesis of Compound 187

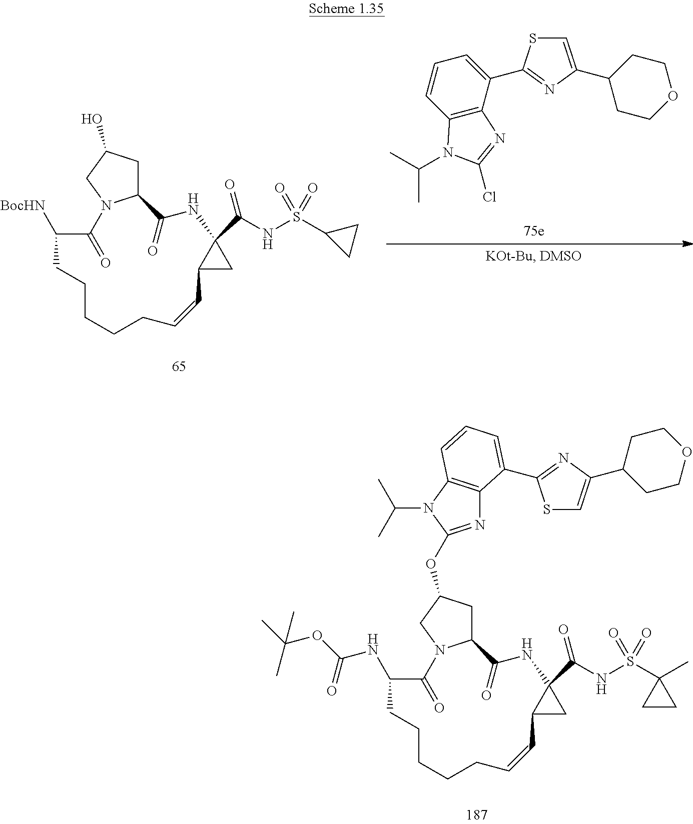

To a solution of compound 65 (2.15 g, 3.79 mmol) in 10 mL of DMSO was added KOt-Bu (1.7 g, 15.14 mmol) in portions at ambient temperature, then the mixture was stirred for 30 min at ambient temperature. After that, compound 75e (1.64 g, 4.54 mmol) was added, the resulting mixture was stirred at rt for 20 hrs, the reaction was monitored by LCMS. The mixture was acidified to pH=5-6 with aq. HCl (0.1M), extracted with EtOAc (50 mL×3). The organic layers were combined, washed with brine, dried over anhydrous sodium sulfate, concentrated under reduced pressure. The residue was purified by prep-HPLC to provide compound 187 (1.02 g, yield 30%).

### 1.36 Synthesis of Compound 188

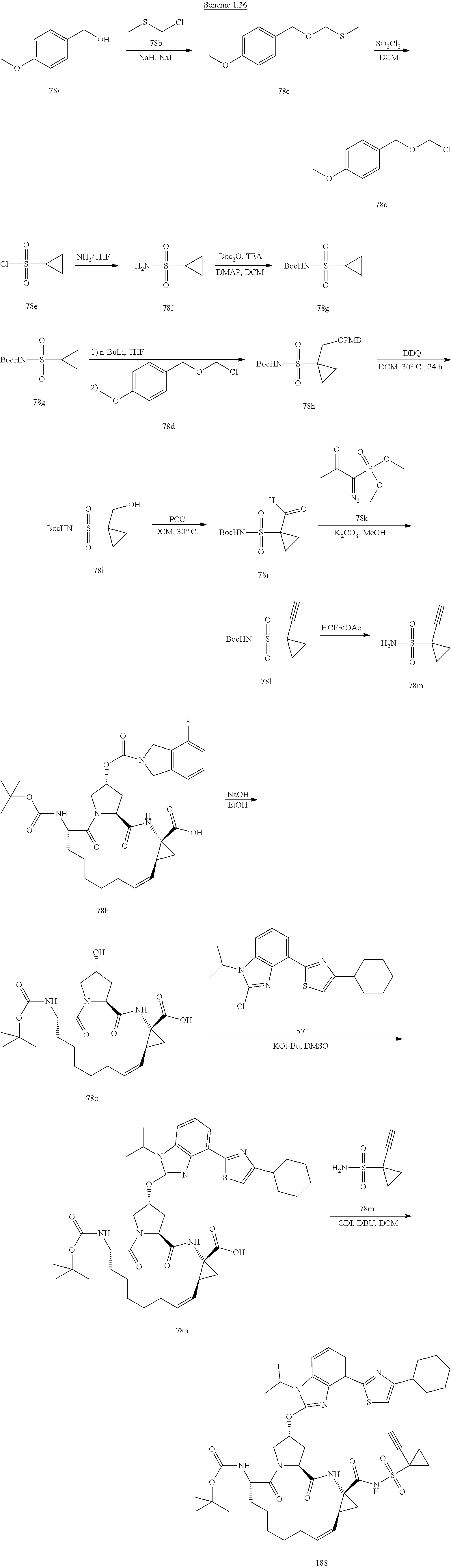

A solution of NaI (14.4 g, 96.4 mmol, 1.0 eq.) and NaH (60%, 7.7 g, 193 mmol, 2.0 eq.) in THF (100 mL) was cooled to 0° C. under nitrogen atmosphere, then compound 78a (12 g, 96.4 mmol, 1.0 eq.) was added dropwise to the solution. The mixture was warmed to rt and stirred for 1 h and cooled to 0° C. again. Compound 78b (8.0 g, 96.4 mmol, 1.0 eq.) was added to the above solution. The resulting mixture was stirred at rt for 12 hrs, quenched with water and extracted with EtOAc (50 mL×3). The combined organic layer was washed with brine, dried over Na₂SO₄, filtered and concentrated in vacuum to give a yellow oil. Compound 78c was purified by column chromatography on silica gel (PE:EtOAc=100:1) as a light yellow oil (14 g, yield 82%).

To a solution of 78c (3 g, 15 mmol, 1.0 eq.) in DCM (50 mL) at −78° C. was added dropwise SO₂Cl₂ (1.54 mL, 16.4 mmol, 1.1 eq.) under nitrogen atmosphere. After addition, the resulting solution was stirred at this temperature for 15 min. Then the reaction mixture was concentrated in vacuum to give the crude compound 78d (2.82 g, crude yield 100%) as a light yellow oil. The oil was used directly without further purification.

To a solution of compound 78e (100 g, 0.71 mmol, 1.0 eq.) in THF (500 mL) was bubbled with ammonium (g) with vigorous stirring at −20° C. for 40 min. After filtration, the filtrate was concentrated in vacuo to give 78f (80 g, yield 93%) as a white crystal.

Cyclopropanesulfonamide (30.0 g, 0.248 mol, 1.0 eq.), trietylamine (37.6 g, 0.372 mol, 1.5 eq.) and dichloromethane (400 mL) were charged in a 1 L round bottom flask. Di-tert-butyldicarbonate (64.0 g, 0.293 mol, 1.2 eq.) and N,N-dimethylaminopyridine (1.5 g, 0.0124 mol, 0.05 eq.) were added portion wise and the reaction mixture stirred at ambient temperature for 12 hours. The solvent was removed in vacuo and the residue was diluted with water. The aqueous phase was acidified to pH=1 with 1M hydrochloric acid, and extracted with ethyl acetate (100 mL×3). The organic extracts were combined, washed with water and brine, dried over sodium sulfate, filtered and the solvent removed in vacuo to give 35 g (65% yield) of the compound 78g as a white solid. ¹H NMR (400 MHz, CDCl₃): δ 7.36 (br. s., 1H), 2.90 (m, 1H) 1.51 (s, 9H) 1.35 (m, 2H) 1.10 (m, 2H).

To a solution of compound 78g (5 g, 22.5 mmol, 3.0 eq.) in anhydrous THF at −78° C. was added n-BuLi (2.5 M in hexane, 22.5 mL, 506 mmol, 7.5 eq.) dropwise. After that, the solution was stirred for 1 h, then a solution of 78d (1.4 g, 7.5 mmol, 1.0 eq.) in THF was added dropwise. After addition, the reaction was quenched with NH₄Cl (aq.), extracted with EtOAc (80 mL×3). The combined organic layer was washed with brine, dried over Na₂SO₄, filtered and concentrated in vacuum to give a yellow oil. Compound 78h was purified by column chromatography on silica gel (PE:EtOAc=10:1) as a white crystal (1.5 g, yield 54%). ¹H NMR (CDCl₃, 400 MHz): δ 7.27-7.23 (m, 2H), 7.19 (brs, 1H), 6.88 (d, J=8.8 Hz, 2H), 4.48 (s, 2H), 3.79 (s, 3H), 3.73 (s, 2H), 1.70 (m, 1H), 1.45 (s, 9H), 1.02 (m, 2H).

A mixture of 78h (800 mg, 2.16 mmol, 1.0 eq.) and DDQ (536 mg, 2.37 mmol, 1.1 eq.) in DCM (20 mL) was stirred at 30° C. The reaction was monitored by TLC, after consumption of the starting material, the mixture was filtered, filtrate was concentrated and purified by column chromatography on silica gel (PE:EtOAc=10:1) as light yellow solid 78i (360 mg, yield 64%). ¹H NMR (CDCl₃, 300 MHz): δ 7.25 (brs, 1H), 3.90 (s, 2H), 2.89 (brs, 1H), 1.67 (m, 2H), 1.45 (s, 9H), 1.10 (m, 2H).

To a stirred solution of 78i (360 mg, 1.43 mmol, 1.0 eq.) in DCM (8 mL) was added PCC (520 mg, 2.76 mmol, 1.9 eq.). The mixture was stirred at 30° C. for 12 hrs, and then it was filtered through a silica gel column with DCM to yield 78j (320 mg, yield 90%) after removal of solvent as a light yellow solid. ¹H NMR (CDCl₃, 400 MHz): δ 9.89 (s, 1H), 1.98 (m, 2H), 1.73 (m, 2H), 1.46 (s, 9H).

To a solution of 78j (310 mg, 1.25 mmol, 1.0 eq.) in methanol was added 78k (287 mg, 1.5 mmol, 1.2 eq.) and K₂CO₃ (344 mg, 2.5 mmol, 2.0 eq.) at 0° C. The mixture was stirred at rt for 12 hrs and then concentrated under reduced pressure. The residue was diluted with water (30 mL), acidified with aq. citric acid to pH=5-6, extracted with EtOAc (50 mL×2). The combined organic layer was washed with brine, dried over Na₂SO₄, filtered, concentrated. The residue was purified by prep-TLC (PE:EtOAc=2:1) to yield 78l (310 mg, yield 85%) as a light yellow solid. ¹H NMR (CDCl₃, 300 MHz): δ 2.35 (s, 1H), 1.90 (m, 2H), 1.50 (s, 9H), 1.47 (m, 2H).

Compound 78l (310 mg, 1.27 mmol, 1.0 eq.) was dissolved in a solution of HCl/EtOAc (4 M, 3 mL) and stirred at rt for 3 hrs. TLC indicated the reaction complete. The reaction solution was concentrated in vacuum, the resulting residue was diluted with EtOAc (100 mL) and washed with saturated aq. NaHCO₃, dried over Na₂SO₄, filtered and solvent was removed in vacuum to provide 78m (180 mg, yield 98%) as a light yellow solid. ¹H NMR (CDCl₃, 300 MHz): δ 4.92 (brs, 2H), 2.36 (s, 1H), 1.67 (m, 2H), 1.39 (m, 2H).

Compound 78o was synthesized using the general procedure.

Intermediate 78o (465 mg, 1 mmol, 1.0 eq.) and dimethylsulfoxide (5 mL) were charged into a 20 mL vial. Potassium tert-Butoxide (504 mg, 4.5 mmol, 4.5 eq.) was added portionwise and the reaction mixture stirred at ambient temperature for 5 minutes. Intermediate 57 (431 mg, 1.2 mmol, 1.2 eq.) was added in one portion. Stirring was continued for another 1.5 hrs, then the reaction mixture was diluted with water (2 mL) and pH was adjusted to 3 with aq. citric acid. The aqueous phase was extracted with ethyl acetate (50 mL×3). The organic extracts were combined, washed with brine, dried over sodium sulfate, filtered and the solvent was removed in vacuo. The residue was purified by prep-TLC (DCM:MeOH=20:1) to afford compound 78p as a pale yellow solid (400 mg, yield 51%). MS (ESI) m/z (M+H)⁺ 789.1.

Compound 78p (162 mg, 0.21 mmol, 1.0 eq.) and dichloromethane (4 mL) were charged in a 10 mL round bottom flask. CDI (68 mg, 0.41 mmol, 2.0 eq.) was added as a single portion and the reaction mixture stirred at reflux for 5 hrs. Then the resulting mixture was cooled to ambient and compound 78m (97 mg, 0.62 mmol, 3.0 eq.), DBU (94 mg, 0.62 mmol, 3.0 eq.) and dichloromethane (1 mL) were mixed together and the resulting solution was added dropwise to the above reaction mixture. After that, the reaction mixture was heated to reflux and stirring was continued for additional 8 hrs. The reaction mixture was concentrated, diluted with EtOAc (80 mL) and washed with brine; the aqueous phase was adjusted to pH 5-6 with citric acid (aq.). The organic extracts were washed with water, dried over sodium sulfate, filtered and the solvent removed in vacuo. The residue was purified by prep-TLC (PE:EtOAc=3:1) to provide compound 188 as a white solid (160 mg, yield 85%). MS (ESI) m/z (M+H)⁺ 916.5.

### 1.37 Synthesis of Compound 189

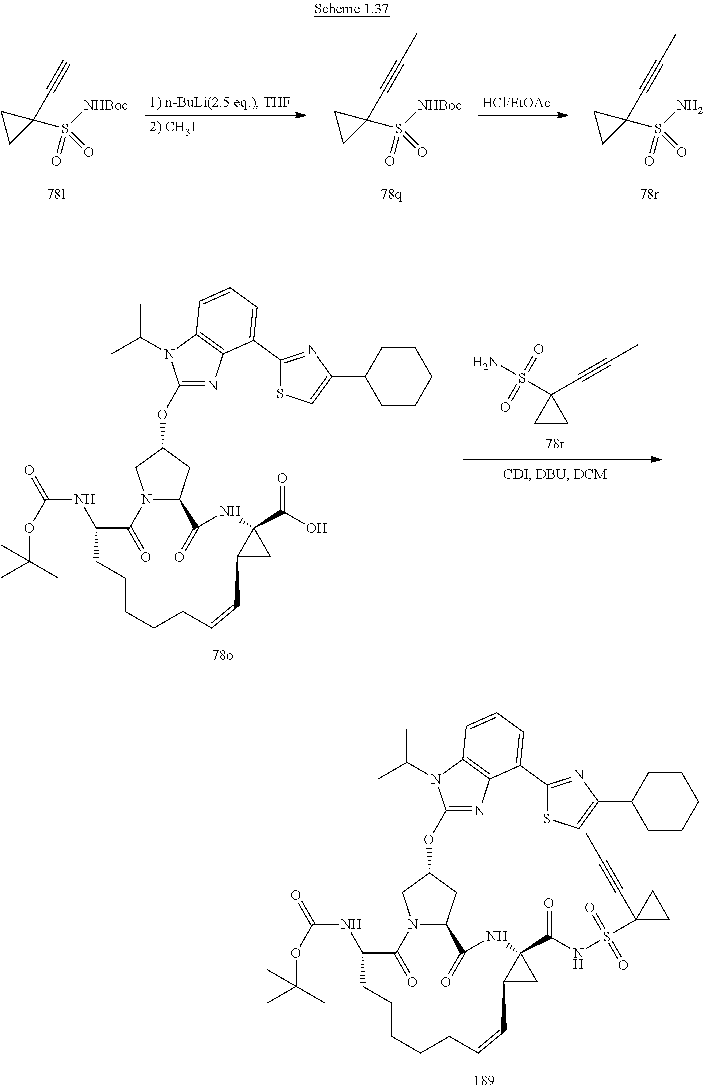

To a solution of compound 78l (310 mg, 1.3 mmol, 1.0 eq.) in anhydrous THF (8 mL) at −78° C. was added n-BuLi (2.5 M in hexane, 1.27 mL, 3.2 mmol, 2.5 eq.) dropwise. After that, the solution was stirred at this temperature for 30 min and slowly warmed to 10° C. and stirred for another 30 min. Then a solution of CH₃I (369 mg, 2.6 mmol, 2.0 eq.) in THF (1 mL) was added dropwise. After addition, the reaction was warmed to 10° C. and monitored by TLC. After 78l was all consumed, the reaction mixture was quenched with NH₄Cl (aq.), extracted with EtOAc and washed with brine. The organic layer was dried over Na₂SO₄, filtered and concentrated by vacuum and purified by prep-TLC. Compound 78q (150 mg, 46% yield) was obtained as a light yellow solid. ¹H NMR (CDCl₃, 300 MHz): δ 1.86 (s, 3H), 1.84 (m, 2H), 1.52 (s, 9H), 1.37 (m, 2H).

Compound 78q (150 mg, 0.58 mmol, 1.0 eq.) was dissolved in HCl/EA (4 M, 3 mL) and stirred at ambient for 3 hrs. TLC indicated the reaction complete. The reaction solution was concentrated by vacuum, diluted with EtOAc and neutralized with NaHCO₃ (aq.), the organic layer was dried over Na₂SO₄, filtered and solvent removed by vacuum to give compound 78r (75 mg, yield 82%) as a light yellow solid. ¹H NMR (CDCl₃, 300 MHz): δ 4.76 (brs, 2H), 1.86 (s, 3H), 1.60 (m, 2H), 1.31 (m, 2H).

Compound 78o (120 mg, 0.15 mmol, 1.0 eq.) and dichloromethane (4 mL) were charged in a 10 mL round bottom flask. 1,1′Carbonyldiimidazole (45 mg, 0.28 mmol, 1.9 eq.) was added as a single portion and the reaction mixture stirred at reflux for 5 hrs. Then the resulting mixture was cooled to ambient and intermediate 78r (75 mg, 0.47 mmol, 3.1 eq.), DBU (71 mg, 0.47 mmol, 3.1 eq.) and dichloromethane (1 mL) were mixed together and the resulting solution was added dropwise to the above reaction mixture. After that, the reaction mixture was heated to reflux and stirring was continued for a further 8 hrs. The reaction mixture was concentrated, diluted with EtOAc and washed with brine, the aqueous phase was adjusted to pH 5-6 with citric acid (aq.). The organic extracts were washed with water, dried over sodium sulfate, filtered and the solvent removed in vacuo. The residue was purified by prep-TLC using ethyl acetate:petroleum (3:1) to give 130 mg (92% yield) of the compound 189 as a white solid. MS (ESI) m/z (M+H)⁺ 930.4.

### 1.38 Synthesis of Compound 190

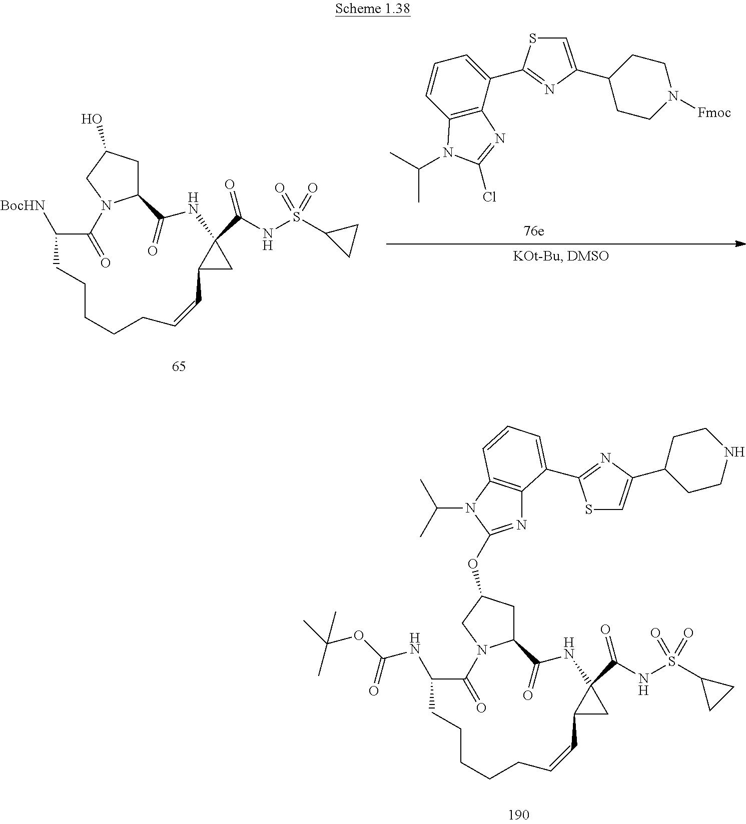

To a solution of compound 65 (1.0 g, 1.76 mmol) in DMSO (5 mL) was added KOt-Bu (1.18 g, 10.56 mmol, 6 e.q.), the mixture was stirred at 0° C. for 0.5 h under nitrogen. After that, compound 76e (1.02 g, 1.76 mmol) was added into the solution, the reaction mixture was stirred at r.t for 1.5 h. After completion of the reaction, the reaction was quenched by ice-water. The mixture was neutralized by aq. HCl (1 M), then extracted with EtOAc (50 mL×3). The combined organic layers were washed with brine, dried over anhydrous Na₂SO₄ and concentrated in vacuo. The residue was purified by prep-HPLC to give compound 190 as white solid (301 mg, yield 16%). MS (ESI) m/z (M+H)⁺ 893.4.

### Example 2

### Benzothiazole Analogs

### 2.1 Synthesis of Compound 201

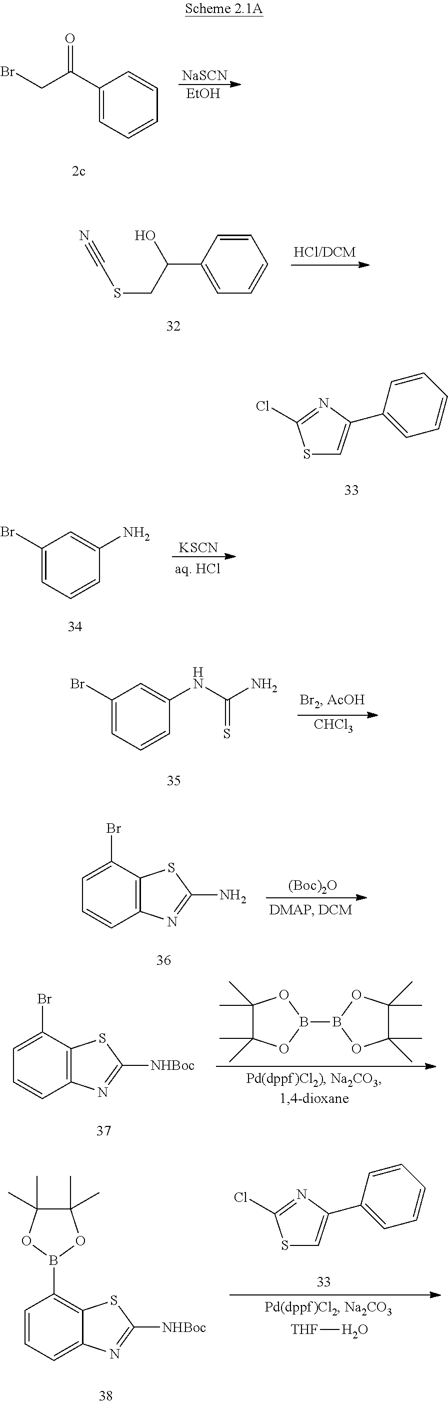

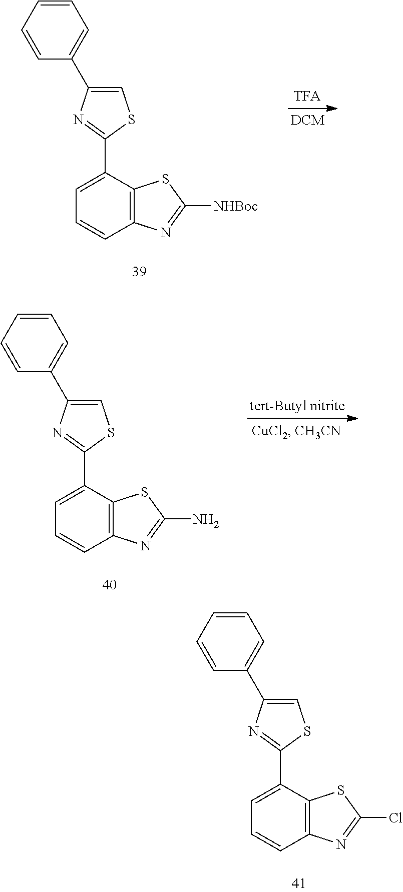

At room temperature, bromoacetophenone 2c (5.0 g, 25.0 mmol) was introduced into 25 mL of ethanol and then treated with sodium thiocyanate (2.4 g, 30 mmol). The mixture was stirred at room temperature. After 3 hrs, TLC (PE:EtOAc=15:1) showed the reaction completed, and then the reaction mixture was dried on a rotary evaporator to give the crude product 32 as a light yellow solid. It was used directly for the next step without further purification.

Crude intermediate 32 were dissolved in 50 mL of dichloromethane and bubbled with hydrogen chloride at 0° C. for 40 min. The mixture was then stirred at room temperature for 2 hrs. For work-up, the solution was treated with a saturated aqueous sodium hydrogen carbonate solution and then extracted with dichloromethane. The combined organic phases were dried over sodium sulfate and the solvent was distilled off on a rotary evaporator. 4 g of compound 33 was obtained with an overall yield 81% of the two steps.

KSCN (12.3 g, 120 mmol) was added to a stirred solution of compound 34 (20 g, 117 mmol) in 1 M HCl (500 mL) at 100° C. and the solution stirred at 100° C. for 16 hrs. The solution was diluted with water (60 mL) and the pH value was adjusted to 8 with ammonia and the mixture at 5° C. for 2 hrs. The precipitate was filtered, washed with cold water (20 mL), washed with ether (5 mL), and dried. The crude solid was purified by column chromatography (petroleum ether/ethyl acetate=4/1), to give thiourea 35 (11 g, yield 41%) as a white powder. MS (ESI) m/z [M+H]⁺ 230.

A solution of compound 35 (8 g, 34.7 mmol) in acetic acid (100 mL) was cooled to 10° C., and a solution bromine (5.5 g, 34.7 mmol) in chloroform (10 mL) was added thereto. Then the mixture was stirred at room temperature overnight. The reaction mixture was distilled to remove acetic acid. After that, the mixture was extracted with EtOAc for three times, the water layer was basified to pH=10 and extracted with EtOAc for three times, the organic layer was combined and washed with brine, dried over sodium sulfate and concentrated in vacuo to give a crude compound 36, which was used directly in the next step. MS (ESI) m/z (M+H)⁺ 228.3.

Compound 36 (5 g, 22 mmol) was dissolved in anhydrous DCM (100 mL), then (Boc)₂O (5.4 g, 25 mmol) was added at 0° C., followed by addition of DMAP (0.5 g, 4 mmol). The mixture was stirred overnight at room temperature. The mixture was directly purified by column chromatography on silica gel to give compound 37 (2 g, yield 27%). MS (ESI) m/z (M+H)⁺ 328.3.

To a solution of compound 37 (2 g, 6 mmol) in 1,4-dioxane (40 mL), Na₂CO₃ (1.28 g, 12 mmol) and Bis(pinacolato)diboron (2.2 g, 9 mmol) were added. The reaction mixture was purged with N₂ for three times, and then Pd(dppf)Cl₂ (200 mg, 0.27 mmol) was added. The mixture was stirred overnight at 100° C. After cooling to rt, the reaction mixture was extracted by EtOAc, and purified by column chromatography on silica gel (PE:EtOAc=4:1) to afford compound 38 (1 g, yield 44%). MS (ESI) m/z (M+H)⁺ 377.3.

Compound 38 (500 mg, 1.33 mmol) and compound 33 (260 mg, 1.33 mmol) were dissolved in THF (15 mL) and H₂O (5 mL), and Na₂CO₃ (282 mg, 2.66 mmol) was added. The reaction mixture was purged with N₂ for three times, and then Pd(dppf)Cl₂ (50 mg, 0.068 mmol) was added. The mixture was stirred overnight at 60° C. LCMS monitored the reaction. After the reaction was completed, the mixture was cooled to rt and extracted with EtOAc, and the organic layer was combined and washed with brine, dried over sodium sulfate and concentrated to 10 mL solvent. The product was filtrated, the solid was dried to give compound 39 (300 mg, yield 55%). ¹H NMR (300 MHz, DMSO) δ 11.7 (s, 1H), 8.27 (s, 1H), 8.13 (d, J=7.2 Hz, 1H), 7.87-7.79 (m, 2H), 7.57-7.40 (m, 4H), 1.54 (s, 9H).

To a solution of compound 39 (300 mg, 0.73 mmol) in DCM (5 mL) was added CF₃COOH (10 mL), the reaction mixture was stirred at room temperature for 3 hrs. While the start material was consumed, the mixture was distilled to remove the solvent. Then the residue was diluted with EtOAc (80 mL), washed with saturated aq. NaHCO₃, the organic layer was dried over sodium sulfate and concentrated in vacuo to yield compound 40 (266 mg, yield 100%).

Copper (II) chloride (102 mg, 0.76 mmol) is dissolved in CH₃CN (3.2 mL) then ter-butyl nitrite (125 uL, 0.95 mmol) is added. The reaction is heated to 60° C., then compound 40 (200 mg, 0.64 mmol) was added. The reaction was stirred at 60° C. for 15 mins, then poured into water and extracted with EtOAc, the crude product was purified by flash column (PE:EtOAc=6:1) to provide compound 41 (150 mg, yield 60%). ¹H NMR (300 MHz, DMSO) δ 8.31 (s, 1H), 8.11-8.08 (m, 4H), 7.72-7.67 (t, 1H), 7.56-7.51 (m, 1H), 7.42-7.37 (m, 1H).

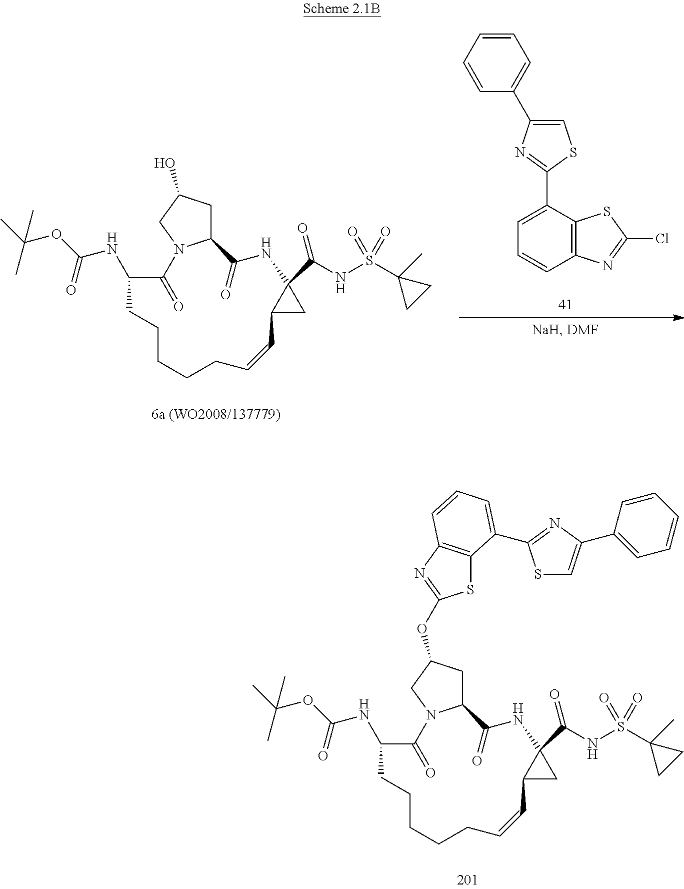

To a suspension of NaH (60%, 28 mg, 0.86 mmol) in 2 mL DMF was added compound 6a (28 mg, 0.086 mmol) at 0° C. After the mixture was stirred for 2 hrs at 0-5° C., compound 41 (50 mg, 0.086 mmol) was added; the resulting mixture was warmed to room temperature and stirred for 3 hrs. After completion of the reaction, the mixture was cooled by ice water, acidified by aq. HCl (1 N) to pH=5-6, then the mixture was extracted with ethyl acetate (30 mL×3), the organic layers were combined, washed by brine, dried over anhydrous sodium sulfate, the solvent was removed under reduced pressure, the residue was purified by prep-HPLC to give compound 201 (33 mg, 40%). MS (ESI) m/z (M+H)⁺ 875.4.

### Example 3

### Tetrahydroquinazolinyl Analogs

### 3.1 Synthesis of Compounds 301 and 302

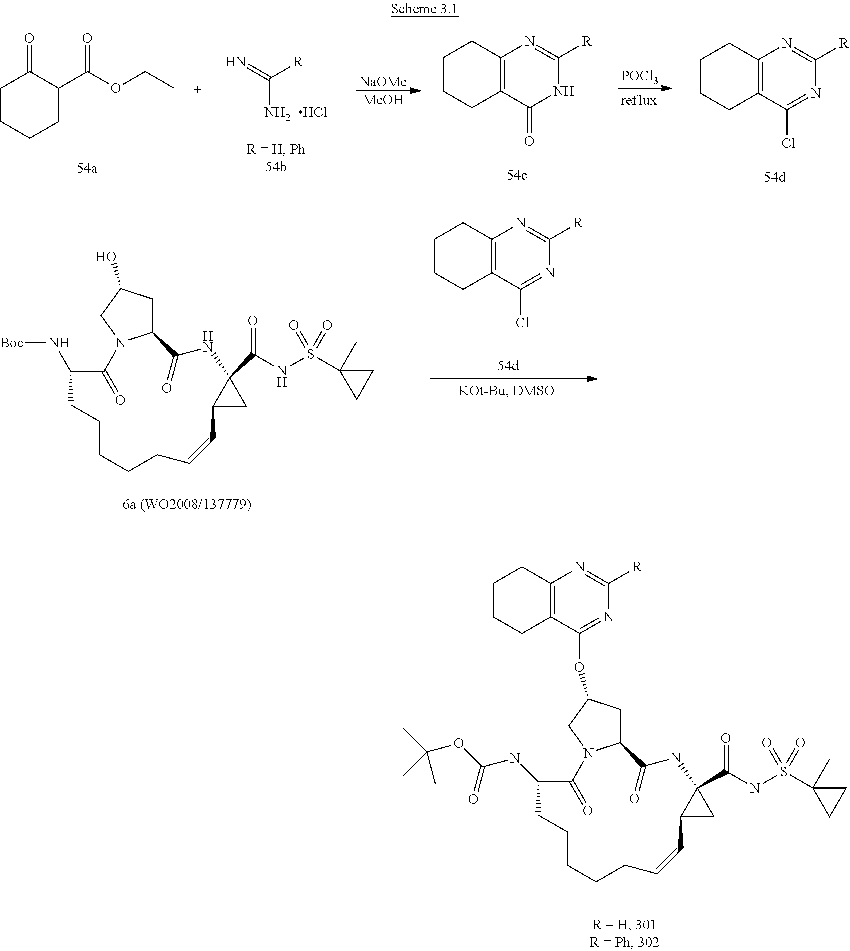

A mixture of compound 54a (1 eq.), amidine 54b (1.5 eq.), and NaOMe (2.5 eq.) in MeOH (c=0.2 mmol/mL) was refluxed for 4 hrs. And then the reaction mixture was concentrated in vacuo, the residue was re-dissolved with EtOAc (100 mL), the resulting solution was washed with water and brine, dried over anhydrous sodium sulfate, concentrated in vacuo. The residue was purified with prep-TLC to provide compound 54c.

The following compounds 54c-1 and 54c-2 were prepared using above general procedure:

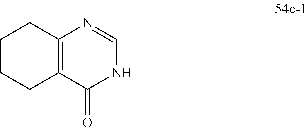

(White solid, 250 mg, yield 83%). ¹H NMR (300 MHz, CDCl₃): 8.04 (s, 1H), 2.66 (s, 2H), 2.52 (t, 2H), 1.82-1.74 (m, 4H).

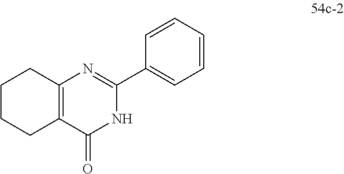

(White solid 400 mg yield 88%). ¹H NMR (300 MHz, CDCl₃): δ 11.85 (s, 1H), 8.11-8.08 (m, 2H), 7.53-7.46 (m, 3H), 2.72 (t, 2H), 2.58 (t, 2H), 1.86-1.76 (m, 4H).

Compound 54c was dissolved in POCl₃ and then the mixture was refluxed for 1 hour. After cooling to rt, most of POCl₃ was removed under reduced pressure. The residue was taken into ice-water, neutralized with saturated aq. NaHCO₃ under cooling, extracted with EtOAc (30 mL×3). The combined organic layer was washed with brine, dried over anhydrous Na₂SO₄ and concentrated under reduced pressure. The crude product was purified with prep-TLC to give compound 54d.

The following compounds were prepared using above general procedure:

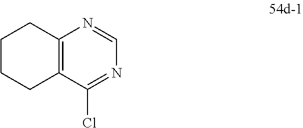

(yellow solid, 182 mg, yield 84%);

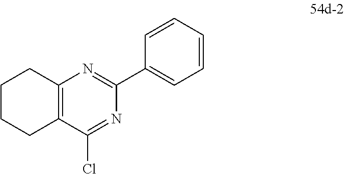

(White solid, 202 mg, yield 95%)

To a solution of compound 6a (1 eq.) in 2 mL of DMSO was added KOt-Bu (4.5 eq.) in portions at ambient temperature, then the mixture was stirred for 2 hrs at ambient temperature. After that, compound 54d (1.1 eq.) was added, the resulting mixture was stirred at rt for 20 hrs, the reaction was monitored by LCMS. The de-Boc product was detected, the mixture was cooled by ice water, acidified by aq. HCl (0.1 M) to pH=7-8. Then Boc₂O (1 eq.) and NaHCO₃ (1.5 eq.) were added. The mixture was stirred for another 2 hrs and then was acidified to pH=5-6 with aq. HCl (0.1M), extracted with ethyl acetate, the organic layers were combined, washed by brine, dried over anhydrous sodium sulfate, concentrated under reduced pressure, the residue was purified by prep-TLC (PE:EtOAc=1:1) to provide compound 301 or 302.

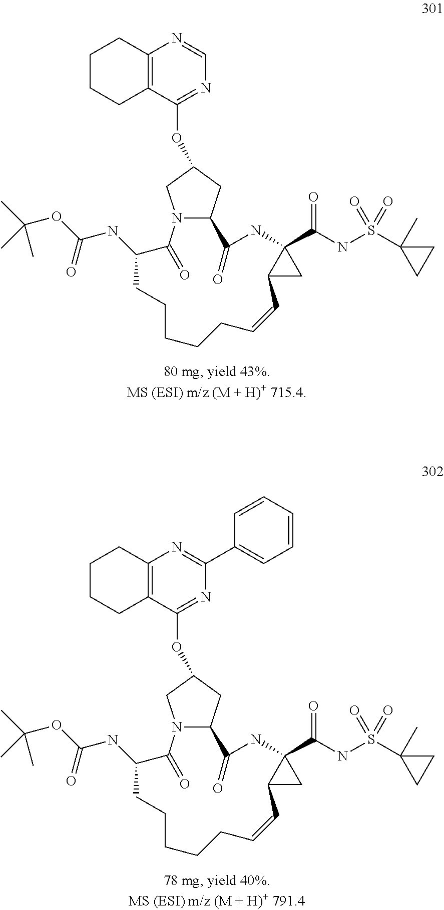

### 3.2 Synthesis of Compounds 303 and 304

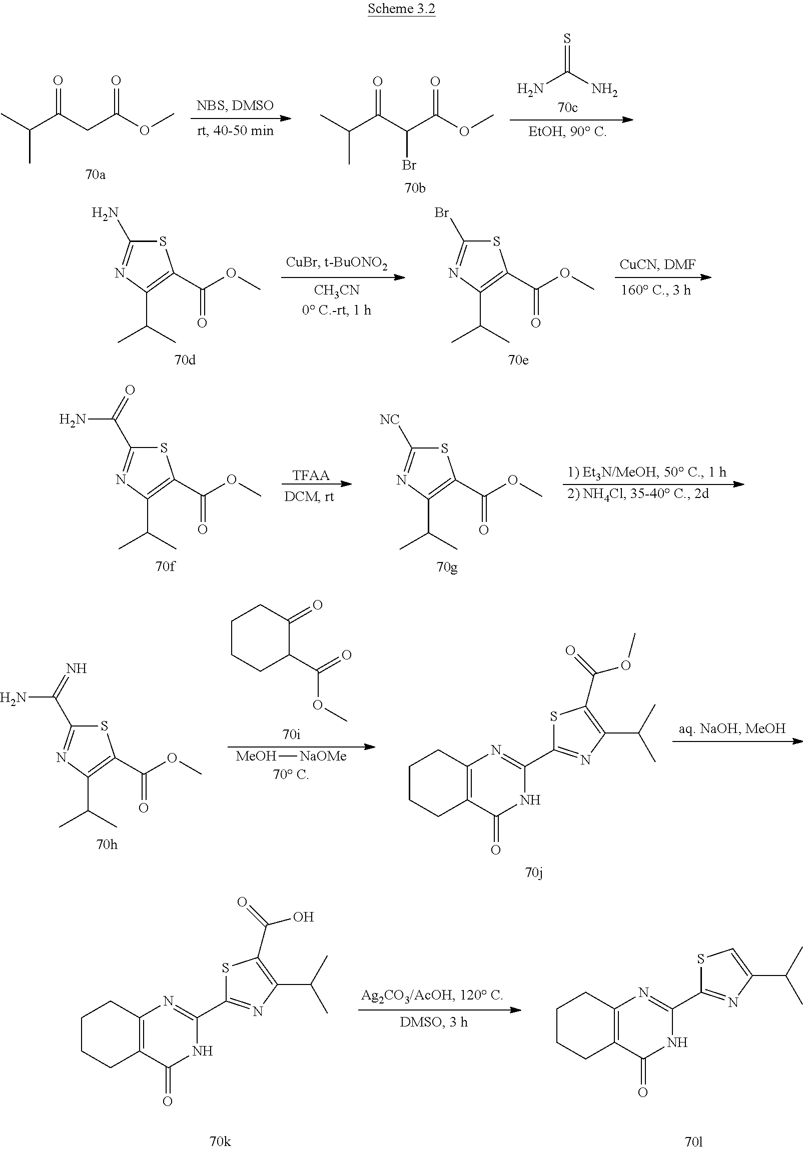

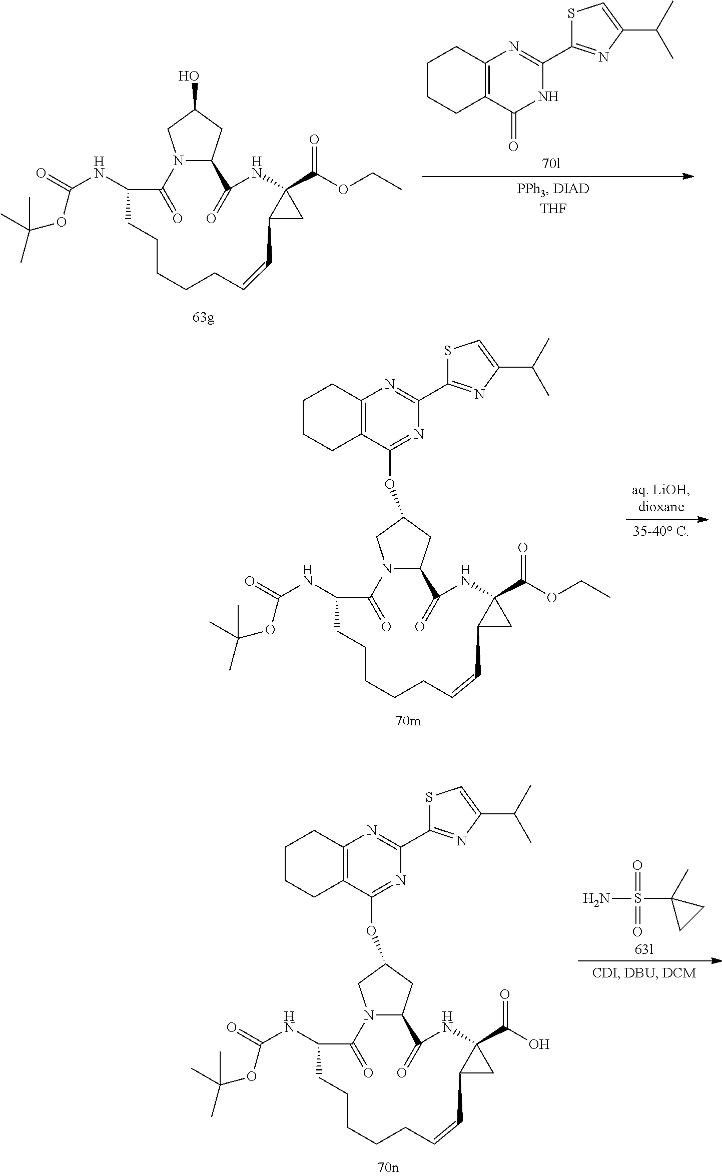

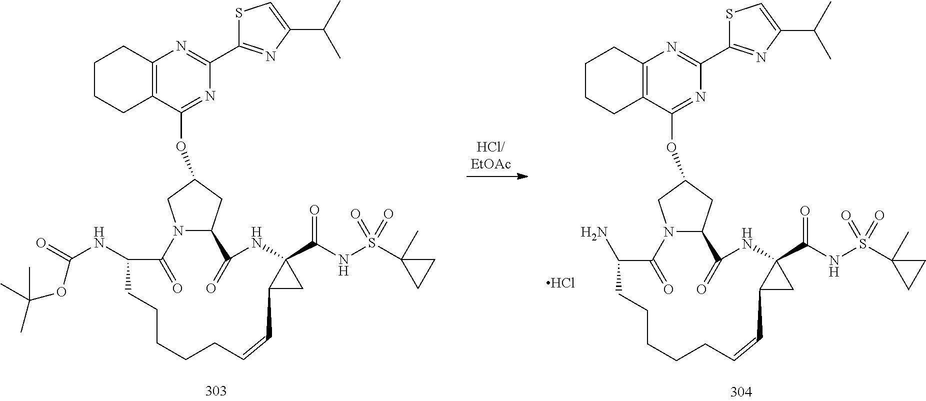

To a solution of compound 70a (20 g, 138.7 mmol, 1 eq) in DMSO (100 mL) was added NBS (25.7 g, 145.6 mmol, 1.05 eq). The resultant mixture was stirred for an appropriate time at room temperature. After completion of the reaction as indicated by thin-layer chromatography (Petroleum ether:EtOAc=5:1), the reaction mixture was washed with sat. aq. NH₄Cl. And then it was extracted with EtOAc (80 mL×3). The combined organic layer was washed with brine, dried over anhydrous Na₂SO₄ and concentrated under reduced pressure to afford compound 70b (30 g, yield 97%) as yellow oil.

The mixture of compound 70b (30 g, 134 mmol, 1 eq) and thiourea (10 g, 134 mmol. 1 eq) in 30 mL of ethanol was heated to reflux for 18 hrs. All the volatiles were removed under reduced pressure. The residual was recrystallized in CH₂Cl₂ to give compound 70d (13 g, yield 48%) as light yellow solid. ¹H NMR (CDCl₃, 300 MHz): δ 9.14 (brs, 2H), 4.02-3.93 (m, 1H), 3.83 (s, 3H), 1.33 (d, J=6.9 Hz, 6H).

A solution of copper (II) bromide (4.5 g, 20 mmol, 1 eq) in acetonitrile (10 mL) was purged with nitrogen and cooled to 0° C. t-Butyl nitrite (3.08 g, 30 mmol, 1.5 eq) was added, followed by a solution of compound 70d (4 g, 20 mmol, 1 eq) in acetonitrile (20 mL). The mixture was stirred at 30° C. and concentrated in vacuo. The residue was dissolved in EtOAc (100 mL), washed with sat. aq. NaHCO₃ and the precipitate was removed by filtration. The organic extract was dried over anhydrous Na₂SO₄ and concentrated under reduced pressure. The crude product compound 70e was used directly for the next step without further purification (3.9 g, yield 74%) as a yellow solid. MS (ESI) m/z (M+H)⁺ 265.7.

The mixture of compound 70e (1.5 g, 5.7 mmol, 1 eq) and CuCN (0.6 g, 6.8 mmol, 1.2 eq) in 15 mL of DMF was heated to 160° C. for 3 h. LCMS analysis showed the reaction completed. The reaction mixture was extracted with EtOAc (50 mL×3) and washed with water and brine. The organic layers was dried over anhydrous Na₂SO₄, and concentrated in vacuo. The residue was purified by flash chromatography (gradient elution: 30%-90% ethyl acetate in Petroleum ether) to give compound 70f (400 mg, yield 31%). MS (ESI) m/z (M+H)⁺ 228.8.

To a mixture of compound 70f (400 mg, 1.75 mmol, 1 eq), Et₃N (796 mg, 7.88 mmol, 4.5 eq) in anhydrous CH₂Cl₂ was added TFAA (339 mg, 3.50 mmol, 2 eq) at 0° C. with stirring. The resulting mixture was warmed to room temperature and stirred for 16 hrs. The reaction was monitored by TLC (PE:EtOAc=5:1) until its completion. The solvent was removed under reduced pressure. The residual was diluted with water (50 mL), extracted with EtOAc (30 mL×3). The organic layer was washed with brine, dried over anhydrous sodium sulfate, filtrate and concentrated to afford the crude compound 70g (120 mg, yield 33%).

To a suspension of compound 70g (890 mg, 4.24 mmol, 1 eq) in 6 mL of methanol was added triethylamine (856 mg, 8.48 mmol, 2 eq). The mixture obtained was heated for 1 h at 50° C. and cooled to 20° C. The precipitate formed was collected carefully. The intermediate was obtained as yellow solid (940 mg). The solid in 10 mL of MeOH was heated for 2 d at 35-40° C. in the presence of ammonium chloride (416 mg, 7.77 mmol, 2 eq). LCMS analysis showed the reaction completed. Solvent was removed under reduced pressure. The residual was washed with water and petroleum ether (20 mL). The crude product compound 70h was used directly for the next step without further purification (800 mg, yield 91%). MS (ESI) m/z (M+H)⁺ 227.7.

A flask was charged with compound 70h (900 mg, 3.7 mmol), compound 70i (632 mg, 3.7 mmol) and NaOMe in 10 mL of MeOH. The reaction mixture was heated at 70° C. for 3 h. LCMS analysis showed the reaction completed. The mixture was cooled down to room temperature. The solvent was removed under reduced pressure. The residue was diluted with water, adjusted to pH=5-6 with 1N HCl. Yellow solid was precipitate out. Collect solid by filtration to yield the compound 70j (1 g, yield 81%).

To the solution of compound 70j (1 g, 3 mmol, 1 eq) in 20 mL of methanol was added NaOH (2.4 g, 60 mmol, 20 eq) in 5 mL of water. The mixture was heated to reflux for 18 hrs. TLC (PE:EtOAc=1:2) analysis showed the reaction completed. The mixture was cooled down to room temperature. The solvent was removed under reduced pressure. The residue was diluted with 3 mL of water, adjusted to pH=4-5. Solid precipitated out. Collect the solid. The crude compound 70k was used directly for the next step without further purification (1 g, crude yield 104%).

A mixture of compound 70k (500 mg, 1.57 mmol, 1 eq.), Ag₂CO₃ (43 mg, 0.16 mmol, 0.1 eq), AcOH (5 mg, 0.08 mmol, 0.05 eq) in 3 mL of DMSO was stirred at 120° C. After 3 hours, the reaction was cooled down to room temperature and quenched with 2 M HCl and the aqueous phase was extracted with EtOAc (50 mL×3). The combined organic layers were washed with brine and dried over anhydrous Na₂SO₄, and concentrated in vacuo. The residue was purified by prep-TLC (Petroleum ether:EtOAc=1:1) to give compound 70l (350 mg, yield 81%). MS (ESI) m/z (M+H)⁺ 275.8.

A flask was charged with compound 63g (100 mg, 0.20 mmol, 1 eq), compound 70l (56, mg, 0.20, mmol, 1 eq), PPh₃ (159 mg, 0.61 mmol, 3 eq) and 5 mL of dry THF and flushed with nitrogen for three times. To the mixture was added DIAD (123 mg, 0.61 mmol, 3 eq) at 0° C. over a period of 10 min. After that, the reaction mixture was warmed to room temperature and stirred for another 18 hrs. LCMS analysis showed the reaction completed. The solvent was removed under reduced pressure. The residue was diluted with water, extracted with EtOAc (30 mL×3). The organic layer was washed with brine, dried over anhydrous sodium sulfate, filtrate, concentrated and purify by prep-TLC (Petroleum ether:EtOAc=1:1) to afford the compound 70m (100 mg, yield 67%). MS (ESI) m/z (M+H)⁺ 751.0.

The preparation of compound 70n were followed the general procedure (100 mg, crude yield 104%). MS (ESI) m/z (M+H)⁺ 723.0.

Compound 303 was prepared by following the general procedure. Purification by prep-TLC (DCM:MeOH=30:1) to give compound 303. 17.5 mg, yield 15%. MS (ESI) m/z (M+Na)⁺ 862.3. Compound 304 was prepared by following the general procedure. 10.2 mg, yield 96%. MS (ESI) m/z (M+Na)⁺ 762.3.

### Example 4

### Tetrahydroisoquinolinyl Analogs

### 4.1 Synthesis of Compound 401

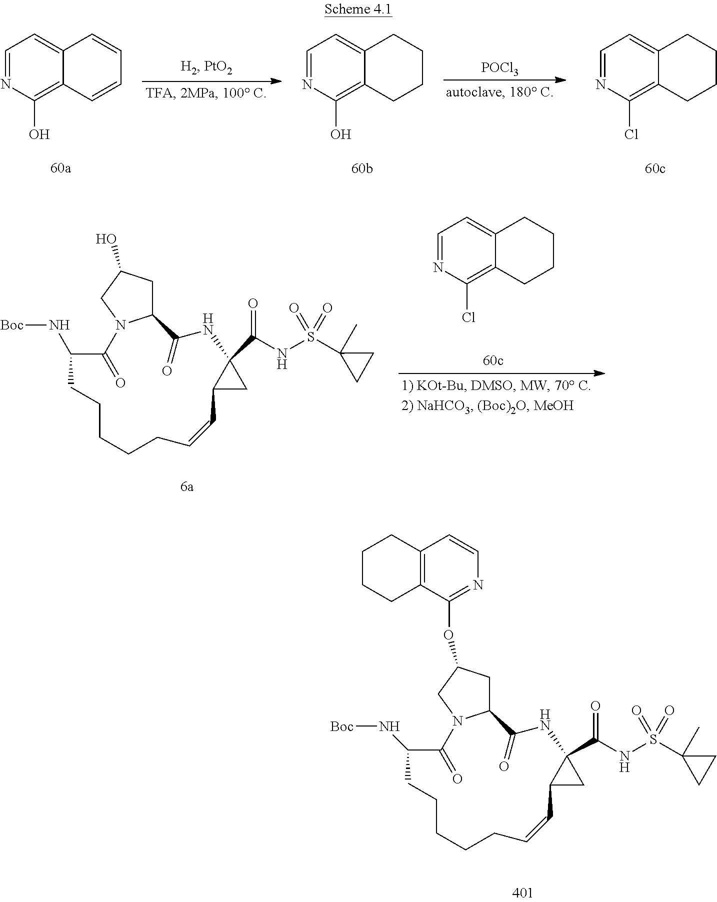

An autoclave was charged with compound 60a (5 g, 34.5 mmol), PtO₂ (0.5 g) and TFA (60 mL), the mixture was hydrogenated under a pressure of 2 MPa of H₂ at 100° C. for 20 hrs. After cooling to rt, the reaction mixture was filtered and the filtrate was concentrated. The residue compound 60b was used directly in next step without further purification. ¹H NMR (300 MHz, CDCl₃): δ 7.16 (d, J=6.6 Hz, 1H), 6.04 (d, J=6.6 Hz, 1H), 2.62-2.51 (m, 4H), 1.78-1.72 (m, 4H). MS (ESI) m/z (M+H)⁺ 149.8.

An autoclave was charged with compound 60b (300 mg, 2 mmol) and POCl₃ (3 mL). The mixture was heated to 180° C. for 5 hrs. After completion of the reaction, the mixture was cooled to rt, taken into ice-water, neutralized with NH₃.H₂O under cooling, extracted with EtOAc (30 mL×3). The combined organic layer was washed with brine, dried over anhydrous Na₂SO₄ and concentrated. The residue was purified by prep-TLC to yield compound 60c as an oil (270 mg, yield 80%). MS (ESI) m/z (M+H)⁺ 168.0.

To a solution of compound 6a (200 mg, 0.345 mmol) in DMSO (4 mL) was added KOt-Bu (175 mg, 1.05 mmol), the mixture was stirred for 0.5 h at r.t under nitrogen. After that, compound 60c (58 mg, 0.345 mmol) was added thereto, The reaction vessel was sealed and heated to 70° C. under microwave for 40 minutes. The reaction was monitored by LCMS. The de-Boc product was detected, then the mixture was cooled to rt, acidified by aq. HCl (1 M) to pH=7-8. Then Boc₂O (75 mg, 0.34 mmol, 1 eq.) and NaHCO₃ (29 mg, 0.345 mmol, 1 eq.) and MeOH (2 mL) were added. The mixture was stirred for additional 2 hrs. The mixture was neutralized by aq. HCl (1 M), and then extracted with EtOAc (30 mL×3). The combined organic layer was washed with brine, dried over anhydrous Na₂SO₄ and concentrated in vacuo. The residue was purified by prep-HPLC to provide compound 401 as white solid (25 mg, yield 10%). MS (ESI) m/z (M+H)⁺ 714.1.

### 4.2 Synthesis of Compound 402

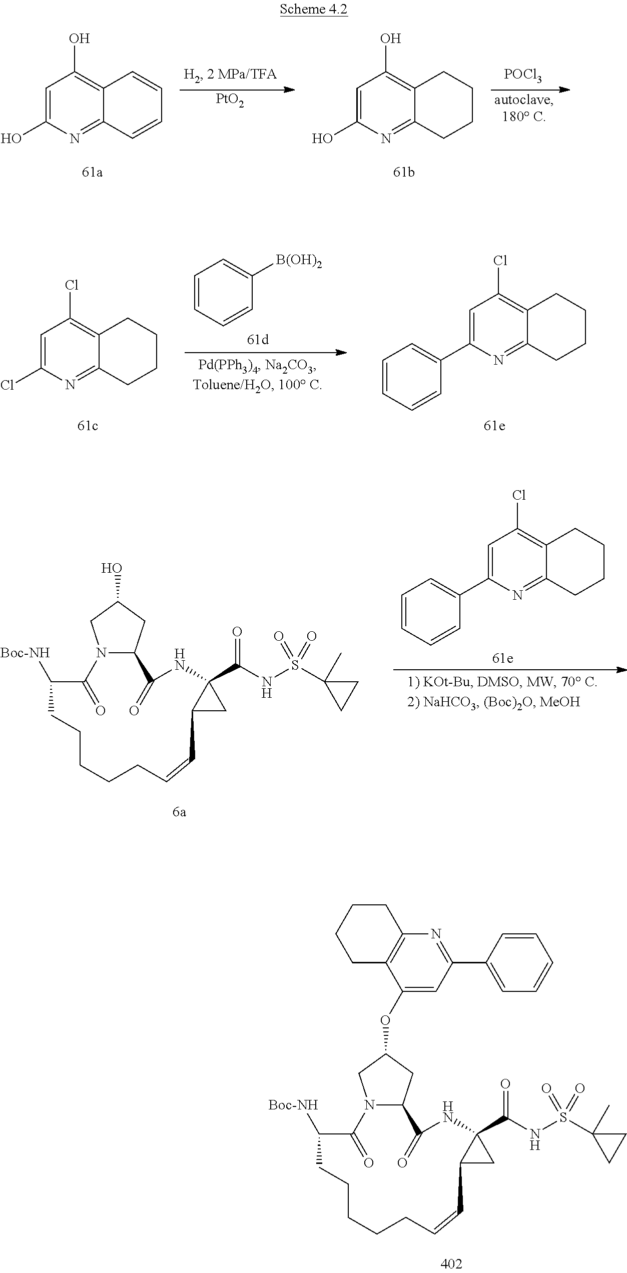

An autoclave was charged with compound 61a (4 g, 24.8 mmol), PtO₂ (0.4 g) and TFA (40 mL), the mixture was hydrogenated under a pressure of 2 MPa of H₂ at 100° C. for 20 hrs. After cooling to rt, the reaction mixture was filtered and the filtrate was concentrated. The residue compound 61b was used directly in next step without further purification (6.0 g, crude yield 120%). MS (ESI) m/z (M+H)⁺ 165.8.

Compound 61b (2 g, 12 mmol) was dissolved in POCl₃ (10 mL) and then the mixture was heated to 180° C. in an autoclave for 5 hrs. After cooling to rt, most of POCl₃ was removed under reduced pressure. The residue was taken into ice-water, neutralized with saturated aq. NaHCO₃, extracted with EtOAc (30 mL×3). The combined organic layer was washed with brine, dried over anhydrous Na₂SO₄ and concentrated. The residue was purified by prep-TLC (PE/EtOAc=2/1) to give compound 61c (700 mg, yield 29%). MS (ESI) m/z (M+H)⁺ 202.

The reaction mixture was diluted with EtOAc (50 mL), washed with water (20 mL) and brine (20 mL*2), and then concentrated. The residue was purified by prep-TLC to give compound 5 (390 mg, yield 71.0%) as white solid. MS (ESI) m/z (M+H)⁺ 243.8.

A flask was charged with compound 61c (450 mg, 2.25 mmol), compound 61d (270 mg, 2.25 mmol), Na₂CO₃ (720 mg, 6.75 mmol), toluene (6 mL) and water (1 mL), and then it was purged with nitrogen for three times. Followed by addition of Pd(PPh₃)₄ (261 mg, 0.225 mmol). The mixture was stirred at 100° C. overnight under N₂ protection. The reaction was monitored by LCMS. Then the mixture was cooled to rt, T diluted with EtOAc (50 mL), washed with water and brine, dried over anhydrous Na₂SO₄, and concentrated in vacuo. The residue was purified by prep-TLC (PE/EtOAc=3/1) to give compound 61e (390 mg, yield 71%) as white solid. MS (ESI) m/z (M+H)⁺ 243.8.

Compound 402 was prepared using a procedure similar to that for preparing compound 401. 20 mg, yield 7%, white solid. MS (ESI) m/z (M+H)⁺ 790.5.

### 4.3 Synthesis of Compound 403

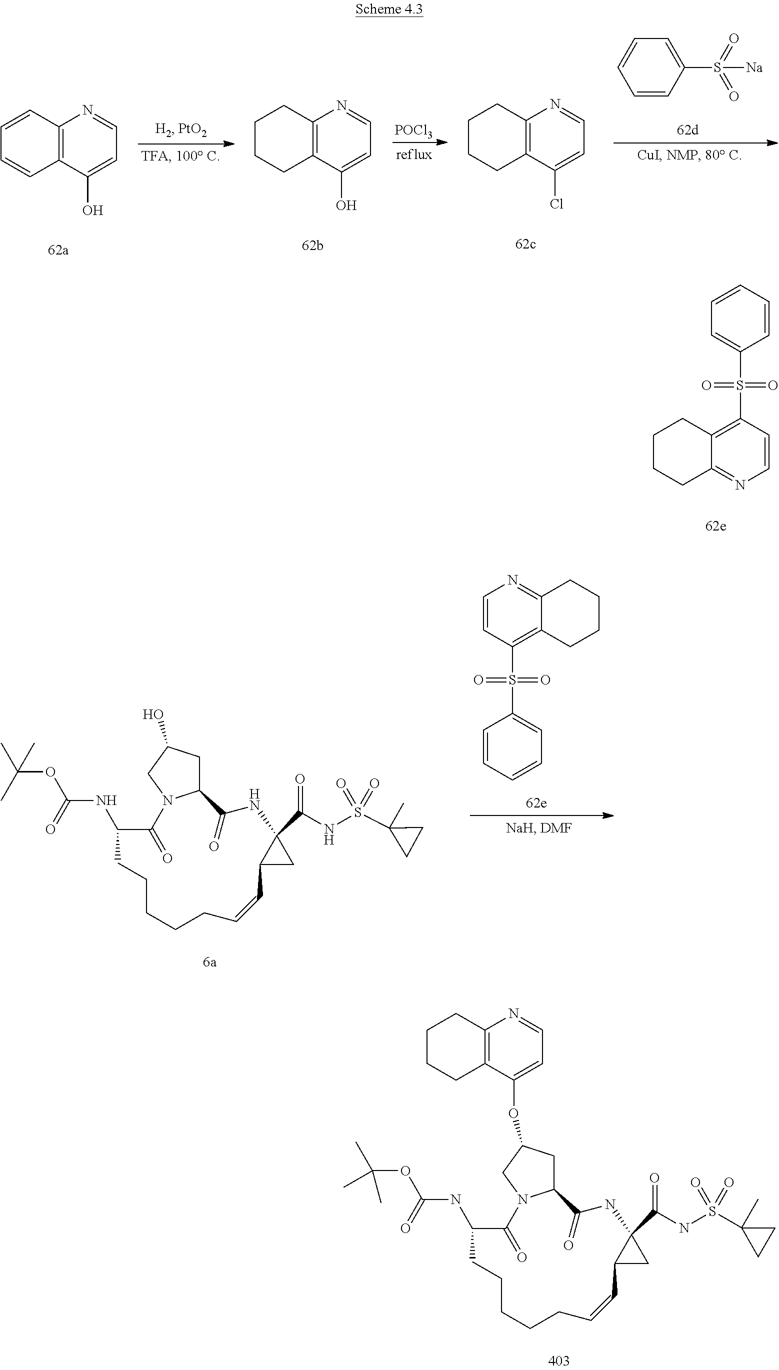

An autoclave was charged with compound 62a (4 g, 27.6 mmol), PtO₂ (0.8 g, 3.5 mmol) and TFA (30 mL), the mixture was hydrogenated under a pressure of 2 MPa at 100° C. for 17 hrs. After cooling to rt, the reaction was diluted with DCM (50 mL) and filtered. The filtrate was concentrated under reduced pressure, neutralized with saturated aq. NaHCO₃ in ice bath, extracted with ethyl acetate (80 mL×3). The organic layer was washed with brine, dried over anhydrous sodium sulfate, filtered and concentrated to give green oil. The crude product was purified by prep-TLC to provide compound 62b as green solid (340 mg, yield 8%). MS (ESI) m/z (M+H)⁺ 149.8.

Compound 62c was prepared following the general procedure (80 mg, yield 44%). MS (ESI) m/z (M+H)⁺ 167.6.

A flask was charged with compound 62c (80 mg, 0.48 mmol), compound 62d (392 mg, 2.39 mmol), CuI (455 mg, 2.39 mmol) and 2 mL of 1-methyl-2-pyrrolidinone. The mixture was heated at 80° C. for 7 hrs. LCMS analysis showed the reaction completed. The mixture was cooled to room temperature, diluted with ethyl acetate (100 mL) and filtered. The filtrate was washed with water and brine, dried over anhydrous sodium sulfate, concentrated in vacuo to provide yellow oil. Purification by prep-TLC afforded a light yellow solid compound 62e (34 mg, yield 26%). MS (ESI) m/z (M+H)⁺ 273.9.

To a solution of compound 6a (100 mg, 0.17 mmol) in DMF (2 mL) was added NaH (60% dispersion in mineral oil, 55 mg, 1.36 mmol) at 0° C. After stirring for 1 hour at 0-5° C., compound 62e (70 mg, 0.255 mmol) was added, the resulting mixture was warmed to room temperature and stirred for 12 hrs. After completion of the reaction, the mixture was quenched with ice-water, acidified with aq HCl (0.1 M) to pH=5-6, then the mixture was extracted with ethyl acetate (30 mL×3). The organic layers were combined, washed with brine, dried over anhydrous sodium sulfate, the solvent was removed under reduced pressure. The residue was purified by prep-HPLC to provide compound 403 (3.1 mg, yield 2.6%). MS (ESI) m/z (M+H)⁺ 714.0.

### 4.4 Synthesis of Compound 404

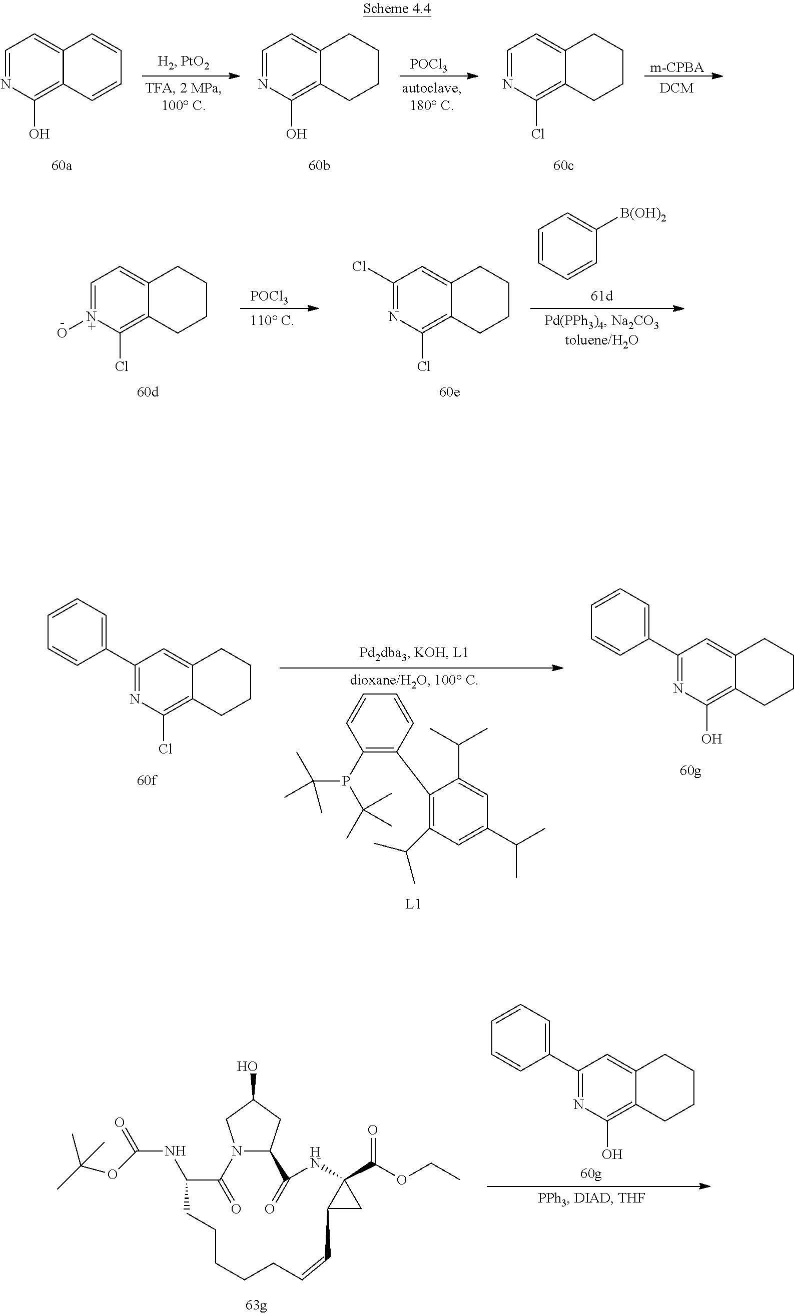

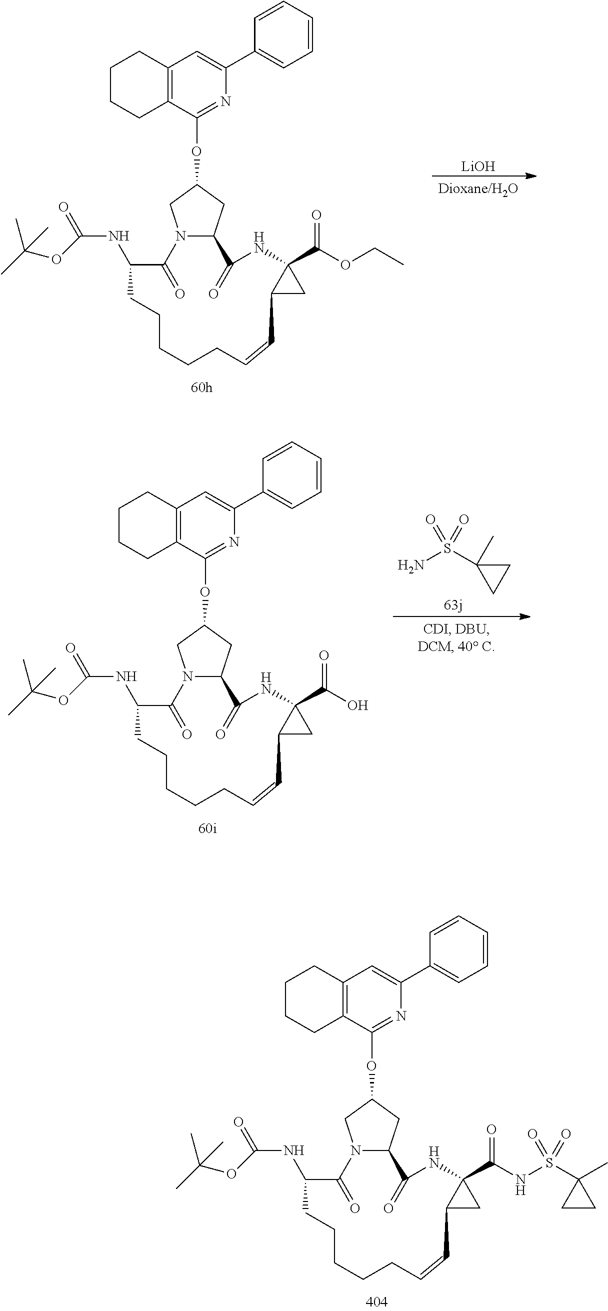

A mixture of compound 60a (5 g, 34.5 mmol) and PtO₂ (0.5 g) in TFA (60 mL) was stirred under 2 MPa of H₂ at 100° C. for 20 hrs. The reaction mixture was filtered and the filtrate was concentrated. The residue compound 60b was used directly for next step without further purification. MS (ESI) m/z (M+H)⁺ 149.8. ¹H NMR (CDCl₃, 300 MHz): 7.16 (d, J=6.6 Hz, 1H), 6.04 (d, J=6.6 Hz, 1H), 2.61-2.50 (m, 4H), 1.82-1.70 (m, 4H). MS (ESI) m/z (M+H)⁺ 149.8.

Compound 60b (300 mg, 2 mmol) was dissolved in POCl₃ (3 mL) and then the mixture was heated to 180° C. in an autoclave for 5 hrs. After completion, The reaction mixture was taken into ice-water, neutralized with ammonia under cooling, extracted with EtOAc (30 mL×3). The combined organic layer was washed with brine, dried over anhydrous Na₂SO₄ and concentrated. The residue was purified by prep-TLC (Petroleum ether:EtOAc=2:1) to give product compound 60c as a liquid (270 mg, Yield 80%). MS (ESI) m/z (M+H)⁺ 167.8.

To a solution of compound 60c (1.8 g, 10.78 mmol) in DCM (70 mL) was added m-CPBA (1.2 g, 12.9 mmol) in portions at 0° C., and then the mixture was stirred at room temperature under an atmosphere of N₂ overnight. The reaction mixture was quenched with aq. Na₂SO₃, diluted with DCM (50 mL), washed with sat. aq. NaHCO₃ and brine, and then concentrated to dryness to give crude compound 60d (1.9 g, crude yield 96%) as solid. MS (ESI) m/z (M+H)⁺ 183.8.

Compound 60d (1.2 g, 6.55 mmol) was dissolved in POCl₃ (5 mL) and then the mixture was heated to 110° C. for 3 hrs. After completion, The reaction mixture was taken into ice-water, neutralized with ammonia under cooling, extracted with EtOAc (40 mL×3). The combined organic layer was washed with brine, dried over anhydrous Na₂SO₄ and concentrated. The residue was purified by flash chromatography (Petroleum ether:EtOAc=3:1) to give the product compound 60e as a white solid. (330 mg, yield 25%). MS (ESI) m/z (M+H)⁺ 201.7.

A flask was charged with compound 60e (350 mg, 1.74 mmol), compound 61d (212 mg, 1.74 mmol), Na₂CO₃ (553 mg, 5.2 mmol), H₂O (1 mL) and toluene (8 mL). It was purged with N₂, and then Pd(PPh₃)₄ (201 mg, 0.174 mmol) was added thereto. The reaction mixture was stirred at 100° C. under an atmosphere of N₂ overnight. After cooled to room temperature, the reaction mixture was diluted with EtOAc (50 mL), washed with water and brine, and then concentrated. The residue was purified by prep-TLC (Petroleum ether:EtOAc=3:1) to give a mixture of compound 60f and compound 60e (170 mg, crude yield 40%) as white solid. MS (ESI) m/z (M+H)⁺ 243.8.

A flask was charged with compound 60f (200 mg, 0.82 mmol), compound L1 (120 mg, 0.28 mmol), KOH (560 mg, 10 mmol), H₂O (10 mL) and toluene (10 mL). It was purged with N₂, and then Pd₂ dba₃ (40 mg, 0.064 mmol) was added thereto. The mixture was stirred at 100° C. under an atmosphere of N₂ overnight. After cooled to rt, the reaction mixture was acidified to pH=3-4, extracted with EtOAc (30 mL×3), washed with water and brine, and then concentrated. The residue was purified by prep-TLC (Petroleum ether:EtOAc=2:1) to give compound 60g (100 mg, yield 54%) as white solid. ¹H NMR (CDCl₃, 400 MHz): 10.01 (brs, 1H), 7.64-7.58 (m, 2H), 7.51-7.42 (m, 3H), 6.25 (s, 1H), 2.61 (dt, J=5.2 Hz, 17.2 Hz, 4H), 1.85-1.75 (m, 4H). MS (ESI) m/z (M+H)⁺ 225.9.

A three neck flask (100 mL) was charged with compound 63g (177 mg, 0.356 mmol, 1.0 eq.), compound 60g (80 mg, 0.356 mmol, 1.0 eq.), triphenylphosphine (280 mg, 1.07 mmol, 3 eq.) and anhydrous tetrahydrofuran (10 mL). The reaction mixture was cooled on top of an ice bath and diisopropylazodicarboxylate (DIAD, 216 mg, 1.07 mmol, 3 eq.) was added dropwise. The cooling bath was removed and stirring was continued at ambient temperature for further 3 hours by which time LCMS analysis showed full consumption of the starting material. Saturated aqueous sodium hydrogen carbonate (10 mL) was added and the reaction mixture stirred for a further 5 minutes, the reaction mixture was then extracted with DCM (30 mL×3). The organic layer was combined, concentrated in vacuo. The residue was purified by prep-TLC (DCM:methanol=45:1) to provide compound 60h (85 mg, yield 34%). MS (ESI) m/z (M+H)⁺ 701.1.

To a solution of compound 60h (100 mg, 0.14 mmol) in 1,4-dioxane (5 mL) was added aq. LiOH (1M, 1.1 mL, 1.14 mmol). The resulting mixture was stirred at ambient temperature overnight. The reaction mixture was acidified to pH=3 with aq. HCl (1N), then extracted with EtOAc (30 mL×3), the combined organic layers was washed with brine. The organic layer was dried in vacuo to give the crude product 60i (100 mg, crude yield 106%). MS (ESI) m/z (M+H)⁺ 673.0.

To a solution of compound 60i (94 mg, 0.14 mmol) in anhydrous DCM (20 mL) was added CDI (91 mg, 0.56 mmol), the resulting solution was refluxed at 40° C. for 4 hours under nitrogen atmosphere. After the intermediate was detected in LCMS, sulfonamide 63j (193 mg, 1.4 mmol) and DBU (152 mg, 1.12 mmol) were added. The reaction mixture was stirred overnight at 40° C. LCMS monitored the reaction. After completion of the reaction, the solution was diluted with EtOAc (100 mL), the solution was washed with aq. HCl, followed by water and brine, dried over anhydrous Na₂SO₄ and concentrated in vacuo. The residue was purified by prep-HPLC to afford compound 404 (20 mg, yield 16%). MS (ESI) m/z (M+H)⁺ 790.3.

### 4.5 Synthesis of Compound 405

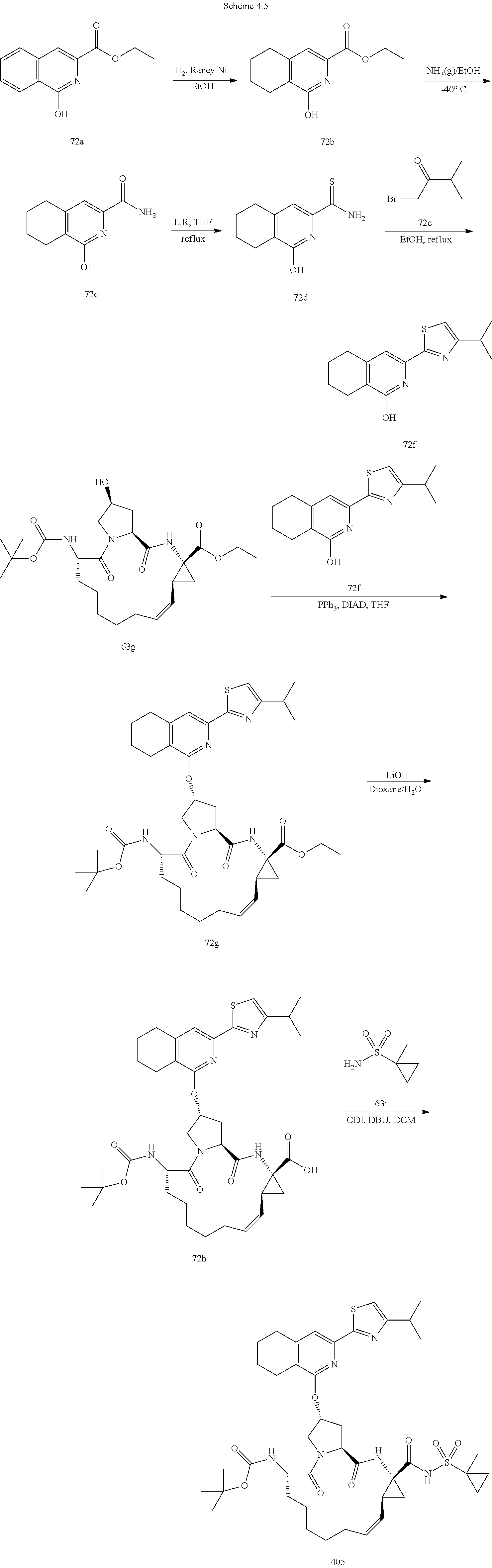

To a solution of compound 72a (200 mg, 0.92 mmol) in EtOH (30 mL) was added Raney Ni (20 mg). The mixture was refluxed under H₂ (3.0 MPa) for 20 hrs. After completion of the reaction, the solvent was filtered and evaporated under reduced pressure to give a crude product, which was purified by prep-HPLC to afford compound 72b (50 mg, yield 25%) as light yellow solid. ¹H NMR (CD₃OD, 400 MHz): δ 6.91 (s, 1H), 4.38 (q, J=7.2 Hz, 2H), 2.66 (m, 2H), 2.51 (m, 2H), 1.78 (m, 4H), 1.38 (t, J=7.2 Hz, 3H). MS (ESI) m/z (M+H)⁺ 222.

To a solution of compound 72b (50 mg, 0.22 mmol) in EtOH (10 mL) was added EtOH-NH₃ (20 mL) under −40° C. for 16 hrs. After completion of the reaction, the solvent was evaporated under reduced pressure to give compound 72c (34 mg, yield 81%). ¹H NMR (CD₃OD, 400 MHz): δ 6.86 (s, 1H), 2.66 (m, 2H), 2.51 (m, 2H), 1.78 (m, 4H). MS (ESI) m/z (M+H)⁺ 193.1.

To a solution of compound 72c (110 mg, 0.57 mmol) in THF (10 mL) was added Lawesson reagent (230 mg, 0.57 mmol). The mixture was refluxed under nitrogen for 6 hrs. After completion of the reaction, the solvent was evaporated under reduced pressure to give a crude compound 72d (114 mg, 96%), which was used directly for next step without further purification. MS (ESI) m/z (M+H)⁺ 209.

To a solution of compound 72d (60 mg, 0.27 mmol) in anhydrous EtOH (10 mL) was added compound 72e (77 mg, 0.5 mmol). The mixture was refluxed under nitrogen for 5 hrs. After completion of the reaction, the solvent was evaporated under reduced pressure to give a crude product, which was purified by prep-HPLC to afford compound 72f (20 mg, yield 28%) as white solid. ¹H NMR (CDCl₃, 400 MHz): δ 6.98 (s, 1H), 6.67 (s, 1H), 3.12 (m, 1H), 2.69 (m, 2H), 2.61 (m, 2H), 1.81 (m, 4H), 1.35 (d, J=7.2 Hz, 6H). MS (ESI) m/z (M+H)⁺ 274.9.

A three neck flask (50 mL) was charged with compound 63g (72 mg, 0.146 mmol, 1.0 eq.), compound 72f (40 mg, 0.146 mmol, 1.0 eq.), triphenylphosphine (115 mg, 0.438 mmol, 3 eq.) and anhydrous tetrahydrofuran (8 mL). The reaction mixture was cooled on top of an ice bath and diisopropylazodicarboxylate (DIAD, 0.1 mL, 0.438 mmol, 3 eq.) was added dropwise. The cooling bath was removed and stirring was continued at ambient temperature for a further 3 hours by which time TLC and LCMS analysis showed full consumption of the starting material. Saturated aqueous sodium hydrogen carbonate (10 mL) was added and the reaction mixture stirred for a further 5 minutes, the reaction mixture was then extracted with DCM (20 mL×3). The organic layer was combined, concentrated in vacuo. The residue was purified by prep-TLC (Petroleum ether:EtOAc=1:1) to give compound 72g (100 mg, yield 91%). MS (ESI) m/z (M+H)⁺ 750.0.

To a solution of compound 72g (100 mg, 0.12 mmol) in 1,4-dioxane (5 mL) was added aq. LiOH (1M, 1.6 mL, 1.6 mmol). The resulting mixture was stirred at ambient temperature overnight. The reaction mixture was acidified to pH=3 with a.q. HCl (1 N), then extracted with EtOAc (30 mL×3), the combined organic layers was washed with brine. The organic layer was dried in vacuo to give the compound 72h (100 mg, crude yield 115%).

To a solution of compound 72h (100 mg, 0.14 mmol) in anhydrous DCM (12 mL) was added CDI (140 mg, 0.86 mmol), the resulting solution was refluxed at 40° C. for 4 hours under nitrogen atmosphere. After the intermediate was detected in LCMS, sulfonamide 63j (200 mg, 1.48 mmol) and DBU (64 mg, 0.42 mmol) was added. The reaction mixture was stirred overnight at 40° C. LCMS monitored the reaction. After completion of the reaction, the solution was diluted with EtOAc (60 mL), the solution was washed with aq. HCl (1 N), water and brine, dried over anhydrous Na₂SO₄ and concentrated in vacuo. The residue was purified by prep-TLC (Petroleum ether:EtOAc=1:1) to afford compound 405 (40 mg, yield 34%). MS (ESI) m/z (M+H)⁺ 839.3.

### 4.6 Synthesis of Compound 406

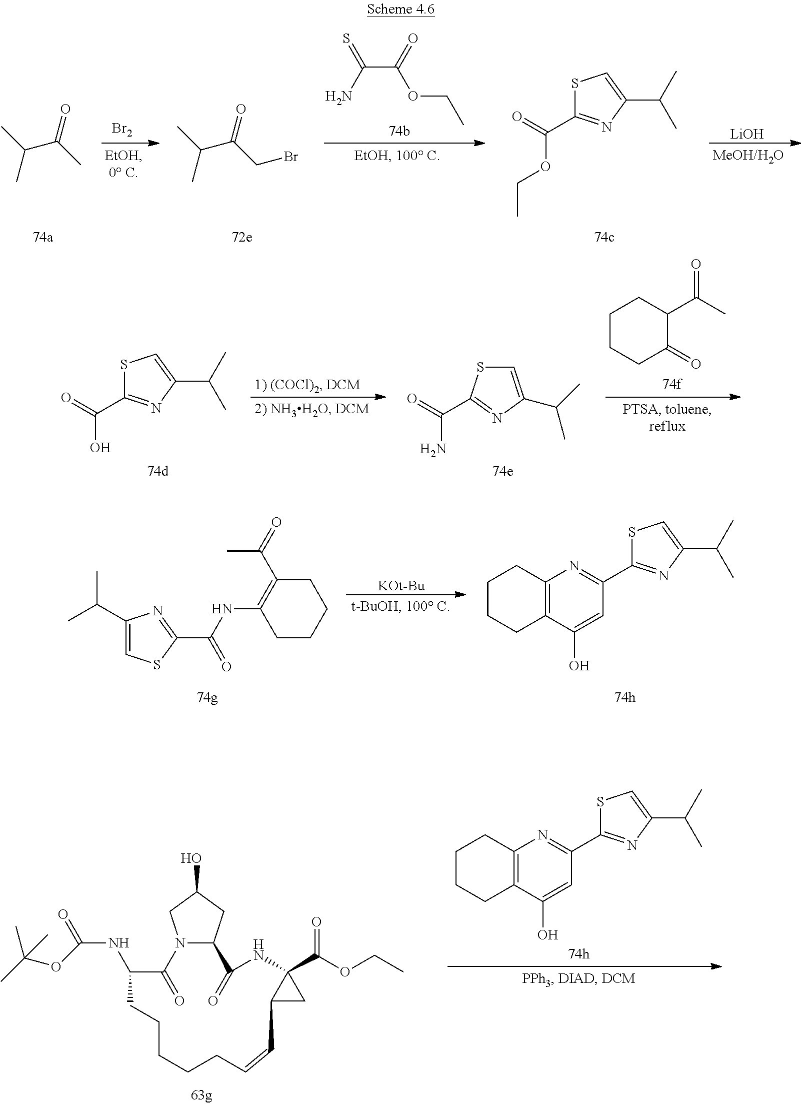

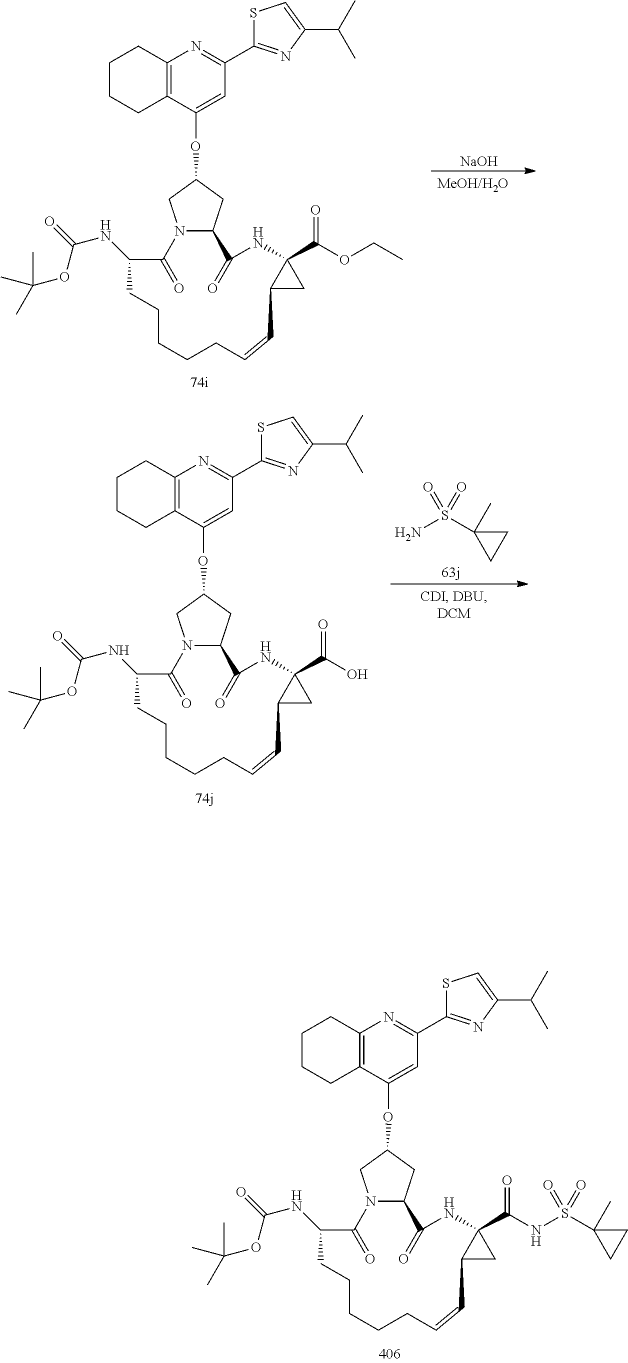

To a solution of compound 74a (12.6 g, 0.146 mmol) in EtOH (80 mL) was added Br₂ (19.4 g, 0.121 mmol) under nitrogen at −5° C. in portions. The reaction mixture was stirred at 0° C. for additional 4 hrs, it become colorless. Then quenched with ice-water and neutralized by saturated aq. NaHCO₃. Extracted with DCM (50 mL×3), the organic layer was washed with water for 2 times, dried over anhydrous Na₂SO₄, concentrated to give compound 72e (15 g, yield 62%). ¹H NMR (CDCl₃, 300 MHz): δ 3.97 (s, 2H), 3.03-2.92 (m, 1H), 1.14 (d, J=6.9 Hz, 6H).

A flask was charged with compound 72e (14 g, 85 mmol), compound 74b (9 g, 67 mmol) and EtOH (100 mL) was refluxed at 100° C. for 1 h, after cooling, the mixture was concentrated, purified by column chromatography on silica gel (PE:EtOAc=5:1) to give compound 74c (15 g, yield 88%). ¹H NMR (DMSO-d₆, 300 MHz): δ 7.69 (s, 1H), 4.37-4.26 (m, 2H), 3.12-3.02 (m, 1H), 1.27 (t, J=7.2 Hz, 3H), 1.20 (d, J=6.9 Hz, 6H).

To a solution of compound 74c (10 g, 50 mmol) in MeOH (70 mL) and H₂O (20 mL) was added LiOH (10.5 g, 250 mmol). The mixture was stirred at room temperature overnight. The mixture was concentrated and added water (20 mL), acidified with diluted HCl solution (2 N). Extracted with EtOAc (50 mL×3), the combined organic layer was washed with brine, dried over anhydrous Na₂SO₄, filtered and concentrated to give compound 74d (8 g, yield 89%) without further purification.

To a solution of compound 74d (6 g, 35 mmol) in DCM (100 mL) was added oxalyl chloride (8.9 g, 70 mmol) and one drop of DMF. The mixture was stirred at room temperature for 1 h, then concentrated and dissolved in DCM (50 mL), added ammonia and stirred for additional 1 h. the mixture was neutralized with diluted HCl, extracted with EtOAc (50 mL×3), the combined organic layer was washed with brine, dried over anhydrous Na₂SO₄, concentrated in vacuo. The residue was purified by column chromatography on silica gel (PE:EtOAc=3:1) to give compound 74e (4 g, yield 65%).

A flask was charged with compound 74e (2.6 g, 18.3 mmol), compound 74f (1.036 g, 6.09 mmol), toluene (20 mL) and PTSA (500 mg, 2.6 mmol). The mixture was stirred over refluxed at 120° C. overnight. After cooling, the mixture was poured into water, neutralized by saturated aq. NaHCO₃, extracted with EtOAc (50 mL×3). The combined organic layer was washed with brine, dried over anhydrous Na₂SO₄, concentrated in vacuo. The residue was purified by column chromatography on silica gel (PE:EtOAc=3:1) to give compound 74g (830 mg, yield 46%). ¹H NMR (CDCl₃, 400 MHz): δ 13.94 (s, 1H), 7.16 (s, 1H), 3.22 (m, 1H), 3.13 (m, 2H), 2.49 (m, 2H), 2.27 (s, 3H), 1.69 (m, 4H), 1.38 (d, J=6.8 Hz, 6H).

A flask was charged with compound 74g (832 mg, 2.85 mmol) and t-BuOH (20 mL), when heated to 60° C., added KOt-Bu (800 mg, 7.12 mmol) and stirred at 100° C. for 3 hrs. After cooling, the mixture was diluted with water (30 mL), neutralized with aq HCl (2 N), and then extracted with EtOAc (50 mL×3). The combined organic layer was washed with brine, dried over anhydrous Na₂SO₄, concentrated in vacuo. The residue was purified by prep-TLC (DCM:MeOH=20:1) to give compound 74h (150 mg, yield 19%). ¹H NMR (CDCl₃, 400 MHz): δ 7.04 (s, 1H), 6.93 (s, 1H), 3.17-3.10 (m, 1H), 2.77 (m, 2H), 2.62 (m, 2H), 2.36-2.30 (m, 4H), 1.35 (d, J=6.8 Hz, 6H).

A flask was charged with compound 63g (100 mg, 0.209 mmol), compound 74h (57 mg, 0.202 mmol), triphenylphosphine (164 mg, 0.627 mmol,) and anhydrous DCM (3 mL). The reaction mixture was cooled on top of an ice bath and diisopropylazodicarboxylate (DIAD, 122 mg, 0.606 mmol) was added in portions. The cooling bath was removed and stirring was continued at ambient temperature for additional 3 hours. The reaction mixture was then extracted with DCM (100 mL×3). The organic layer was combined, washed with brine, dried over anhydrous Na₂SO₄, concentrated in vacuo. The residue was purified by prep-TLC (PE:EtOAc=1:1) to give compound 74i (80 mg, yield 51%). MS (ESI) m/z (M+H)⁺ 750.1

A flask was charged with compound 74i (80 mg, 0.107 mmol), MeOH (4 mL) and H₂O (2 mL). NaOH (40 mg, 0.98 mmol) was added in portions thereto. The mixture was stirred at room temperature overnight. The mixture was concentrated and added water (20 mL), acidified with aq. HCl (1 N) to pH=4-5. Extracted with EtOAc (50 mL×3), The organic layer was combined, washed with brine, dried over anhydrous Na₂SO₄, concentrated in vacuo to provide compound 74j (70 mg, yield 90%).

To a solution of compound 74j (70 mg, 0.087 mmol) in anhydrous DCM (5 mL) was added CDI (94 mg, 0.58 mmol). The resulting solution was refluxed at 60° C. for 4 hours under nitrogen atmosphere. After the intermediate was detected in LCMS, sulfonamide 63j (104 mg, 0.777 mmol) and DBU (36 mg, 0.243 mmol) was added. The reaction mixture was stirred overnight at 60° C. LCMS monitored the reaction. After completion of the reaction, the solution was diluted with EtOAc (100 mL), the solution was washed with brine, dried over anhydrous Na₂SO₄ and concentrated in vacuo. The residue was purified by prep-HPLC (PE:EtOAc=1:1) to afford compound 406 (24 mg, yield 33%). MS (ESI) m/z (M+H)⁺ 839.2

### Example 5

### Quinolinyl Analogs

### 5.1 Synthesis of Compounds 501 and 502

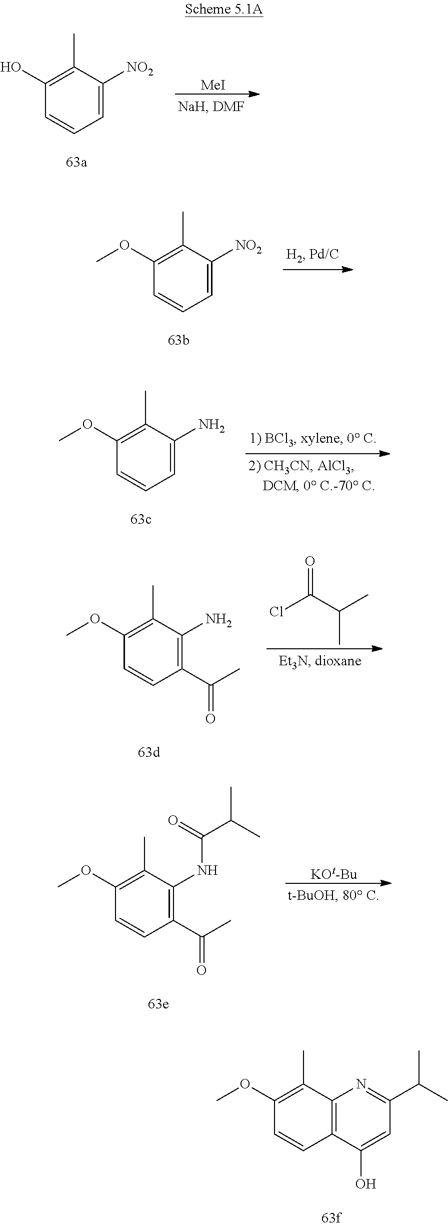

To a solution of compound 63a (10.0 g, 65.3 mmol) in DMF (70 mL) was added NaH (60% dispersion in mineral oil, 3.13 g, 78.4 mmol) in portions carefully at 0° C. The resulting mixture was stirred for 30 min at 0° C. After that, MeI (6.1 mL, 13.9 g, 98 mmol) was added dropwise. The reaction mixture was stirred at 0° C. for 3 hrs. After the reaction was completed, the reaction was quenched by water carefully. The mixture was diluted with water (100 mL) and extracted with EtOAc (100 mL×3), the combined organic layer was washed with brine, dried over Na₂SO₄, concentrated in vacuo to afford compound 63b (9.8 g, yield 90%), which was used directly in next step without further purification.

A flask was charged with compound 63b (9.8 g, 58.7 mmol), MeOH (30 mL) and Pd/C (0.5 g). The mixture was hydrogenated under a pressure of 50 psi room temperature. After stirring for 12 hrs, the mixture was filtered and concentrated in vacuo to give compound 63c (8 g, yield 100%), which was used directly in the next step without further purification.

Compound 63c (8 g, 58.4 mmol) was dissolved in xylene (80 mL). To the resulting solution was added a solution of BCl₃ (61 ml, 61 mmol) in CH₂Cl₂ (60 mL) slowly under nitrogen. The temperature was monitored during the addition and kept below 10° C. The reaction mixture was stirred at 5° C. for 30 min. And then dry acetonitrile (3.9 ml, 75 mmol) was added at 5° C. After stirring for 30 min at 5° C., the solution was transferred into a dropping funnel and slowly added to a suspension of AlCl₃ (8.1 g, 60.9 mmol) in CH₂Cl₂ (60 mL) at 5° C. After addition was completed, the mixture was stirred for 45 min at 5° C. Then the reaction mixture was heated at 70° C. under a nitrogen stream. After evaporation of CH₂Cl₂, the temperature of mixture reached 65° C. and stirred for 12 hrs. After being cooled to room temperature, the mixture was poured into water and heated to reflux for 7 hrs. After cooling, the mixture was adjusted to pH=2-3 by aq. NaOH (6 N). The xylene layer was decanted. The aqueous layer was extracted with DCM (80 mL×3). The xylene layer and DCM layer were combined, washed by water, aq. NaOH (1 N), and brine. The organic layer was separated, dried over Na₂SO₄, concentrated in vacuo. The crude product was purified by column chromatography on silica gel (PE/EA=10/1) to give compound 63d (2 g, yield 25%).

A flask was charged with isobutyryl chloride (806 mg, 7.6 mmol), dioxane (10 mL) and TEA (767 mg, 7.6 mmol). The mixture was cooled to 0° C., then compound 63d (1.36 g, 7.6 mmol) was added in portions. The reaction mixture was stirred for 2 hrs at room temperature. The mixture was poured into water and neutralized by saturated aq. NaHCO₃. The aqueous layer was extracted with EtOAc (50 mL×3). The combined organic layer was washed with brine, dried over Na₂SO₄, concentrated in vacuo. The residue was purified by column chromatography on silica gel (PE/EA=6/1) to afford compound 63e (500 mg, yield 26%).

KOt-Bu (579.5 mg, 5.22 mmol) was added to a suspension of compound 63e (650 mg, 2.61 mmol) in t-BuOH (10 mL). The resulting mixture was stirred at 80° C. overnight, and then cooled to room temperature. There were white solid separated out, the reaction mixture was poured into water, acidified with aq. HCl (2 N), filtered and successively washed with water. The solid was collected and dried in vacuo to give compound 63f (350 mg, yield 58%).

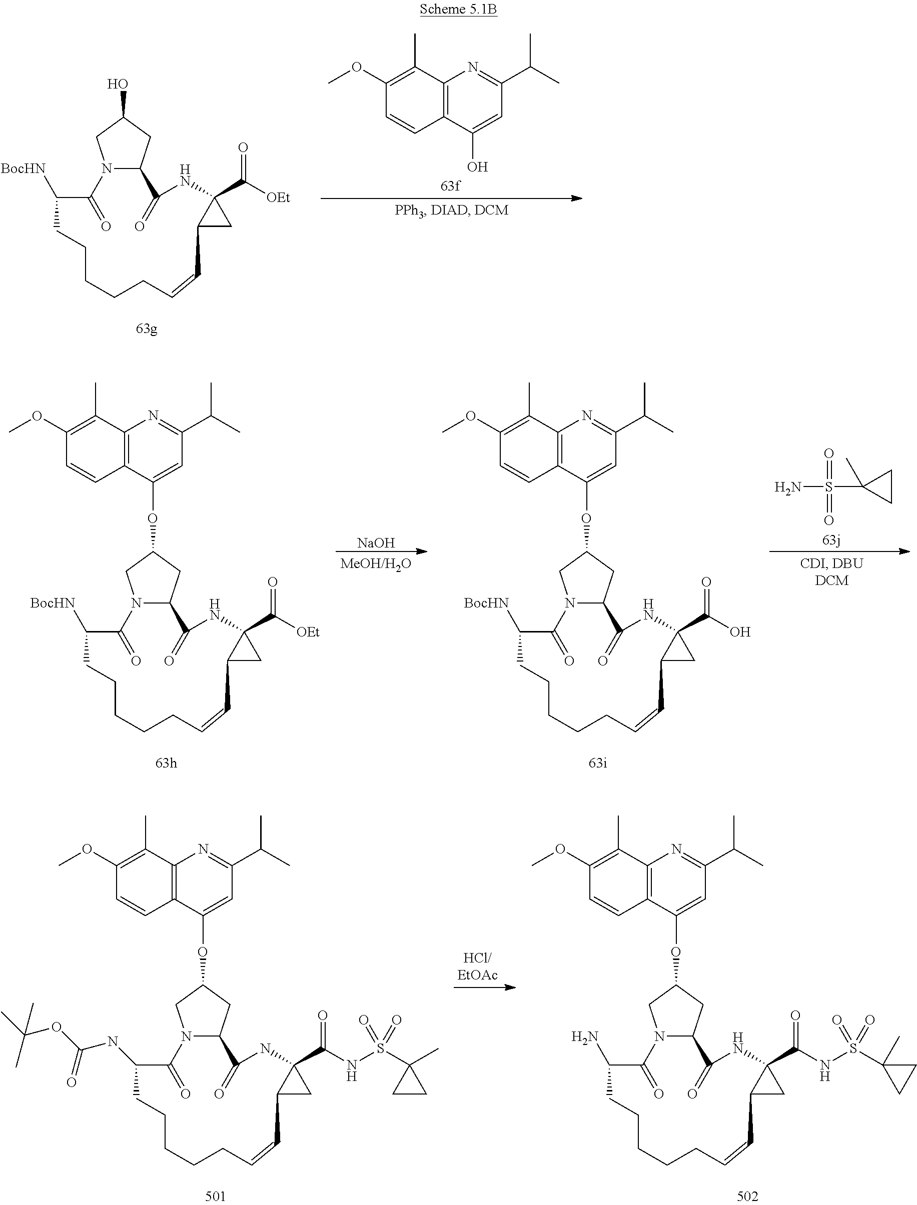

A flask was charged with compound 63g (100 mg, 0.202 mmol), compound 63f (46 mg, 0.202 mmol), triphenylphosphine (159 mg, 0.606 mmol) and anhydrous DCM (4 mL). The reaction mixture was cooled on top of an ice bath and diisopropylazodicarboxylate (DIAD, 122 mg, 0.606 mmol) was added dropwise. The cooling bath was removed and stirring was continued at ambient temperature for 3 hours. The reaction mixture was then extracted with DCM (100 mL×3). The organic layer was combined, washed with brine, dried over Na₂SO₄, concentrated in vacuo. The residue was purified by prep-TLC (PE/EA=1/1) to afford compound 63h (116 mg, yield 81%).

A flask was charged with compound 63h (116 mg, 0.164 mmol) and MeOH (4 mL), a solution of NaOH (40 mg, 0.98 mmol) in H₂O (2 mL) was added thereto. The mixture was stirred at room temperature overnight. And then the mixture was concentrated under reduced pressure and diluted with water (20 mL), neutralized with aq. HCl (1 M). The aqueous layer was extracted with EtOAc (30 mL×3). The combined organic layer was washed with brine, dried over Na₂SO₄, concentrated in vacuo to give compound 63i (100 mg, yield 90%), which was used in next step without further purification.

To a solution of compound 63i (90 mg, 0.1325 mmol) in anhydrous DCM (6 mL) was added CDI (86 mg, 0.53 mmol), the resulting solution was refluxed at 40° C. for 4 hours under nitrogen atmosphere. After the intermediate was detected in LCMS, sulfonamide 63j (125 mg, 0.927 mmol) and DBU (120 mg, 0.795 mmol) was added. The reaction mixture was stirred overnight at 40° C. LCMS monitored the reaction. After completion of the reaction, the solution was diluted with EtOAc (100 mL), and washed with brine, dried over anhydrous Na₂SO₄ and concentrated in vacuo. The residue was purified by Prep-HPLC to afford compound 501 (10 mg, yield 10%). MS (ESI) m/z (M+H)⁺ 796.3.

Compound 501 (10 mg, 0.0126 mmol) was dissolved in a solution of HCl (g) in EtOAC (1 mL). The mixture was stirred at rt for 2 hrs. After completion of the reaction, the solvents were removed under reduced pressure to afford crude product, which was purified by Prep-HPLC (TFA 0.3% condition) to afford compound 502 (TFA salt, 4.4 mg, yield 45%) as a white solid. MS (ESI) m/z (M+H)⁺ 696.2.

### Example 6

### Quinolinyl Analogs

### 6.1 Synthesis of Compounds 601 and 602

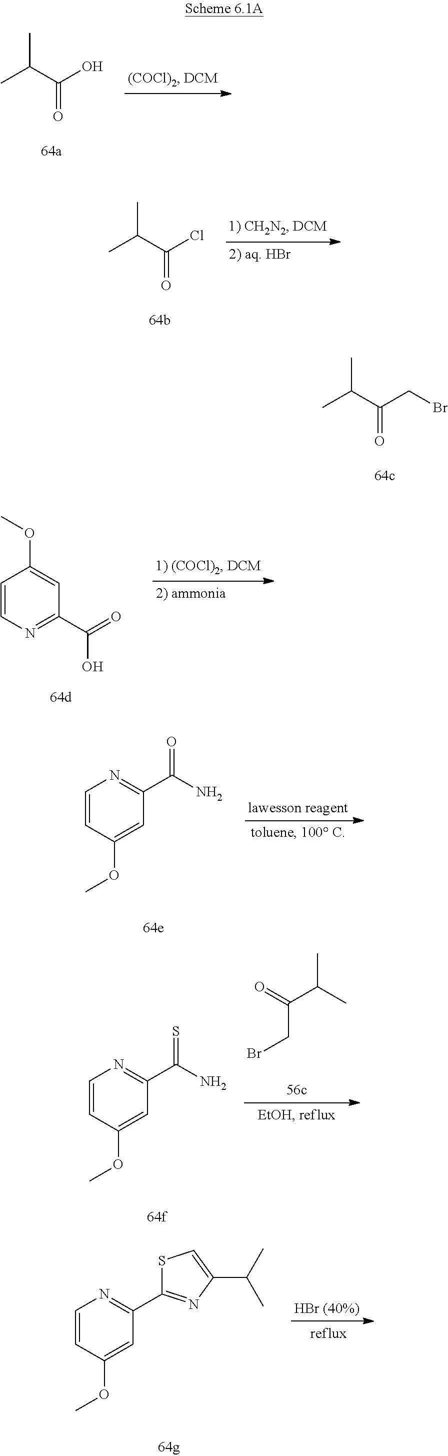

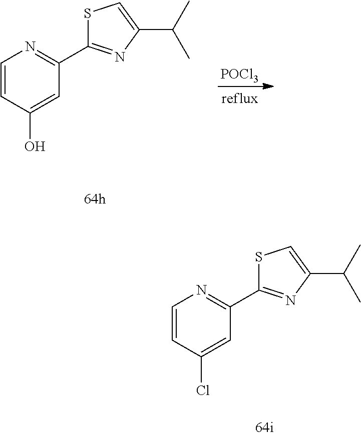

To a solution of compound 64a (10 g, 0.11 mmol) in 20 mL of DCM was added oxalyl chloride (10 g, 0.11 mol) dropwise at 0° C., followed by addition of two drops of DMF. The mixture was stirred overnight at rt And then the mixture was distilled, and collected the fraction in the range 66-72° C. to yield compound 64b (6 g, yield 50%).

To the solution of CH₂N₂ in ether (100 mL, 70 mmol) was added a solution of compound 64b (2.5 g, 23.5 mmol) in 10 mL of DCM dropwise at −5° C. The reaction mixture was stirred at 0° C. for 1 hour. And then aqueous HBr (40%, 14 mL, 70.4 mmol) was added dropwise. The reaction mixture was stirred at the same temperature for 1 hour, and then it was washed with saturated aqueous NaHCO₃ and brine. The aqueous layer was extracted with DCM (30 mL×2). The combined organic layers was dried over anhydrous Na₂SO₄, and concentrated to give compound 64c as yellow oil (3.0 g, yield 79%). ¹H NMR (300 MHz, CDCl₃): δ 3.97 (s, 2H), 3.00-2.92 (m, 1H), 1.14 (d, J=6.9 Hz, 6H).

To a solution of compound 64d (200 mg, 1.3 mmol) in DCM (10 mL) was added oxalyl chloride (200 mg, 1.5 mmol) (adding two drops of DMF). The mixture was stirred at rt for 1 hour. All the volatiles were removed under reduced pressure. The residue was dissolved in 10 mL of DCM. To the resulting solution was added ammonia (0.5 mL). The mixture was stirred at rt for 1 hour and diluted with EtOAc (50 mL), washed with brine, dried over anhydrous sodium sulfate, concentrated under reduced pressure. The residue was purified by prep-TLC (PE/EA=3/1) to provide compound 64e (90 mg, yield 47%). MS (ESI) m/z (M+H)⁺ 152.9.

A flak was charged with compound 64e (370 mg, 2.43 mmol), Lawesson reagent (591 mg, 1.46 mmol) and 8 mL of toluene, and it was flushed with nitrogen for three times. The mixture was heated to reflux for 6 hrs. TLC analysis showed the reaction completed. The mixture was cooled down to rt, and then it was diluted with ethyl acetate (100 mL), washed with brine, dried over anhydrous sodium sulfate, concentrated under reduced pressure. The residue was purified by prep-TLC (PE/EA=3/1) to provide compound 64f (200 mg, yield 49%). MS (ESI) m/z (M+H)⁺ 168.8.

To a solution of compound 64f (170 mg, 1.01 mmol) in EtOH (5 mL) was added compound 64c (217 mg, 1.31 mmol). The mixture was refluxed under nitrogen for 3 hrs. After completion of the reaction, the solvent was evaporated under reduced pressure to provide a crude product, which was purified by prep-TLC (PE/EA=5/1) to afford compound 64g (130 mg, yield 55%). MS (ESI) m/z (M+H)⁺ 234.9.

A flask was charged with compound 64g (120 mg, 0.43 mmol) and 2 mL of aq. HBr (40%). The mixture was heated to reflux overnight. TLC analysis showed the reaction completed. The mixture was cooled down to the room temperature, neutralized with saturated aq. NaHCO₃ and extracted with EtOAc (30 mL×3). The combined organic layer was washed with brine, dried over anhydrous sodium sulfate, filtered and concentrated to give a yellow solid. The crude product 64h was used directly in the next step without further purification (100 mg, crude yield 104%). MS (ESI) m/z (M+H)⁺ 220.9.

Compound 64i was prepared followed the general procedure as described in the scheme (100 mg, yield 93%). MS (ESI) m/z (M+H)⁺ 239.

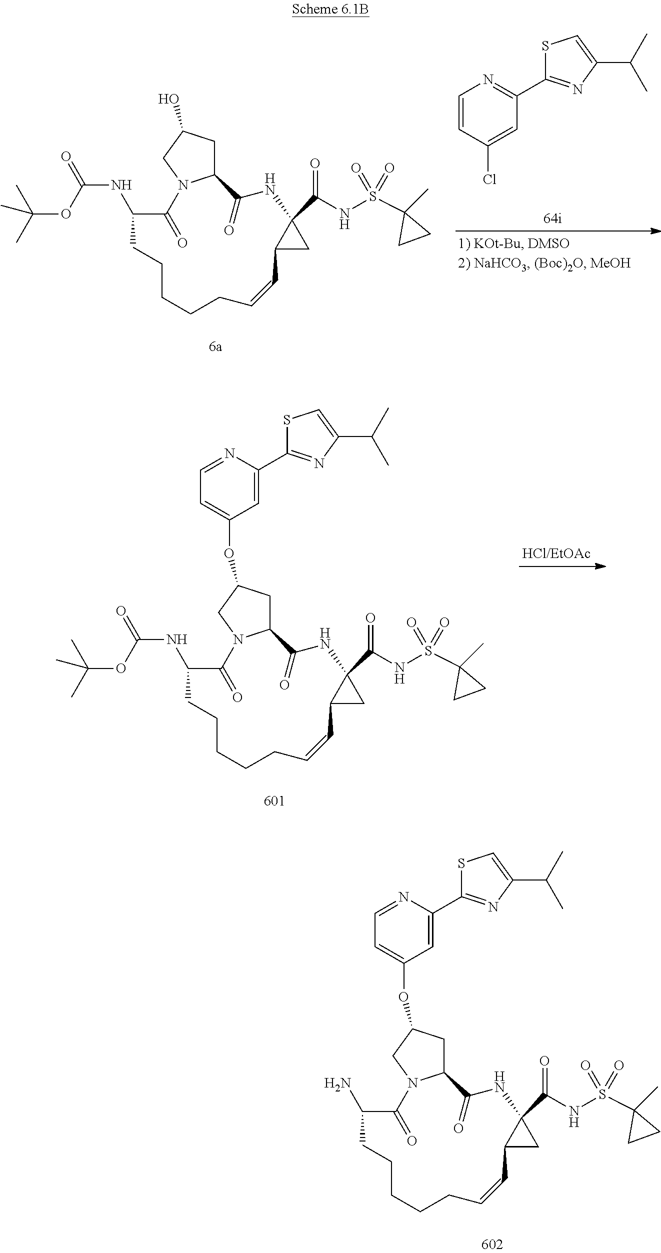

Compound 601 was prepared followed the general procedure using compound 64i (57.7 mg, yield 29%). MS (ESI) m/z (M+H)⁺ 785.4. The preparation of compound 602 was same as that of compound 502 (8.7 mg, yield 77%). MS (ESI) m/z (M+Na)⁺ 707.1.

### Example 7

### Quinoxaline Analogs

### 7.1 Synthesis of Compound 701

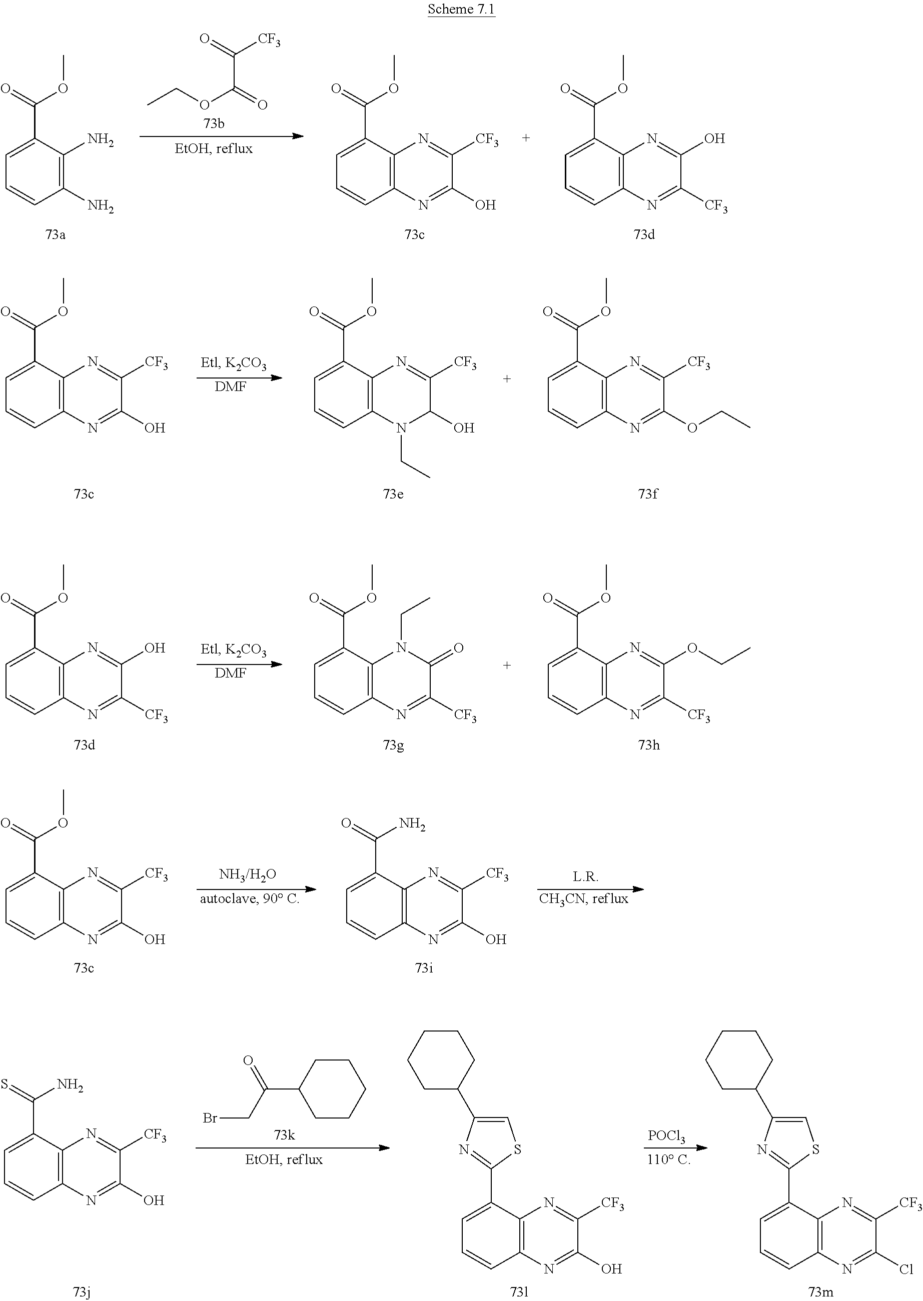

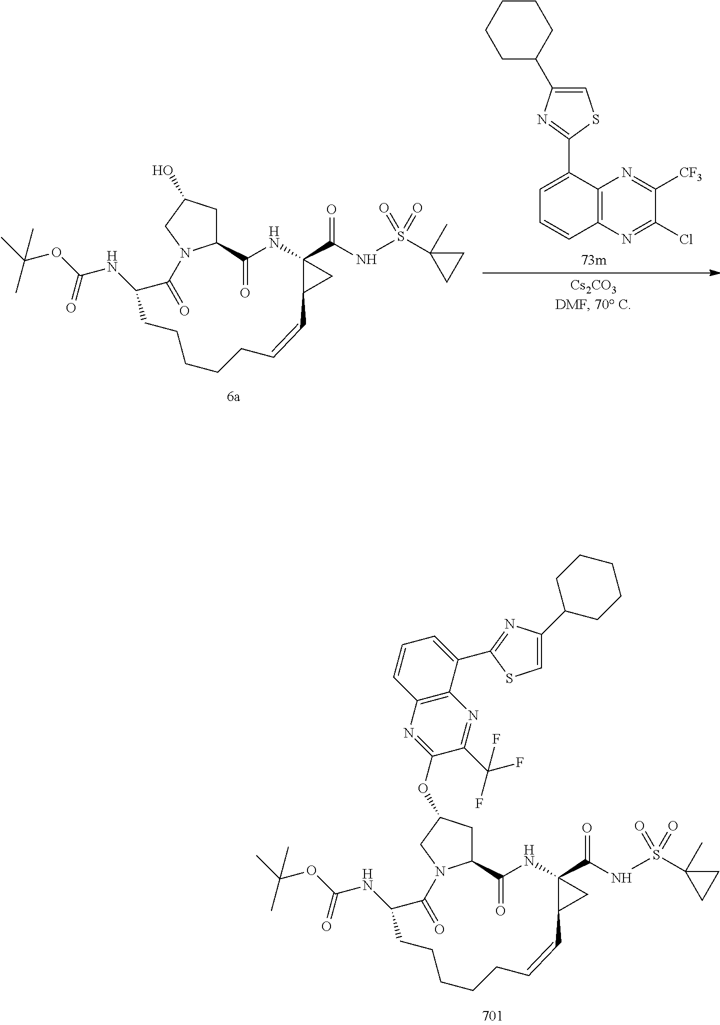

To a solution of compound 73a (3 g, 18.0 mmol) in ethanol (30 mL) was added compound 73b (3.4 g, 18.0 mmol). The reaction mixture was heated to reflux for 18 hours. LCMS analysis showed the reaction completed. The reaction mixture was cooled down and the solvent was removed under reduced pressure. The residual was washed with EtOAc. The filtrate cake was compound 73c (1.9 g). NMR (DMSO-d₆, 300 MHz,): δ 13.15 (brs, 1H), 7.72 (t, J=7.8 Hz, 1H), 7.55 (d, J=7.2 Hz, 1H), 7.47 (d, J=7.8 Hz, 1H), 3.84 (s, 3H). The filtrate was concentrated and purified by column chromatography on silica gel (Petroleum ether:EtOAc=1:1) to afford compound 73d (1.2 g, yield 24%). ¹H NMR (DMSO-d₆, 300 MHz,): δ 11.81 (brs, 1H), 8.24 (d, J=7.8 Hz, 1H), 8.15 (d, J=7.8 Hz, 1H), 7.46 (t, J=7.8 Hz, 1H), 3.92 (s, 3H). MS (ESI) m/z (M+H)⁺ 272.8

A flask was charged with 73c (100 mg, 0.37 mmol, 1 eq), K₂CO₃ (61 mg, 0.56 mmol, 1.2 eq) and 2 mL of DMF. To the resulting mixture was added EtI (69 mg, 0.56 mmol, 1.2 eq). The mixture was stirred at room temperature for 18 hrs. TLC analysis showed the reaction completed. The reaction mixture was diluted with water, extracted with EtOAc (30 mL×3). The organic layer was washed with brine, dried over anhydrous sodium sulfate, filtered, concentrated and purified by prep-TLC (Petroleum ether:EtOAc=2:1) to afford compound 73e (50 mg, yield 45%). ¹H NMR (CDCl₃, 400 MHz,): δ 7.76 (t, J=7.6 Hz, 1H), 7.64 (d, J=6.8 Hz, 1H), 7.53 (d, J=8.0 Hz, 1H), 4.38 (q, J=7.2 Hz, 2H), 4.01 (s, 3H), 1.41 (d, J=7.2 Hz, 3H).

A flask was charged with 73d (50 mg, 0.18 mmol, 1 eq), K₂CO₃ (30 mg, 0.22 mmol, 1.2 eq) and 2 mL of DMF. To the resulting mixture was added EtI (34 mg, 0.22 mmol, 1.2 eq). The mixture was stirred at room temperature for 18 hrs. TLC analysis showed the reaction completed. The reaction mixture was diluted with water, extracted with EtOAc (30 mL×3). The organic layer was washed with brine, dried over anhydrous sodium sulfate, filtered, concentrated and purified by prep-TLC (Petroleum ether:EtOAc=4:1) to afford the compound 73g (6 mg, yield 11%). ¹H NMR (CDCl₃, 400 MHz,): δ 8.08 (d, J=8.0 Hz, 1H), 7.76 (d, J=8.0 Hz, 1H), 7.44 (t, J=8.0 Hz, 1H), 4.24 (q, J=7.2 Hz, 2H), 4.01 (s, 3H), 1.36 (d, J=7.2 Hz, 3H).

The mixture of compound 73c (1.9 g, 7.02 mmol) and ammonia solution (50 mL) was heated to 90° C. in autoclave overnight. The reaction mixture was concentrated to give crude compound 73i (1.7 g, yield 80%). MS (ESI) m/z (M+H)⁺ 257.9.

To a solution of compound 73i (257 mg, 1.0 mmol) in acenitrile (20 mL) was added lawesson's reagent (242 mg, 0.6 mmol). The reaction mixture was heated to reflux for 5 hours. The reaction mixture was concentrated to give crude compound 73j (273 mg) which was used directly for next step reaction. MS (ESI) m/z (M+H)⁺ 273.8.

To a solution of compound 73j (257 mg, 1.0 mmol) in ethanol (20 mL) was added compound 73k (204 mg, 1.0 mmol). The reaction mixture was heated to reflux overnight. The reaction mixture was concentrated and the residue was purified by prep-TLC (PE:EtOAc:MeOH=100:160:4) to give crude compound 73l (60 mg, yield: 16%). MS (ESI) m/z (M+H)⁺ 380.1.

The mixture compound 73l (151 mg, 0.4 mmol) and POCl₃ (19.1 g) was heated to 110° C. for 5 hours. The mixture was stirred overnight at rt. The reaction mixture was concentrated and the residue was dissolved in EtOAc (50 mL). The solution was neutralized to pH=8.0 with saturated aq. NaHCO₃. The mixture was extracted with EtOAc (50 mL×3). The combined organic phase was washed with brine, dried over Na₂SO₄ and concentrated. The residue was purified by prep-TLC (PE:EtOAc=5:1) to give compound 73m (63 mg, yield 40%).

To a solution of compound 6a (140 mg, 0.24 mmol) in N,N-Dimethylformamide (20 mL) was added cesium carbonate (260 mg, 0.8 mmol). The reaction mixture was heated to 70° C. for 5 minutes. Then compound 73m (78 mg, 0.2 mmol) was added. The resulting mixture was stirred at 70° C. under nitrogen overnight. The reaction was cooled down and poured into ice-water. The aqueous phase was acidified to pH=4.0 with 3 N hydrochloride solution at 0° C. The mixture was extracted with EtOAc (50 mL×3). The combined organic phase was washed with brine, dried over Na₂SO₄ and concentrated. The residue was purified by prep-TLC (PE:EtOAc=2:1 100 mL mixed with DCM:MeOH=10:1 20 mL) to give compound 701 (70 mg, yield 37%). MS (ESI) m/z [M+H]⁺ 944.4.

### 7.2 Synthesis of Compound 702

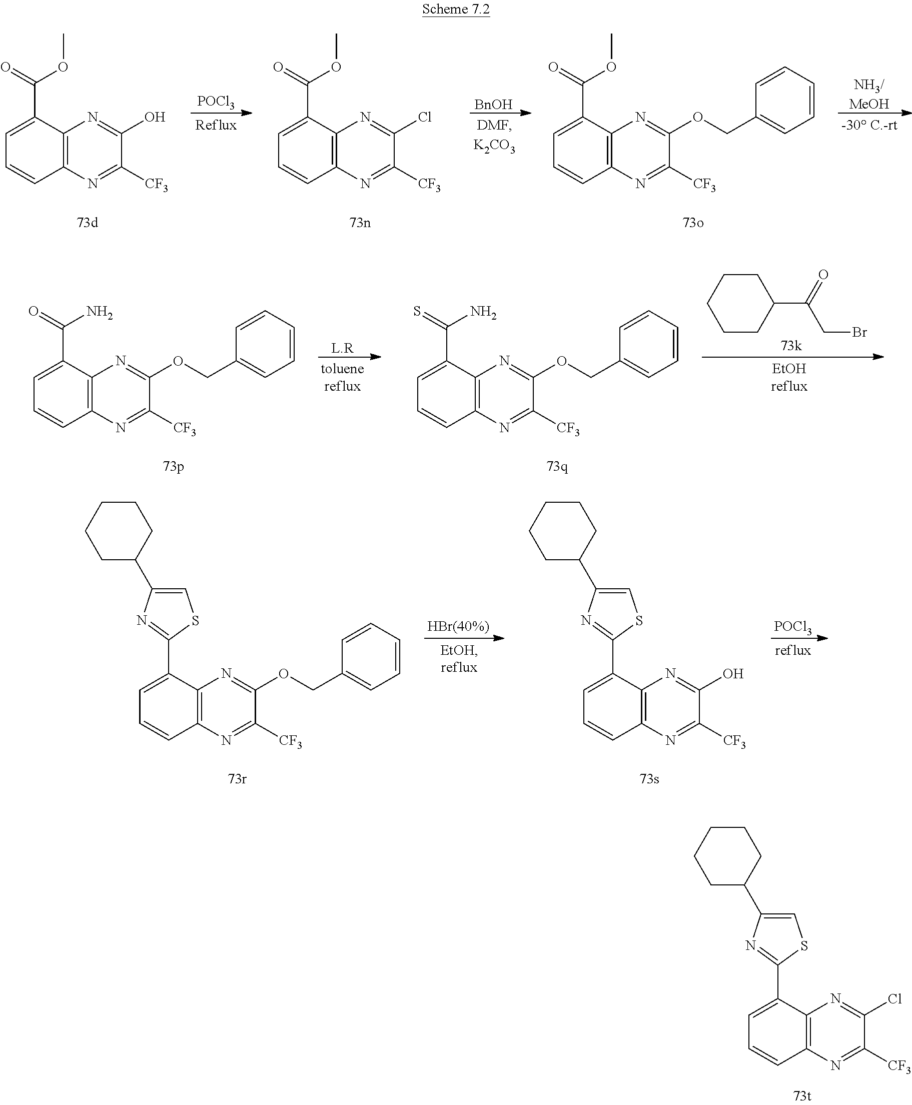

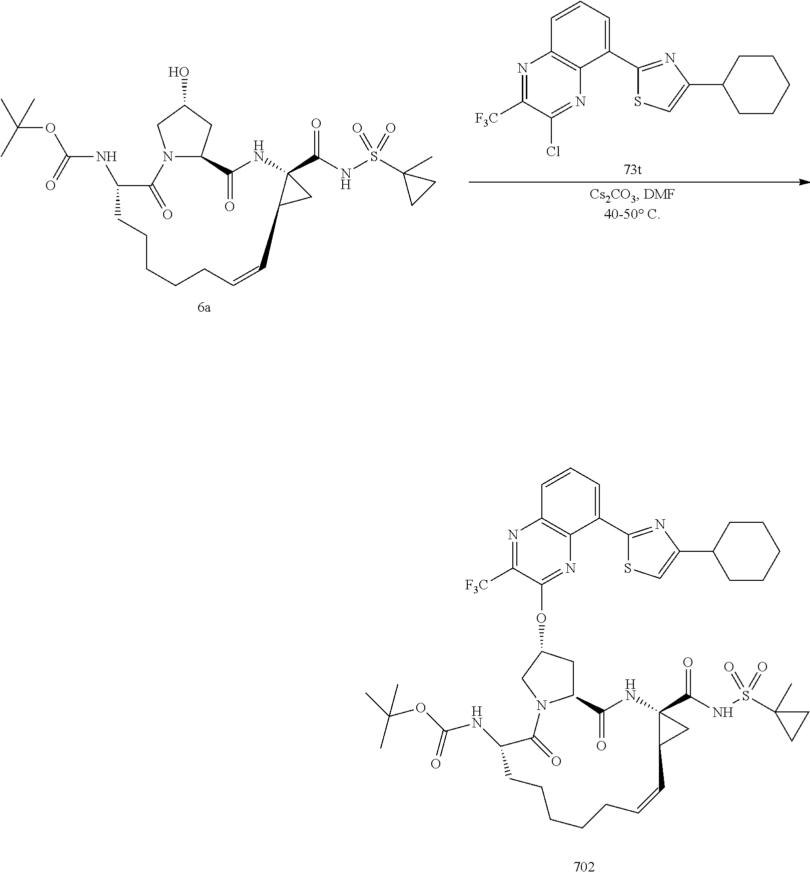

Compound 73n was prepared following the general procedure. 450 mg, yield 86%. MS (ESI) m/z (M+H)⁺ 290.8.

To a solution of compound 73n (450 mg, 1.55 mmol, 1 eq) in 6 mL of DMF was added K₂CO₃ (322 mg, 2.33 mmol, 1.5 eq) and BnOH (251 mg, 2.33 mmol, 1.5 eq). The mixture was stirred for 18 hrs at room temperature. TLC analysis showed the reaction completed. The reaction mixture was diluted with water, extracted with EtOAc (100 mL×3). The combined organic layer was washed with brine, dried over anhydrous sodium sulfate, filtered and concentrated under reduced pressure. The residue was purified by prep-TLC (PE:EtOAc=5:1) to give compound 73o as yellow solid (400 mg, yield 71%). MS (ESI) m/z (M+H)⁺ 362.9.

The solution of compound 73o (400 mg, 1.1 mmol) in 50 mL of MeOH saturated with NH₃ was stirred for 5 hrs at −30° C., and then the reaction mixture was allowed to warm to room temperature and continued stirring for 18 hrs. LCMS analysis showed the reaction completed. The solvent was removed under reduced pressure. The residual was used directly for the next step without further purification (350 mg yield 92%). MS (ESI) m/z (M+H)⁺ 347.7.

A flask was charged with compound 73p (400 mg, 1.15 mmol, 1 eq), lawesson's reagent (279 mg, 0.69 mmol, 0.6 eq) and 15 mL of toluene, flushed with nitrogen for three times. The mixture was heated to reflux for 4 hrs. LCMS analysis showed the reaction completed. All the volatiles were removed under reduced pressure to give yellow solid. The crude compound 73q was used directly for the next step without further purification (400 mg, yield 96%). MS (ESI) m/z (M+H)⁺ 363.9.

To a solution of compound 73q (400 mg, 1.1 mmol, 1 eq) in ethanol (20 mL) was added compound 73k (226 mg, 1.1 mmol, 1 eq). The reaction mixture was heated to reflux overnight. The reaction mixture was cooled to room temperature. All the volatiles were removed under reduced pressure. The residue was diluted with water, extracted with EtOAc (100 mL×3). The combined organic layer was washed with brine, dried over anhydrous sodium sulfate, filtered and concentrated under reduced pressure. The residue was purified by prep-TLC (PE:EtOAc=10:1) to provide compound 73r as yellow solid (90 mg, yield 17%). MS (ESI) m/z (M+H)⁺ 469.9.

To a solution of compound 73r (90 mg, 0.19 mmol, 1 eq) in 2 mL of EtOH was added 1 mL of aq. HBr (40%). The mixture was heated to reflux for 18 hrs. TLC (PE:EtOAc=10:1) analysis showed the reaction completed. The solvent was removed under reduced pressure. The residue was diluted with water, adjusted to pH=7 with saturated aq. NaHCO₃, extracted with EtOAc (100 mL×3). The combined organic layer was washed with brine, dried over anhydrous sodium sulfate, filtered and concentrated under reduced pressure to give compound 73s as yellow solid (70 mg, yield 96%). MS (ESI) m/z (M+H)⁺ 379.8.

The preparation of compound 73t was followed the general procedure. 70 mg, yield 96%. ¹H NMR (400 MHz, CDCl₃): δ 9.06 (d, J=7.2 Hz, 1H), 8.17 (d, J=7.6 Hz, 1H), 7.99 (t, J=8.0 Hz, 1H), 7.16 (s, 1H), 2.89 (m, 1H), 2.19 (d, J=11.2 Hz, 2H), 1.88 (d, J=12.4 Hz, 2H), 1.78 (d, J=12.4 Hz, 1H), 1.50 (m, 4H), 1.31 (m, 1H).

To a solution of compound 6a (115 mg, 0.19 mmol) in DMF (5 mL) was added cesium carbonate (260 mg, 0.8 mmol). The reaction mixture was heated to 40-50° C. for 5 minutes. Then compound 73t (78 mg, 0.2 mmol) was added. The resulting mixture was stirred at 40-50° C. under nitrogen overnight. The reaction was cooled down and poured into ice-water. The aqueous phase was acidified to pH=4.0 with 3 N hydrochloride solution at 0° C. The mixture was extracted with EtOAc (50 mL×3). The combined organic phase was washed with brine, dried over Na₂SO₄ and concentrated. The residue was purified by prep-HPLC to give compound 702 (79.3 mg, yield 44%.). MS (ESI) m/z [M+H]⁺ 944.9.

### 7.3 Synthesis of Compound 703

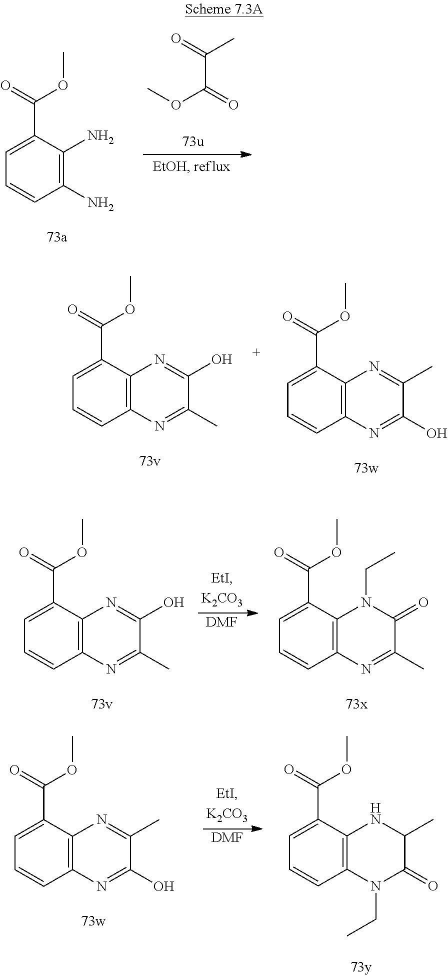

To a solution of compound 73a (3 g, 18 mmol) in ethanol (30 mL) was added compound 73u (3.4 g, 18.0 mmol). The reaction mixture was heated to reflux for 18 hours. LCMS analysis showed the reaction completed. The reaction mixture was cooled down and the solvent was removed under reduced pressure. The residual was washed with EtOAc. The filtrate cake was compound 73v. (1.2 g, yield 30%). ¹H NMR (DMSO, 300 MHz,): δ 11.46 (s, 1H), 8.12 (d, J=8.0 Hz, 1H), 8.01 (d, J=8.0 Hz, 1H), 7.41 (t, J=8.0 Hz, 1H), 3.95 (s, 3H), 2.44 (s, 3H). MS (ESI) m/z (M+H)⁺ 218.8. The filtrate was concentrated and purified by prep-HPLC to give 60 mg of compound 73w.

Compound 73x was prepared following the general procedure. 7 mg, yield 12.5%. ¹H NMR (CDCl₃, 400 MHz,): δ 7.89 (d, J=8.0 Hz, 1H), 7.57 (d, J=8.0 Hz, 1H), 7.32 (t, J=8.0 Hz, 1H), 4.19 (q, J=7.2 Hz, 2H), 3.98 (s, 3H), 2.59 (s, 3H), 1.31 (t, J=7.2 Hz, 3H). MS (ESI) m/z (M+H)⁺ 246.8.

Compound 73y was prepared following the general procedure. 10 mg, yield 15%. ¹H NMR (CDCl₃, 400 MHz,): δ 7.50 (m, 2H), 7.41 (d, J=8.4 Hz, 1H), 4.32 (q, J=7.2 Hz, 2H), 4.00 (s, 3H), 2.60 (s, 3H), 1.35 (t, J=7.2 Hz, 3H). MS (ESI) m/z (M+H)⁺ 246.9.

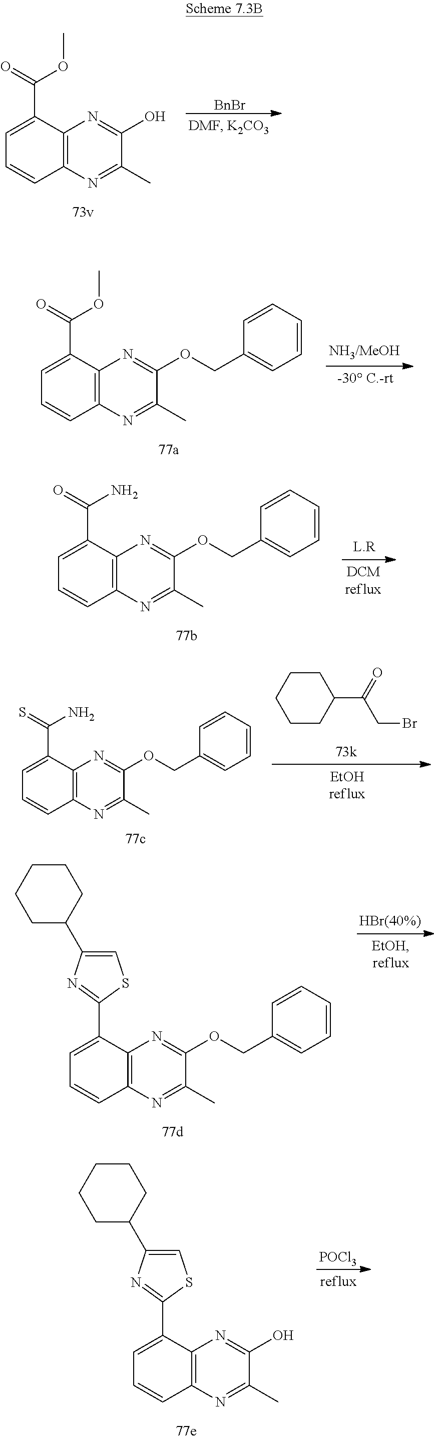

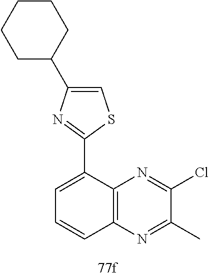

To a solution of compound 73v (200 mg, 0.83 mmol, 1 eq) in 10 mL of DMF was added K₂CO₃ (228 mg, 1.65 mmol, 2 eq) and BnBr (282 mg, 1.65 mmol, 2 eq). The mixture was stirred for 18 h at room temperature. TLC analysis showed the reaction completed. The reaction mixture was diluted with water, extracted with EtOAc (100 mL×3). The combined organic layer was washed with brine, dried over anhydrous sodium sulfate, filtered and concentrated under reduced pressure. Purification by prep-TLC (PE:EtOAc=3:1) gave compound 77a as yellow solid (200 mg, yield 78%). MS (ESI) m/z (M+H)⁺ 308.9.

The preparation of compound 77b was followed the general procedure. 200 mg, crude yield 105%. MS (ESI) m/z (M+H)⁺ 293.8. ¹H NMR (400 MHz, DMSO): δ 9.06 (d, J=7.2 Hz, 1H), 8.17 (d, J=7.6 Hz, 1H), 7.99 (t, J=8.0 Hz, 1H), 7.16 (s, 1H), 2.89 (m, 1H), 2.19 (d, J=11.2 Hz, 2H), 1.88 (d, J=12.4 Hz, 2H), 1.78 (d, J=12.4 Hz, 1H), 1.50 (m, 4H), 1.31 (m, 1H).

A flask was charged with compound 77b (500 mg, 1.7 mmol, 1 eq), lawesson's reagent (415 mg, 1.0 mmol, 0.6 eq) and 10 mL of DCM, flushed with nitrogen for three times. The mixture was heated to reflux for 4 h. LCMS analysis showed the progress of the reaction had stopped. All the volatiles were removed under reduced pressure to give compound 77c as yellow solid. The crude was used directly for the next step without purification (525 mg, crude yield 100%). MS (ESI) m/z (M+H)⁺ 309.9.

Compound 77d was prepared following the general procedure. 120 mg, yield 43%. Compound 77e was prepared following the general procedure. 90 mg, yield 96%. Compound 77f was prepared following the general procedure. 80 mg, yield 84%. ¹H NMR (400 MHz, CDCl₃): δ 8.82 (d, J=7.6 Hz, 1H), 8.17 (d, J=8.0 Hz, 1H), 7.83 (t, J=8.0 Hz, 1H), 7.10 (s, 1H), 2.88 (m, 4H), 2.18 (d, J=10.8 Hz, 2H), 1.87 (d, J=12.4 Hz, 2H), 1.77 (d, J=12.4 Hz, 1H), 1.52 (m, 4H), 1.29 (m, 1H).

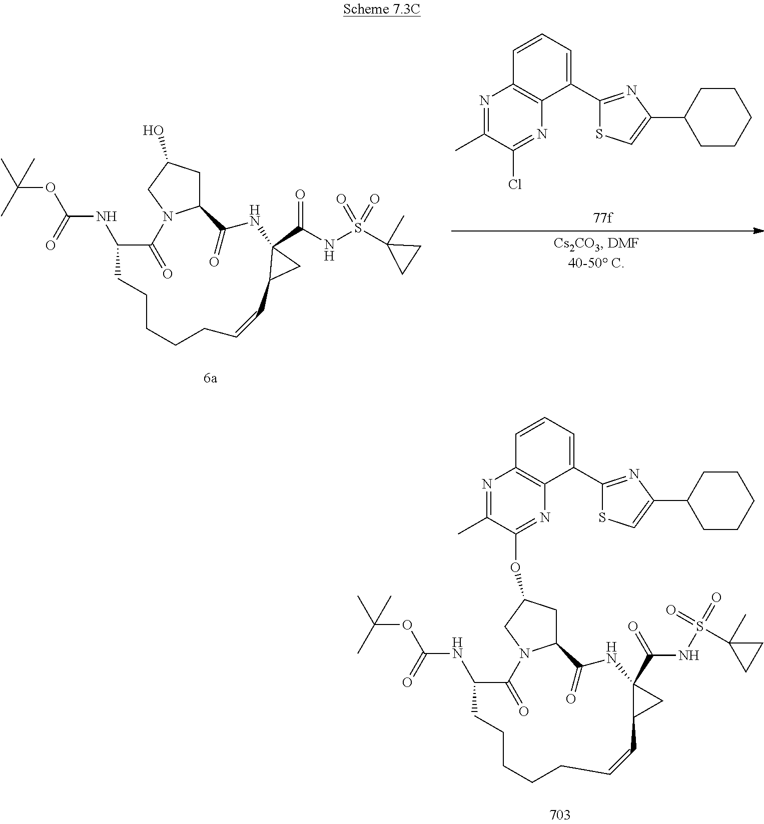

The preparation of compound 703 was done following the general procedure. 82.1 mg, yield 40%.

### Example 8

### Examples of NS3-NS4 Activity

NS3-NS4 inhibition activity can be determined using known assay methods. For example, NS3/NS4 complexes may be formed and inhibitory concentrations of test compounds determined as described in U.S. Patent Application Publication Number 2007/0054842 paragraph numbers 1497-1509, which is incorporated herein by reference in its entirety. Similarly, hepatitis C replicon EC₅₀ may be determined using known assay methods such as described in U.S. Patent Application Publication Number 2007/0054842 paragraph numbers 1510-1515, which is incorporated herein by reference in its entirety. Assays may be conducted at ambient temperature (23° C.) in assay buffer containing 50 mM Tris-HCl, pH 7.5, 15% glycerol, 0.6 mM Lauryldimethylamine Oxide (LDAO), 25 μM NS4A peptide, and 10 mM Dithiothreitol (DTT).

Inhibition of NS3/NS4 activity was determined for several compounds exemplified herein and is presented in Table 5.

TABLE 5
Examples of NS3-NS4 activity.

| Compound | EC₅₀ (nM) | IC₅₀ (nM) |
|---|---|---|
| 101 | B | C |
| 102 | C | C |
| 103 | C | C |
| 104 | B | C |
| 105 | B | C |
| 106 | A | C |
| 107 | B | C |
| 108 | C | C |
| 109 | B | C |
| 110 | A | C |
| 111 | B | C |
| 112 | C | C |
| 113 | B | C |
| 114 | C | C |
| 115 | C | C |
| 116 | B | C |
| 117 | B | C |
| 118 | C | C |
| 119 | B | C |
| 120 | B | C |
| 121 | B | C |
| 122 | A | C |
| 123 | C | C |
| 124 | C | C |
| 125 | C | C |
| 126 | C | C |
| 127 | C | C |
| 128 | C | C |
| 129 | C | C |
| 130 | B | C |
| 131 | B | C |
| 132 | B | C |
| 133 | B | C |
| 134 | B | C |
| 135 | A | C |
| 136 | A | C |
| 137 | A | C |
| 138 | A | C |
| 139 | B | C |
| 140 | C | C |
| 141 | A | C |
| 142 | B | C |
| 143 | B | C |
| 144 | C | C |
| 145 | C | C |
| 146 | C | C |
| 147 | C | C |
| 148 | B | C |
| 149 | B | C |
| 150 | B | C |
| 151 | C | C |
| 152 | C | C |
| 153 | B | C |
| 154 | B | C |
| 155 | B | C |
| 156 | C | C |
| 157 | C | C |
| 158 | C | C |
| 159 | C | C |
| 160 | C | C |
| 161 | C | C |
| 162 | C | C |
| 163 | C | C |
| 164 | A | B |
| 165 | C | C |
| 166 | C | C |
| 167 | B | C |
| 168 | B | C |
| 169 | A | C |
| 170 | B | C |
| 171 | A | C |
| 172 | C | C |
| 173 | A | C |
| 174 | C | C |
| 175 | B | C |
| 176 | C | C |
| 177 | A | C |
| 178 | A | C |
| 179 | C | C |
| 180 | NA | NA |
| 181 | C | C |
| 182 | B | C |
| 183 | C | C |
| 184 | C | C |
| 185 | C | C |
| 186 | C | C |
| 187 | C | C |
| 188 | C | C |
| 189 | C | C |
| 190 | C | C |
| 201 | C | C |
| 301 | B | C |
| 302 | C | C |
| 303 | B | B |
| 304 | A | B |
| 401 | B | C |
| 402 | C | C |
| 403 | B | C |
| 404 | A | C |
| 405 | A | C |
| 406 | C | C |
| 501 | C | C |
| 502 | A | C |
| 601 | C | C |
| 602 | A | C |
| 701 | A | C |
| 702 | C | C |
| 703 | C | C |

A indicates an EC₅₀ or IC₅₀ > 100 nM
B indicates an EC₅₀ or IC₅₀ between 10 and 100 nM
C indicates an EC₅₀ or IC₅₀ of less than 10 nM
NA means the data is not available

What is claimed is:

1. A compound having the structure of Formula I: 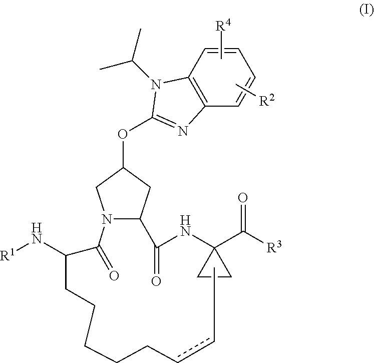
or a pharmaceutically acceptable salt or prodrug thereof wherein:
(a) R¹ is selected from —H (hydrogen), —C(O)OR^{1e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{1a}R^{1b}, —NHC(O)NR^{1a}R^{1b}, —C(O)OR^{1c}, and heteroaryl;
R^{1e} is selected from the group consisting of C₁₋₆ alkyl, cycloalkyl, and heterocyclyl;
R^{1a} and R^{1b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{1c}, —C(O)R^{1d}, optionally substituted aryl, and optionally substituted heteroaryl;
R^{1c} and R^{1d} are each separately selected from the group consisting of —H (hydrogen), C₁₋₄ alkoxy, C₁₋₆ alkyl, C₃₋₇ cycloalkyl, aryl, arylalkyl and heteroaryl;
(b) R² is selected from the group consisting of 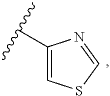
pyrazinyl, and pyrimidinyl, each optionally substituted with R^{2a}; or R² is 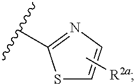
R^{2a} is phenyl substituted with one or more R^{2b} or benzyl optionally substituted with one or more R^{2b}; wherein R^{2b} is halo, —CF₃, —OCF₃, C₁₋₆ alkyl, C₁₋₆ alkoxy, or phenyl;
or R^{2a} is optionally substituted C₁₋₆ alkyl, optionally substituted C₃₋₇ cycloalkyl, optionally substituted dihydrobenzodioxinyl, optionally substituted piperidinyl, optionally substituted piperazinyl, optionally substituted tetrahydropyranyl, or optionally substituted pyrrolidinyl;
(c) R³ is —OH, —NHS(O)₂R^{3a}, —NHS(O)₂OR^{3a} or —NHS(O)₂NR^{3b}R^{3c};
where R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro;
R^{3b} and R^{3c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{3b} and R^{3c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2;
(d) R⁴ is selected from the group consisting of hydrogen, halo, optionally substituted C₁₋₆ alkyl, optionally substituted C₁₋₆ alkoxy, and optionally substituted C₂₋₆ alkenyl;
(e) any bond represented by a dashed and solid line represents a bond selected from the group consisting of a single bond and a double bond;
(f) provided that when R¹ is —C(O)O-t-butyl, R³ is —NHS(O)₂-methylcyclopropyl or —NHS(O)₂N(CH₃)₂, R⁴ is H, and R² is 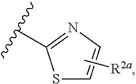
then R^{2a} is not cyclopropyl, cyclobutyl, cyclohexyl, unsubstituted benzyl, 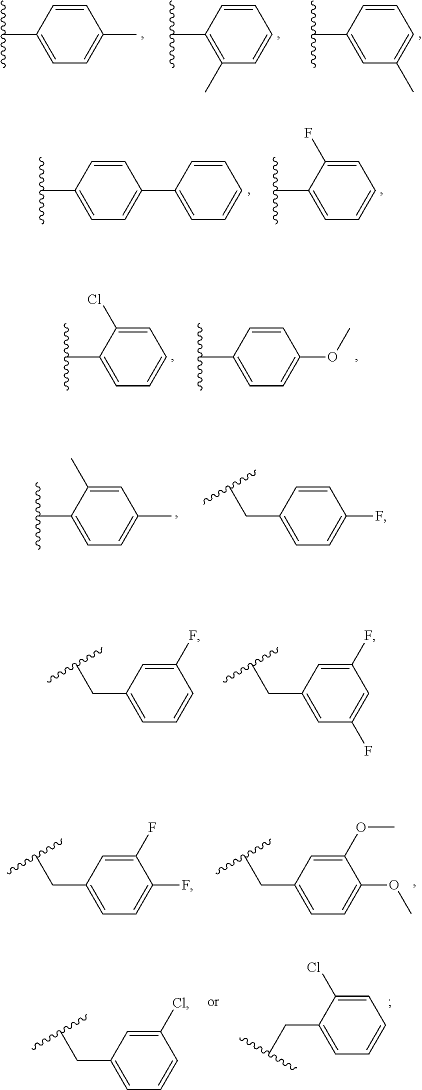
and
(f) provided that the compound is not selected from the group consisting of

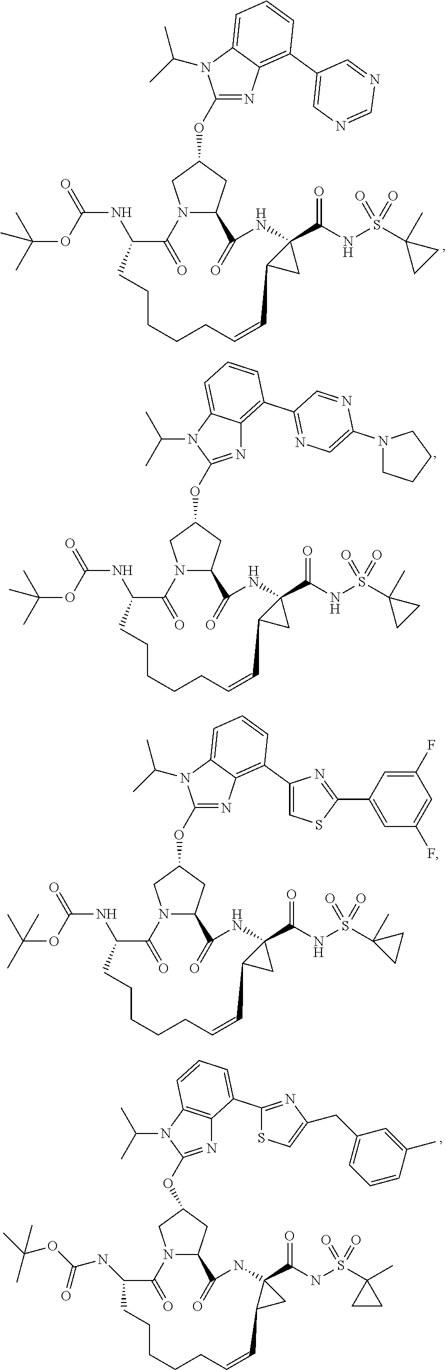

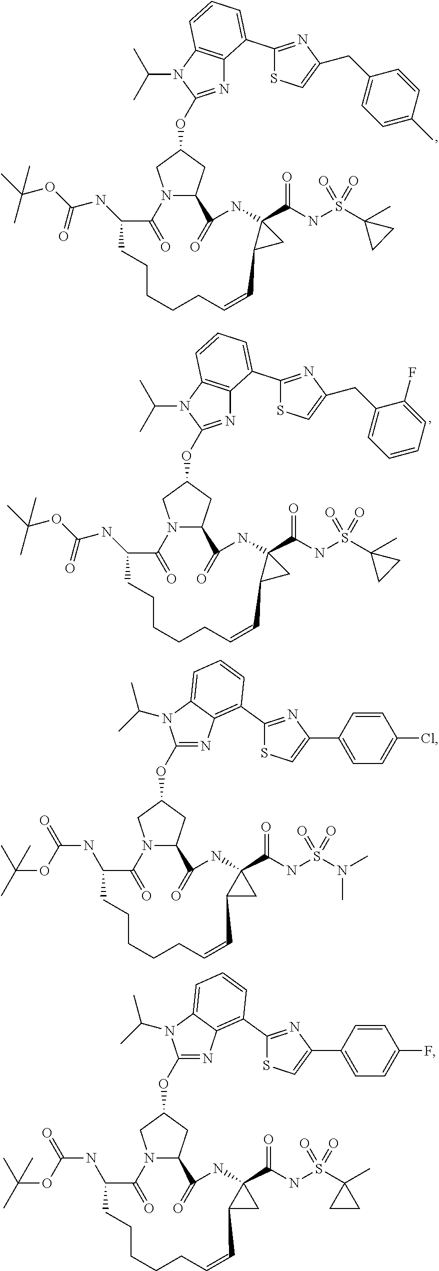

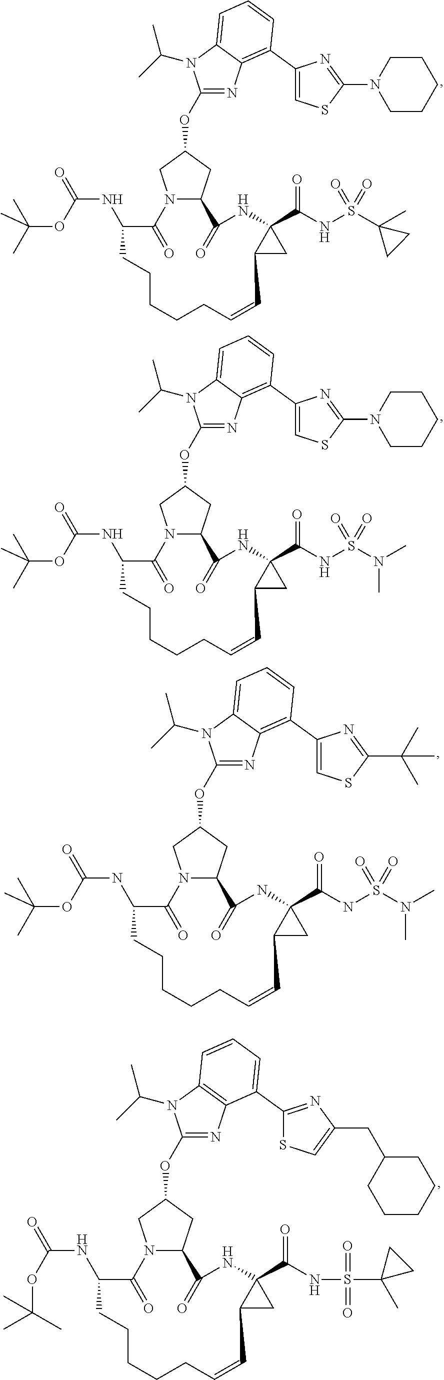

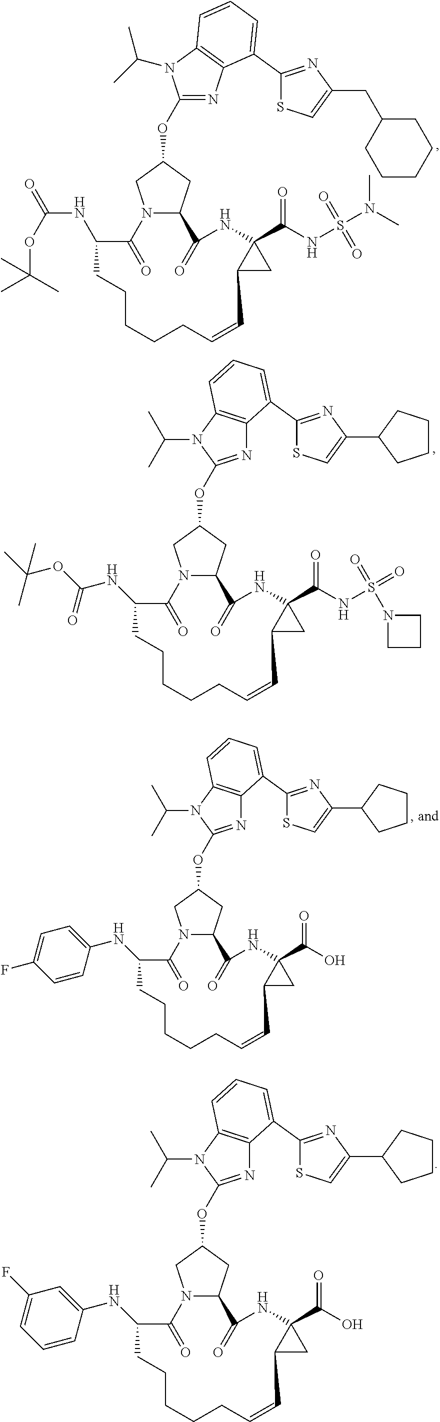

2. The compound of claim 1 having the following structure:

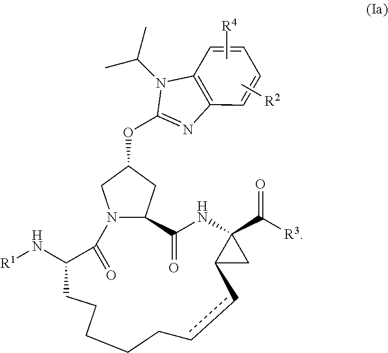

3. The compound of claim 1, wherein R^{2a} is optionally substituted C₁₋₆ alkyl, optionally substituted C₃₋₇ cycloalkyl, optionally substituted dihydrobenzodioxinyl, optionally substituted piperidinyl, optionally substituted piperazinyl or optionally substituted pyrrolidinyl; and R^{3a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

4. The compound of claim 1, wherein R^{2a} is phenyl substituted with one or more R^{2b} or benzyl optionally substituted with one or more R^{2b}; wherein R^{2b} is halo, —CF₃, —OCF₃, methyl, propyl, butyl, methoxy, or phenyl.

5. The compound of claim 1, wherein R^{2a} is C₁₋₆ alkyl, C₃₋₇ cycloalkyl, dihydrobenzodioxinyl, optionally substituted piperazinyl, or optionally substituted pyrrolidinyl.

6. The compound of claim 1, wherein R¹ is —C(O)OR^{1e}, wherein R^{1e} is t-butyl, C₃₋₇ cycloalkyl, or pyrrolidinyl; and R³ is selected from the group consisting of —NHS(O)₂-methylcyclopropyl, —NHS(O)₂-cyclopropyl, —NHS(O)₂-ethynylcyclopropyl, —NHS(O)₂-propynylcyclopropyl and —NHS(O)₂—N(CH₃)₂.

7. The compound of claim 1, wherein R¹ is —C(O)O-t-butyl and R³ is —NHS(O)₂-methylcyclopropyl or —NHS(O)₂—N(CH₃)₂.

8. The compound of claim 1, wherein R² is 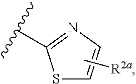
R^{2a} is optionally substituted C₁₋₆ alkyl or optionally substituted C₃₋₇ cycloalkyl, and R⁴ is halo, optionally substituted C₁₋₆ alkyl, or optionally substituted C₁₋₆ alkoxy.

9. The compound of claim 1 selected from the group consisting of compounds 101-190.

10. A compound having the structure of Formula II: 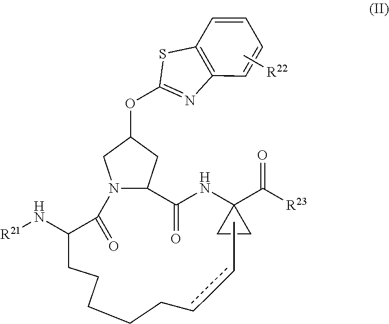
or a pharmaceutically acceptable salt or prodrug thereof wherein:
(a) R²¹ is selected from —C(O)OR^{21e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{21a}R^{21b}, —NHC(O)NR^{21a}R^{21b}, —C(O)OR^{21c}, and heteroaryl;
R^{21e} is selected from the group consisting of C₁₋₆ alkyl, cycloalkyl, and heterocyclyl;
R^{21a} and R^{21b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{21c}, —C(O)R^{21d}, optionally substituted aryl, and optionally substituted heteroaryl;
R^{21c} and R^{21d} are each separately selected from the group consisting of —H (hydrogen), C₁₋₄ alkoxy, C₁₋₆ alkyl, C₃₋₇ cycloalkyl, aryl, arylalkyl and heteroaryl;
(b) R²² is heteroaryl optionally substituted with one or more R^{22a};
each R^{22a} is independently selected from the group consisting of C₁₋₆ alkyl, C₃₋₇ cycloalkyl, C₁₋₆ alkoxy, heteroaryl, heterocyclyl, arylalkyl, aryl, halo, —CN, —CF₃, —C(O)NR′R″ and —NR′R″, wherein said C₁₋₆ alkyl, C₃₋₇ cycloalkyl, C₁₋₆ alkoxy, heteroaryl, heterocyclyl, arylalkyl, and aryl are each optionally substituted with one or more R^{22b};
each R^{22b} is independently selected from the group consisting of halo, —CF₃, —OCF₃, C₁₋₆ alkyl, C₁₋₆ alkoxy, and aryl;
each NR′R″ is separately selected wherein R′ and R″ are each independently selected from the group consisting of —H (hydrogen), halo, —C(O)NR′R″, optionally substituted C₁₋₆ alkyl, optionally substituted C₂₋₆ alkenyl, optionally substituted C₁₋₆ alkoxy, optionally substituted aryl, optionally substituted arylalkyl and optionally substituted heteroaryl; or R′ and R″ are taken together with the nitrogen to which they are attached to form heterocyclyl;
(c) R²³ is —OH, —NHS(O)₂R^{23a}, —NHS(O)₂OR^{23a} or —NHS(O)₂NR^{23b}R^{23c};
where R^{23a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro;
R^{23b} and R^{23c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{23b} and R^{23c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

11. The compound of claim 10 having the following structure:

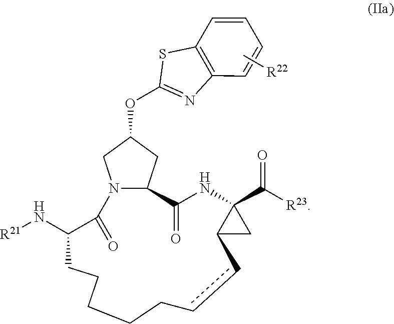

12. The compound of claim 10, wherein R^{23a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

13. The compound of claim 10, wherein R²² is thiazyl, pyrazinyl or pyrimidinyl, each optionally substituted with one or more R^{22a}.

14. The compound of claim 13, wherein each R^{22a} is independently selected from the group consisting of C₃₋₇ cycloalkyl, aryl, heteroaryl, arylalkyl, and heterocyclyl, each optionally substituted with one or more R^{22b}.

15. The compound of claim 10, wherein R²¹ is —C(O)OR^{21e}, wherein R^{21e} is t-butyl, C₃₋₇ cycloalkyl, or pyrrolidinyl; and R²³ is selected from the group consisting of —NHS(O)₂-methylcyclopropyl, —NHS(O)₂-cyclopropyl, and —NHS(O)₂—N(CH₃)₂.

16. The compound of claim 10 having the following formula:

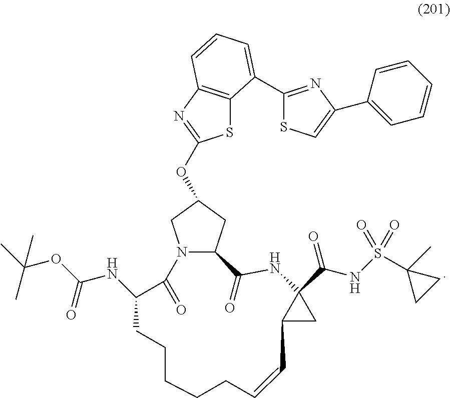

17. A compound having the structure of Formula III: 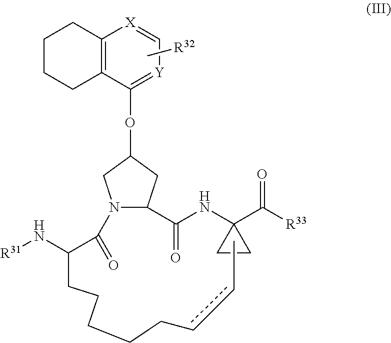
or a pharmaceutically acceptable salt or prodrug thereof wherein:
(a) X and Y are each N or CH;
(b) R³¹ is selected from —C(O)OR^{31e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{31a}R^{31b}, —NHC(O)NR^{31a}R^{31b}, —C(O)OR^{31c}, and heteroaryl;
R^{31e} is selected from the group consisting of C₁₋₆ alkyl, cycloalkyl, and heterocyclyl;
R^{31a} and R^{31b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{31c}, —C(O)R^{31d}, optionally substituted aryl, and optionally substituted heteroaryl;
R^{31c} and R^{31d} are each separately selected from the group consisting of —H (hydrogen), C₁₋₄ alkoxy, C₁₋₆ alkyl, C₃₋₇ cycloalkyl, aryl, arylalkyl and heteroaryl;
(c) R³² is hydrogen, optionally substituted C₁₋₆ alkyl, optionally substituted aryl, or optionally substituted heteroaryl;
(d) R³³ is —OH, —NHS(O)₂R^{33a}, —NHS(O)₂OR^{33a} or —NHS(O)₂NR^{33b}R^{33c};
where R^{33a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro;
R^{33b} and R^{33c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{33b} and R^{33c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(e) any bond represented by a dashed and solid line represents a bond selected from the group consisting of a single bond and a double bond.

18. The compound of claim 17 having the following structure:

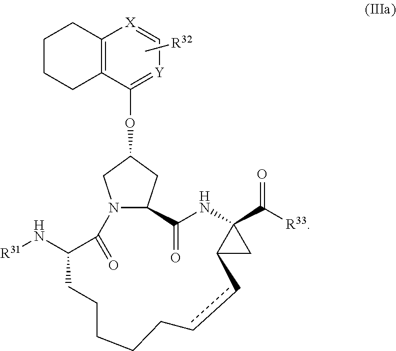

19. The compound of claim 17, wherein R^{33a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

20. The compound of claim 17, wherein R³² is hydrogen, aryl, or substituted heteroaryl.

21. The compound of claim 17, wherein at least one of X and Y is N.

22. The compound of claim 17, wherein R³¹ is —C(O)OR^{31e}, wherein R^{31e} is t-butyl, C₃₋₇ cycloalkyl, or pyrrolidinyl; and R³³ is selected from the group consisting of —NHS(O)₂-methylcyclopropyl, —NHS(O)₂-cyclopropyl, and —NHS(O)₂—N(CH₃)₂.

23. The compound of claim 17 selected from the group consisting of:

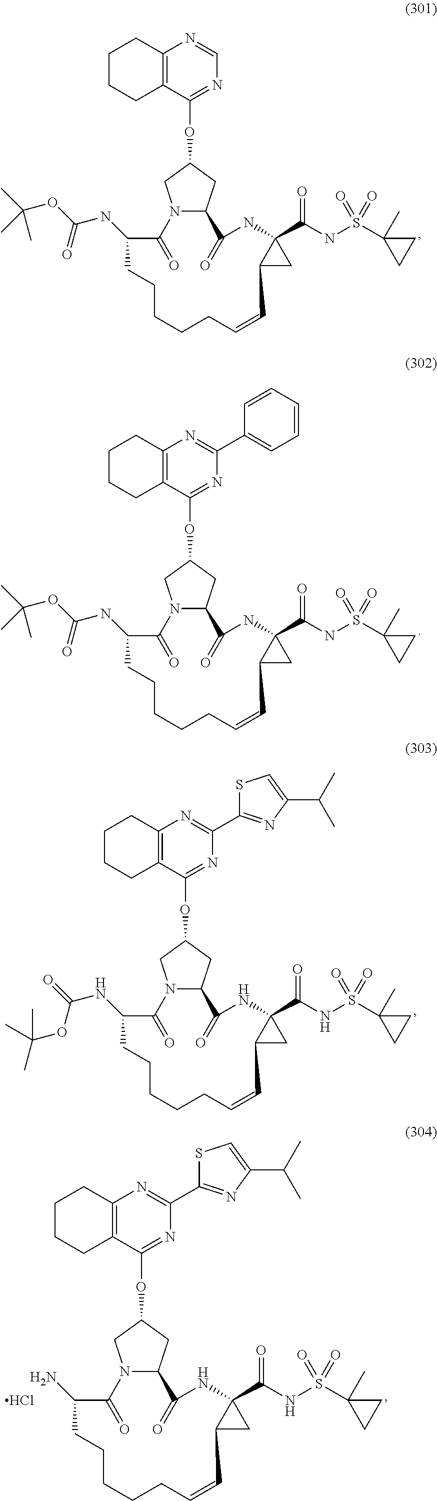

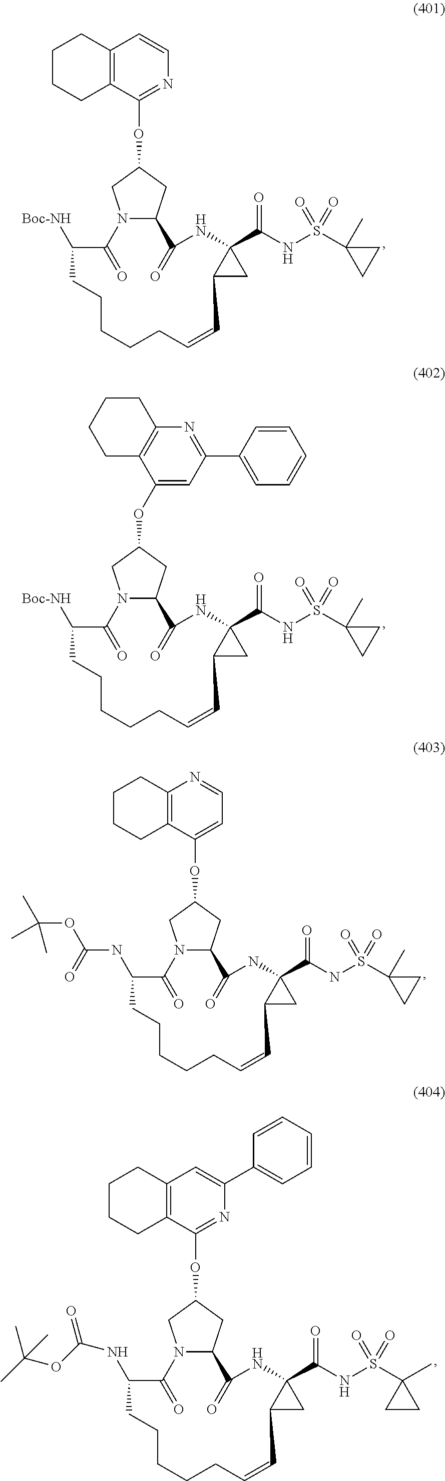

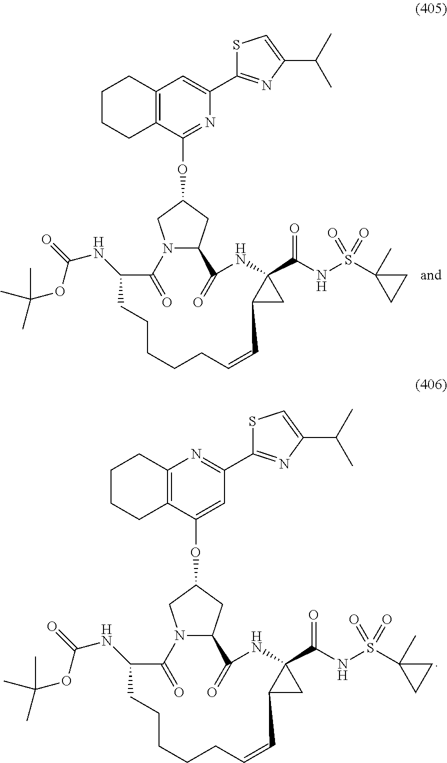

24. A compound having the structure of Formula IV or Formula V: 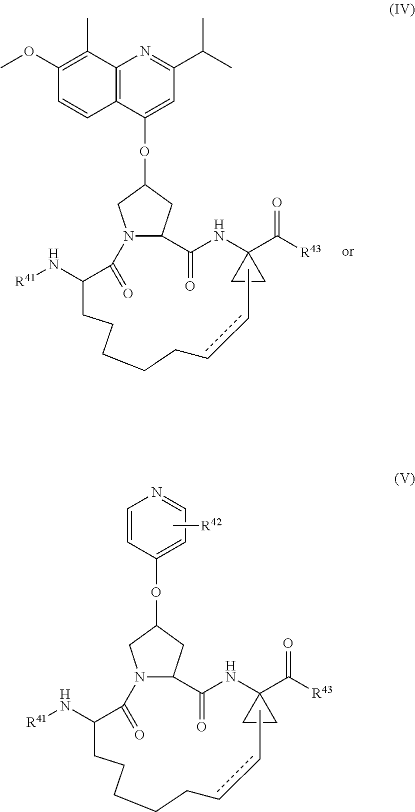
or a pharmaceutically acceptable salt or prodrug thereof wherein:
(a) R⁴¹ is selected from —C(O)OR^{41e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{41a}R^{41b}, —NHC(O)NR^{41a}R^{41b}, —C(O)OR^{41c}, and heteroaryl;
R^{41e} is selected from the group consisting of C₁₋₆ alkyl, cycloalkyl, and heterocyclyl;
R^{41a} and R^{41b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{41c}, —C(O)R^{41d}, optionally substituted aryl, and optionally substituted heteroaryl;
R^{41c} and R^{41d} are each separately selected from the group consisting of —H (hydrogen), C₁₋₄ alkoxy, C₁₋₆ alkyl, C₃₋₇ cycloalkyl, aryl, arylalkyl and heteroaryl;
(b) R⁴² is heteroaryl or aryl, optionally substituted by one or more R^{42a};
wherein each R^{42a} is independently selected from the group consisting of —H (hydrogen), optionally substituted C₁₋₆ alkyl, optionally substituted C₁₋₆ alkoxy, optionally substituted aryl, optionally substituted arylalkyl, optionally substituted heteroaryl, optionally substitute C₃₋₇ cycloalkyl, and optionally substituted heterocycloalkyl;
(c) R⁴³ is —OH, —NHS(O)₂R^{43a}, —NHS(O)₂OR^{43a} or —NHS(O)₂NR^{43b}R^{43c};
where R^{43a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro;
R^{43b} and R^{43c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{43b} and R^{43c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2; and
(d) any bond represented by a dashed and solid line represents a bond selected from the group consisting of a single bond and a double bond.

25. The compound of claim 24 having the structure of Formula IVa or Formula Va:

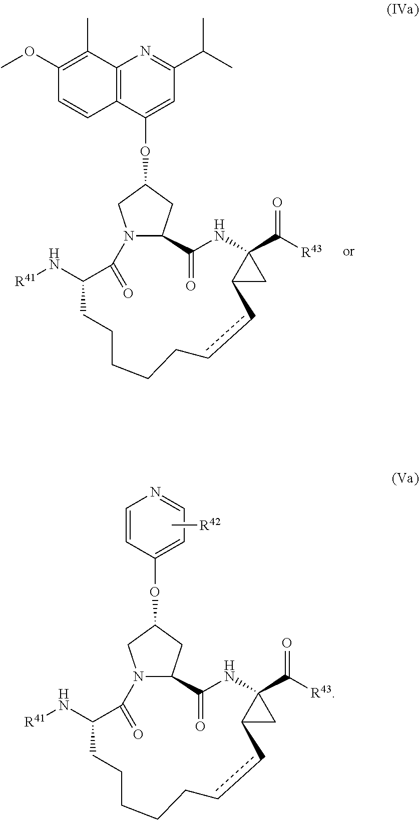

26. The compound of claim 24, wherein R^{43a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, hydroxy-C₁₋₆alkyl, C₁₋₆ alkyl optionally substituted with up to 9 fluoro, and C₁₋₆ alkoxy optionally substituted with up to 9 fluoro.

27. The compound of claim 24, wherein the compound has the Formula V or Va, and R⁴² is heteroaryl optionally substituted by R^{42a}.

28. The compound of claim 27, wherein R⁴² is thiazyl optionally substituted by R^{42a}.

29. The compound of claim 24, wherein R⁴¹ is hydrogen or —C(O)OR^{41e}, wherein R^{41e} is t-butyl, C₃₋₇ cycloalkyl, or pyrrolidinyl; and R⁴³ is selected from the group consisting of —NHS(O)₂-methylcyclopropyl, —NHS(O)₂-cyclopropyl, and —NHS(O)₂—N(CH₃)₂.

30. The compound of claim 24 selected from the group consisting of:

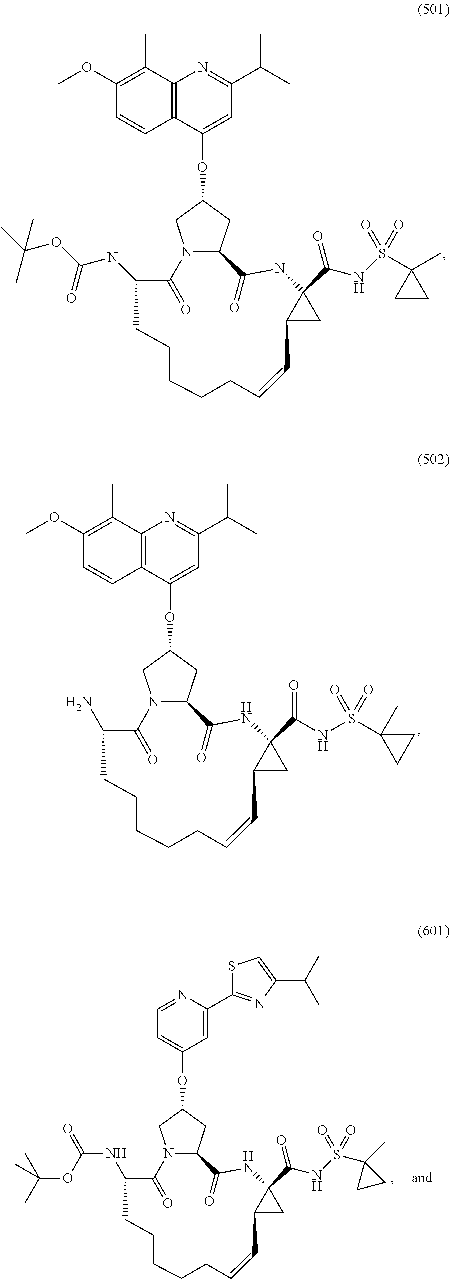

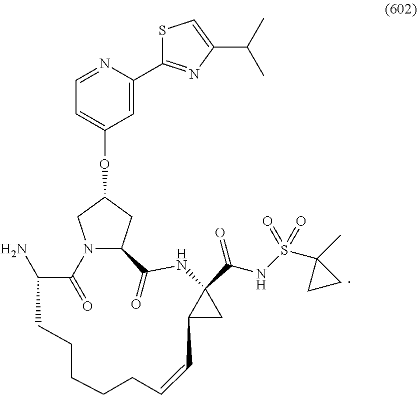

31. A compound having the structure of Formula VI: 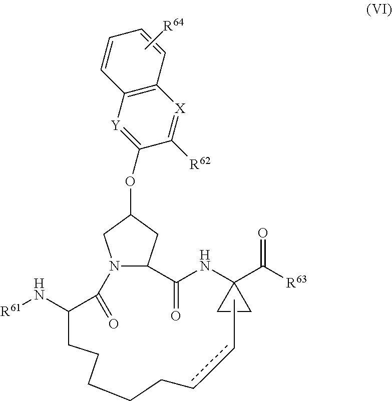
or a pharmaceutically acceptable salt or prodrug thereof wherein:
(a) X and Y are each N or CH;
(b) R⁶¹ is selected from —C(O)OR^{61e}, heteroaryl, or aryl, wherein heteroaryl and aryl are each optionally substituted with one or more substituents each independently selected from the group consisting of halo, amino, C₁₋₆ alkyl optionally substituted with up to 9 fluoro, C₁₋₆ alkoxy optionally substituted with up to 9 fluoro, C₂₋₆ alkenyl, C₂₋₆ alkynyl, —C(O)NR^{61a}R^{61b}, —NHC(O)NR^{61a}R^{61b}, —C(O)OR^{61c}, and heteroaryl;
R^{61e} is selected from the group consisting of C₁₋₆ alkyl, cycloalkyl, and heterocyclyl;
R^{61a} and R^{61b} are taken together with the nitrogen to which they are attached to form piperazinyl or morpholinyl, each optionally substituted with one or more substituents independently selected from optionally substituted C₁₋₆ alkyl, C₂₋₆ alkenyl, C₂₋₆ alkynyl, —C(O)OR^{61c}, —C(O)R^{61d}, optionally substituted aryl, and optionally substituted heteroaryl;
R^{61c} and R^{61d} are each separately selected from the group consisting of —H (hydrogen), C₁₋₄ alkoxy, C₁₋₆ alkyl, C₃₋₇ cycloalkyl, aryl, arylalkyl and heteroaryl;
(c) R⁶² is selected from the group consisting of —H, —C(O)OR^{62a}, C₁₋₆ alkyl optionally substituted with up to 5 fluoro, C₂₋₆ alkenyl, C₃₋₇ cycloalkyl, optionally substituted aryl and optionally substituted heteroaryl;
wherein R^{62a} is selected from the group consisting of —H (hydrogen), C₁₋₄ alkoxy, C₁₋₆ alkyl, C₃₋₇ cycloalkyl, aryl, arylalkyl and heteroaryl;
(d) R⁶³ is —OH, —NHS(O)₂R^{63a}, —NHS(O)₂OR^{63a} or —NHS(O)₂NR^{63b}R^{63c};
where R^{63a} is selected from the group consisting of C₁₋₆ alkyl, —(CH₂)_{q}C₃₋₇cycloalkyl, —(CH₂)_{q}C_{6 or 10}aryl, and a heteroaryl, each optionally substituted with one or more substituents each independently selected from the group consisting of halo, cyano, nitro, hydroxy, —COOH, —(CH₂)ₜC₃₋₇cycloalkyl, C₂₋₆ alkenyl, C₂₋₆ alkynyl, hydroxy-C₁₋₆alkyl, C₁₋₆ alkyl optionally substituted with up to 9 fluoro, and C₁₋₆ alkoxy optionally substituted with up to 9 fluoro;
R^{63b} and R^{63c} are each separately a hydrogen atom, or separately selected from the group consisting of C₁₋₆ alkyl, —(CH₂)_{q}C₃₋₇cycloalkyl, and C_{6 or 10} aryl, each optionally substituted with one or more substituents each independently selected from the group consisting of halo, cyano, nitro, hydroxy, —(CH₂)ₜC₃₋₇cycloalkyl, C₂₋₆ alkenyl, hydroxy-C₁₋₆alkyl, phenyl, C₁₋₆ alkyl substituted with up to 5 fluoro, and C₁₋₆ alkoxy substituted with up to 5 fluoro;
or R^{63b} and R^{63c} are taken together with the nitrogen to which they are attached to form a three- to six-membered heterocyclic ring bonded to the parent structure through a nitrogen, and where the heterocylic ring is optionally substituted with one or more substituents each independently selected from the group consisting of halo, cyano, nitro, C₁₋₆ alkyl, C₁₋₆ alkoxy, and phenyl;
each t is independently 0, 1 or 2;
each q is independently 0, 1 or 2;
(e) R⁶⁴ is selected from the group consisting of optionally substituted C₁₋₆ alkyl, optionally substituted aryl, or optionally substituted heteroaryl; and
(f) any bond represented by a dashed and solid line represents a bond selected from the group consisting of a single bond and a double bond.

32. The compound of claim 31 having the following structure:

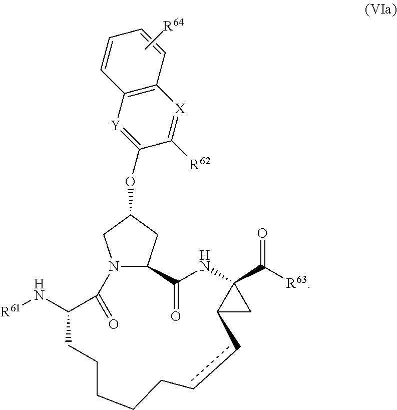

33. The compound of claim 31, wherein R⁶² is C₁₋₆ alkyl optionally substituted with up to 5 fluoro.

34. The compound of claim 31, wherein at least one of X and Y is N.

35. The compound of claim 31, wherein R⁶¹ is C(O)OR^{61e}, wherein R^{61e} is t-butyl, C₃₋₇ cycloalkyl, or pyrrolidinyl; and R⁶³ is selected from the group consisting of —NHS(O)₂-methylcyclopropyl, —NHS(O)₂-cyclopropyl, and —NHS(O)₂—N(CH₃)₂.

36. The compound of claim 31, wherein R⁶⁴ is optionally substituted heteroaryl.

37. The compound of claim 31 selected from the group consisting of:

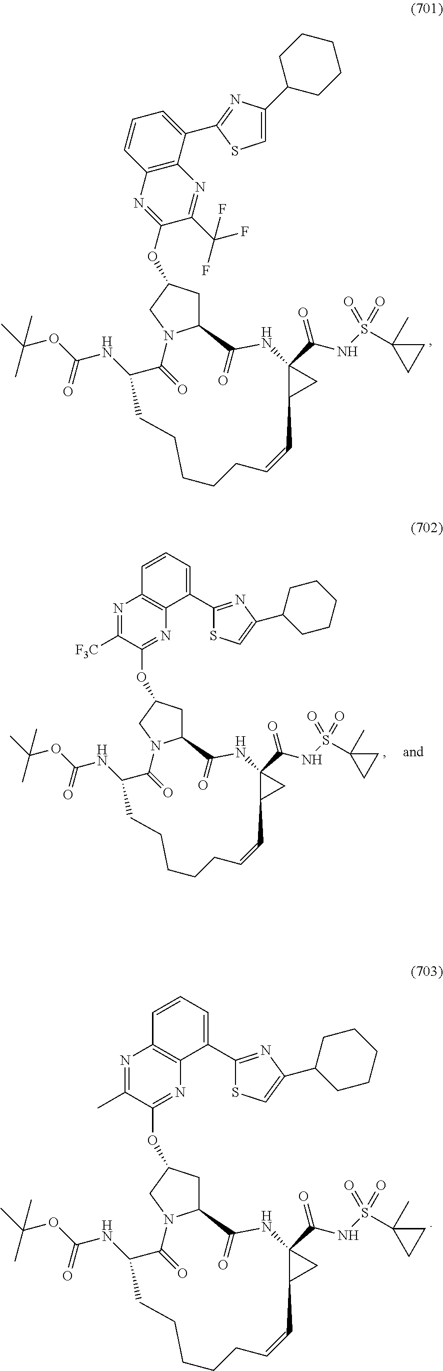

38. A pharmaceutical composition comprising a pharmaceutically acceptable excipient and a compound of claim 1.

39. A method of inhibiting NS3/NS4 protease activity comprising contacting a NS3/NS4 protease with a compound of claim 1.

40. The method of claim 39 in which the contacting is conducted in vivo.

41. The method of claim 40, further comprising identifying a subject suffering from a hepatitis C infection and administering the compound to the subject in an amount effective to treat the infection.

42. The method of claim 41, wherein a sustained viral response is achieved.

43. The method of claim 39, in which the contacting is conducted ex vivo.

44. A method of treating liver fibrosis in an individual, the method comprising administering to the individual an effective amount of a compound of claim 1.

45. A method of increasing liver function in an individual having a hepatitis C virus infection, the method comprising administering to the individual an effective amount of a compound of claims 1.
